(19) United States
(12) Patent Application Publication
(10) Pub. No.: US 2026/0216024 A1
(43) Pub. Date: Jul. 30, 2026

(54) PROCESS FOR TREATING KERATIN FIBRES USING AT LEAST ONE OIL BEARING ACETOACETATE FUNCTIONS

(71) Applicant: L'OREAL, PARIS (FR)

(72) Inventors: Boris STEVANT, AULNAY-SOUS-BOIS (FR); Laurent SABATIE, AULNAY-SOUS-BOIS (FR); Etienne SAVONNET, AULNAY-SOUS-BOIS (FR)

(21) Appl. No.: 19/142,424

(22) PCT Filed: Dec. 26, 2023
(86) PCT No.: PCT/EP2023/087791

§ 371 Date: Jun. 23, 2025

(30) Foreign Application Priority Data
Dec. 27, 2022 (FR) FR2214557

(51) Int. Cl. A61K 8/37; A61K 8/19; A61K 8/31; A61K 8/41; A61K 8/46; A61K 8/60; A61K 8/891; A61K 8/898; A61Q 5/06

(52) U.S. Cl. CPC A61K 8/375; A61K 8/19; A61K 8/31; A61K 8/41; A61K 8/46; A61K 8/60; A61K 8/891; A61K 8/898; A61Q 5/065; A61K 2800/4322

(57) ABSTRACT

The present invention relates to a process for treating keratin fibres, in particular for caring for, styling and/or colouring keratin fibres, preferably the hair, comprising the application to the keratin fibres, in one or more successive steps, of at least i) one or more compounds of formula (I), and also the optical or geometrical isomers thereof and/or the solvates thereof, such as hydrates, or a composition containing same:

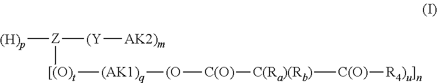

in which formula (I) Z, AK1, AK2, Y, R₄, Rₐ, R_{b}, p, m, n, t, q and u are as defined in the description, and optionally ii) at least one crosslinking agent.

### TECHNICAL FIELD

The present invention relates more particularly to the cosmetic field of keratin fibres, and notably to that of caring for, styling and/or dyeing keratin fibres and preferably the hair.

The aim is thus notably to propose novel treatment processes, notably cosmetic treatment processes, comprising the application to keratin fibres of an oil functionalized with acetoacetate functions, and optionally at least one crosslinking agent, which are most particularly advantageous in terms of their technical performance, notably in terms of the persistence of the deposits they make it possible to obtain, and the sensorial experience they give the user.

### PRIOR ART

Cosmetic products conventionally require the use of one or more film-forming polymers in order to obtain a quality deposit of these products on keratin materials, notably on keratin fibres, and in particular to satisfy the expectations detailed below.

Thus, in the field of haircare, it is most particularly expected that the deposit formed should have good persistence on contact with water, notably rain or during showering or even perspiration, and also with sebum. Moreover, this deposit must be aesthetically pleasing, transparent if not coloured, or even glossy. It must also allow the head of hair to move without changing the appearance of the hair, i.e. without cracking and/or flaking. Deposits that can withstand shampoo washing several times are also sought.

For this purpose, dispersions of polymer particles of nanometric size are used in hair application products, and more particularly in their organic and notably oily phases, as film-forming agent.

However, the compositions of the prior art have unsatisfactory persistence, in particular styling hold, over time.

Moreover, in the field of haircare, a new range of products known as “Hair Makeup” has recently been developed. These products guarantee temporary hair dyeing that lasts after 1 to 3 shampoo washes.

They are thus a particularly attractive alternative for consumers to permanent hair dyeing, provided, of course, that the colouring effect is effectively guaranteed to persist after contact with water and a few shampoo washes.

This requirement is also notably satisfied by the use of effective film-forming agents.

Thus, FR 2 741 530 proposes for this purpose, for the temporary dyeing of keratin fibres, the use of a dispersion of film-forming polymer particles including at least one acidic function and at least one pigment dispersed in the continuous phase of said dispersion. The colourings obtained via this dyeing method nevertheless have the drawback of being removed easily on shampoo washing.

It is moreover proposed in FR 2 907 678 to perform coloured coating of the hair using a composition comprising a polysiloxane/polyurea block copolymer and a pigment. However, with such a composition, the coatings obtained are not always very homogeneous and the individualization of the hair strands is not always very good.

Compositions are known, notably cosmetic, film-forming compositions, comprising at least one fatty phase and at least one copolymer obtained by polymerization of ethylenic monomers, some of which bear particular groups (see, for example, WO 2022/136104). Such compositions nevertheless require the presence of a fatty phase, which may reduce the field to certain applications. Furthermore, these compositions do not necessarily meet the need for naturalness expressed by certain consumers.

It is also known practice from patent EP 1 392 222 to use a cosmetic composition for caring for and/or treating keratin materials, comprising a supramolecular polymer including a polymer backbone and at least two groups that are capable of forming at least three hydrogen bonds, and from patent EP 1 435 900 to use a hair composition comprising a supramolecular polymer including a polymer backbone and at least two groups that are capable of forming at least three hydrogen bonds and a surfactant or a hair-conditioning agent. However, with these two composition alternatives, the obtained performance with respect to styling hold over time or with respect to water persistence remains insufficient.

Processes for preparing compositions comprising modified polysaccharides are also known from WO 2020/026143. Moreover, US 2006/0079599 describes a polymer-based tissue adhesive for medical use, including polyvinyl alcohol polymers bearing ACAC groups combined with polyamines in the form of a hydrogel. U.S. Pat. Nos. 3,342,806 and 3,361,585 describe acetoacetylated starch compounds.

Moreover, WO 06/042169 describes tissue adhesives, in medical and veterinary applications, formed by the reaction of polyhydroxy compounds derived with acetoacetate groups and/or polyamine compounds derived with acetoacetamide groups with an amino-functional crosslinking compound.

The synthesis of acetoacetate diester triglycerides is also described in WO 2016/164870.

In general, the treatment processes described above do not make it possible to obtain deposits on keratin fibres which satisfy all the abovementioned requirements, namely very good water persistence, in particular to shampoo washing for the hair, and/or to fatty substances, which allow the head of hair to move without cracking and/or flaking, which make it possible to adjust the gloss or invisibility qualities and which, in the case of haircare use, provide very satisfactory styling hold or colour persistence.

Moreover, there is considerable consumer demand for compositions with a reduced environmental impact.

### DISCLOSURE OF THE INVENTION

There is thus still a need for a treatment process, notably a cosmetic treatment process, intended for application to the keratin fibres, which makes it possible to obtain a deposit that is non-tacky, which transfers little, if at all, and which is glossy and persistent.

There is also still a need for a treatment process, notably a cosmetic treatment process, which allows deposits to be obtained that are resistant to water and fatty substances, in particular sebum.

There is also still a need for a treatment process, notably a cosmetic treatment process, which affords deposits that have shiny and/or invisibility qualities.

There is also a need for a colouring treatment process, notably a colouring cosmetic treatment process, which is intended for application to the hair and which affords deposits that have good resistance to water and shampoo washing in order to ensure a colour persistence over time that is comparable to direct dyeing.

There is also a need for a treatment process, notably cosmetic, notably a non-colouring treatment process, which is intended for application to the hair and which affords water-resistant styling properties, notably curl hold.

The present invention is specifically directed toward meeting all or some of these needs.

### SUMMARY OF THE INVENTION

These problems are solved by performing a process for treating, notably a cosmetic process for treating, keratin fibres, in particular for caring for, styling and/or colouring keratin fibres, preferably the hair, comprising the application to said keratin fibres, in one or more successive steps, of at least:
i) one or more compounds of formula (I), and also the optical or geometrical isomers thereof, and/or solvates thereof, such as hydrates, or a composition containing same:

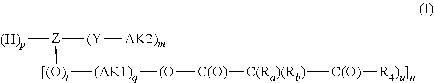

in which formula (I):
Z represents a linear or branched, saturated or unsaturated, conjugated or non-conjugated, acyclic or cyclic, aromatic or non-aromatic multivalent C₂ to C₄₀, in particular C₃ to C₃₆, hydrocarbon-based radical, Z possibly being interrupted with one or more groups or heteroatoms chosen from —O—, —N(Rₐ)—, —S—, —C(O)—, (hetero)cycles, and combinations thereof;
AK1 represents a divalent saturated or unsaturated, linear or branched C₁-C₁₂, preferably C₁-C₆, more preferentially saturated C₁-C₆, hydrocarbon-based chain;
AK2 represents a linear or branched, saturated or unsaturated, C₁-C₄₀ monovalent hydrocarbon-based chain, optionally substituted with one or more identical or different radicals chosen from —OR, —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), and combinations thereof, in particular —O—C(O)—, —C(O)—O— and/or -(hetero)cycle-O-(hetero)cycle-;
AK3 represents a divalent linear or branched, saturated or unsaturated C₁-C₁₂, preferably C₁-C₆, more preferentially saturated C₁-C₆, even more preferentially C₁-C₄, hydrocarbon-based chain;
R represents a hydrogen atom, a linear or branched, saturated or unsaturated C₁-C₆, preferably C₁-C₄, alkyl radical, in particular a methyl radical, or a radical —C(O)—C(Rₐ)(R_{b})—C(O)—R₄;
X represents —O—, —S—, —O—C(O)— or —C(O)—O—;
Y represents —O—C(O)— or —C(O)—O—;
R₄ represents a linear or branched, saturated or unsaturated C₁-C₆ monovalent hydrocarbon-based radical, preferably a (C₁-C₄)alkyl group, in particular methyl or tert-butyl, more preferentially methyl;
Rₐ and R_{b}, which may be identical or different, represent a hydrogen atom or a (C₁-C₄)alkyl group, preferably a hydrogen atom;
p denotes an integer equal to 0 or 1;
m denotes an integer equal to 0, 1, 2, 3 or 4; it being understood that when m is greater than 1, then the radicals —Y-AK₂ are identical or different;
n denotes an integer equal to 0, 1, 2, 3 or 4; it being understood that when n is greater than 1, then the radicals —(O)ₜ-(AK1)_{q}-(O—C(O)—C(Rₐ)(R_{b})—C(O)—R₄)ᵤ are identical or different;
t denotes an integer equal to 0 or 1;
q denotes an integer equal to 0 or 1;
u denotes an integer equal to 1, 2 or 3;
v denotes an integer equal to 1, 2 or 3;
it being understood that if q is equal to 0 then t is equal to 0, that (m+n) is greater than or equal to 1, and that the compounds of formula (I) contain at least two groups —C(O)—C(Rₐ)(R_{b})—C(O)—R₄; and
ii) optionally at least one crosslinking agent.

According to a preferred embodiment, the treatment process of the invention also comprises the application of at least iii) a cosmetic active agent, to said keratin fibres.

According to a preferred embodiment, the process according to the invention comprises the application i) of at least one compound of formula (I) as defined previously, the optical or geometrical isomers thereof, and/or the solvates thereof, such as hydrates, or of a composition containing same, and optionally the application ii) of at least one crosslinking agent, and/or iii) of at least one cosmetic active agent, to said keratin fibres.

When the cosmetic active agent(s) iii) are present, the cosmetic active agent(s) iii) may be applied at the same time as i) and/or as ii) when ii) is used, or even the cosmetic active agent(s) iii) may be applied before i) and/or before ii), when ii) is present.

The keratin fibres may be damp or wet before application of i), and possibly ii). By way of example, steps i), and optionally ii), may be performed on damp keratin fibres, such as fibres just wrung out, for example after a shampoo wash.

According to one embodiment, the process comprises the application of i), optionally ii) and optionally iii) to damp or wet keratin fibres.

According to another embodiment, the process comprises the application of i), optionally ii) and optionally iii) to dry keratin fibres.

The inventors thus found, surprisingly, that the application to keratin fibres of the ingredients i), and optionally ii) and iii), makes it possible to obtain deposits on keratin fibres which have good resistance to external attacking factors, for instance water, oils, notably food oils, sweat and/or sebum, and which are thus endowed with very good staying power over time. Advantageously, the deposits obtained via the processes according to the invention are not tacky and do not transfer. Applying ingredients i), and possibly ii) and/or iii) to keratin fibres also affords deposits on the keratin fibres which show good resistance to shampoo washing and to water.

Applying ingredients i), and possibly ii) and/or iii) to keratin fibres also affords shiny deposits.

### Definitions

For the purposes of the present invention, throughout the description, including the claims, and unless otherwise indicated:
For the purposes of the present invention, the term “keratin fibres” is intended to denote keratin fibres, preferably human keratin fibres, and more preferentially the hair.
The term “cosmetic active agent” means an organic or organosilicon compound or a mineral compound which can be incorporated into a cosmetic composition to give an effect on keratin materials, whether this effect is immediate or provided by repeated applications. As examples of cosmetic active agents, mention may be made of coloured or uncoloured, fluorescent or non-fluorescent compounds such as optical brighteners, or UVA and/or UVB screening agents, active agents intended for providing a benefit to keratin fibres, antioxidant active agents, moisturizing active agents, sebum-regulating active agents, active agents intended for combating the effects of pollution, antimicrobial or bactericidal active agents, fragrances and dyestuffs such as direct dyes or pigments, preferably pigments.

Preferentially, the cosmetic active agents are chosen from a) dyestuffs chosen from pigments, direct dyes, and mixtures thereof, b) active agents for caring for keratin fibres, c) UV-screening agents, and d) mixtures thereof.
For the purposes of the present invention, the term “fatty substance” means an organic compound that is insoluble in water at ordinary temperature (25° C.) and at atmospheric pressure (760 mmHg) (solubility of less than 5%, preferably less than 1% and even more preferentially less than 0.1%); in addition, the fatty substances are soluble in organic solvents under the same temperature and pressure conditions, for instance in halogenated solvents such as chloroform or dichloromethane, lower alcohols such as ethanol or aromatic solvents such as benzene or toluene.
The term “(hetero)aryl” means aryl or heteroaryl groups.
The term “(hetero)cycloalkyl” means cycloalkyl or heterocycloalkyl groups.
The “aryl” or “heteroaryl” radicals or the aryl or heteroaryl part of a radical may be substituted with at least one substituent borne by a carbon atom, chosen from:
   a C₁-C₆ and preferably C₁-C₄ (poly)(hydroxy)alkyl radical;
   a halogen atom such as chlorine, fluorine or bromine;
   a hydroxyl group;
   a C₁-C₂ alkoxy radical; a C₂-C₄ (poly)hydroxyalkoxy radical;
   an amino radical;
   an amino radical substituted with one or two identical or different C₁-C₆, C₁-C₆, and preferably C₁-C₄ alkyl radicals;
   an acylamino radical (—N(R)—C(O)—R′) in which the radical R is a hydrogen atom;
   a C₁-C₄ alkyl radical and the radical R′ is a C₁-C₄ alkyl radical; a carbamoyl radical ((R)₂N—C(O)—) in which the radicals R, which may be identical or different, represent a hydrogen atom or a C₁-C₄ alkyl radical;
   an alkylsulfonylamino radical (R′—S(O)₂—N(R)—) in which the radical R represents a hydrogen atom or a C₁-C₄ alkyl radical and the radical R′ represents a C₁-C₄ alkyl radical, or a phenyl radical;
   an aminosulfonyl radical ((R)₂N—S(O)₂—) in which the radicals R, which may be identical or different, represent a hydrogen atom or a C₁-C₄ alkyl radical;
   a carboxylic radical in acid form or salified (preferably with an alkali metal or a substituted or unsubstituted ammonium) form;
   a cyano group (CN);
   a polyhalo(C₁-C₄)alkyl group, preferentially trifluoromethyl (CF₃).
The cyclic or heterocyclic part of a non-aromatic radical may be substituted with at least one substituent borne by a carbon atom, chosen from the following groups:
   hydroxyl,
   C₁-C₄ alkoxy, C₂-C₄ (poly)hydroxyalkoxy;
   alkylcarbonylamino ((R—C(O)—NR′—), in which the radical R′ is a hydrogen atom or a C₁-C₄ alkyl radical and the radical R is a C₁-C₂ alkyl radical, amino substituted with one or two identical or different C₁-C₄ alkyl groups;
   alkylcarbonyloxy ((R—C(O)—O—), in which the radical R is a C₁-C₄ alkyl radical, amino substituted with one or two identical or different C₁-C₄ alkyl groups;
   alkoxycarbonyl ((R—O—C(O)—) in which the radical R is a C₁-C₄ alkyl radical, amino substituted with one or two identical or different C₁-C₄ alkyl groups;
A cyclic or heterocyclic radical, or a non-aromatic part of an aryl or heteroaryl radical, may also be substituted with one or more oxo groups.
A hydrocarbon-based chain is unsaturated when it includes one or more double bonds and/or one or more triple bonds.
An “aryl” radical represents a monocyclic or fused or non-fused polycyclic hydrocarbon-based group comprising from 6 to 14 carbon atoms, and at least one ring of which is aromatic; preferentially, the aryl radical is a phenyl, biphenyl, naphthyl, indenyl, anthracenyl or tetrahydronaphthyl.
A “heteroaryl” radical represents a monocyclic or fused or non-fused polycyclic, 5- to 14-membered group, comprising from 1 to 6 heteroatoms chosen from nitrogen, oxygen, sulfur and selenium atoms, and at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridyl, tetrazolyl, dihydrothiazolyl, imidazopyridyl, imidazolyl, indolyl, isoquinolyl, naphthoimidazolyl, naphthooxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridyl, thiazoylimidazolyl, thiopyrylyl, triazolyl and xanthylyl.
A “cyclic” or “cycloalkyl” radical is a monocyclic or fused or non-fused polycyclic, non-aromatic cyclic hydrocarbon-based radical containing from 5 to 14 carbon atoms, which may include one or more unsaturations; the cycloalkyl is preferably a cyclohexyl group.
A “heterocyclic” or “heterocycloalkyl” radical is a monocyclic or fused or non-fused polycyclic 3- to 9-membered non-aromatic cyclic radical, including from 1 to 4 heteroatoms chosen from nitrogen, oxygen, sulfur and selenium atoms; preferably, the heterocycloalkyl is chosen from epoxide, piperazinyl, piperidyl, morpholinyl and dithiolane.
An “alky” radical is a linear or branched, in particular C₁-C₆ and preferably C₁-C₄ saturated hydrocarbon-based radical.
An “alkoxy” radical is an alkyl-oxy radical for which the alkyl radical is a linear or branched C₁-C₆ and preferentially C₁-C₄ hydrocarbon-based radical.
A “(poly)(hydroxy)alkyl” radical denotes a C₁-C₆ and preferably C₁-C₄ alkyl radical optionally substituted with one or more hydroxyl radicals, preferably substituted with from 1 to 4 hydroxyl groups, more particularly between 1 and 3.
A “sugar” radical is a monosaccharide or disaccharide radical. Sugar radicals that may be mentioned include: sucrose, glucose, galactose, ribose, fucose, maltose, fructose, mannose, arabinose, xylose, lactose.
The term “monosaccharide” refers to a mono-oside sugar comprising at least 5 carbon atoms of formula Cₓ(H₂O)ₓ with x an integer greater than or equal to 5, preferably x is greater than or equal to 6, in particular x is between 5 and 7 inclusive, preferably x is equal to 6; they may be of D or L configuration, and of alpha or beta anomer, and also the salts thereof and the solvates thereof such as hydrates.
The term “disaccharide” refers to a di-oside sugar which is a compound constituted of two saccharides bonded together via O-oside bonds, said compounds being constituted of two monosaccharide units (also known as mono-osides) as defined previously, said monosaccharide units comprising at least 5 carbon atoms, preferably 6; in particular, the mono-oside units are linked together via a 1,4 or 1,6 bond as a (alpha) or R (beta) anomer, it being possible for each oside unit to be of L or D configuration, and also the salts thereof and the solvates thereof such as the hydrates of said monosaccharides; more particularly, they are polymers formed from two saccharides (or monosaccharides) having the general formula: —[Cₓ(H₂O)_{y})]₂— or —[(CH₂O)ₓ]₂—, with x being an integer greater than or equal to 5, preferably x is greater than or equal to 6, in particular x is between 5 and 7 inclusive, preferably x is equal to 6, and y is an integer representing x−1.
The term “polysaccharide” refers to a polysaccharide sugar which is a polymer constituted of several saccharides bonded together via O-oside bonds, said polymers being constituted of monosaccharide units (also known as mono-osides) as defined previously, said monosaccharide units comprising at least 5 carbon atoms, preferably 6; in particular, the monosaccharide units are linked together via a 1,4 or 1,6 bond as α (alpha) or β (beta) anomer, it being possible for each oside unit to be of L or D configuration, and also the salts thereof and the solvates thereof such as the hydrates of said monosaccharides; more particularly, they are polymers formed from a certain number of saccharides (or monosaccharides) having the general formula: —[Cₓ(H₂O)_{y})]_{w}— or —[(CH₂O)ₓ]_{w}—, where x is an integer greater than or equal to 5, preferably x is greater than or equal to 6, in particular x is between 5 and 7 inclusive and preferably x is equal to 6, and y is an integer which represents x−1, and w is an integer greater than or equal to 2, particularly between 3 and 3000 inclusive, more particularly between 5 and 2500, preferentially between 10 and 2300, particularly between 15 and 1000 inclusive, more particularly between 20 and 500, preferentially between 25 and 200.
An “anionic counterion” is an anion or an anionic group associated with the cationic charge; more particularly, the anionic counterion is chosen from: i) halides such as chloride or bromide; ii) nitrates; iii) sulfonates, including C₁-C₆ alkylsulfonates: Alk-S(O)₂O⁻ such as methanesulfonate or mesylate and ethanesulfonate; iv) arylsulfonates: Ar—S(O)₂O⁻ such as benzenesulfonate and toluenesulfonate or tosylate; v) citrate; vi) succinate; vii) tartrate; viii) lactate; ix) alkyl sulfates: Alk-O—S(O)O⁻ such as methyl sulfate and ethyl sulfate; x) aryl sulfates: Ar—O—S(O)O⁻ such as benzene sulfate and toluene sulfate; xi) alkoxy sulfates: Alk-O—S(O)₂O⁻ such as methoxy sulfate and ethoxy sulfate; xii) aryloxy sulfates: Ar—O—S(O)₂O⁻; xiii) phosphate; xiv) acetate; xv) triflate; and xvi) borates such as tetrafluoroborate.
The “solvates” represent hydrates and also the combination with linear or branched C₁-C₄ alcohols such as ethanol, isopropanol or n-propanol.
The term “UV-A screening agent” means a chromophore derived from a compound which screens out (or absorbs) UV-A ultraviolet rays at a wavelength of between 320 and 400 nm. A distinction may be made between short UV-A screening agents (which absorb rays at a wavelength of between 320 and 340 nm) and long UV-A screening agents (which absorb rays at a wavelength of between 340 and 400 nm).
The term “UV-B screening agent” means a chromophore derived from a compound which screens out (or absorbs) UV-B ultraviolet rays at a wavelength of between 280 and 320 nm.
The term “chromophore” means a radical derived from a colourless or coloured compound that is capable of absorbing UV and/or visible radiation at a wavelength λ_{abs} of between 250 and 800 nm. Preferably, the chromophore is coloured, i.e. it absorbs wavelengths in the visible range, i.e. preferably between 400 and 800 nm. Preferably, the chromophores appear coloured to the eye, particularly between 400 and 700 nm (Ullmann's Encyclopedia, 2005, Wiley-VcH, Verlag “Dyes, General Survey”, § 2.1 Basic Principle of Color);
The term “anhydrous composition” means that said composition contains an amount of less than 5% by weight of water, preferentially less than 3% by weight of water, better still less than 1% by weight of water, relative to the total weight of the composition in question; even more preferentially, the composition under consideration is free of water.
The expression “including a” or “comprising a” should be understood as being synonymous with “including at least one” or “including one or more”, unless otherwise mentioned.

The expressions “between . . . and . . . ”, “comprises from . . . to . . . ”, “formed from . . . to . . . ” and “ranging from . . . to . . . ” should be understood as being inclusive of the limits, unless otherwise specified.
The terms “colouring agent” and “dyestuff” are equivalent.

It is understood that the treatment processes according to the invention, and also the compositions used, are non-therapeutic.

### DETAILED DESCRIPTION

### Treatment Method

The first subject of the invention is a treatment process in one or more steps using, on keratin fibres, preferably the hair:
i) at least one or more compounds of formula (I), as defined previously and hereinbelow, or a composition containing same;
ii) optionally at least one crosslinking agent, in particular as defined hereinbelow; and
iii) optionally at least one cosmetic active agent, in particular as defined hereinbelow.

According to a particular embodiment of the invention, the treatment process according to the invention involves the simultaneous application of the ingredients i), ii) and/or iii).

According to another particular embodiment of the invention, the treatment process according to the invention comprises at least two steps in which the ingredients i), ii) and optionally iii) are applied in separate and successive steps to the keratin fibres, preferably the hair, it being understood that the cosmetic active agent(s) iii) may be present together with the ingredient(s) i) and/or with the ingredient(s) ii).

According to a particular embodiment, the ingredient(s) i) are applied to the keratin fibres, preferably the hair, and the ingredient(s) ii) are then applied to the keratin fibres, preferably the hair, it being understood that ingredient iii), when it is present, may be applied with i) and/or with ii).

According to another particular embodiment, the ingredient(s) ii) are applied to the keratin fibres, preferably the hair, and the ingredient(s) i) are then applied to the keratin fibres, preferably the hair, it being understood that ingredient iii), when it is present, may be applied with i) and/or with ii).

According to another particular embodiment of the invention, the treatment process according to the invention comprises at least two steps in which the ingredients i) and iii) are applied in separate and successive steps to the keratin fibres, preferably the hair, it being understood that the cosmetic active agent(s) iii) may be present together with the ingredient(s) i) and/or with the ingredient(s) ii) when they are present.

According to a particular embodiment of the invention, the process for treating keratin fibres is a cosmetic process for caring for, styling and/or colouring keratin fibres, preferably the hair.

According to one aspect of the invention, the process for treating keratin fibres according to the invention using ingredients i), optionally ii) and optionally iii), is a treatment for colouring keratin fibres, preferably the hair. According to this embodiment, the process in particular comprises at least one cosmetic active agent chosen from dyestuffs, preferably pigments.

According to another aspect of the invention, the process for treating keratin fibres according to the invention using the ingredients i), optionally ii) and optionally iii), is a process for styling keratin fibres, preferably the hair.

When the treatment process involves several steps including a step of using one or more compounds of formula (I), (Ia), (Ib), (Ic), (Id), (Ie), (Iic), (Iid) and/or (Iie) without crosslinking agent, the compound(s) of formula (I), (Ia), (Ib), (Ic), (Id), (Ie), (Iic), (Iid) and/or (Iie) may be used on their own (pure), or in a composition “C1” as described hereinbelow.

According to one embodiment of the invention, the process according to the invention is a process for treating, notably for cosmetically treating, keratin fibres, in particular for caring for, styling and/or colouring keratin fibres, preferably the hair, comprising the application to said keratin fibres of at least:
a composition, termed “C1”, comprising i) at least one compound of formula (I), or (Ia), (Ib), (Ic), (Id) and/or (Ie), as defined previously and hereinbelow, and optionally iii) at least one cosmetic active agent, in particular as defined previously and hereinbelow; preferably, composition “C1” does not comprise ii) a crosslinking agent;
a composition, termed “C2”, comprising i) at least one compound of formula (I), or (Ia), (Ib), (Ic), (Id) and/or (Ie), as defined previously and hereinbelow, ii) at least one crosslinking agent, notably as defined hereinbelow, and optionally iii) at least one cosmetic active agent, in particular as defined previously and hereinbelow; preferably, composition “C2” does not comprise iii) a cosmetic active agent;
a composition, termed “C3”, comprising i) at least one compound of formula (I), or (Ia), (Ib), (Ic), (Id) and/or (Ie), as defined previously and hereinbelow, ii) at least one crosslinking agent, in particular as defined hereinbelow, and iii) at least one cosmetic active agent, in particular as defined previously and hereinbelow;
a composition, termed “C4”, comprising ii) at least one crosslinking agent, in particular as defined hereinbelow, and optionally iii) at least one cosmetic active agent, in particular as defined previously and hereinbelow; preferably, composition “C4” does not comprise i) at least one compound of formula (I), or (Ia), (Ib), (Ic), (Id) and/or (Ie) as defined previously and hereinbelow; and/or
a composition, termed “C5”, comprising iii) at least one cosmetic active agent, in particular as defined previously and hereinbelow; preferably, composition “C5” does not comprise i) at least one compound of formula (I), or (Ia), (Ib), (Ic), (Id) and/or (Ie) as defined previously and hereinbelow, and does not comprise ii) at least one crosslinking agent; it being understood that the process uses i) at least one compound of formula (I), or (Ia), (Ib), (Ic), (Id) and/or (Ie) as defined previously and hereinbelow, and that the compositions “C1”, “C2”, “C3”, “C4” and “C5” may be anhydrous, aqueous such as aqueous-alcoholic and/or comprise one or more fatty substances iv), in particular as described hereinbelow.

According to one embodiment of the invention, the process according to the invention is a process for treating, notably for cosmetically treating, keratin fibres, in particular for caring for, styling and/or colouring keratin fibres, preferably the hair, comprising the application to said keratin fibres of at least:
at least one compound of formula (I), or (Ia), (Ib), (Ic), (Id) and/or (Ie), as defined previously and hereinbelow, and optionally iii) at least one cosmetic active agent, in particular as defined previously and hereinbelow;
a composition, termed “C2”, comprising i) at least one compound of formula (I), or (Ia), (Ib), (Ic), (Id) and/or (Ie), as defined previously and hereinbelow, ii) at least one crosslinking agent, notably as defined hereinbelow, and optionally iii) at least one cosmetic active agent, in particular as defined previously and hereinbelow; preferably, composition “C2” does not comprise iii) a cosmetic active agent;
a composition, termed “C3”, comprising i) at least one compound of formula (I), or (Ia), (Ib), (Ic), (Id) and/or (Ie), as defined previously and hereinbelow, ii) at least one crosslinking agent, in particular as defined hereinbelow, and iii) at least one cosmetic active agent, in particular as defined previously and hereinbelow;
a composition, termed “C4”, comprising ii) at least one crosslinking agent, in particular as defined hereinbelow, and optionally iii) at least one cosmetic active agent, in particular as defined previously and hereinbelow; preferably, composition “C4” does not comprise i) at least one compound of formula (I), or (Ia), (Ib), (Ic), (Id) and/or (Ie) as defined previously and hereinbelow; and/or
a composition, termed “C5”, comprising iii) at least one cosmetic active agent, in particular as defined previously and hereinbelow; preferably, composition “C5” does not comprise i) at least one compound of formula (I), or (Ia), (Ib), (Ic), (Id) and/or (Ie) as defined previously and hereinbelow, and does not comprise ii) at least one crosslinking agent; it being understood that the process uses i) at least one compound of formula (I), or (Ia), (Ib), (Ic), (Id) and/or (Ie) as defined previously and hereinbelow, and that the compositions “C2”, “C3”, “C4” and “C5” may be anhydrous, aqueous such as aqueous-alcoholic and/or comprise one or more fatty substances iv), in particular as described hereinbelow.

According to one aspect of the invention, the process for treating keratin fibres, in particular for caring for, styling and/or colouring keratin fibres, preferably the hair, uses i) at least one compound of formula (I), or (Ia), (Ib), (Ic), (Id), (Ie), (Iic), (Iid) and/or (Iie) as defined previously and hereinbelow or a composition, termed “C1”, comprising same, said composition “C1” optionally comprising iii) at least one cosmetic active agent, in particular as defined previously and hereinbelow, notably chosen from a) colouring agents, such as pigments, direct dyes, and mixtures thereof, b) active agents for caring for keratin fibres, c) UV-screening agents, and d) mixtures thereof, and notably at least one colouring agent, more particularly at least one pigment.

According to another aspect, the process for treating keratin fibres, in particular for caring for, styling and/or colouring keratin fibres, preferably the hair, uses a composition, termed “C2”, comprising i) at least one compound of formula (I), or (Ia), (Ib), (Ic), (Id), (Ie), (Iic), (Iid) and/or (Iie), as defined previously and hereinbelow, ii) at least one crosslinking agent, notably as defined hereinbelow, and optionally iii) at least one cosmetic active agent, in particular as defined previously and hereinbelow, and notably at least one dyestuff, more particularly at least one pigment.

According to one aspect of the invention, the process for treating keratin fibres, in particular for caring for, styling and/or colouring keratin fibres, preferably the hair, uses a composition termed “C3”, comprising i) at least one compound of formula (I), or (Ia), (Ib), (Ic), (Id), (Ie), (Iic), (Iid) and/or (Iie) as defined previously and hereinbelow, ii) at least one crosslinking agent, in particular as defined previously and hereinbelow, and iii) at least one cosmetic active agent, in particular as defined hereinbelow, notably chosen from a) colouring agents, such as pigments, direct dyes, and mixtures thereof, b) active agents for caring for keratin fibres, c) UV-screening agents, and d) mixtures thereof, and notably at least one colouring agent, more particularly at least one pigment.

According to another aspect, the process for treating keratin fibres, in particular for caring for, styling and/or colouring keratin fibres, preferably the hair, uses a composition termed “C1” or i) at least one compound of formula (I) or (Ia), (Ib), (Ic), (Id), (Ie), (Iic), (Iid) and/or (Iie), as defined previously and hereinbelow, and a composition termed “C4”, said composition “C1” comprising i) at least one compound of formula (I) or (Ia), (Ib), (Ic), (Id), (Ie), (Iic), (Iid) and/or (Iie), as defined previously and hereinbelow, and optionally iii) at least one cosmetic active agent, in particular as defined previously and hereinbelow, and said composition “C4” comprising ii) at least one crosslinking agent, in particular as defined hereinbelow, and optionally iii) at least one cosmetic active agent, in particular as defined previously and hereinbelow.

According to another aspect, the process for treating keratin fibres, in particular for caring for, styling and/or colouring keratin fibres, preferably the hair, uses a composition termed “C1”, or i) at least one compound of formula (I) or (Ia), (Ib), (Ic), (Id), (Ie), (Iic), (Iid) and/or (Iie), as defined previously and hereinbelow, and a composition termed “C4” and a composition termed “C5”, said composition “C1” comprising i) at least one compound of formula (I), or (Ia), (Ib), (Ic), (Id), (Ie), (Iic), (Iid) and/or (Iie), as defined previously and hereinbelow, and optionally iii) at least one cosmetic active agent, in particular as defined previously and hereinbelow, said composition “C4” comprising ii) at least one crosslinking agent, in particular as defined hereinbelow, and optionally iii) at least one cosmetic active agent, in particular as defined previously and hereinbelow, and said composition “C5” comprising iii) at least one cosmetic active agent, in particular as defined previously and hereinbelow.

According to another aspect, the process for treating keratin fibres, in particular for caring for, styling and/or colouring keratin fibres, preferably the hair, uses a composition termed “C1” or i) at least one compound of formula (I), or (Ia), (Ib), (Ic), (Id), (Ie), (Iic), (Iid) and/or (Iie), as defined previously and hereinbelow, and a composition termed “C5”, said composition “C1” comprising i) at least one compound of formula (I), or (Ia), (Ib), (Ic), (Id), (Ie), (Iic), (Iid) and/or (Iie), as defined previously and hereinbelow, and optionally iii) at least one cosmetic active agent, in particular as defined previously and hereinbelow, and said composition “C5” comprising iii) at least one cosmetic active agent, in particular as defined previously and hereinbelow.

According to another aspect, the process for treating keratin fibres, in particular for caring for, styling and/or colouring keratin fibres, preferably the hair, uses a composition termed “C2” and a composition termed “C5”, said composition “C2” comprising i) at least one compound of formula (I), or (Ia), (Ib), (Ic), (Id), (Ie), (Iic), (Iid) and/or (Iie), as defined previously and hereinbelow, ii) at least one crosslinking agent, in particular as defined hereinbelow, and optionally iii) at least one cosmetic active agent, in particular as defined previously and hereinbelow, and said composition “C5” comprising iii) at least one cosmetic active agent, in particular as defined previously and hereinbelow.

According to a variant of the process of the invention, the ingredient(s) i) and optionally iii) are applied together, i.e. simultaneously, to the keratin fibres, preferably the hair, during a first step, and then, during a subsequent step, the ingredient(s) ii) and/or iii) are applied to the keratin fibres, preferably the hair.

According to a particular embodiment, the process according to the invention comprises two successive steps in which two different compositions are applied to said keratin fibres.

According to a particular embodiment of the process, during the first step a composition “C1” or i) at least one compound of formula (I), or (Ia), (Ib), (Ic), (Id), (Ie), (Iic), (Iid) and/or (Iie) as defined previously and hereinbelow is applied to the keratin fibres, and a composition “C4” is then applied to said keratin fibres.

According to yet another variant of the process of the invention, ingredient(s) ii) and optionally iii) are applied to the keratin fibres, and then ingredient(s) i), and optionally iii) at least one cosmetic active agent, in particular as defined previously, are applied to the keratin fibres. In particular, composition “C4” is applied to the keratin fibres, and composition “C1” or i) at least one compound of formula (I), or (Ia), (Ib), (Ic), (Id), (Ie), (Iic), (Iid) and/or (Iie) as defined previously and hereinbelow is then applied.

According to another variant of the process according to the invention, i) at least one compound of formula (I), or (Ia), (Ib), (Ic), (Id), (Ie), (Iic), (Iid) and/or (Iie) as defined previously and hereinbelow or a composition “C1” containing same and optionally containing iii) is applied, and sequentially a composition “C4” containing ii) and optionally iii) is applied, preferably i) or composition “C1” being applied before composition “C4”.

According to another variant of the process according to the invention, a composition “C2” containing i), ii) at least one crosslinking agent, and optionally iii) at least one cosmetic active agent, is applied, and sequentially a composition “C4” containing ii) at least one crosslinking agent, optionally iii) at least one cosmetic active agent, is applied, composition “C2” preferably being applied before composition “C4”, and it being understood that the optional crosslinking agent(s) contained in composition “C2” may be identical to or different from the crosslinking agent(s) contained in composition “C4”; preferably, the crosslinking agent(s) are different.

Compositions “C1”, “C2”, “C3”, “C4” and/or “C5” of the process according to the invention, which comprise at least one fatty substance iv), notably at least one oil, may be in the form of a direct or inverse emulsion.

Compositions “C1”, “C2”, “C3”, “C4” and/or “C5” of the process according to the invention may thus be applied directly as such to the keratin fibres or may even be formed directly on the surface of these keratin fibres.

According to the invention, three application methods known as the “one-gesture application mode”, the “two-gesture application mode” and the “three-gesture application mode” are thus distinguished.

According to one embodiment of the process of the invention, the process is performed in one gesture by applying composition “C1” or “C2” or “C3”, as defined previously, to the keratin fibres.

Thus, according to one embodiment, the treatment process according to the invention comprises a single step of applying i) at least one compound of formula (I), or (Ia), (Ib), (Ic), (Id), (Ie), (Iic), (Iid) and/or (Iie) as defined previously and hereinbelow, or of composition “C1”, or of composition “C2” or of composition “C3”, to the keratin fibres.

The term “one-gesture application mode” means the direct application to the target keratin fibres of a single composition in accordance with the invention, namely composition “C1” or composition “C2” or composition “C3”.

The term “one-gesture application mode” also means the single application of i) at least one compound of formula (I), or (Ia), (Ib), (Ic), (Id), (Ie), (Iic), (Iid) and/or (Iie) as defined previously and hereinbelow.

After application of composition “C1”, or of i) at least one compound of formula (I), or (Ia), (Ib), (Ic), (Id), (Ie), (Iic), (Iid) and/or (Iie) as defined previously and hereinbelow, or of composition “C2”, or of composition “C3”, a persistent, non-tacky deposit is advantageously obtained. The deposit obtained is also resistant to food oils, water, shampoo washing, sebum and/or friction.

According to another embodiment of the process of the invention, the process is performed in two gestures.

The term “two-gesture application mode” means the successive application, to keratin fibres, of two different compositions, for example “C1” and “C4”, or “C2” and “C4”, or “C3” and “C4”, preferably “C1” and then “C4”, “C2” and then “C4”, or “C3” and then “C4”.

The term “two-gesture application mode” also means the sequential application, to keratin fibres, preferably the hair, of i) at least one compound of formula (I), or (Ia), (Ib), (Ic), (Id), (Ie), (Iic), (Iid) and/or (Iie) as defined previously and hereinbelow, and of a composition “C4”, preferably i) and then “C4”.

According to another embodiment, the process of the invention is performed in three gestures.

The term “three-gesture application mode” means the sequential application of three different compositions “C1” to “C5”. According to this application mode, for example, according to one embodiment, successive application is performed on the keratin fibres, a) of a composition “C1”, then 3) of a composition “C4”, then y) of a composition “C5”, preferably “C1” then “C4” or, respectively, “C5” or “C5” or, respectively, “C4”.

According to another embodiment, sequential application is performed on the keratin fibres, a) of a composition, for example “C4”, and 3) of a composition “C1” or of a composition “C2”, and also y) of a composition “C5”.

The term “three-gesture application mode” also means the sequential application of i) at least one compound of formula (I), or (Ia), (Ib), (Ic), (Id), (Ie), (Iic), (Iid) and/or (Iie) as defined previously and hereinbelow, of a composition “C4” and of a composition “C5”.

In the two- or three-gesture application modes, the composition applied first, for example “C1”, or i), or “C2”, is conventionally referred to as the “base coat”, and the composition(s) superposed thereon, for example “C4”, are generally referred to as the “top coat”.

After application of the various compositions “C1”, or i), and “C2” to “C5”, a persistent, non-tacky deposit is advantageously obtained. The deposit obtained is also resistant to food oils, water and shampoo washing.

According to a particular embodiment, the compositions “C1”, or i), and “C2” to “C5” are applied to dry keratin fibres.

According to a particular embodiment, the compositions “C1”, or i), and “C2” to “C5” are applied to damp or wet keratin fibres, i.e. keratin fibres containing water on the surface.

According to a particular embodiment, the keratin fibres are dried after application of compositions “C1”, or i), and “C2” to “C5”, in particular after application of each different composition. The drying step can be implemented with a drying device such as a helmet, a hair dryer or a climazon. When the drying step is implemented with a helmet or a hair dryer, the drying temperature is higher than 40° C. After the drying step, a step of shaping the keratin fibers can be implemented with a hair straightener (“fer à lisser”), a straightening or curling iron or a steam iron, preferably a hair straightener or a steam iron. Preferably, the step of shaping the keratin fibres is carried out at a temperature higher than 100° C. The iron can be applied to the keratin fibers in successive separate touches of a few seconds, or by progressive movement or sliding along the hair. Preferably, the application of the iron is done in continuous movement from the root to the tip of the hair, in one or more passes.

According to one aspect, the present invention relates to a process, notably a cosmetic process, for keratin fibres, in particular for caring for, styling and/or colouring keratin fibres, preferably the hair, comprising a step of applying to the keratin fibres i) at least one compound of formula (I), or (Ia), (Ib), (Ic), (Id), (Ie), (Iic), (Iid) and/or (Iie) as defined previously or of a composition “C1” containing same, of a composition “C2” or of a composition “C3”, notably containing at least one dyestuff, in particular as defined previously, and more particularly at least one pigment.

According to another of its aspects, the present invention relates to a process for the cosmetic treatment of keratin fibres, in particular for caring for, styling and/or colouring keratin fibres, preferably the hair, comprising the successive application to said keratin fibres of at least:
i) at least one compound of formula (I), or (Ia), (Ib), (Ic), (Id), (Ie), (Iic), (Iid) and/or (Iie), as defined previously and hereinbelow, or of a composition termed “C1” containing same and optionally comprising iii) at least one cosmetic active agent, in particular as defined previously and hereinbelow; and then
a composition, termed “C4”, comprising ii) at least one crosslinking agent, in particular as defined hereinbelow, and optionally iii) at least one cosmetic active agent, in particular as defined previously and hereinbelow;
at least one of the compositions “C1” and/or “C4” containing at least one dyestuff, in particular as defined previously and hereinbelow, preferably at least one pigment, and more preferentially composition “C1” comprises at least one pigment.

According to another of its aspects, the present invention relates to a cosmetic process for treating keratin fibres, in particular for caring for, styling and/or colouring keratin fibres, preferably the hair, comprising the successive application of at least:
i) at least one compound of formula (I), or (Ia), (Ib), (Ic), (Id), (Ie), (Iic), (Iid) and/or (Iie), as defined previously and hereinbelow, or a composition “C1” or “C2” or “C3” as defined previously; and/or
a composition “C4” as defined previously;
   it being understood that the process uses all or separately i) at least one compound of formula (I), or (Ia), (Ib), (Ic), (Id), (Ie), (Iic), (Iid) and/or (Iie), as defined previously and hereinbelow, and that the compositions “C1”, “C2”, “C3” and/or “C4” may be anhydrous, aqueous such as aqueous-alcoholic and/or comprise one or more fatty substances iv), in particular as described hereinbelow.

According to another of its aspects, the present invention relates to a cosmetic process for treating keratin fibres, in particular for caring for, styling and/or colouring keratin fibres, preferably the hair, comprising the sequential application of at least:
i) at least one compound of formula (I), or (Ia), (Ib), (Ic), (Id), (Ie), (Iic), (Iid) and/or (Iie), as defined previously and hereinbelow, or a composition “C1” as defined previously; and then
a composition “C4” as defined previously; and then
a composition “C5” as defined previously;
   it being understood that compositions “C1” and/or “C4” and/or “C5” contain at least one dyestuff in particular as defined hereinbelow, preferably at least one pigment. Preferably, composition “C5” comprises at least one pigment.

According to another of its aspects, the present invention relates to a cosmetic process for treating keratin fibres, in particular for caring for, styling and/or colouring keratin fibres, preferably the hair, comprising the successive application of at least:
a composition “C2” or “C3” as defined previously; and
a composition “C4” as defined previously;
   it being understood that compositions “C2” or “C3” contain at least iv) one dyestuff, in particular as defined hereinbelow, and/or composition “C4” contains at least one dyestuff, in particular as defined hereinbelow; preferably composition “C2” or “C3” comprises at least one pigment.
   Compounds i): Oils Functionalized with Acetoacetate Functions

As indicated above, the treatment process according to the invention involves applying to keratin fibres, preferably the hair, at least one or more compounds of formula (I), or a composition containing same.

Thus, the treatment process according to the invention comprises the application to keratin fibres, preferably the hair, of at least one or more compounds of formula (I) and also the optical and geometrical isomers thereof, and/or the solvates thereof, such as the hydrates, or a composition containing same:

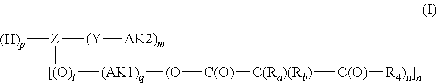

in which formula (I):
Z represents a linear or branched, saturated or unsaturated, conjugated or non-conjugated, acyclic or cyclic, aromatic or non-aromatic multivalent C₂ to C₄₀, in particular C₃ to C₃₆, hydrocarbon-based radical, Z possibly being interrupted with one or more groups or heteroatoms chosen from —O—, —N(Rₐ)—, —S—, —C(O)—, (hetero)cycle(s), and/or combinations thereof;
AK1 represents a divalent saturated or unsaturated, linear or branched C₁-C₁₂, preferably C₁-C₆, more preferentially saturated C₁-C₆, hydrocarbon-based chain;
AK2 represents a linear or branched, saturated or unsaturated, C₁-C₄₀ monovalent hydrocarbon-based chain, optionally substituted with one or more identical or different radicals chosen from —OR, —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), and combinations thereof, in particular O—(CO)—, —(CO)—O— or -(hetero)cycle-O-(hetero)cycle-;
AK3 represents a divalent linear or branched, saturated or unsaturated C₁-C₁₂, preferably C₁-C₆, more preferentially saturated C₁-C₆, even more preferentially C₁-C₄, hydrocarbon-based chain;
R represents a hydrogen atom, a linear or branched, saturated or unsaturated C₁-C₆, preferably C₁-C₄, alkyl radical, in particular a methyl radical, or a radical —C(O)—C(Rₐ)(R_{b})—C(O)—R₄;
X represents —O—, —S—, —O—C(O)— or —C(O)—O—;
Y represents —O—C(O)— or —C(O)—O—;
R₄ represents a linear or branched, saturated or unsaturated C₁-C₆ monovalent hydrocarbon-based radical, preferably a (C₁-C₄)alkyl group, in particular methyl or tert-butyl, more preferentially methyl;
Rₐ and R_{b}, which may be identical or different, represent a hydrogen atom or a (C₁-C₄)alkyl group, preferably a hydrogen atom;
p denotes an integer equal to 0 or 1;
m denotes an integer equal to 0, 1, 2, 3 or 4; it being understood that when m is greater than 1, then the radicals —Y-AK₂ are identical or different;
n denotes an integer equal to 0, 1, 2, 3 or 4; it being understood that when n is greater than 1, then the radicals —(O)ₜ-(AK₁)_{q}-(O—C(O)—C(Rₐ)(R_{b})—C(O)—R₄)ᵤ are identical or different;
t denotes an integer equal to 0 or 1;
q denotes an integer equal to 0 or 1;
u denotes an integer equal to 1, 2 or 3;
v denotes an integer equal to 1, 2 or 3;
   it being understood that if q is equal to 0 then t is equal to 0, that (m+n) is greater than or equal to 1, and that the compounds of formula (I) contain at least two groups —C(O)—C(Rₐ)(R_{b})—C(O)—R₄.

According to a preferred embodiment, the compound(s) of formula (I), and also the optical or geometrical isomers thereof and/or the solvates thereof, such as hydrates, are such that Z denotes a radical chosen from:
a) divalent radicals, preferably C₂-C₃₀ radicals, optionally interrupted with one or more heteroatoms or groups chosen from —O—, —N(Rₐ)—, —S—, —C(O)— and/or (hetero)cycle(s), in particular radicals —CH₂—(CH₂)_{f}, in which f denotes an integer between 1 and 10, preferably between 1 and 5, or such as the radicals —(CH₂)_{g}-(heterocycle)-O—(CH₂)ₕ-(heterocycle)-(CH₂)ⱼ—, in which h denotes 0, 1 or 2, preferably 0 or 1, and g and j, independently of each other, denote an integer between 1 and 3, preferably 1;
b) trivalent radicals, preferably C₃-C₆, in particular CH₂—CH—CH₂—; and
c) tetravalent radicals, preferably C₃-C₁₂, in particular —CH₂—C—CH₂—CH₂—CH₂— or —CH₂—C—CH₂— or the radicals —(CH₂)ₐ—CH—CH—(CH₂)_{b}—, in which a and b independently denote an integer equal to 1, 2, 3, 4, 5, 6, 7, 8, 9 or 10; preferably, a is greater than b.

According to a preferred embodiment, a (hetero)cycle denotes a heterocyclic divalent radical (i.e. a heterocycle), preferably a heterocycloalkyl radical, more particularly a divalent 3-, 5- or 6-membered heterocycloalkyl radical such as a divalent epoxide radical, a divalent tetrahydrofuran radical or a divalent tetrahydropyran radical, said (hetero)cycle being optionally substituted with one or more radicals chosen from —OR with R as defined previously, and in particular —OH or —O—C(O)—C(Rₐ)(R_{b})—C(O)—R₄.

According to a preferred embodiment, AK2 represents a linear or branched, saturated or unsaturated, C₁₂-C₂₈ monovalent hydrocarbon-based chain, optionally substituted with one or more identical or different radicals chosen from —OR, —X-AK3-(O—C(O)—C(Rₐ)(R_{b})—C(O)—R₄), and/or said chain being optionally interrupted with —O—C(O)— or —C(O)—O—.

According to a particular embodiment, the compound(s) of formula (I), or (Ia), (Ib), (Ic), (Id) and (Ie), or alternatively (Iic), (Iid) or (Iie), as defined hereinabove and hereinbelow, are present in a composition in a content ranging from 0.1% to 99% by weight, preferably from 1% to 98% by weight, and more preferably from 5% to 96% by weight, relative to the total weight of the composition under consideration.

According to another particular embodiment, when the treatment process according to the invention comprises several steps, at least one of which does not use a crosslinking agent ii) or a cosmetic active agent iii), the compound(s) of formula (I), or (Ia), (Ib), (Ic), (Id), (Ie), (Iic), (Iid) or (Iie), as defined hereinabove and hereinbelow, may be used alone (pure) in this step or these steps.

According to a first embodiment variant, the compound(s) of formula (I), and also the optical or geometrical isomers and/or solvates thereof, such as hydrates, are such that:
Z represents the trivalent radical —CH₂—CH—CH₂—;
p is equal to 0;
n is equal to 0;
m is equal to 3;
AK2 represents a linear or branched, saturated or unsaturated, C₁₂-C₂₄ monovalent hydrocarbon-based chain, optionally substituted with one or more identical or different radicals chosen from —OR, —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, epoxides, and combinations thereof, in particular O—(CO)— or (CO)—O—;
Y, AK3, v, R, X, R₄, Rₐ and R_{b} being as defined previously for formula (I); it being understood that the compounds contain at least two radicals —C(O)—C(Rₐ)(R_{b})—C(O)—R₄.

According to a preferred embodiment, AK2 represents a linear or branched, saturated or unsaturated, C₁₂-C₂₄ monovalent hydrocarbon-based chain, optionally substituted with one or more identical or different radicals chosen from —OR, —X-AK₃-(O—C(O)—C(Rₐ)(R_{b})—C(O)—R₄)ᵥ, and/or said chain being optionally interrupted with one or more heteroatoms or groups chosen from —O—, —S—, —C(O)—, and combinations thereof, in particular —O—C(O)— or —C(O)—O—.

According to this embodiment variant, the compound(s) of formula (I) are in particular chosen from compounds (1), (2), (3), (4), (7), (8), (9), (11) and (16) hereinbelow, and also the optical or geometrical isomers and/or solvates thereof, such as hydrates:

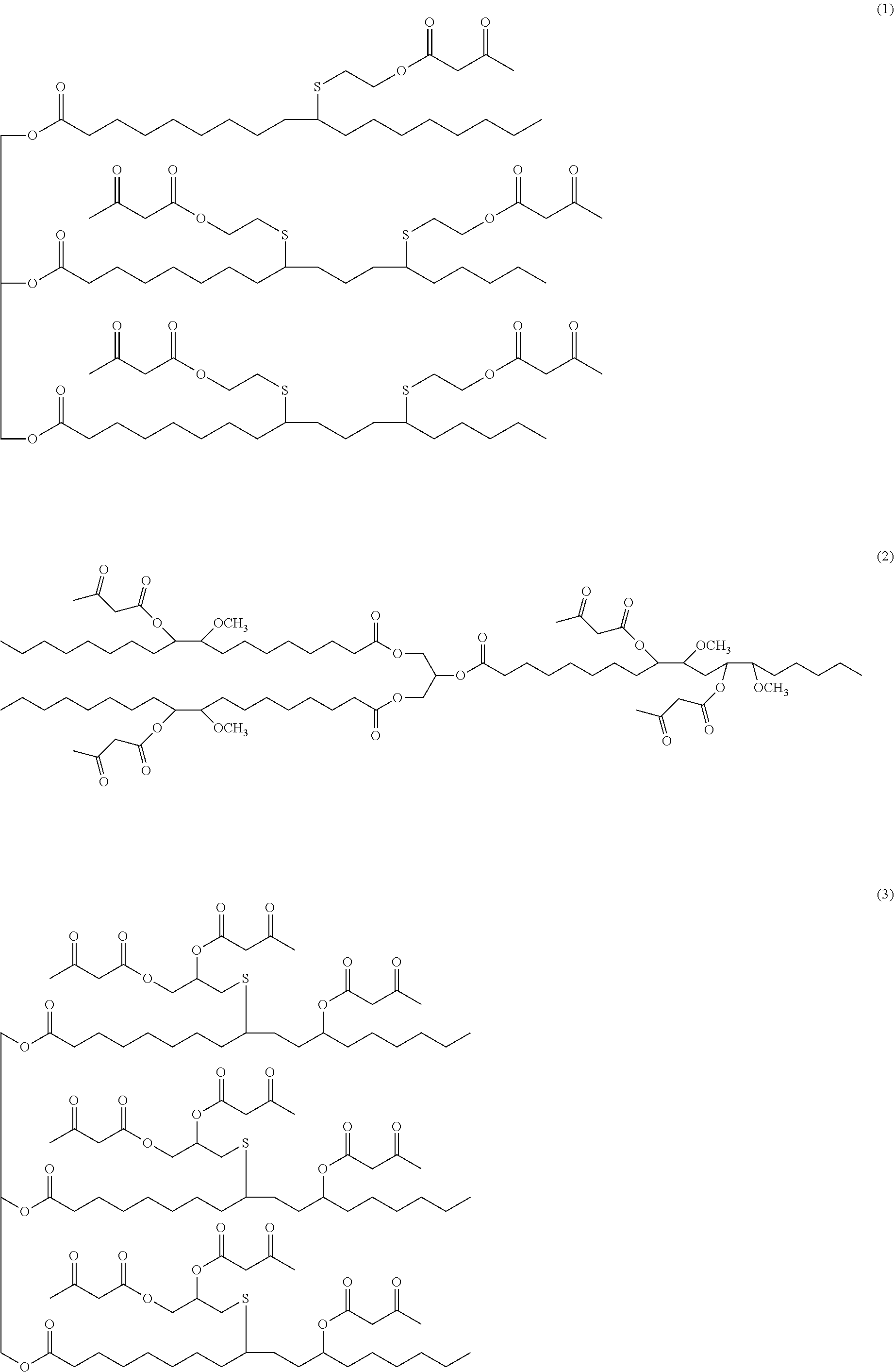

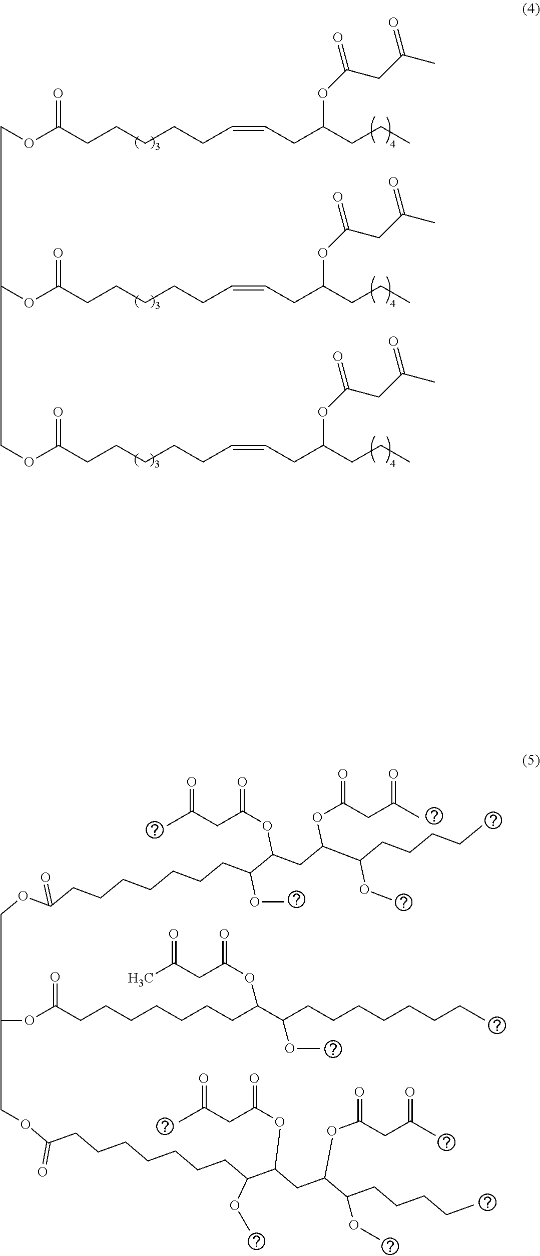

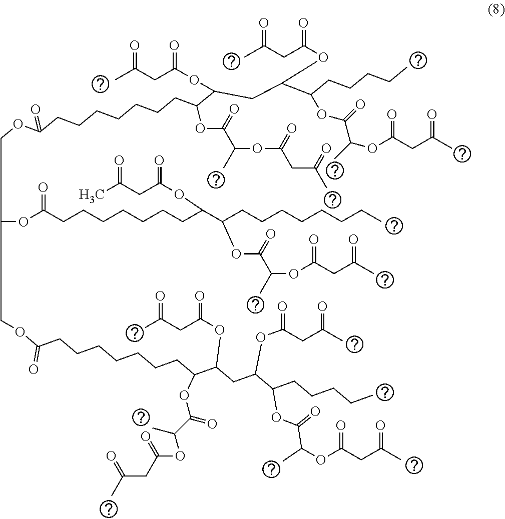 [SMILES: CCCCCCCCC(OC(=O)C(C)OC(=O)CC(C)=O)C(CCCCCCCC(=O)OC(COC(=O)CCCCCCCC(OC(=O)C(C)OC(=O)CC(C)=O)C(CC(OC(=O)CC(C)=O)C(CCCCC)OC(=O)C(C)OC(=O)CC(C)=O)OC(=O)CC(C)=O)COC(=O)CCCCCCCC(OC(=O)C(C)OC(=O)CC(C)=O)C(CC(OC(=O)CC(C)=O)C(CCCCC)OC(=O)C(C)OC(=O)CC(C)=O)OC(=O)CC(C)=O)OC(=O)CC(C)=O]

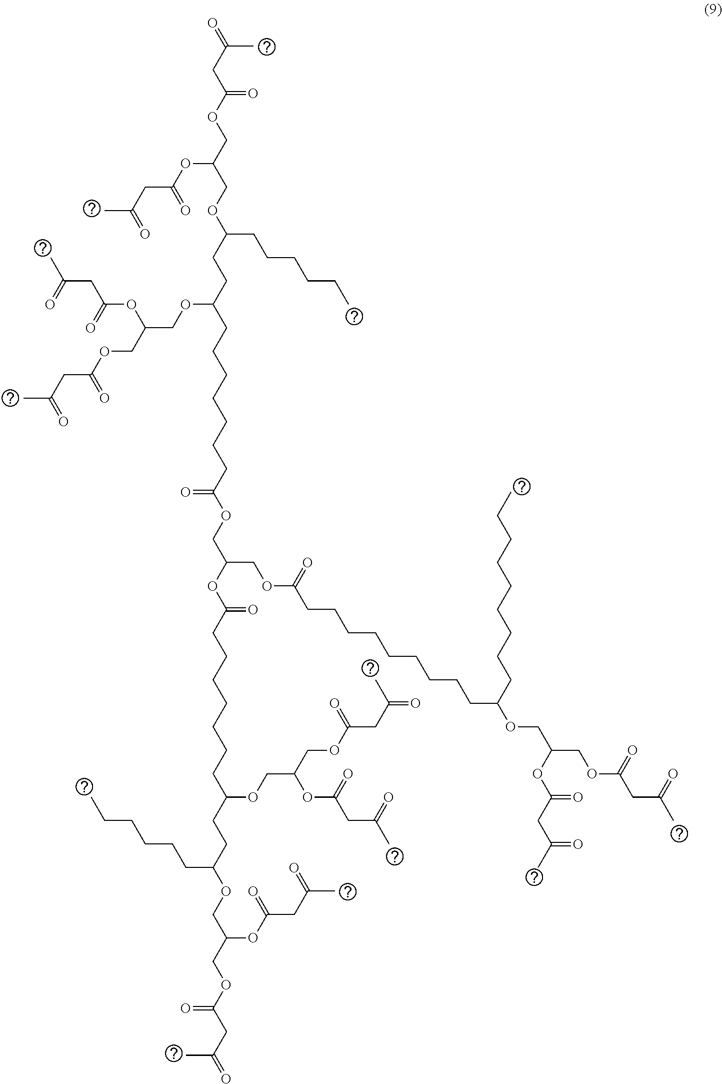 [SMILES: CCCCCCCCCC(CCCCCCCCCC(=O)OCC(COC(=O)CCCCCCCC(CCC(CCCCCC)OCC(COC(=O)CC(C)=O)OC(=O)CC(C)=O)OCC(COC(=O)CC(C)=O)OC(=O)CC(C)=O)OC(=O)CCCCCCCC(CCC(CCCCCC)OCC(COC(=O)CC(C)=O)OC(=O)CC(C)=O)OCC(COC(=O)CC(C)=O)OC(=O)CC(C)=O)OCC(COC(=O)CC(C)=O)OC(=O)CC(C)=O]

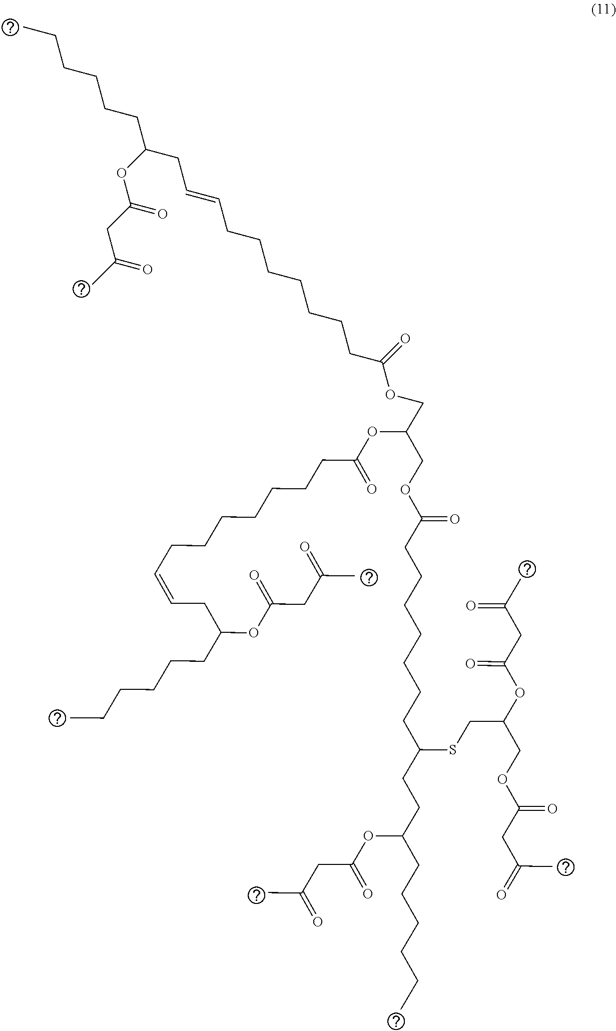 [SMILES: CCCCCCC(C/C=C\CCCCCCCC(=O)OC(COC(=O)CCCCCCC/C=C/CC(CCCCCC)OC(=O)CC(C)=O)COC(=O)CCCCCCCC(CCC(CCCCCC)OC(=O)CC(C)=O)SCC(COC(=O)CC(C)=O)OC(=O)CC(C)=O)OC(=O)CC(C)=O]

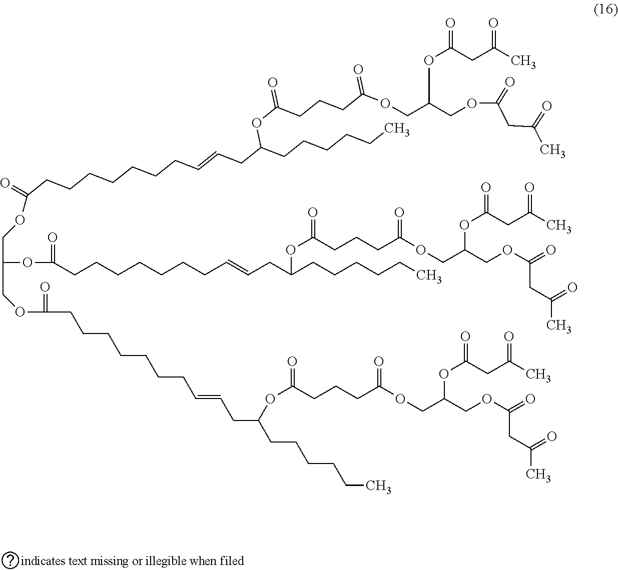 [SMILES: CCCCCCC(C/C=C/CCCCCCCC(=O)OCC(COC(=O)CCCCCCC/C=C/CC(CCCCCC)OC(=O)CCCC(=O)OCC(COC(=O)CC(C)=O)OC(=O)CC(C)=O)OC(=O)CCCCCCC/C=C/CC(CCCCCC)OC(=O)CCCC(=O)OCC(COC(=O)CC(C)=O)OC(=O)CC(C)=O)OC(=O)CCCC(=O)OCC(COC(=O)CC(C)=O)OC(=O)CC(C)=O]

According to a second embodiment variant, the compound(s) of formula (I), and also the optical or geometrical isomers and/or solvates thereof, such as hydrates, are such that:
p is equal to 0;
Z represents a linear or branched, saturated or unsaturated, preferably saturated, acyclic, multivalent C₄ to C₂₄, particularly C₆ to C₂₀, more particularly C₁₄ to C₂₀, in particular C₁₇, hydrocarbon-based radical, Z also being optionally substituted with one or more radicals —[(O)ₜ-(AK1)_{q}—(O—C(O)—C(Rₐ)(R_{b})—C(O)—R₄)ᵤ]ₙ;
n denotes an integer equal to 0, 1, 2, 3 or 4; it being understood that when n is greater than 1, then the radicals —(O)ₜ-(AK₁)_{q}—(O—C(O)—C(Rₐ)(R_{b})—C(O)—R₄)ᵤ are identical or different;
preferably, n is non-zero;
m is equal to 1;
AK2 represents a linear or branched, saturated or unsaturated, preferably saturated, C₃-C₈, in particular C₄ or C₆, monovalent hydrocarbon-based chain, substituted with one or more identical or different radicals chosen from —OR, —X-AK₃-(O—C(O)—C(Rₐ)(R_{b})—C(O)—R₄)ᵥ, preferably substituted with one or more radicals —OR; and/or said chain being optionally interrupted with one or more heteroatoms or groups chosen from —O—, —S—, —C(O)—, (hetero)cycle(s), and combinations thereof, in particular —O—C(O)—, —C(O)—O—, -(hetero)cycle-O-(hetero)cycle-, and preferably not being interrupted;
Y, AK1, AK3, t, q, u, v, R, X, R₄, Rₐ and R_{b} being as defined previously for formula (I);
   it being understood that said compound contains at least two radicals —C(O)—C(Rₐ)(R_{b})—C(O)—R₄.

According to this embodiment variant, the compound(s) of formula (I) are in particular chosen from compounds (5) and (6) hereinbelow, and also the optical or geometrical isomers and/or solvates thereof, such as hydrates:

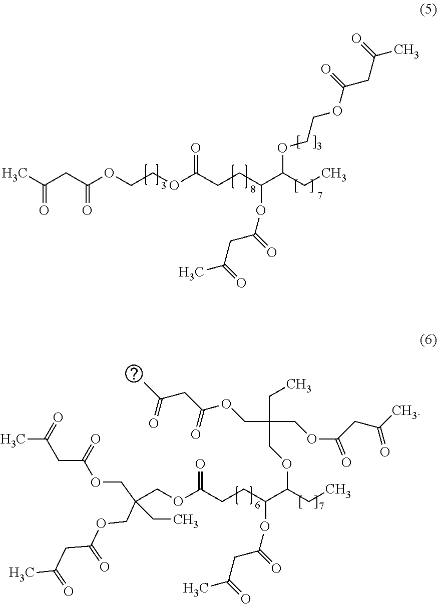

According to a third embodiment variant, the compound(s) of formula (I), and also the optical or geometrical isomers and/or solvates thereof, such as hydrates, are such that:
p is equal to 0;
n+m is equal to 4, m being non-zero;
Z represents an unsubstituted and uninterrupted, linear or branched, saturated or unsaturated, preferably saturated and acyclic, C₂ to C₂₄, particularly C₃ to C₁₀, in particular C₃, tetravalent hydrocarbon-based radical;
AK2 independently represent a linear or branched, saturated or unsaturated, preferably saturated, C₁₂-C₂₄, preferably C₁₆-C₂₀, in particular C₁₈, monovalent hydrocarbon-based chain, optionally substituted with one or more identical or different radicals chosen from —OR and —X-AK3-(O—C(O)—C(Rₐ)(R_{b})—C(O)—R₄)ᵥ, preferably substituted with one or more radicals —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), and combinations thereof, in particular —O—C(O)—, —C(O)—O—, -(hetero)cycle-O-(hetero)cycle-, and preferably not being interrupted;
AK3 represents a linear or branched, saturated or unsaturated C₁-C₁₂, preferably C₁-C₆, more preferentially saturated C₁-C₆, even more preferentially C₂-C₄, better still C₃, divalent hydrocarbon-based chain;
v is as defined previously for formula (I); preferably v is equal to 2;
X is as defined previously for formula (I); preferably, X denotes a sulfur atom;
Y, AK1, u, t, q, R, R₄, Rₐ and R_{b} being as defined previously for formula (I);
   it being understood that said compound contains at least two radicals —C(O)—C(Rₐ)(R_{b})—C(O)—R₄.

According to a preferred embodiment, m is equal to 3, n is equal to 1 and u is equal to 1.

According to a preferred embodiment, t is equal to 0.

According to a preferred embodiment, q is equal to 0.

According to a preferred embodiment, Y denotes —C(O)—O.

According to a preferred embodiment, none of the radicals AK2 are interrupted.

According to a preferred embodiment, the radicals AK2 are saturated.

According to a preferred embodiment, the radicals AK2 are not substituted with —OR.

According to a preferred embodiment, AK3 represents a divalent linear or branched, saturated or unsaturated C₁-C₆, more preferentially saturated C₁-C₆, even more preferentially saturated C₁-C₄, better saturated C₃ hydrocarbon-based chain.

According to this embodiment variant, the compound(s) of formula (I) are in particular chosen from the compounds (10) hereinbelow, and also the optical or geometrical isomers thereof and/or the solvates thereof, such as hydrates:

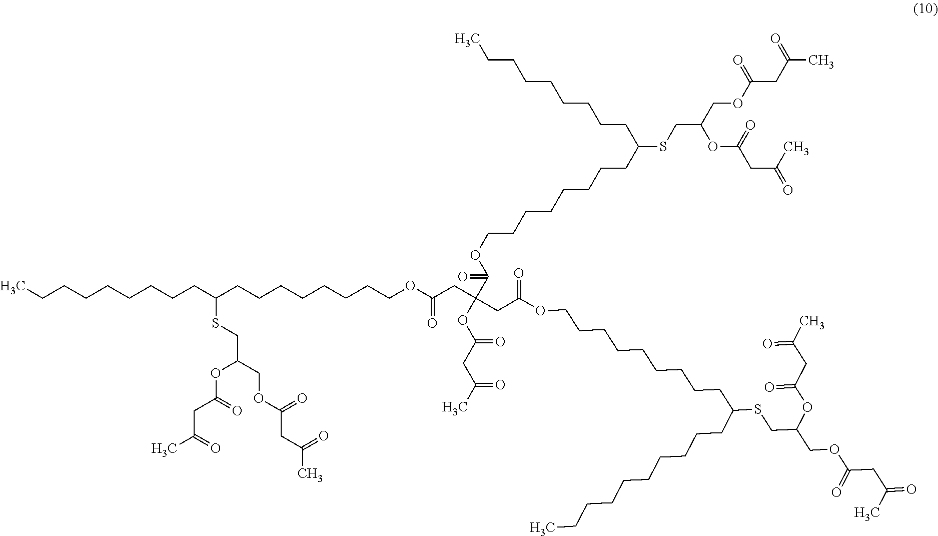 [SMILES: CCCCCCCCCCC(CCCCCCCCCOC(=O)CC(CC(=O)OCCCCCCCCC(CCCCCCCCC)SCC(COC(=O)CC(C)=O)OC(=O)CC(C)=O)(OC(=O)CC(C)=O)C(=O)OCCCCCCCCC(CCCCCCCCC)SCC(COC(=O)CC(C)=O)OC(=O)CC(C)=O)SCC(COC(=O)CC(C)=O)OC(=O)CC(C)=O]

According to a fourth embodiment variant, the compound(s) of formula (I), and also the optical or geometrical isomers and/or solvates thereof, such as hydrates, are such that:
p is equal to 1;
Z represents a linear or branched, saturated or unsaturated, preferably saturated and acyclic, multivalent C₁₄ to C₂₄, particularly C₁₆ to C₂₀, more particularly C₁₇, hydrocarbon-based radical, Z also being optionally substituted with one or more radicals —[(O)ₜ-(AK1)_{q}—(O—C(O)—C(Rₐ)(R_{b})—C(O)—R₄)ᵤ]ₙ, preferably being unsubstituted;
AK2 represents a linear or branched, saturated or unsaturated, preferably saturated, C₁₄-C₃₀, in particular C₂₀-C₂₉, monovalent hydrocarbon-based chain, substituted with one or more identical or different radicals chosen from —OR, —X-AK₃-(O—C(O)—C(Rₐ)(R_{b})—C(O)—R₄)ᵥ, preferably substituted with one or more radicals —OR; and/or said chain being optionally interrupted with one or more heteroatoms or groups chosen from —O—, —S—, —C(O)—, (hetero)cycle(s), and combinations thereof, in particular —O—C(O)—, —C(O)—O—, -(hetero)cycle-O-(hetero)cycle-, and preferably being interrupted with —O—C(O)— or —C(O)—O—;
X is as defined previously for formula (I); preferably, X denotes a sulfur atom;
Y, AK1, AK3, n, m, v, t, q, u, R, R₄, Rₐ and R_{b} being as defined previously for formula (I);
   it being understood that said compound contains at least two radicals —C(O)—C(Rₐ)(R_{b})—C(O)—R₄.

According to a preferred embodiment, n is equal to 0 and m is equal to 1.

According to a preferred embodiment, Z is a divalent radical.

According to a preferred embodiment, if Z is an unsaturated chain, AK2 is an unsaturated chain, and if Z is a saturated chain, AK2 is a saturated chain.

According to this embodiment variant, the compound(s) of formula (I), and also the optical or geometrical isomers thereof and/or the solvates thereof, such as hydrates, are in particular of formula (Iic):

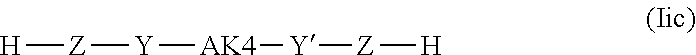

in which formula (Iic):
Y is as defined previously for formula (I);
Z represents a linear or branched, saturated or unsaturated, preferably saturated, C₁₄ to C₂₄, in particular C₁₆ to C₂₀, preferably C₁₇, divalent hydrocarbon-based radical;
Y′ represents —C(O)—O— or —O—C(O)— it being understood that if Y represents —O—C(O)—, Y′ represents —C(O)—O—, and if Y represents —C(O)—O—, Y′ represents —O—C(O)—;
AK4 denotes a saturated divalent hydrocarbon-based radical of 5 or 6 carbon atoms, substituted with at least two radicals —OR with R being as defined previously in formula (I);
   it being understood that the compounds of formula (Iic) contain at least two radicals —C(O)—C(Rₐ)(R_{b})—C(O)—R₄, and preferably four radicals —C(O)—C(Rₐ)(R_{b})—C(O)—R₄.

According to this embodiment variant, the compound(s) of formula (I) are in particular chosen from the compounds (12) hereinbelow, and also the optical or geometrical isomers thereof and/or the solvates thereof, such as hydrates:

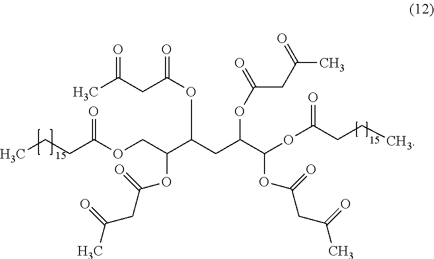

According to a fifth embodiment variant, the compound(s) of formula (I), and also the optical or geometrical isomers and/or solvates thereof, such as hydrates, are such that:
p is equal to 0;
Z represents a linear or branched, saturated or unsaturated, preferably saturated and acyclic, C₂ to C₂₄, in particular C₃ to C₁₀, preferably C₄ or C₅, tetravalent hydrocarbon-based radical, Z also being substituted with one or more radicals —[(O)ₜ-(AK1)_{q}-(O—C(O)—C(Rₐ)(R_{b})—C(O)—R₄)ᵤ]ₙ, n preferably being equal to 1;
n+m is equal to 4, and preferably m is equal to 3;
AK2 independently represents a linear or branched, saturated or unsaturated, preferably saturated, C₁₄ to C₃₀, preferably C₁₆ to C₂₄, preferably C₁₇, hydrocarbon-based chain, said chain being optionally substituted with one or more identical or different radicals chosen from —OR, —X-AK₃-(O—C(O)—C(Rₐ)(R_{b})—C(O)—R₄)ᵥ, preferably substituted with one or more radicals —OR; and/or said chain being optionally interrupted with one or more heteroatoms or groups chosen from —O—, —S—, —C(O)—, (hetero)cycle(s), and combinations thereof, in particular —O—C(O)—, —C(O)—O—, -(hetero)cycle-O-(hetero)cycle-, and preferably not being interrupted;
X is as defined previously for formula (I); preferably, X denotes a sulfur atom;
Y, AK1, AK3, u, t, q, v, n, m R, R₄, Rₐ and R_{b} being as defined previously for formula (I);
   it being understood that said compound contains at least two radicals —C(O)—C(Rₐ)(R_{b})—C(O)—R₄.

According to a preferred embodiment, t is equal to 0 and u is equal to 1.

According to a preferred embodiment, all the radicals AK2 are identical, and preferably all the radicals Y are identical.

According to a preferred embodiment, Y denotes —O—C(O)—.

According to a particular embodiment, the compounds according to this variant are of general formula (Iid), and also the optical or geometrical isomers thereof and/or the solvates thereof such as hydrates:

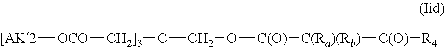

in which formula (Iid) AK′2 represents a linear or branched, preferably linear, saturated or unsaturated, preferably saturated, C₁₄-C₃₀, preferably C₁₆-C₂₄, preferably C₁₇, monovalent hydrocarbon-based chain, said chain being substituted with one or more identical or different radicals chosen from —OR, —S-AK3-(O—C(O)—C(Rₐ)(R_{b})—C(O)—R₄)ᵥ, preferably substituted with one or more radicals —OR; AK3, v, R, R₄, Rₐ and R_{b} being as defined previously for formula (I), it being understood that said compounds contain at least two radicals C(O)—C(Rₐ)(R_{b})—C(O—R₄.

According to a sixth embodiment variant, the compound(s) of formula (I), and also the optical or geometrical isomers and/or solvates thereof, such as hydrates, are such that:
p is equal to 0;
m is non-zero;
Z represents a linear or branched, saturated or unsaturated, conjugated or non-conjugated, preferably non-conjugated, acyclic or cyclic, aromatic or non-aromatic, preferably non-aromatic, divalent C₂ to C₂₄, in particular C₃ to C₁₂, hydrocarbon-based radical, said radical Z also being interrupted with one or more heteroatoms or groups chosen from —O—, —N(Rₐ)—, —S—, —C(O)—, 5- or 6-membered heterocycloalkyl(s), and combinations thereof;
AK2 represents a linear or branched, saturated or unsaturated, preferably saturated, C₁₄-C₃₀, preferably C₁₄-C₂₄, preferably C₁₇, monovalent hydrocarbon-based chain, said chain being optionally substituted with one or more identical or different radicals chosen from —OR and —X-AK3-(O—C(O)—C(Rₐ)(R_{b})—C(O)—R₄)ᵥ, preferably unsubstituted; and/or said chain being optionally interrupted with one or more heteroatoms or groups chosen from —O—, —S—, —C(O)—, (hetero)cycles, and combinations thereof, in particular —O—C(O)—, —C(O)—O— or -(hetero)cycle-O-(hetero)cycle-, preferably optionally interrupted with —O—C(O)— or —C(O)—O—, and more preferentially not being interrupted;
X is as defined previously for formula (I); preferably, X denotes a sulfur atom;
Y, AK1, AK3, v, t, q, u, R, R₄, Rₐ and R_{b} being as defined previously for formula (I);
   it being understood that said compound contains at least two radicals —C(O)—C(Rₐ)(R_{b})—C(O)—R₄.

According to a preferred embodiment, according to this variant, Z denotes a divalent radical —(CH₂)_{g}-(heterocycle)-O—(CH₂)ₕ-(heterocycle)-(CH₂)ⱼ— in which h is equal to 0, 1 or 2, preferably is equal to 0 or 1, g and j independently denote an integer between 1 and 3, preferably 1, (heterocycle) denotes a divalent 5- or 6-membered heterocycloalkyl radical, in particular a divalent tetrahydrofuran radical or a divalent tetrahydropyran radical, said heterocycle being optionally substituted with one or more radicals chosen from —OR, with R as defined previously for formula (I), and notably —OH or —O—C(O)—C(Rₐ)(R_{b})—C(O)—R₄.

According to a particular embodiment, the compounds according to this variant are of general formula (Iie), and also the optical or geometrical isomers thereof and/or the solvates thereof such as hydrates:

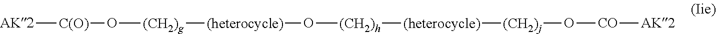

in which formula (Iie):
h is equal to 0, 1 or 2 and preferably is equal to 0;
g and j independently denote an integer between 1 and 3, preferably 1;
(heterocycle) denotes a divalent 5- or 6-membered heterocycloalkyl radical substituted with one or more identical or different radicals chosen from —OR, with R as defined previously for formula (I), and notably —OH or —O—C(O)—C(Rₐ)(R_{b})—C(O)—R₄;
AK″2 represents a linear or branched, saturated or unsaturated, preferably saturated, C₁₄-C₃₀, preferably C₁₄-C₂₄, preferably C₁₇, monovalent hydrocarbon-based chain, said chain being optionally substituted with one or more identical or different radicals chosen from —OH, —O—C(O)—C(Rₐ)(R_{b})—C(O)—R₄ and —S-AK₃-(O—C(O)—C(Rₐ)(R_{b})—C(O)—R₄)ᵥ, preferably unsubstituted; and/or said chain being optionally interrupted with one or more heteroatoms or groups chosen from —O—, —S—, —C(O)—, (hetero)cycle(s), and combinations thereof, in particular —O—C(O)—, —C(O)—O— or -(hetero)cycle-O-(hetero)cycle-, preferably optionally interrupted with —O—C(O)— or —C(O)—O—, and more preferentially not being interrupted;
   it being understood that said compound contains at least two radicals —C(O)—C(Rₐ)(R_{b})—C(O)—R₄.

According to this embodiment variant, the compounds of formula (I) or (Iie) are in particular chosen from the compounds (14) hereinbelow, and also the optical and geometrical isomers thereof and/or the solvates thereof, such as hydrates:

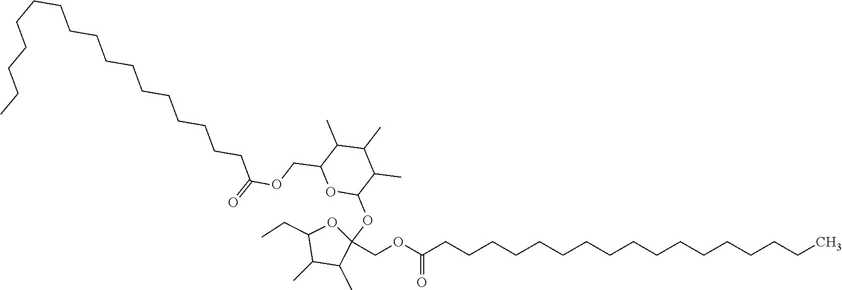 [SMILES: CCCCCCCCCCCCCCCCCC(=O)OCC1OC(OC2(COC(=O)CCCCCCCCCCCCCCCCC)OC(CC)C(C)C2C)C(C)C(C)C1C]

with W being a hydrogen atom or —C(O)—C(Rₐ)(R_{b})—C(O)—R₄, and said compound containing at least two groups —C(O)—C(Rₐ)(R_{b})—C(O)—R₄.

According to a seventh embodiment variant, the compound(s) of formula (I), and also the optical or geometrical isomers and/or solvates thereof, such as hydrates, are such that:
p is equal to 0;
Z represents a linear or branched, saturated or unsaturated, preferably saturated and acyclic, C₂ to C₂₄, in particular C₃ to C₁₀, preferably C₄ or C₅, tetravalent hydrocarbon-based radical, Z also being substituted with one or more radicals —[(O)ₜ-(AK1)_{q}-(O—C(O)—C(Rₐ)(R_{b})—C(O)—R₄)ᵤ]ₙ;
n+m is equal to 6, and preferably m is equal to 2;
AK2 represents a linear or branched, saturated or unsaturated, preferably saturated, C₃-C₈, in particular C₄, C₅ or C₆, monovalent hydrocarbon-based chain;
   Y, AK1, AK3, t, q, u, v, R, X, R₄, Rₐ and R_{b} being as defined previously for formula (I);
   it being understood that said compound contains at least two radicals —C(O)—C(Rₐ)(R_{b})—C(O)—R₄.

According to this embodiment variant, the compounds of formula (I) are in particular chosen from the compound (15) hereinbelow, and also the optical and geometrical isomers thereof and/or the solvates thereof, such as hydrates:

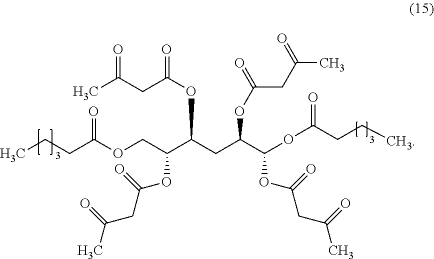

According to a preferred embodiment, the compound(s) of formula (I) are chosen from compounds (1) to (16) below, and also the optical or geometrical isomers thereof, and/or the solvates thereof, such as hydrates:

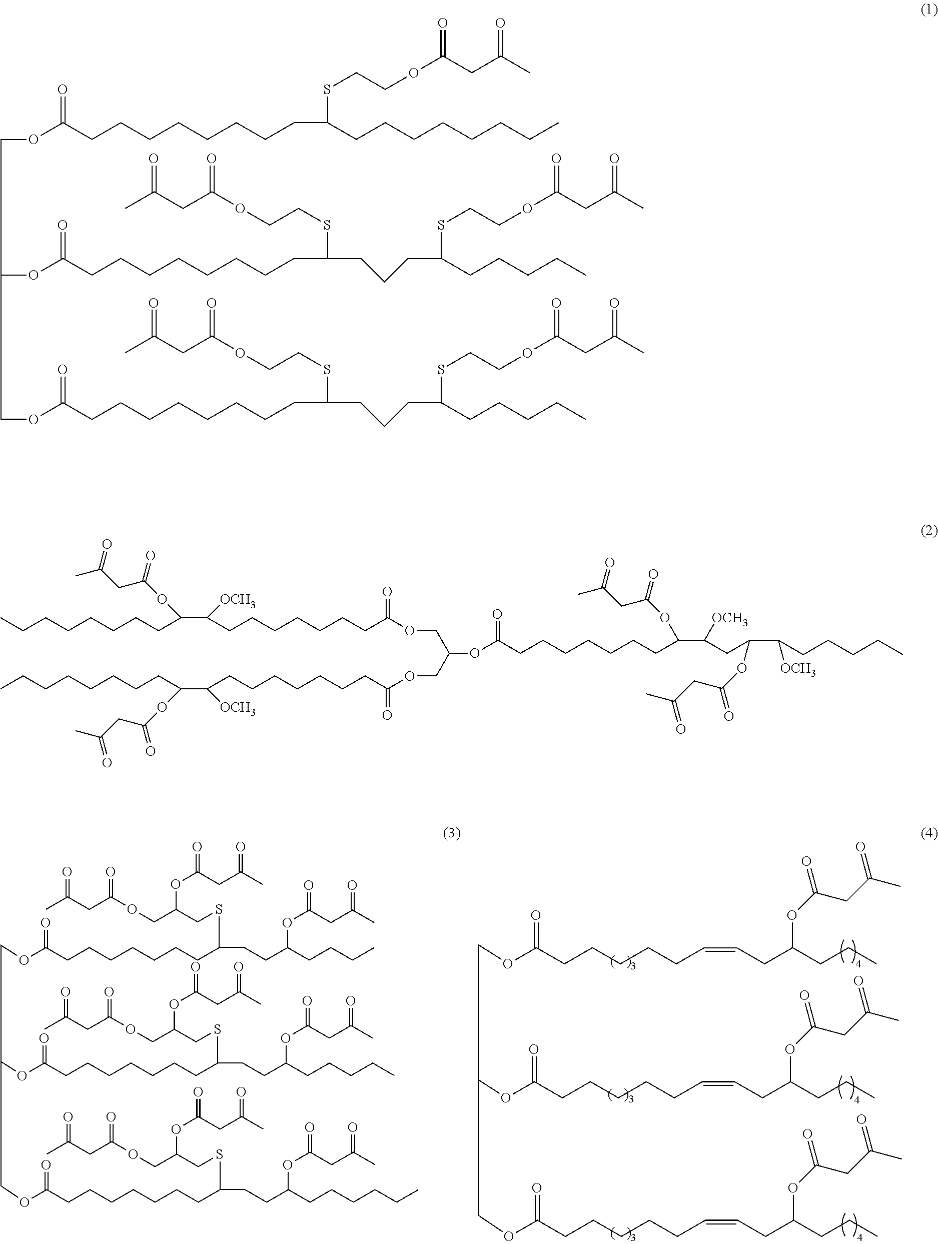

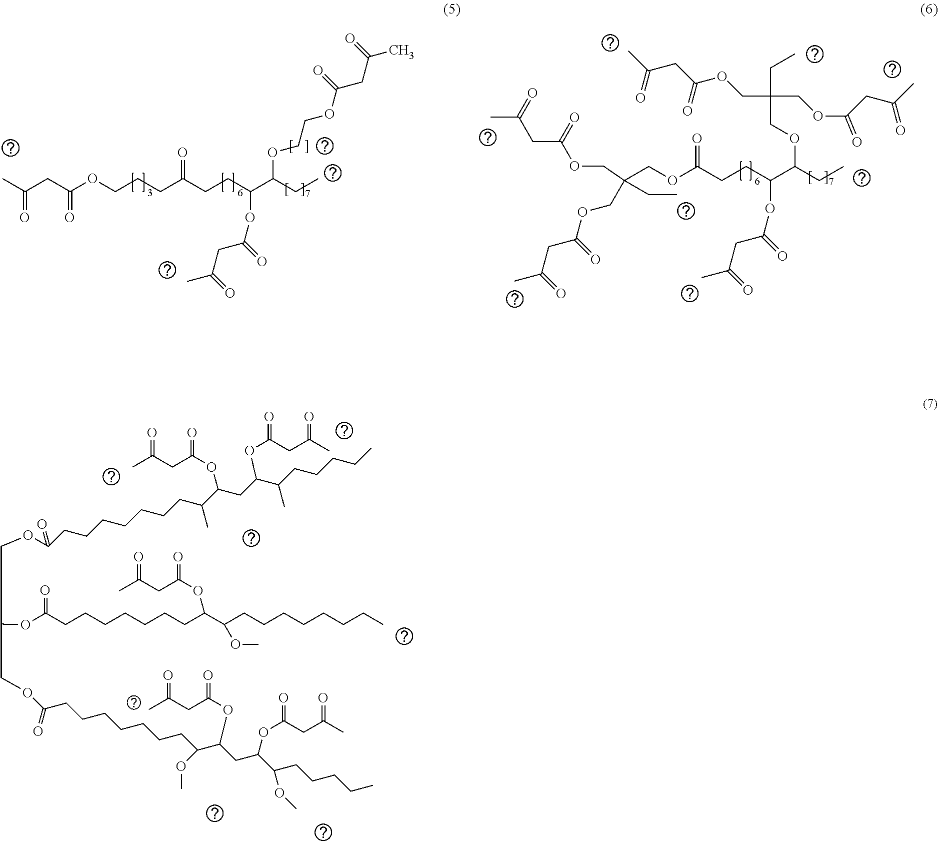

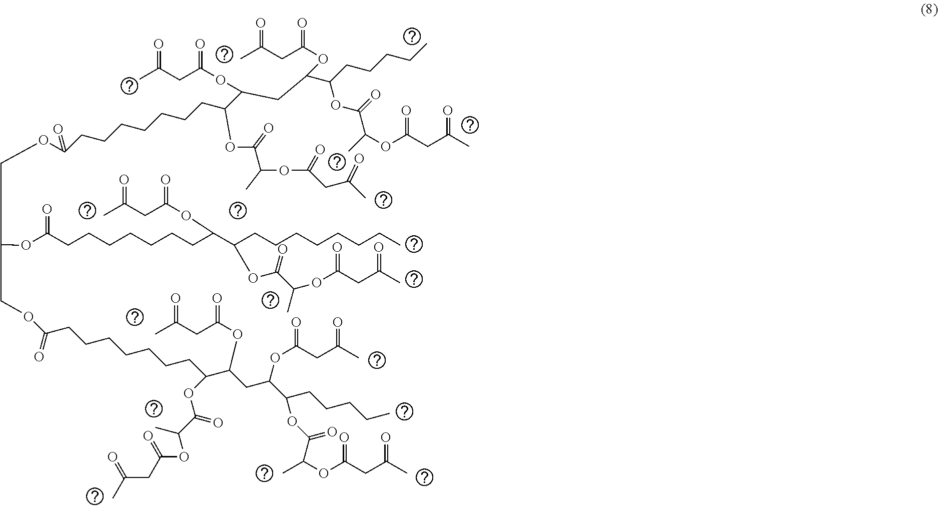 [SMILES: CCCCCCCCC(OC(=O)C(C)OC(=O)CC(C)=O)C(CCCCCCCC(=O)OC(COC(=O)CCCCCCCC(OC(=O)C(C)OC(=O)CC(C)=O)C(CC(OC(=O)CC(C)=O)C(CCCCC)OC(=O)C(C)OC(=O)CC(C)=O)OC(=O)CC(C)=O)COC(=O)CCCCCCCC(OC(=O)C(C)OC(=O)CC(C)=O)C(CC(OC(=O)CC(C)=O)C(CCCCC)OC(=O)C(C)OC(=O)CC(C)=O)OC(=O)CC(C)=O)OC(=O)CC(C)=O]

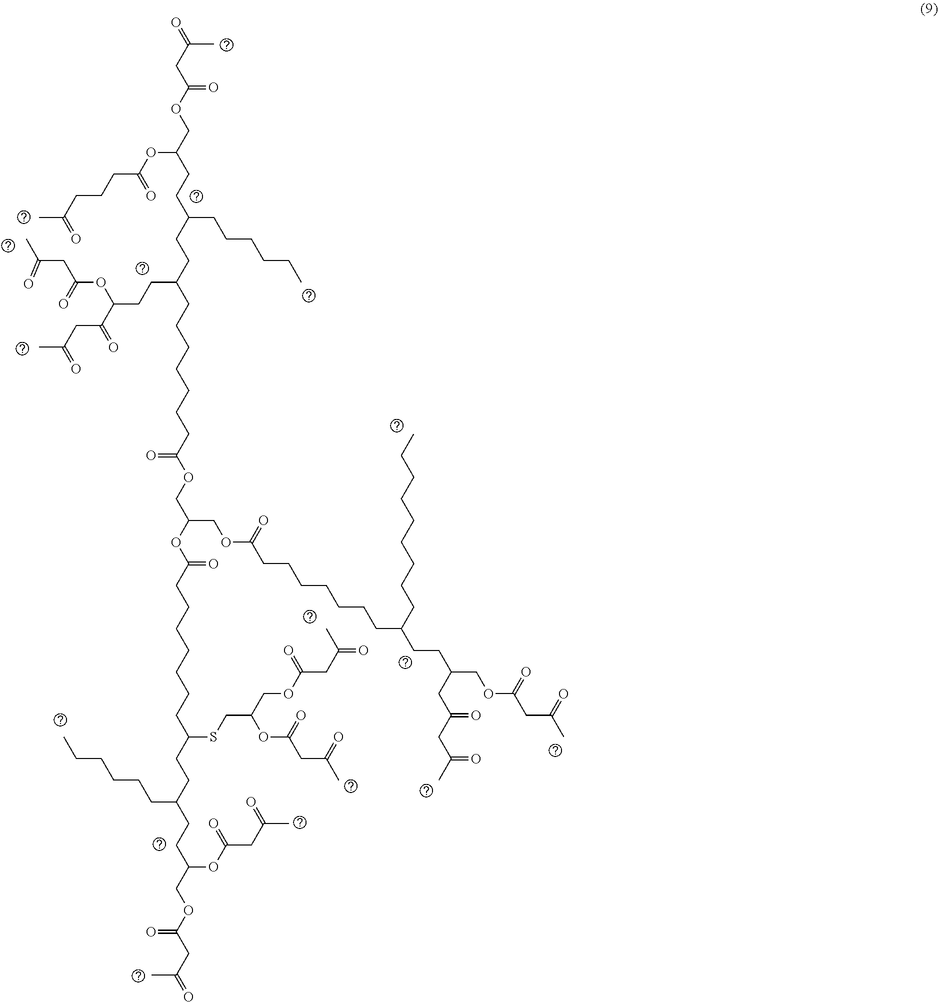 [SMILES: CCCCCCCCCC(CCCCCCCC(=O)OCC(COC(=O)CCCCCCCC(CCC(CCCCCC)CCC(COC(=O)CC(C)=O)OC(=O)CCCC(C)=O)CCC(OC(=O)CC(C)=O)C(=O)CC(C)=O)OC(=O)CCCCCCCC(CCC(CCCCCC)CCC(COC(=O)CC(C)=O)OC(=O)CC(C)=O)SCC(COC(=O)CC(C)=O)OC(=O)CC(C)=O)CCC(COC(=O)CC(C)=O)CC(=O)CC(C)=O]

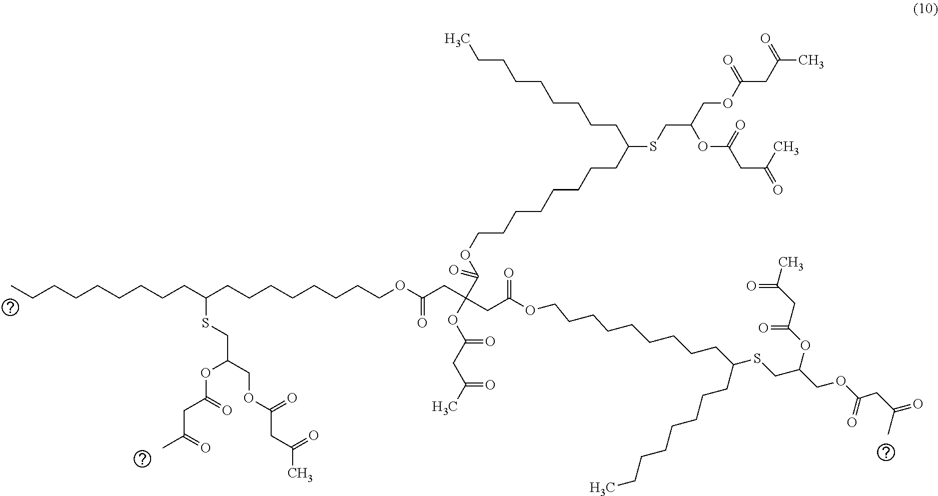 [SMILES: CCCCCCCCCC(CCCCCCCCOC(=O)CC(CC(=O)OCCCCCCCCCC(CCCCCCCC)SCC(COC(=O)CC(C)=O)OC(=O)CC(C)=O)(OC(=O)CC(C)=O)C(=O)OCCCCCCCCC(CCCCCCCCC)SCC(COC(=O)CC(C)=O)OC(=O)CC(C)=O)SCC(COC(=O)CC(C)=O)OC(=O)CC(C)=O]

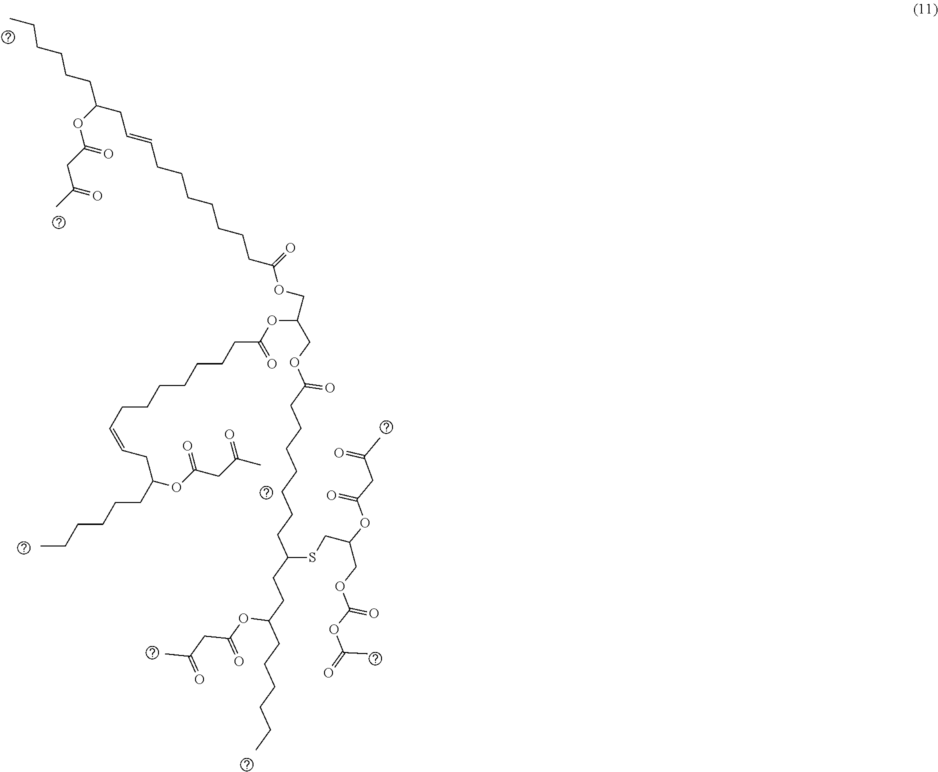 [SMILES: CCCCCCC(C/C=C\CCCCCCCC(=O)OC(COC(=O)CCCCCCC/C=C/CC(CCCCCC)OC(=O)CC(C)=O)COC(=O)CCCCCCCC(CCC(CCCCCC)OC(=O)CC(C)=O)SCC(COC(=O)OC(C)=O)OC(=O)CC(C)=O)OC(=O)CC(C)=O]

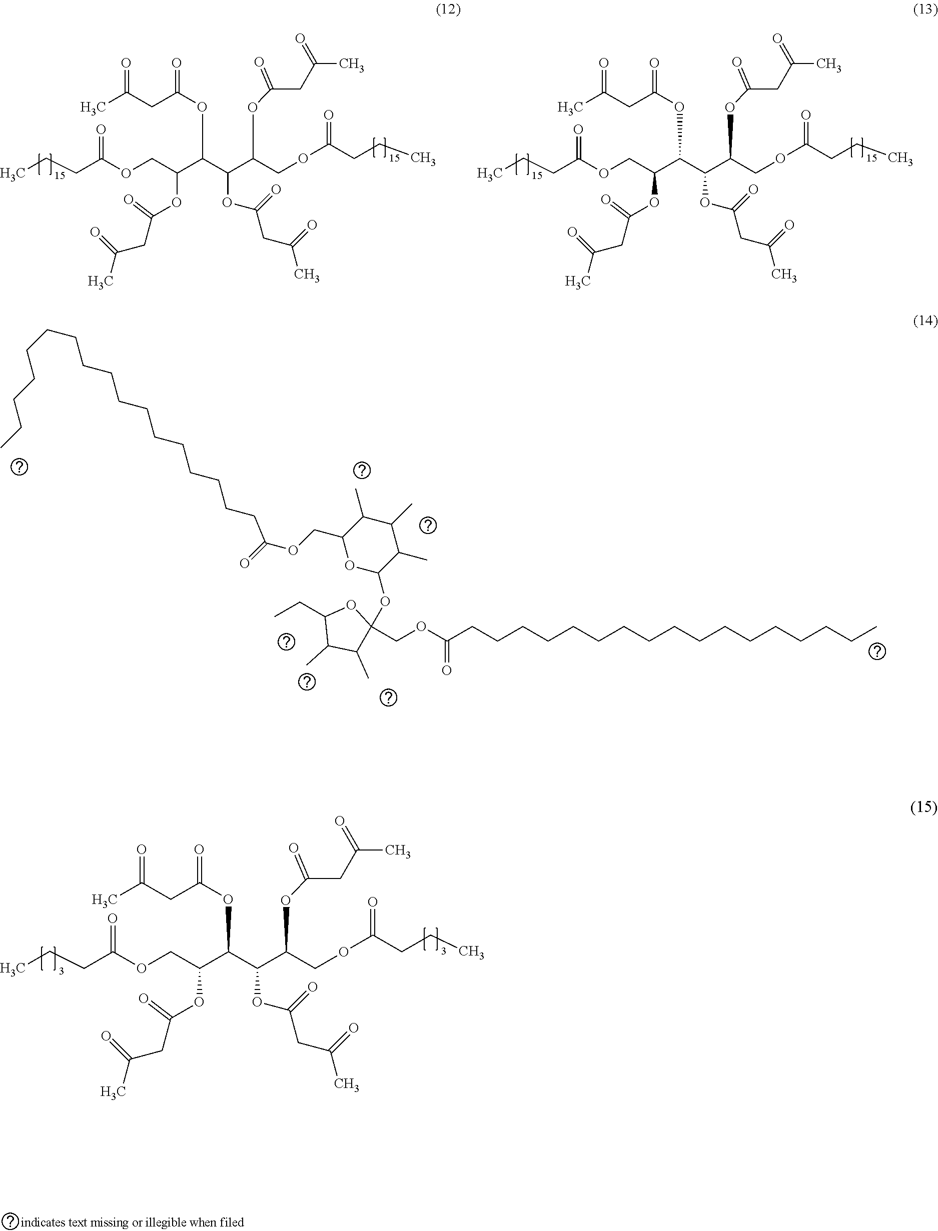

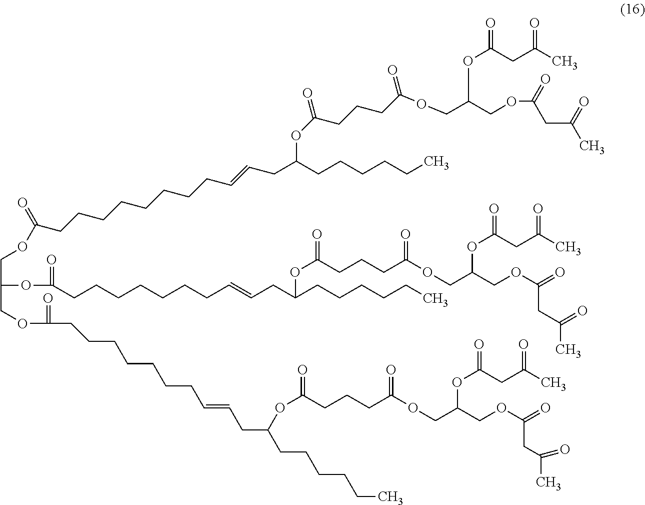 [SMILES: CCCCCCC(C/C=C/CCCCCCCCC(=O)OCC(COC(=O)CCCCCCC/C=C/CC(CCCCCC)OC(=O)CCCC(=O)OCC(COC(=O)CC(C)=O)OC(=O)CC(C)=O)OC(=O)CCCCCCC/C=C/CC(CCCCCC)OC(=O)CCCC(=O)OCC(COC(=O)CC(C)=O)OC(=O)CC(C)=O)OC(=O)CCCC(=O)OCC(COC(=O)CC(C)=O)OC(=O)CC(C)=O]

with W being a hydrogen atom or —C(O)—C(Rₐ)(R_{b})—C(O)—R₄, and said compound containing at least two groups —C(O)—C(Rₐ)(R_{b})—C(O)—R₄.

According to a preferred embodiment, the compound(s) of formula (I) are chosen from compounds (1) to (14) above, and also the optical or geometrical isomers thereof, and/or the solvates thereof, such as hydrates.

According to a preferred embodiment, the compound(s) of formula (I) are in particular chosen from compounds (1), (3), (4), (8), (9), (10), (11), (15) and (16) hereinbelow, and also the optical or geometrical isomers thereof and/or the solvates thereof, such as hydrates:

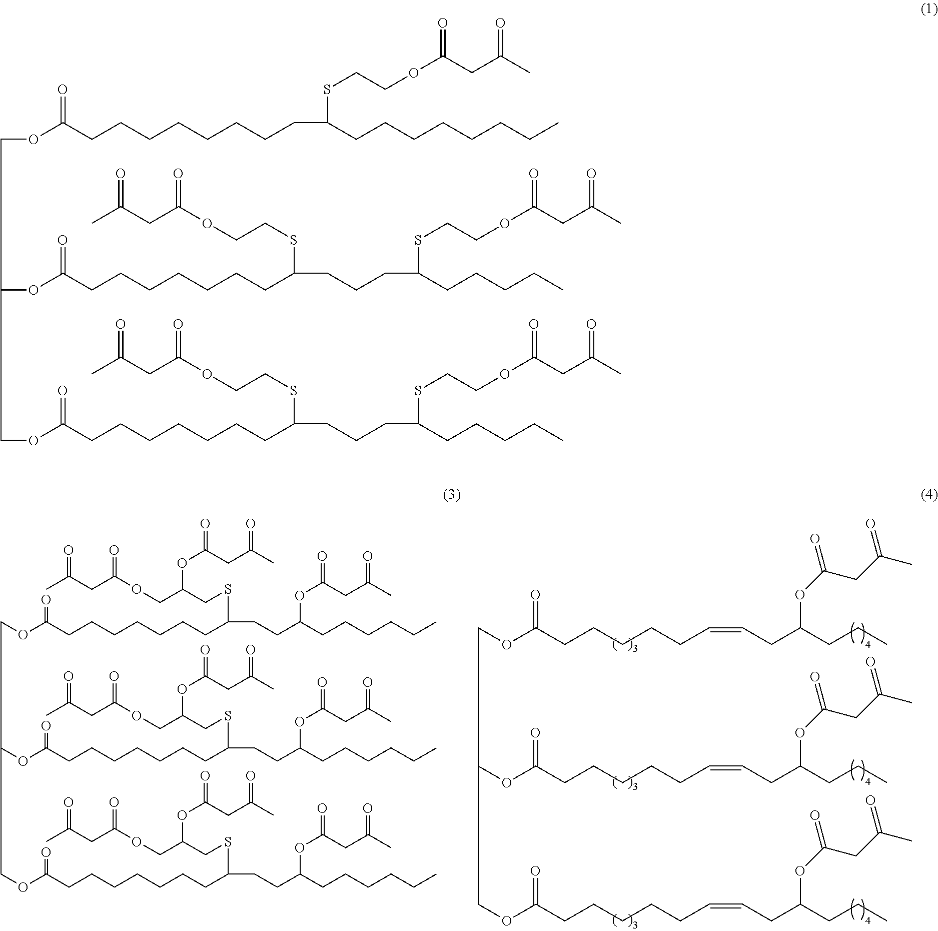

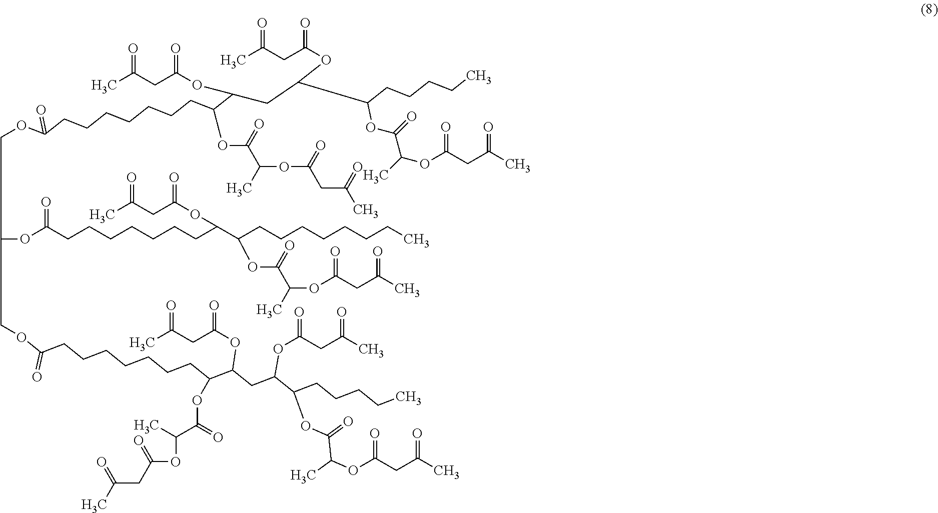 [SMILES: CCCCCCCCC(OC(=O)C(C)OC(=O)CC(C)=O)C(CCCCCCCC(=O)OC(COC(=O)CCCCCCCC(OC(=O)C(C)OC(=O)CC(C)=O)C(CC(OC(=O)CC(C)=O)C(CCCCC)OC(=O)C(C)OC(=O)CC(C)=O)OC(=O)CC(C)=O)COC(=O)CCCCCCCC(OC(=O)C(C)OC(=O)CC(C)=O)C(CC(OC(=O)CC(C)=O)C(CCCCC)OC(=O)C(C)OC(=O)CC(C)=O)OC(=O)CC(C)=O)OC(=O)CC(C)=O]

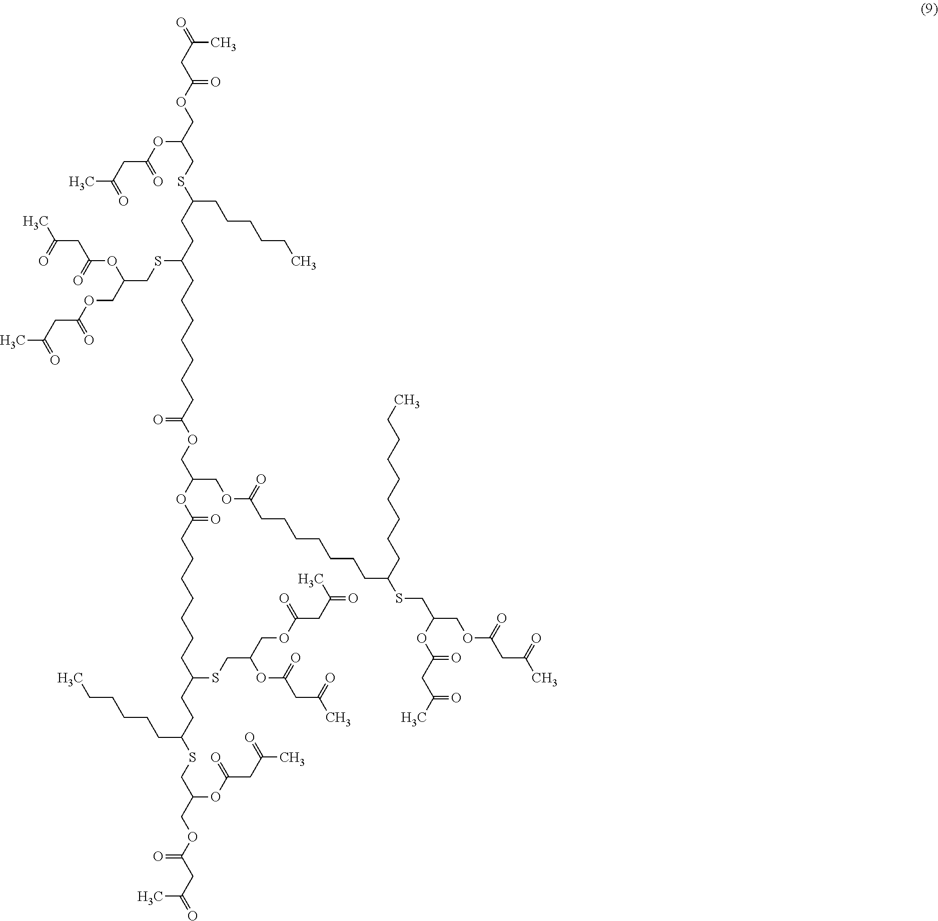 [SMILES: CCCCCCCCCC(CCCCCCCC(=O)OCC(COC(=O)CCCCCCCC(CCC(CCCCCC)SCC(COC(=O)CC(C)=O)OC(=O)CC(C)=O)SCC(COC(=O)CC(C)=O)OC(=O)CC(C)=O)OC(=O)CCCCCCCC(CCC(CCCCCC)SCC(COC(=O)CC(C)=O)OC(=O)CC(C)=O)SCC(COC(=O)CC(C)=O)OC(=O)CC(C)=O)SCC(COC(=O)CC(C)=O)OC(=O)CC(C)=O]

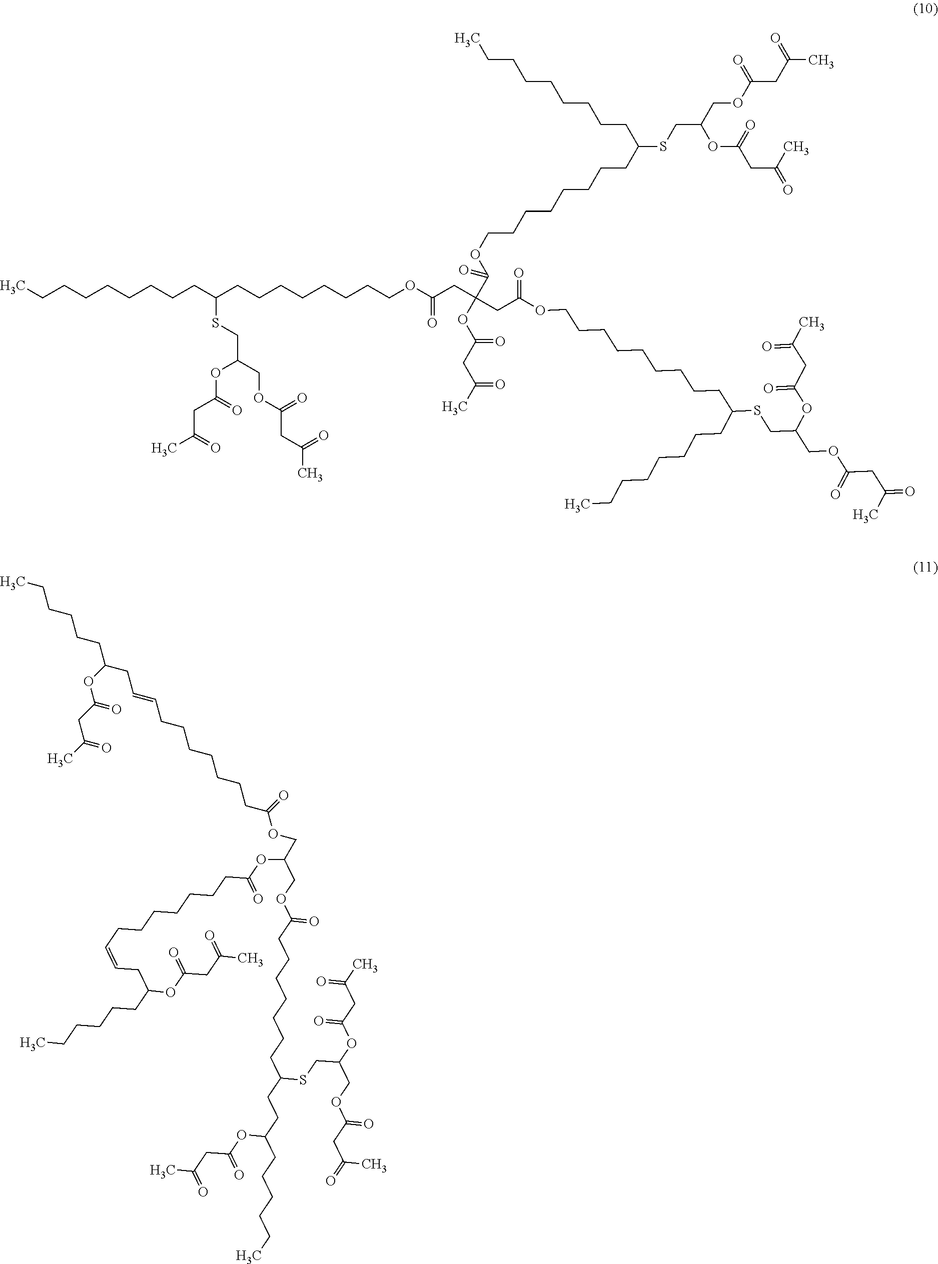 [SMILES: CCCCCCCCCC(CCCCCCCCOC(=O)CC(CC(=O)OCCCCCCCCCC(CCCCCCCC)SCC(COC(=O)CC(C)=O)OC(=O)CC(C)=O)(OC(=O)CC(C)=O)C(=O)OCCCCCCCCC(CCCCCCCCC)SCC(COC(=O)CC(C)=O)OC(=O)CC(C)=O)SCC(COC(=O)CC(C)=O)OC(=O)CC(C)=O ; CCCCCCC(C/C=C\CCCCCCCC(=O)OC(COC(=O)CCCCCCC/C=C/CC(CCCCCC)OC(=O)CC(C)=O)COC(=O)CCCCCCCC(CCC(CCCCCC)OC(=O)CC(C)=O)SCC(COC(=O)CC(C)=O)OC(=O)CC(C)=O)OC(=O)CC(C)=O]

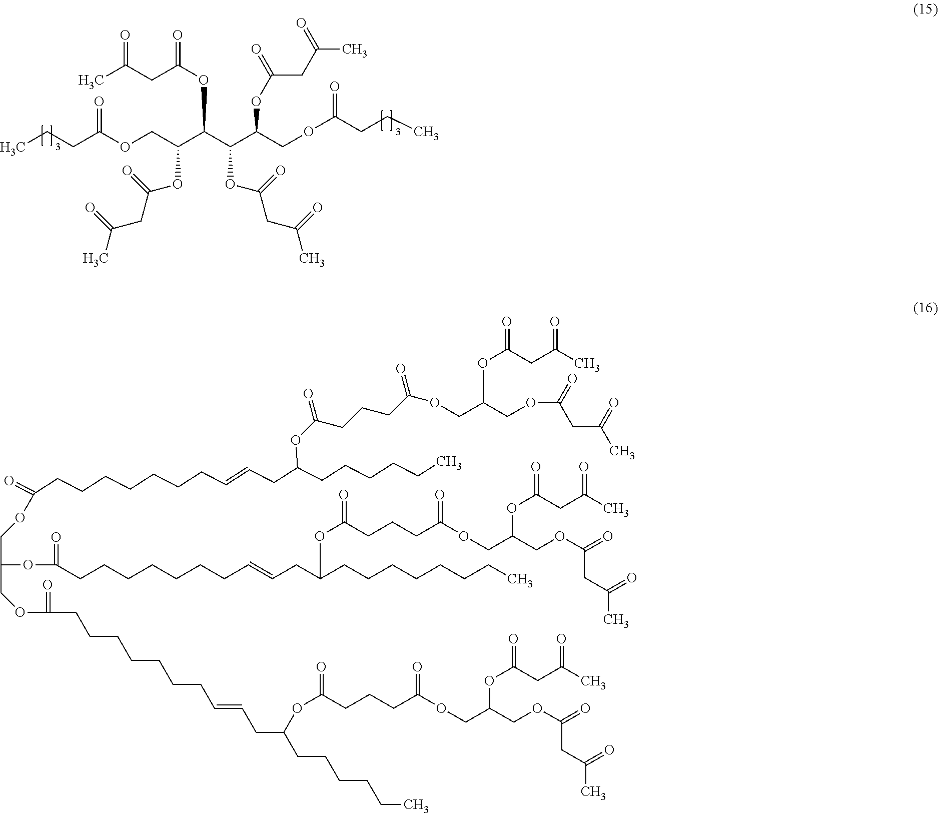

According to a preferred embodiment, the compound(s) of formula (I) are in particular chosen from compounds (1), (3), (4), (8), (9), (10) and (11) above, and also the optical or geometrical isomers thereof and/or the solvates thereof, such as hydrates.

Another subject of the invention is the compounds of formula (I), and also the optical or geometrical isomers thereof and/or the solvates thereof, such as hydrates, and a composition, notably a cosmetic composition, in particular for treating keratin fibres, in particular for caring for, styling and/or colouring keratin fibres, preferably the hair, containing same:

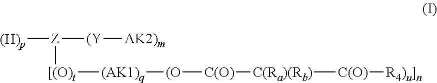

in which formula (I):
Z represents a linear or branched, saturated or unsaturated, conjugated or non-conjugated, acyclic or cyclic, aromatic or non-aromatic multivalent C₂ to C₄₀, in particular C₃ to C₃₆, hydrocarbon-based radical. Z possibly being interrupted with one or more groups or heteroatoms chosen from —O—, —N(Rₐ)—, —S—, —C(O)—, (hetero)cycles, and combinations thereof;
AK1 represents a divalent, linear or branched, saturated or unsaturated C₁-C₁₂, preferably C₁-C₆, more preferentially saturated C₁-C₆, hydrocarbon-based chain;
AK2 represents a linear or branched, saturated or unsaturated, C₁-C₄₀ monovalent hydrocarbon-based chain, optionally substituted with one or more identical or different radicals chosen from —OR, —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), and combinations thereof, in particular —O—C(O)—, —C(O)—O— and/or -(hetero)cycle-O-(hetero)cycle-;
AK3 represents a divalent linear or branched, saturated or unsaturated C₁-C₁₂, preferably C₁-C₆, more preferentially saturated C₁-C₆, even more preferentially C₁-C₄, hydrocarbon-based chain;
R represents a hydrogen atom, a linear or branched, saturated or unsaturated C₁-C₆, preferably C₁-C₄, alkyl radical, in particular a methyl radical, or a radical —C(O)—C(Rₐ)(R_{b})—C(O)—R₄;
X represents —O—, —S—, —O—C(O)— or —C(O)—O—;
Y represents —O—C(O)— or —C(O)—O—;
R₄ represents a linear or branched, saturated or unsaturated C₁-C₆ monovalent hydrocarbon-based radical, preferably a (C₁-C₄)alkyl group, in particular methyl or tert-butyl, more preferentially methyl;
Rₐ and R_{b}, which may be identical or different, represent a hydrogen atom or a (C₁-C₄)alkyl group, preferably a hydrogen atom;
p denotes an integer equal to 0 or 1;
m denotes an integer equal to 0, 1, 2, 3 or 4; it being understood that when m is greater than 1, then the radicals —Y-AK₂ are identical or different;
n denotes an integer equal to 0, 1, 2, 3 or 4; it being understood that when n is greater than 1, then the radicals —(O)ₜ-(AK₁)_{q}—(O—C(O)—C(Rₐ)(R_{b})—C(O)—R₄)ᵤ are identical or different;
t denotes an integer equal to 0 or 1;
q denotes an integer equal to 0 or 1;
u denotes an integer equal to 1, 2 or 3;
v denotes an integer equal to 1, 2 or 3;
   it being understood that if q is equal to 0 then t is equal to 0, that (m+n) is greater than or equal to 1, and that the compounds of formula (I) contain at least two groups —C(O)—C(Rₐ)(R_{b})—C(O)—R₄;
   with the exception of the following compounds:

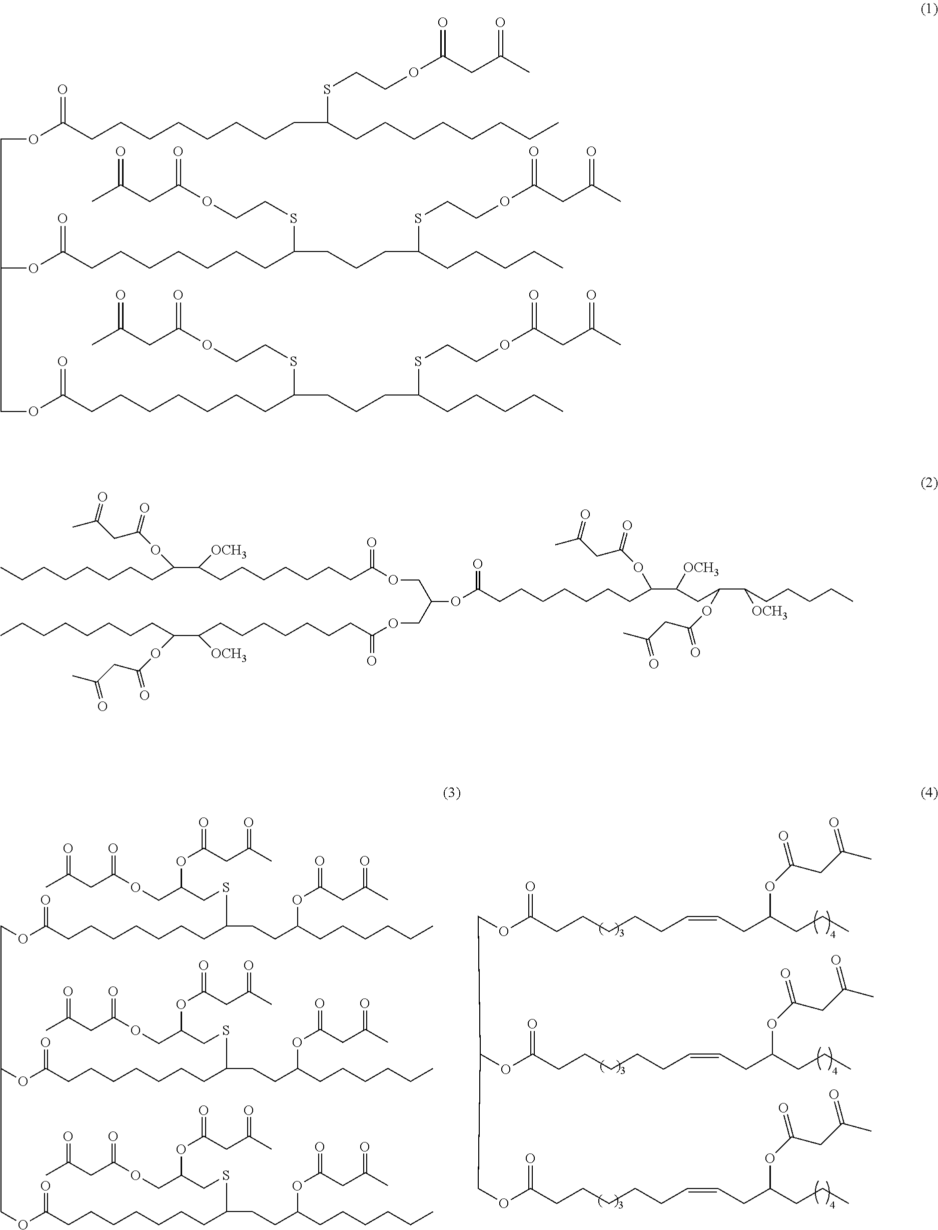

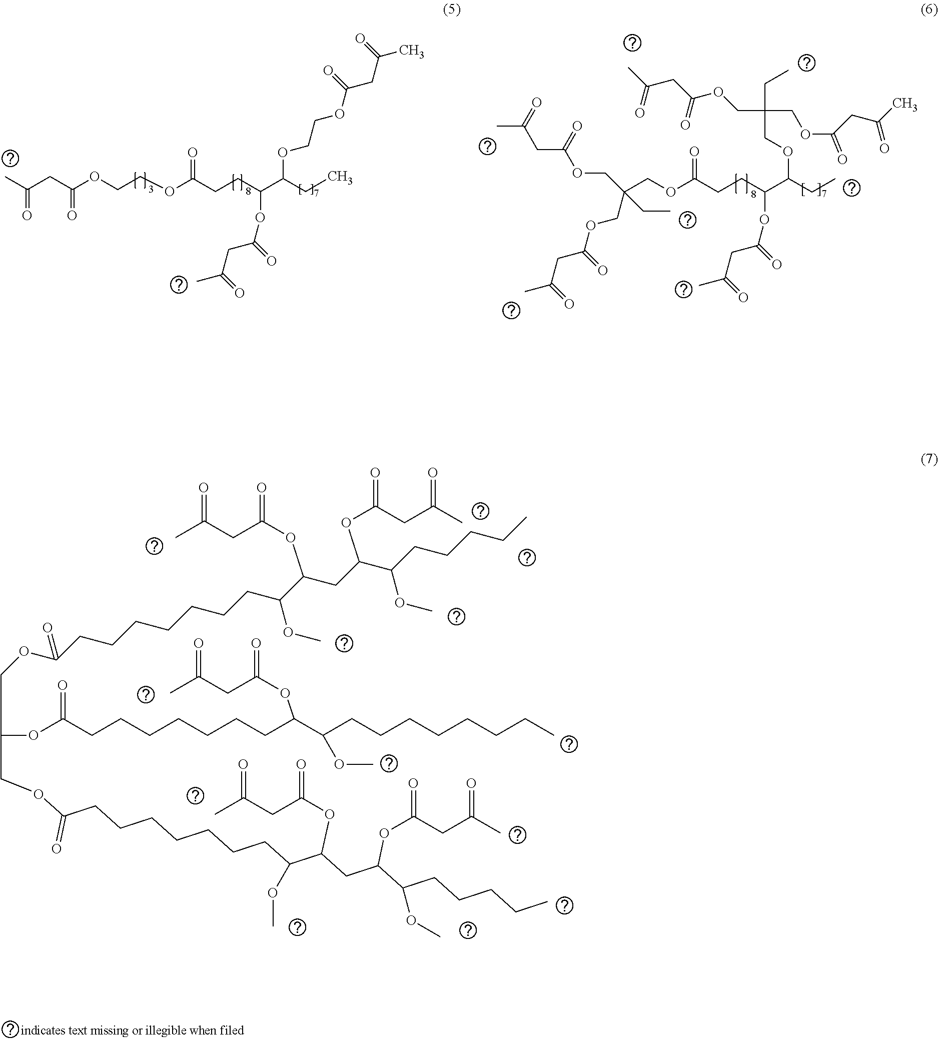

The subject of the present invention is also at least one compound of formula (I), and also the optical or geometrical isomers thereof and/or the solvates thereof, such as the hydrates, and a composition, notably a cosmetic composition, in particular for treating keratin fibres, in particular for caring for, styling and/or colouring keratin fibres, preferably the hair, containing same, said compound(s) being chosen from compounds (8), (9), (10), (11), (12), (13) and (14), and also the optical or geometrical isomers thereof and/or the solvates thereof, such as the hydrates.

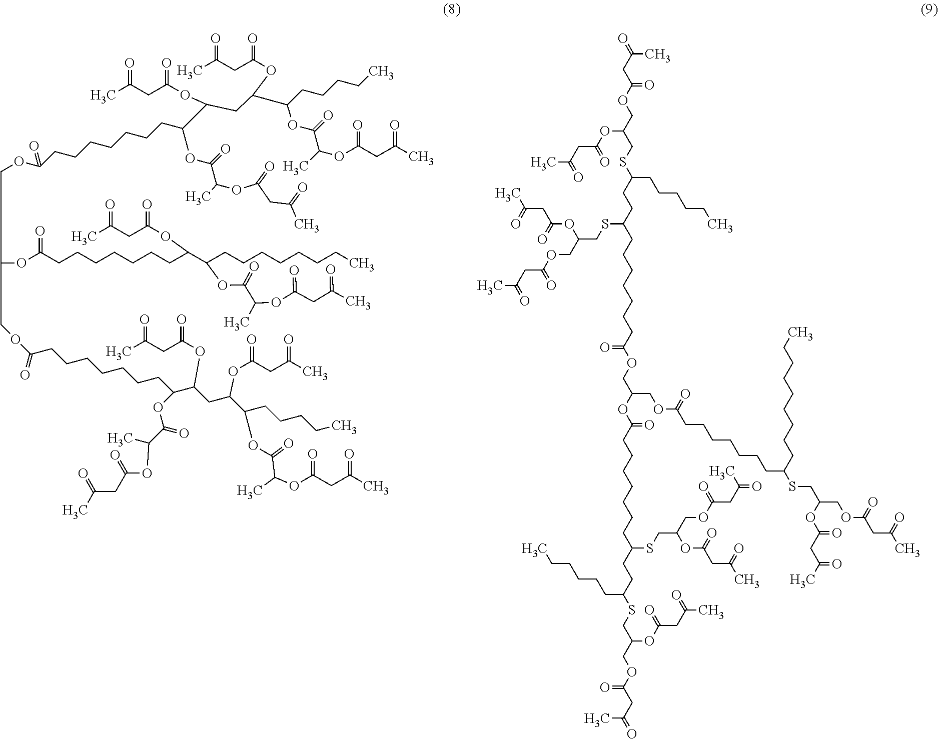

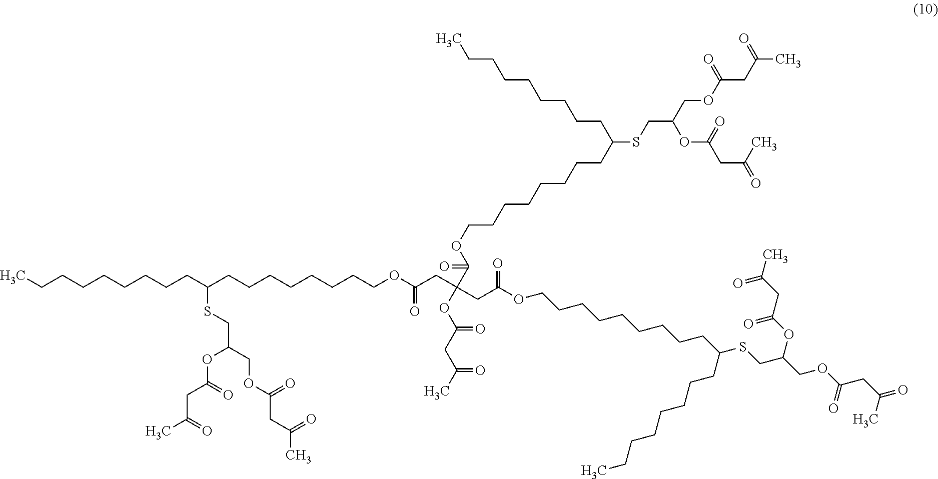 [SMILES: CCCCCCCCCC(CCCCCCCCOC(=O)CC(CC(=O)OCCCCCCCCCC(CCCCCCCC)SCC(COC(=O)CC(C)=O)OC(=O)CC(C)=O)(OC(=O)CC(C)=O)C(=O)OCCCCCCCCC(CCCCCCCCC)SCC(COC(=O)CC(C)=O)OC(=O)CC(C)=O)SCC(COC(=O)CC(C)=O)OC(=O)CC(C)=O]

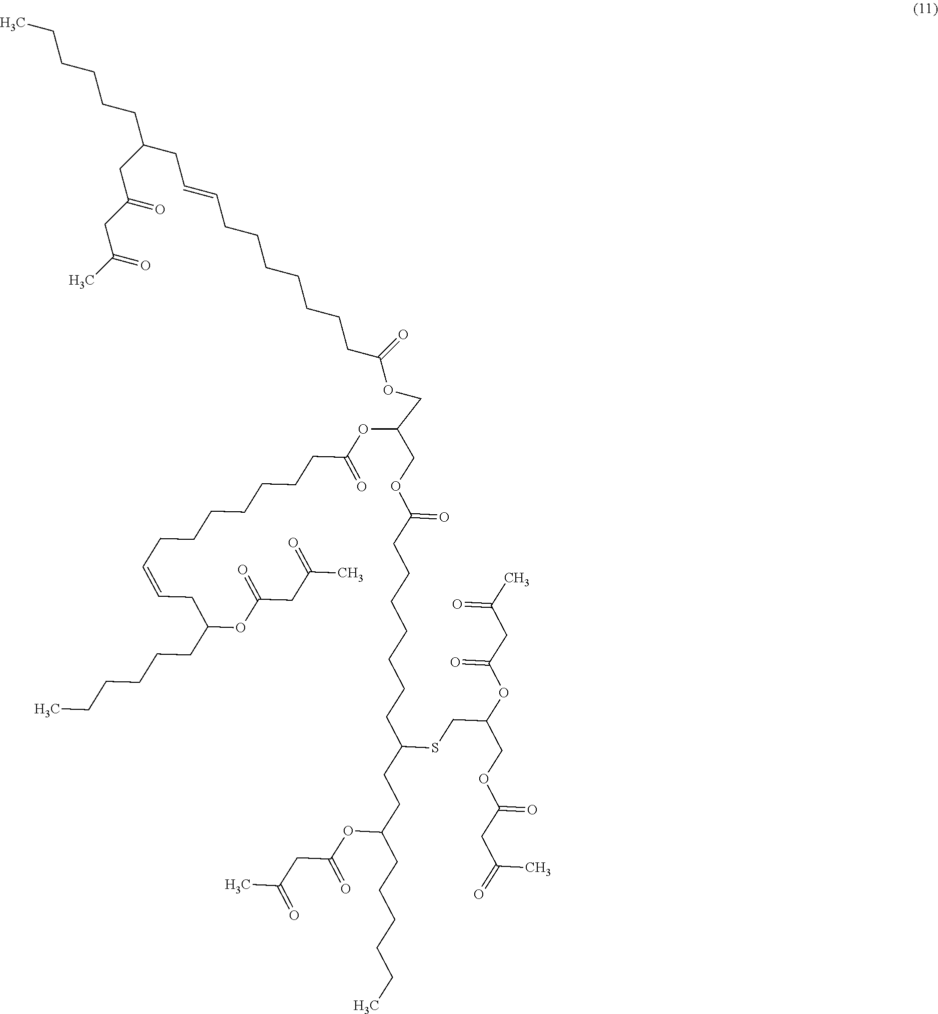 [SMILES: CCCCCCC(C/C=C/CCCCCCCC(=O)OCC(COC(=O)CCCCCCCC(CCC(CCCCCC)OC(=O)CC(C)=O)SCC(COC(=O)CC(C)=O)OC(=O)CC(C)=O)OC(=O)CCCCCCC/C=C\CC(CCCCCC)OC(=O)CC(C)=O)CC(=O)CC(C)=O]

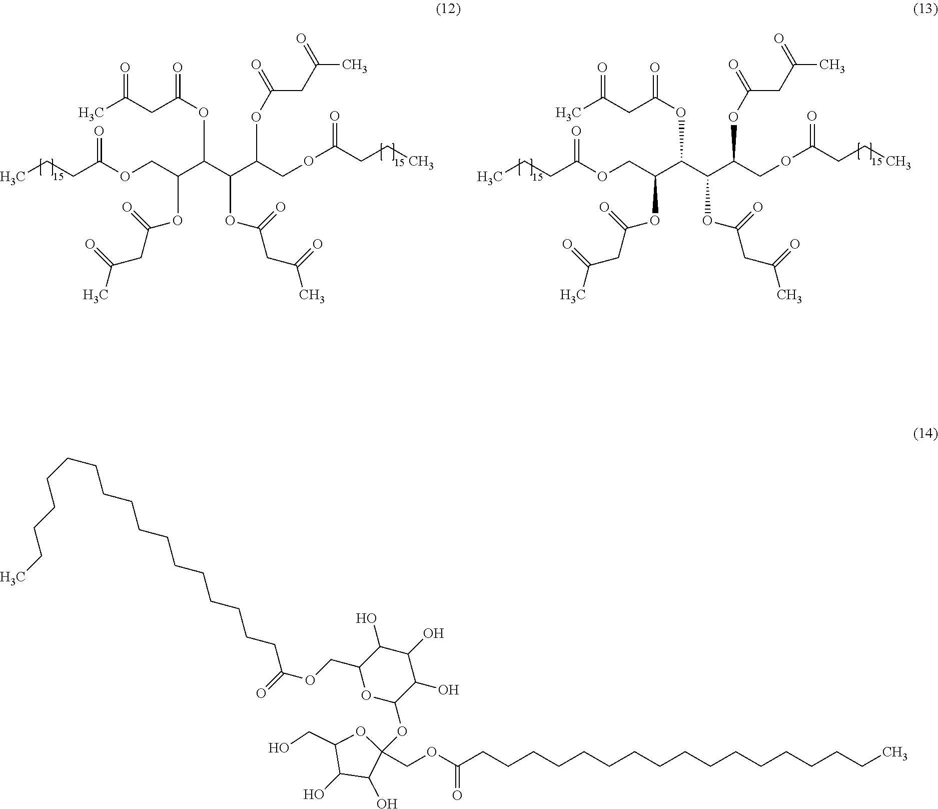

with W being a hydrogen atom or —C(O)—C(Rₐ)(R_{b})—C(O)—R₄, and said compound containing at least two groups —C(O)—C(Rₐ)(R_{b})—C(O)—R₄.

Another subject of the invention is the compounds of formula (I), and also the optical or geometrical isomers thereof and/or the solvates thereof, such as hydrates, chosen from the following compounds of formula (Ia), and also the optical or geometrical isomers thereof and/or the solvates thereof, such as hydrates, and a composition, notably a cosmetic composition, in particular for treating keratin fibres, in particular for caring for, styling and/or colouring keratin fibres, preferably the hair, containing same:

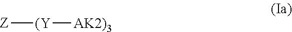

in which formula (Ia):
Z represents —CH₂—CH—CH₂—;
AK2 independently denotes a linear or branched, saturated or unsaturated, C₁₂-C₂₄ monovalent hydrocarbon-based chain, optionally substituted with one or more identical or different radicals chosen from —OR and —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, epoxides, and combinations thereof, in particular O—C(O)— and —C(O)—O—;
Y, AK3, v, R, X, R₄, Rₐ and R_{b} being as defined previously in formula (I); preferably, Y denotes —O—C(O);
it being understood that said compound contains at least two radicals —C(O)—C(Rₐ)(R_{b})—C(O)—R₄;
   in particular with the exception of the compounds (1), (2), (3), (4) and (7) as defined previously.

According to a preferred embodiment, the compound(s) of formula (Ia) are in particular chosen from compounds (8), (9), (11) and (16) as defined previously, and also the optical or geometrical isomers thereof and/or the solvates thereof, such as hydrates.

According to a particular embodiment, the compound(s) of formula (Ia), and also the optical and geometrical isomers thereof and/or the solvates thereof, such as hydrates, are compounds for which:
a) at least one of the three radicals AK2 denotes a branched, saturated or unsaturated C₁₂-C₂₄ monovalent hydrocarbon-based chain optionally substituted with one or more identical or different radicals chosen from —OR and —X-AK₃-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), in particular epoxides, and combinations thereof, in particular —O—C(O)— or —C(O)—O—; AK3, v, R, X, R₄, Rₐ and R_{b} being as defined previously; it being understood that said compound contains at least two radicals C(O)—C(Rₐ)(R_{b})—C(O)—R₄; preferably Y represents —O—C(O); preferably X represents S; preferably the three chains AK2 contain from 15 to 21 carbon atoms, more preferably 17 carbon atoms; and preferably none of the three radicals AK2 is interrupted; or
b) the three radicals AK2 independently represent a linear, saturated, C₁₂-C₂₄ hydrocarbon-based chain substituted with at least one radical chosen from —OH and OR′, and optionally substituted with one or more identical or different radicals chosen from —O—C(O)—C(Rₐ)(R_{b})—C(O)—R₄ and —X′-AK3-(O—C(O)—C(Rₐ)(R_{b})—C(O)—R₄)ᵥ; R′ denoting a C₂-C₆ alkyl radical, in particular ethyl; AK3, v, R, R₄, Rₐ and R_{b} being as defined previously, preferably, Y denotes —O—C(O)—; and X′ represents —O— or —C(O)—O—; it being understood that said compound contains at least two radicals —C(O)—C(Rₐ)(R_{b})—C(O)—R₄; preferably, the three chains AK2 comprise from 15 to 21 carbon atoms, more preferably 17 carbon atoms; and preferably none of the three radicals AK2 is interrupted; or
c) the radicals AK2 independently represent a linear, saturated, C₁₂-C₂₄ monovalent hydrocarbon-based chain, optionally substituted with one or more identical or different radicals chosen from —OR and —X-AK3-(O—C(O)—C(Rₐ)(R_{b})—C(O)—R₄)ᵥ; said chain being interrupted with one or more heteroatoms or groups chosen from —O— and/or —S— and/or —C(O)—, (hetero)cycle(s), in particular one or more epoxides, and combinations thereof, preferably —O—C(O)—, —C(O)—O— and -(hetero)cycle-O-(hetero)cycle-; AK3, v, R, X, R₄, Rₐ and R_{b} being as defined previously; and it being understood that said compound contains at least two radicals C(O)—C(Rₐ)(R_{b})—C(O)—R₄; preferably Y represents —O—C(O)—; preferably, X represents S; preferably, the three chains AK2 comprise from 15 to 21 carbon atoms, more preferentially 17 carbon atoms; or
d) the three radicals AK2 are such that at least one of the three radicals AK2 represents a linear or branched, unsaturated C₁₂-C₂₄ monovalent hydrocarbon-based chain, optionally substituted with one or more identical or different radicals chosen from —OR, said chain optionally being interrupted with one or more heteroatoms or groups chosen from —O—, —S—, —C(O)—, (hetero)cycle(s), and combinations thereof, in particular —O—C(O)—, —C(O)—O— or (hetero)cycle-O-(hetero)cycle; and at least one of the three radicals AK2 represents a linear or branched, saturated C₁₂-C₂₄ monovalent hydrocarbon-based chain, substituted with one or more identical or different radicals chosen from —S-AK3-(O—C(O)—C(Rₐ)(R_{b})—C(O)—R₄)ᵥ and optionally substituted with one or more identical or different radicals chosen from —OR; AK3, v, R, R₄, Rₐ and R_{b} being as defined previously; preferably, Y denotes —O—C(O)—; it being understood that said compound contains at least two radicals —C(O)—C(Rₐ)(R_{b})—C(O)—R₄; preferably, Y represents —O—C(O)—; preferably, X represents S; preferably, the three chains AK2 comprise from 15 to 21 carbon atoms, more preferentially 17 carbon atoms; preferably, none of the three radicals AK2 is interrupted; or
e) the three radicals AK2 are such that AK2 represents a saturated linear monovalent C₁₂-C₂₄ hydrocarbon-based chain substituted with one or more identical or different radicals chosen from —S-AK3-(O—C(O)—C(Rₐ)(R_{b})—C(O)—R₄)ᵥ; said chain being optionally interrupted with one or more heteroatoms or groups chosen from —O—, —S—, —C(O)—, (hetero)cycle(s), and combinations thereof, such as —O—C(O)—, —C(O)—O—, (hetero)cycle-O-(hetero)cycle; AK3 represents a divalent, linear or branched, saturated or unsaturated C₃-C₁₂, preferably C₃-C₆, more preferentially saturated C₃-C₆, in particular C₃ hydrocarbon-based chain; v, R₄, Rₐ and R_{b} being as defined previously; preferably, Y denotes —O—C(O)—; it being understood that said compound contains at least two radicals —C(O)—C(Rₐ)(R_{b})—C(O)—R₄; preferably, Y represents —O—C(O)—; preferably, v is equal to 2; preferably, the three chains AK2 comprise from 15 to 21 carbon atoms, more preferentially 17 carbon atoms; preferably, none of the three radicals AK2 is interrupted; preferably, the radicals AK2 are saturated; preferably, AK3 denotes a trivalent radical —CH₂—CH—CH₂—;
or
f) the three radicals AK2 represent a linear, saturated, C₁₂-C₂₄ monovalent hydrocarbon-based chain substituted with one or more identical or different radicals —O—C(O)-AK3-(O—C(O)—C(Rₐ)(R_{b})—C(O)—R₄)ᵥ, said chain also being optionally substituted with one or more identical or different radicals chosen from —OR and —S-AK3-(O—C(O)—C(Rₐ)(R_{b})—C(O)—R₄), and/or said chain being optionally interrupted with one or more heteroatoms or groups chosen from —O—, —S—, —C(O)—, (hetero)cycle(s) and combinations thereof, in particular —O—C(O)—, —C(O)—O— or -(hetero)cycle-O-(hetero)cycle-; AK3, v, R₄, Rₐ and R_{b} being as defined previously; preferably, Y denotes —O—C(O)—; it being understood that said compound contains at least two radicals —C(O)—C(Rₐ)(R_{b})—C(O)—R₄; preferably, Y represents —O—C(O)—; preferably, v is equal to 1 or 2, more preferentially equal to 1; preferably, the three chains AK2 comprise from 15 to 21 carbon atoms, more preferentially 17 carbon atoms; preferably, none of the three radicals AK2 is interrupted; preferably, the radicals AK2 are saturated; preferably, the radicals AK2 are substituted with at least one radical —OR, preferably at least one radical —O—C(O)—C(Rₐ)(R_{b})—C(O)—R₄; preferably, AK2 is not substituted with —S-AK₃-(O—C(O)—C(Rₐ)(R_{b})—C(O)—R₄); preferably, AK3 represents a linear or branched, saturated or unsaturated C₁-C₆, more preferentially saturated C₁-C₆, even more preferentially C₂, hydrocarbon-based chain.

Another subject of the invention is the compounds of formula (I), and also the optical or geometrical isomers thereof and/or the solvates thereof, such as the hydrates, chosen from the following compounds of formula (Ib), and also the optical or geometrical isomers thereof and/or the solvates thereof, such as the hydrates, and a composition, notably a cosmetic composition, in particular for treating keratin fibres, in particular for caring for, styling and/or colouring keratin fibres, preferably the hair, containing same:

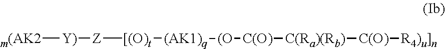

in which formula (Ib):
n+m is equal to 4, m being non-zero; m and n being as defined previously for the compounds of formula (I);
Z represents an unsubstituted and uninterrupted, linear or branched, saturated or unsaturated, preferably saturated and acyclic, C₂ to C₂₄, in particular C₃ to C₁₀ preferably C₃, tetravalent hydrocarbon-based radical;
AK2 independently represent a linear or branched, saturated or unsaturated, preferably saturated, C₁₂-C₂₄, preferably C₁₆-C₂₀, preferably C₁₈, monovalent hydrocarbon-based chain, optionally substituted with one or more identical or different radicals chosen from —OR and —X-AK3-(O—C(O)—C(Rₐ)(R_{b})—C(O)—R₄)ᵥ, preferably substituted with one or more radicals —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), and combinations thereof, in particular —O—C(O)—, —C(O)—O— or -(hetero)cycle-O-(hetero)cycle-, and preferably not being interrupted;
AK3 represents a linear or branched, saturated or unsaturated C₁-C₁₂, preferably C₁-C₆, more preferentially saturated C₁-C₆, even more preferentially C₂-C₄, preferably C₃, divalent hydrocarbon-based chain;
v is as defined previously; preferably, v is equal to 2;
X is as defined previously; preferably, X denotes a sulfur atom;
Y, AK1, t, q, u, R, R₄, Rₐ and R_{b} being as defined previously;
   it being understood that said compound contains at least two radicals —C(O)—C(Rₐ)(R_{b})—C(O)—R₄.

According to a preferred embodiment, m is equal to 3, n is equal to 1 and u is equal to 1.

According to a preferred embodiment, t is equal to 0 and q is equal to 0.

According to a preferred embodiment, Y denotes —C(O)—O—.

According to a preferred embodiment, none of the radicals AK2 are interrupted. Preferably, the radicals AK2 are saturated. Preferably, the radicals AK2 are not substituted with —OR.

According to a preferred embodiment, AK3 represents a linear or branched, saturated or unsaturated C₁-C₆, more preferentially saturated C₁-C₆, even more preferentially C₁-C₄, better still C₃, hydrocarbon-based chain.

According to this embodiment variant, the compounds of formula (I) or (Ib) are in particular chosen from the compounds (10) as defined previously, and also the optical or geometrical isomers thereof and/or the solvates thereof, such as hydrates.

Another subject of the invention is the compounds of formula (I), and also the optical or geometrical isomers thereof and/or the solvates thereof, such as the hydrates, chosen from the following compounds of formula (Ic), and also the optical or geometrical isomers thereof and/or the solvates thereof, such as the hydrates, and a composition, notably a cosmetic composition, in particular for treating keratin fibres, in particular for caring for, styling and/or colouring keratin fibres, preferably the hair, containing same:

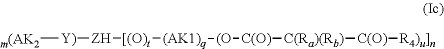

in which formula (Ic):
Z represents a linear or branched, saturated or unsaturated, preferably saturated and acyclic, multivalent C₁₄ to C₂₄, particularly C₁₆ to C₂₀, such as C₁₇, hydrocarbon-based radical, Z also being optionally substituted with one or more radicals —[(O)ₜ-(AK1)_{q}-(O—C(O)—C(Rₐ)(R_{b})—C(O)—R₄)ᵤ]n, preferably unsubstituted;
AK2 represents a linear or branched, saturated or unsaturated, preferably saturated, C₁₄-C₃₀, preferably C₂₀-C₂₉, monovalent hydrocarbon-based chain, substituted with one or more identical or different radicals chosen from —OR and —X-AK3-(O—C(O)—C(Rₐ)(R_{b})—C(O)—R₄)ᵥ, preferably substituted with one or more radicals —OR; and/or said chain being optionally interrupted with one or more heteroatoms or groups chosen from —O—, —S—, —C(O)—, (hetero)cycle(s), and combinations thereof, in particular —O—C(O)—, —C(O)—O—, -(hetero)cycle-O-(hetero)cycle-, and preferably interrupted with —O—C(O)— or —C(O)—O—;
X is as defined previously, and X preferably represents a sulfur atom;
Y, AK1, AK3, n, m, v, t, q, u, R, R₄, Rₐ and R_{b} being as defined previously;
   it being understood that said compound contains at least two radicals —C(O)—C(Rₐ)(R_{b})—C(O)—R₄.

According to a preferred embodiment, n is equal to 0 and m is equal to 1.

According to a preferred embodiment, Z is a divalent radical.

According to a preferred embodiment, if Z is an unsaturated chain, AK2 is an unsaturated chain, and if Z is a saturated chain, AK2 is a saturated chain.

According to this embodiment variant, the compound(s) of formula (Ic), and also the optical or geometrical isomers thereof and/or the solvates thereof, such as hydrates, are in particular of formula (Iic) as defined previously.

According to this embodiment variant, the compound(s) of formula (Iic) are in particular chosen from the compounds (12) as defined previously, and also the optical or geometrical isomers thereof and/or the solvates thereof, such as hydrates.

According to a preferred embodiment, the treatment process according to the invention comprises the application to keratin fibres, preferably the hair, of at least one or more compounds of formula (I) and also the optical and geometrical isomers thereof, and/or the solvates thereof, such as the hydrates, or a composition containing same, chosen from the following compounds of formula (Id), and also the optical or geometrical isomers thereof and/or the solvates thereof, such as the hydrates:

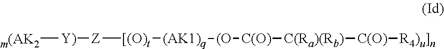

in which formula (Id):
n+m is equal to 4, and preferably m is equal to 3;
Z represents a linear or branched, saturated or unsaturated, preferably saturated or acyclic, C₂ to C₂₄, in particular C₃ to C₁₀, in particular C₄ or C₅, tetravalent hydrocarbon-based radical, Z also being substituted with one or more radicals —[(O)ₜ-(AK1)_{q}—(O—C(O)—C(Rₐ)(R_{b})—C(O)—R₄)ᵤ]ₙ, n preferably being equal to 1;
AK2 independently represents a linear or branched, saturated or unsaturated, preferably saturated, C₁₄-C₃₀, preferably C₁₆-C₂₄, preferably C₁₇, monovalent hydrocarbon-based chain, said chain being optionally substituted with one or more identical or different radicals chosen from —OR and —X-AK3-(O—C(O)—C(Rₐ)(R_{b})—C(O)—R₄)ᵥ, preferably substituted with one or more radicals —OR; and/or said chain being optionally interrupted with one or more heteroatoms or groups chosen from —O—, —S—, —C(O)—, (hetero)cycle(s), and combinations thereof, in particular —O—C(O)—, —C(O)—O— or -(hetero)cycle-O-(hetero)cycle-, and preferably not being interrupted; X is as defined previously and preferably X denotes a sulfur atom; Y, AK1, AK3, u, v, R, R₄, Rₐ and R_{b} being as defined previously,
   it being understood that these compounds contain at least two radicals —C(O)—C(Rₐ)(R_{b})—C(O)—R₄.

According to a preferred embodiment, t is equal to 0 and u is equal to 1.

According to a preferred embodiment, all the radicals AK2 are identical. Preferably, all the radicals Y are identical.

According to a preferred embodiment, Y denotes —O—C(O)—.

According to this embodiment variant, the compounds of formula (Id), and also the optical or geometrical isomers thereof and/or the solvates thereof, such as hydrates, are in particular of formula (Iid) as defined previously.

According to a preferred embodiment, the treatment process according to the invention comprises the application to keratin fibres, preferably the hair, of at least one or more compounds of formula (I) and also the optical and geometrical isomers thereof, and/or the solvates thereof, such as the hydrates, or a composition containing same, chosen from the following compounds of formula (Ie), and also the optical or geometrical isomers thereof and/or the solvates thereof, such as the hydrates:

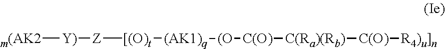

in which formula (Ie):
n+m is equal to 2, m being non-zero, and m preferably being equal to 2; n is as defined previously for the compounds of formula (I); preferably, n is zero;
Z represents a linear or branched, saturated or unsaturated, conjugated or non-conjugated, preferably non-conjugated, acyclic or cyclic, aromatic or non-aromatic, preferably non-aromatic, C₂ to C₂₄, in particular C₃ to C₁₂, divalent hydrocarbon-based radical, said radical Z also being interrupted with one or more heteroatoms or groups chosen from —O—, —N(Rₐ)—, —S—, —C(O)—, (hetero)cycle(s), and combinations thereof; preferably, Z denotes a divalent radical —(CH₂)_{g}-(heterocycle)-O—(CH₂)ₕ-(heterocycle)-(CH₂)ⱼ— in which h denotes 0, 1 or 2, preferably 0 or 1, g and j independently denote an integer between 1 and 3, preferably 1; (heterocycle) denotes a divalent heterocyclic radical (i.e. a heterocycle), preferably a heterocycloalkyl radical, more particularly a divalent 5- or 6-membered heterocycloalkyl radical, in particular a divalent tetrahydrofuran radical or a divalent tetrahydropyran radical, said heterocycle being optionally substituted with one or more radicals chosen from —OR with R as defined previously, and notably —OH or —O—C(O)—C(Rₐ)(R_{b})—C(O)—R₄;
AK2 represents a linear or branched, saturated or unsaturated, preferably saturated, C₁₄-C₃₀, preferably C₁₄-C₂₄, preferably C₁₇, monovalent hydrocarbon-based chain, said chain being optionally substituted with one or more identical or different radicals chosen from —OR and —X-AK3-(O—C(O)—C(Rₐ)(R_{b})—C(O)—R₄)ᵥ, preferably unsubstituted; and/or said chain being optionally interrupted with one or more heteroatoms or groups chosen from —O—, —S—, —C(O)—, (hetero)cycles, and combinations thereof, in particular —O—C(O)—, —C(O)—O— or -(hetero)cycle-O-(hetero)cycle-, preferably optionally interrupted with —O—C(O)— or —C(O)—O—, and more preferentially not being interrupted;
X is as defined previously; preferably, X denotes a sulfur atom;
Y, AK1, AK3, v, t, q, u, R, R₄, Rₐ and R_{b} being as defined previously;
   it being understood that said compound contains at least two radicals —C(O)—C(Rₐ)(R_{b})—C(O)—R₄.

According to this embodiment variant, the compounds of formula (Ie), and also the optical or geometrical isomers thereof and/or the solvates thereof, such as hydrates, are in particular of formula (Iie) as defined previously.

Another subject of the invention is the compounds of formula (I) as described previously, and also the salts, isomers and/or solvates thereof, such as hydrates, different from compounds (1) to (7) as described previously.

Another subject of the invention is the compounds of formulae (Ia), (Ib), (Ic), (Id), and (Ie) as described previously, and also the salts, isomers and/or solvates thereof such as the hydrates, more particularly the compounds of formulae (Iic), (Iid), and (Iie) as described previously.

In particular, another subject of the invention is compounds (8), (9), (10), (11), (12), (13), and (14) as described previously, and also the salts, isomers and solvates thereof.

### Preparation of the Compounds of Formula (I)

In particular, the compound(s) of formula (I), or (Ia), (Ib), (Ic), (Id), (Ie), (Iic), (Iid) or (Iie), may be prepared via the following sequence of reactions:

### First Step

In a first step, a commercial polyol, which is preferably natural, of formula (H)ₚ—Z(OH)_{m′}, is reacted with m equivalents of fatty derivatives R′CO-AK′₂ with m′ greater than or equal to m, and m being as defined previously, under conditions known to those skilled in the art, to form an ester bond Y according to the scheme below:

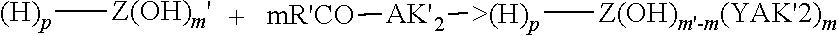

in which scheme:
Y represents —O—C(O)—;
R′ denoting in particular —OH or —Cl; preferably, R′ denotes —OH;
AK′2 represents a linear or branched, unsaturated, C₁-C₄₀, preferably C₁₄-C₂₀, hydrocarbon-based chain, said chain being optionally interrupted with one or more heteroatoms or groups chosen from —O—, —S—, —C(O)—, (hetero)cycle(s), and combinations thereof, in particular —O—C(O)—, —C(O)—O— or -(hetero)cycle-O-(hetero)cycle-, preferably interrupted with —O— or —S—, more preferentially not being interrupted.

According to a preferred embodiment, m is equal to m′.

The polyols (H)ₚ—Z(OH)_{m′} may be cyclic or acyclic.

By way of example of cyclic polyols (H)ₚ—Z(OH)_{m′}, mention may be made of maltose, lactose, raffinose, sucrose, trehalose, melibiulose, melibiose, mannobiose, kojibiose, nigerose, isomaltose, rutinose, rutinulose, xylobiose or sorbitan.

As examples of acyclic polyols (H)ₚ—Z(OH)_{m′}, mention may be made of ethylene glycol, propylene glycol, butylene glycol, pentylene glycol, hexylene glycol, butanediol, glycerol, pentaerythritol, dipentaerythritol, trimethylolethane, mannitol or trimethylolpropane.

According to a preferred embodiment, the polyol (H)ₚ—Z(OH)_{m′} is chosen from glycerol, mannitol, sucrose, ethylene glycol, sorbitan or trimethylolpropane, and more preferentially the polyol (H)ₚ—Z(OH)_{m′} denotes glycerol.

The compounds R′CO-AK′₂ are preferably unsaturated fatty acids containing from 15 to 36 carbon atoms, preferably from 15 to 24 carbon atoms. They may be mono- or polyunsaturated.

The monounsaturated fatty acids are in particular chosen from palmitoleic acid, oleic acid, 9-eicosenoic acid, 11-eicosenoic acid, erucic acid and nervonic acid.

The polyunsaturated fatty acids are notably chosen from linoleic acid, α-linolenic acid, γ-linolenic acid, dihomo-γ-linolenic acid, arachidonic acid, eicosapentaenoic acid, docosahexaenoic acid, ruminic acid and hydroxyl monounsaturated fatty acids notably chosen from 10-hydroxy-cis-12-octadecenoic acid, 10-hydroxy-trans-11-octadecenoic acid, 12-hydroxy-cis-9-octadecenoic acid, 13-hydroxy-cis-9-octadecenoic acid, ricinoleic acid, isoricinoleic acid, and polyunsaturated hydroxyl fatty acids notably chosen from densipolic acid, auricolic acid, dimorphecolic acid.

More preferentially, the unsaturated fatty acid is chosen from ricinoleic acid, oleic acid, palmitoleic acid, erucic acid and mixtures thereof.

### Second Step

In a second step, all or some of the unsaturation(s) of the radical(s) AK′2 are converted a) via an epoxidation reaction into epoxide to yield the derivatives (III), such as the starting compound al illustrated in the scheme described below, or b) via a thiol-ene reaction with a (poly)hydroxylated thiol or a thiol bearing acid function(s) to yield the derivatives (IV), for instance compound b3 illustrated in the scheme described below.

The thiol-ene reaction b) may be performed using (poly)hydroxylated thiols such as linear hydroxylated thiols of formula HS—(CH₂)ₖ—OH with k being an integer from 1 to 12, preferably from 1 to 6, such as 2-mercaptoethanol, 3-mercapto-1-propanol, 6-mercapto-1-hexanol, 4-mercapto-1-butanol, or such as branched hydroxylated thiols preferably including from 1 to 6 carbon atoms, such as 3-mercapto-3-methylbutanol, 3-mercapto-2-butanol, or such as polyhydroxylated thiols, preferably including from 1 to 6 carbon atoms, such as 3-mercapto-1,2-propanediol HS—CH(OH)—CH₂OH.

The compounds (IV) obtained are thus substituted with a radical —S-AK3-(OH), with v being equal to 1 if the thiol is hydroxylated and v being greater than 1, such as 2, if the thiol is polyhydroxylated, AK3 and v being as defined previously for the compounds of formula (I).

### Third Step

In a third step, all or some of the epoxide groups of the compounds of formula (III) are opened with nucleophilic compounds, in particular i) alcohols preferably of C₁-C₄ such as methanol or ethanol, or even a polyol to give compounds (IIIi), such as compound b2 illustrated in the scheme hereinbelow, ii) carboxylic acids, preferably of C₁-C₆, such as lactic acid, pyruvic acid, benzoic acid, glycolic acid or 2,2-bishydroxymethylpropionic acid, more preferentially carboxylic acids substituted with at least one hydroxyl radical such as lactic acid, to yield the compounds (IIIii), such as compound b1 illustrated in the scheme hereinbelow.

According to a preferred embodiment, the carboxylic acid ii) is chosen from lactic acid, glycolic acid or 2,2-bishydroxymethylpropionic acid (epoxy-acid reaction) and the reaction is performed in the presence of a catalyst such as imidazole, 1-methylimidazole, N,N-dimethylbenzylamine or caffeine.

According to a preferred embodiment, the alcohol i) is chosen from ethylene glycol, propylene glycol, butylene glycol, pentylene glycol, hexylene glycol, butanediol, glycerol, pentaerythritol, dipentaerythritol, trimethylolethane and trimethylolpropane, more preferentially butylene glycol or trimethylolethane.

### Fourth Step

In a fourth step, all or some of the alcohol functions —OH of the compounds (IIIi) or (IIIii) and all or some of the alcohol functions which may possibly still be present originating from the polyols (H)ₚ—Z(OH)_{m′} used in the first step are converted into radicals —O—C(O)—C(Rₐ)(R_{b})—C(O)—R₄, for example via a (trans)esterification reaction with an ester Gp-C(O)—C(Rₐ)(R_{b})—C(O)—R₄, in which Gp preferably denotes a hydroxyl radical or a (C₁-C₄)alkoxy radical, such as methoxy, ethoxy, isobutoxy or t-butoxy.

According to a preferred embodiment, Gp-C(O)—C(Rₐ)(R_{b})—C(O)—R₄ denotes an ester chosen from ethyl acetoacetate, ethyl 2-methylacetoacetate and tert-butyl acetoacetate, preferably tert-butyl acetoacetate.

The compounds of structures (Ia), (Ic), such as the compounds of structure (Iic), and the compounds of structures (Id) and (Ie) may be obtained according to this process.

By way of example, step 4 may be performed according to the protocol described in Trevino, A. S., and Trumbo, D. L. (2002). Acetoacetylated castor oil in coating applications. Progress in Organic Coatings, 44(1), 49-54:

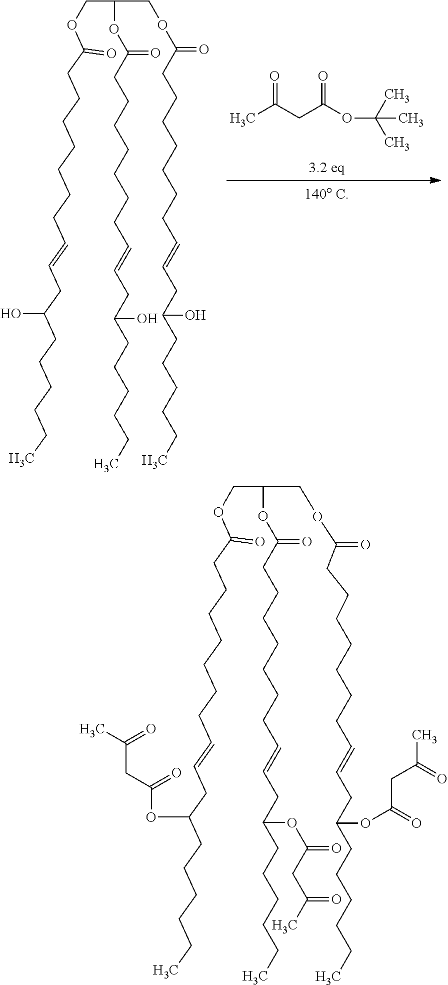 [SMILES: CCCCCCC(O)C/C=C/CCCCCCCC(=O)OCC(COC(=O)CCCCCCC/C=C/CC(O)CCCCCC)OC(=O)CCCCCCC/C=C/CC(O)CCCCCC ; CC(=O)CC(=O)OC(C)(C)C ; CCCCCCC(C/C=C/CCCCCCCC(=O)OCC(COC(=O)CCCCCCC/C=C/CC(CCCCCC)OC(=O)CC(C)=O)OC(=O)CCCCCCC/C=C/CC(CCCCCC)OC(=O)CC(C)=O)OC(=O)CC(C)=O]

In certain cases, the compounds (H)ₚ—Z(OH)_{m′-m} (YAK′₂)ₘ resulting from the first step are commercial; in particular, certain compounds for which m′ is equal to m, p is equal to 0, Z preferably denoting —CH₂—CH—CH₂—, are commercial. When p is equal to 0 and m′ is equal to m, the compounds are esters of optionally hydroxylated unsaturated fatty acids, in particular triglycerides of castor oil, soybean oil, canola oil or linseed oil. The synthesis of compounds (Ia) to (Ie) then begins from the second step described above.

For example, starting with an unsaturated triglyceride, a thiol-ene reaction is performed with a (poly)hydroxyl thiol such as SH—(CH₂)ₓOH with x being an integer preferably from 1 to 4, and then a transesterification reaction (as described in Desroches, M., Caillol, S., Lapinte, V., Auvergne, R. and Boutevin, B. (2011). Synthesis of biobased polyols by thiol-ene coupling from vegetable oils. Macromolecules, 44 (8), 2489-2500:

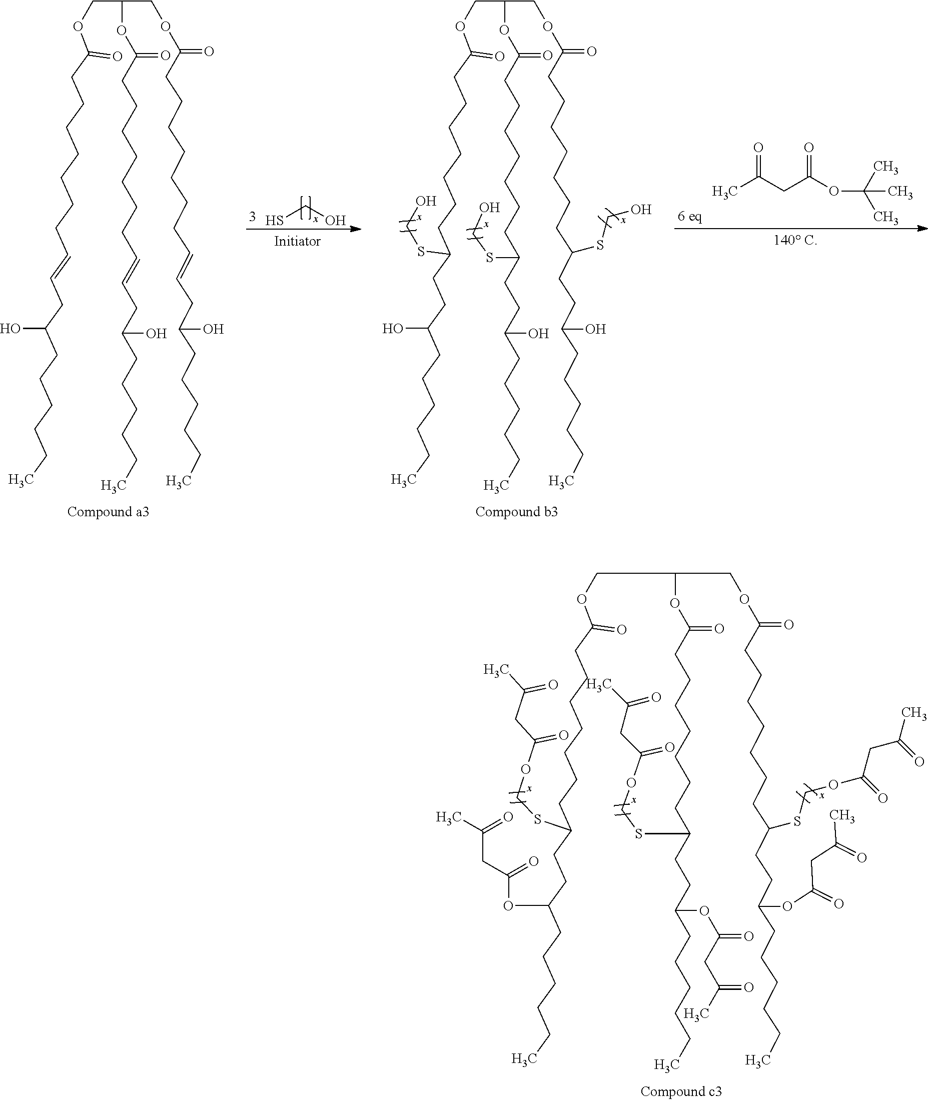

For example, starting with an unsaturated triglyceride, a total or partial epoxidation reaction is performed, followed by opening of the epoxide(s) formed, which generates alcohol functions and (trans)esterification. A commercial epoxidized triglyceride, such as the products sold by Arkema under the trade names Vikoflex 7170 or Vikoflex 7190, may also be used as starting material.

By way of example, the reaction scheme below may be used, in which all the epoxides are opened with lactic acid and each hydroxyl is then esterified with t-Bu-OCOCH₂CH₃, according to the scheme below:

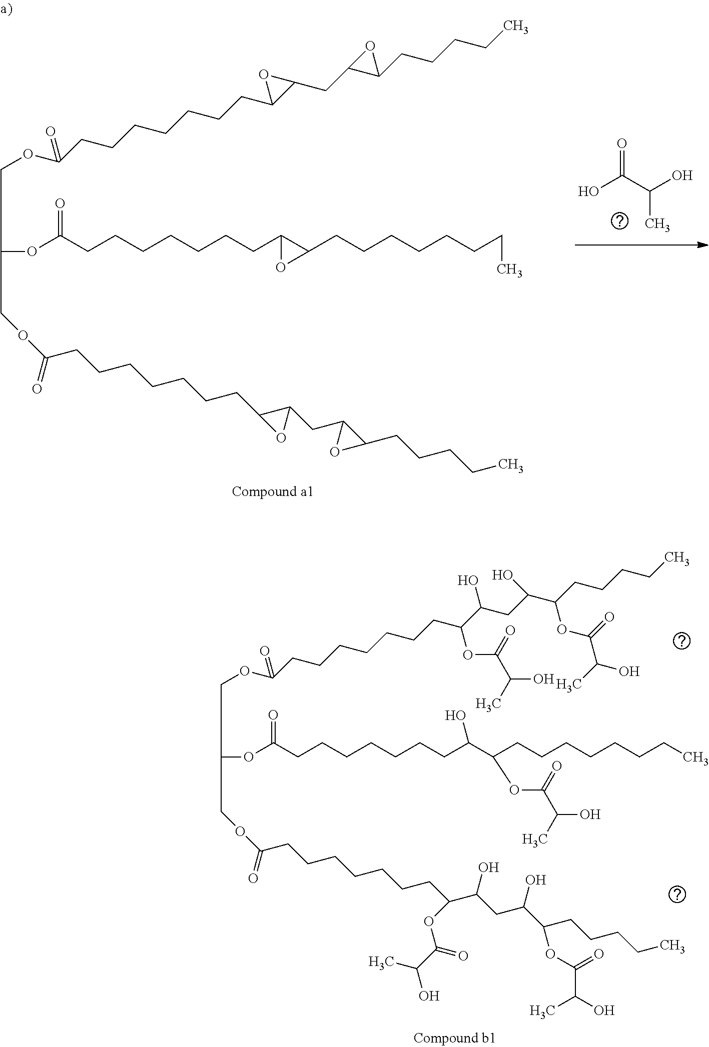

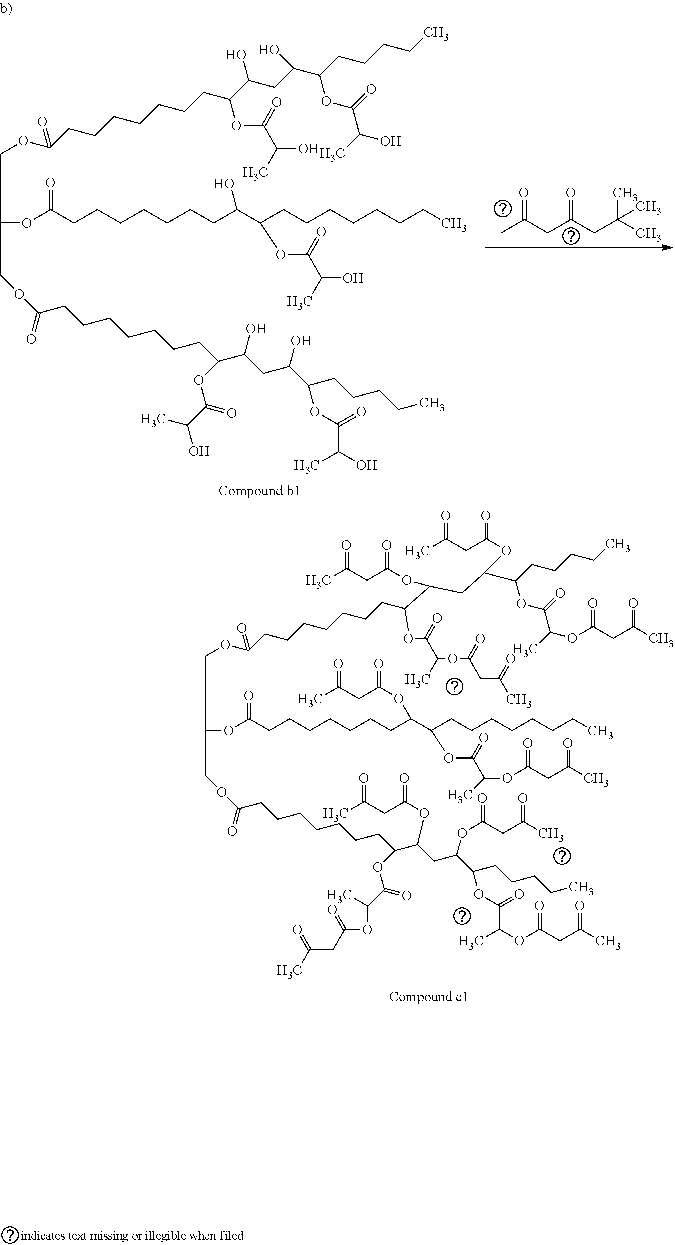 [SMILES: CCCCCCCCC(OC(=O)C(C)O)C(O)CCCCCCCC(=O)OC(COC(=O)CCCCCCCC(OC(=O)C(C)O)C(O)CC(O)C(CCCCC)OC(=O)C(C)O)COC(=O)CCCCCCCC(OC(=O)C(C)O)C(O)CC(O)C(CCCCC)OC(=O)C(C)O ; CC(=O)CC(=O)CC(C)(C)C ; CCCCCCCCC(OC(=O)C(C)OC(=O)CC(C)=O)C(CCCCCCCC(=O)OC(COC(=O)CCCCCCCC(OC(=O)C(C)OC(=O)CC(C)=O)C(CC(OC(=O)CC(C)=O)C(CCCCC)OC(=O)C(C)OC(=O)CC(C)=O)OC(=O)CC(C)=O)COC(=O)CCCCCCCC(OC(=O)C(C)OC(=O)CC(C)=O)C(CC(OC(=O)CC(C)=O)C(CCCCC)OC(=O)C(C)OC(=O)CC(C)=O)OC(=O)CC(C)=O)OC(=O)CC(C)=O]

By way of example, the reaction scheme below may also be used, in which all the epoxides are opened with an alcohol, methanol in the example, and each hydroxyl is then esterified with t-Bu-OCOCH₂CH₃:

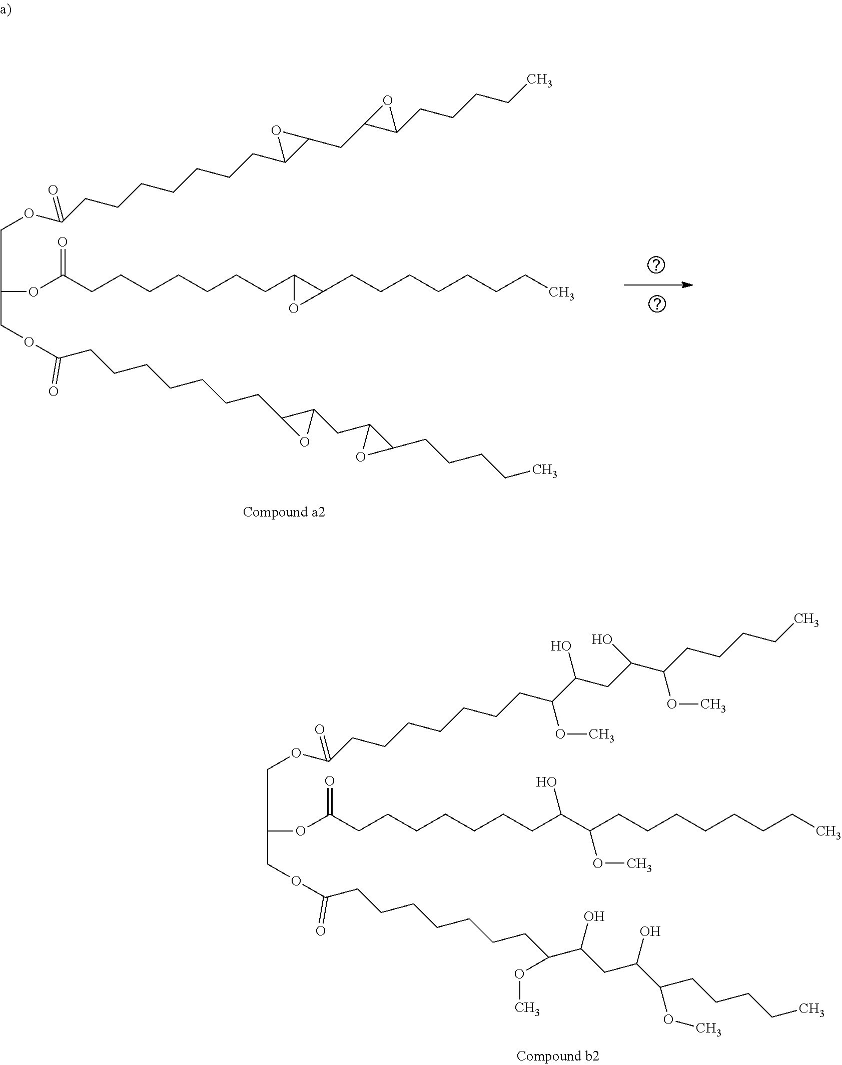 [SMILES: CCCCCCCCC1OC1CCCCCCCC(=O)OC(COC(=O)CCCCCCCC1OC1CC1OC1CCCCC)COC(=O)CCCCCCCC1OC1CC1OC1CCCCC ; CCCCCCCCC(OC)C(O)CCCCCCCC(=O)OC(COC(=O)CCCCCCCC(OC)C(O)CC(O)C(CCCCC)OC)COC(=O)CCCCCCCC(OC)C(O)CC(O)C(CCCCC)OC]

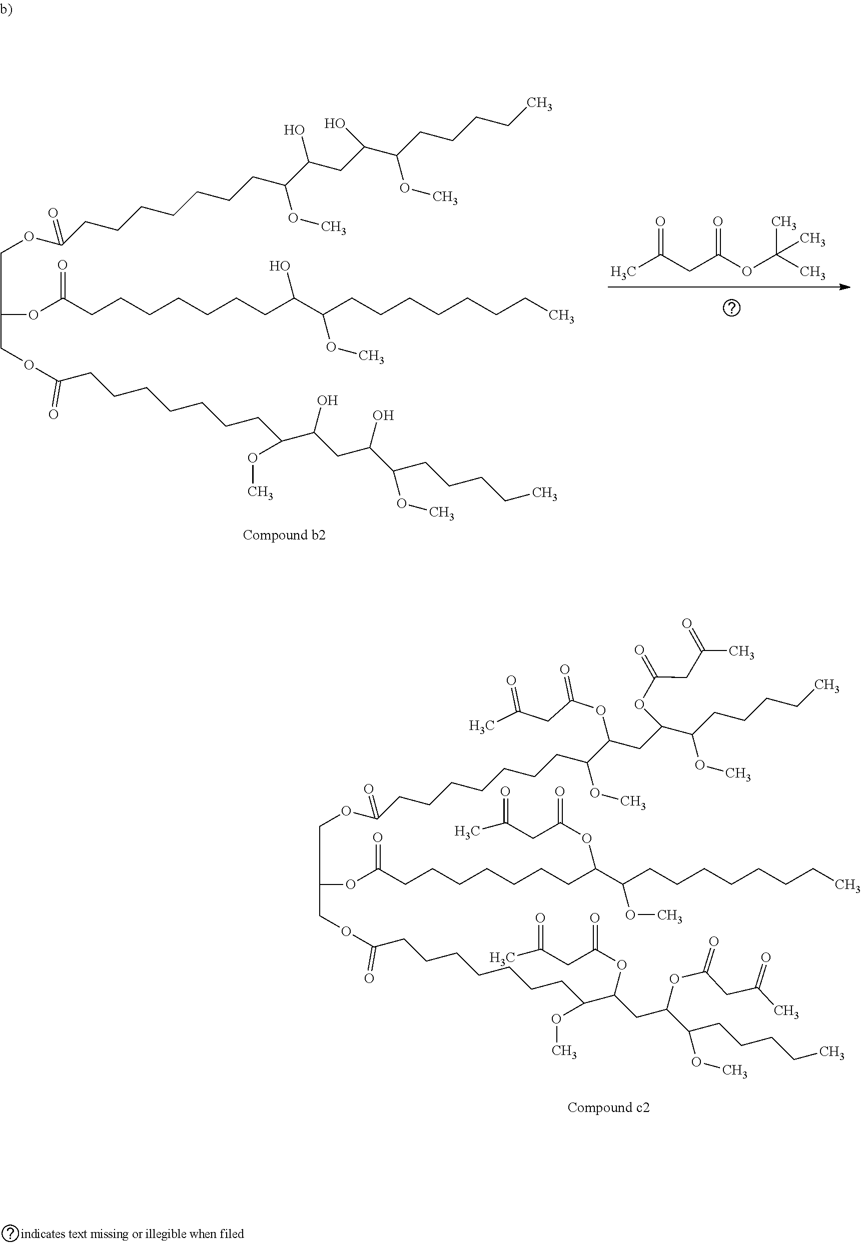 [SMILES: CCCCCCCCC(OC)C(O)CCCCCCCC(=O)OC(COC(=O)CCCCCCCC(OC)C(O)CC(O)C(CCCCC)OC)COC(=O)CCCCCCCC(OC)C(O)CC(O)C(CCCCC)OC ; CC(=O)CC(=O)OC(C)(C)C ; CCCCCCCCC(OC)C(CCCCCCCC(=O)OC(COC(=O)CCCCCCCC(OC)C(CC(OC(=O)CC(C)=O)C(CCCCC)OC)OC(=O)CC(C)=O)COC(=O)CCCCCCCC(OC)C(CC(OC(=O)CC(C)=O)C(CCCCC)OC)OC(=O)CC(C)=O)OC(=O)CC(C)=O]

The compounds of formulae (Ia) to (Ie) may also be prepared via the following sequence of reactions.
In a first step, a preferably natural commercial polyacid (H)ₚ—Z(COOH)_{m′} is reacted with m equivalents of fatty alcohol HO-AK′2 with m′ greater than or equal to m, and m being as defined previously, under conditions known to those skilled in the art to form an ester bond Y according to the scheme below.

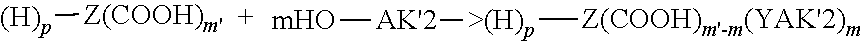

in which Y denotes —C(O)—O—, and AK′2 represents a monovalent linear or branched, unsaturated, C₁-C₄₀, preferably C₁₄-C₂₀, hydrocarbon-based chain, said chain optionally being interrupted with one or more heteroatoms or groups chosen from —O—, —S—, —C(O)—, (hetero)cycle(s), and combinations thereof, in particular —O—C(O)—, —C(O)—O—, -(hetero)cycle-O-(hetero)cycle-, preferably interrupted with —O— or —S—, and more preferentially not being interrupted.

According to a preferred embodiment, m is equal to m′.

The polyacids (H)ₚ—Z(COOH)_{m′} may be cyclic or acyclic.

The polyacids may preferably denote optionally hydroxylated dicarboxylic acids, optionally hydroxylated tricarboxylic acids, or optionally hydroxylated tetracarboxylic acids.

The non-hydroxylated dicarboxylic acids are notably chosen from maleic acid, propanedioic acid, butanedioic acid, pentanedioic acid, hexanedioic acid, decanedioic acid, dodecanedioic acid, cyclopropanedicarboxylic acid, cyclohexanedicarboxylic acid, cyclobutanedicarboxylic acid, naphthalene-1,4-dicarboxylic acid, naphthalene-2,3-dicarboxylic acid, naphthalene-2,6-dicarboxylic acid, suberic acid, oxalic acid, malonic acid, succinic acid, phthalic acid, terephthalic acid, isophthalic acid, tetrahydrophthalic acid, hexahydrophthalic acid, pimelic acid, sebacic acid, azelaic acid, glutaric acid, adipic acid, fumaric acid and itaconic acid, and preferably propanedioic acid, butanedioic acid and pentanedioic acid.

The hydroxylated dicarboxylic acids are notably chosen from tartaric acid, malic acid, aleuritic acid, 7,18-dihydroxytetracos-15-enoic acid and avenic acid B, and preferably malic acid and tartaric acid.

The (hydroxy)tricarboxylic acids are notably chosen from 2-oxaloglutaric acid, oxalosuccinic acid, 3-carboxymethylmuconic acid, 2-methylcitric acid, citric acid, isocitric acid, aconitic acid, transaconitic acid, 1,2,3-propanetricarboxylic acid, 1,2,5-pentanetricarboxylic acid, 1,3,5-benzenetricarboxylic acid, 1,3,5-cyclohexanetricarboxylic acid, 3-butene-1,2,3-tricarboxylic acid, 3-butene-1,1,3-tricarboxylic acid, trimellitic acid, 1,2,3-benzenetricarboxylic acid, 1,3,5-benzenetricarboxylic acid and agaric acid, preferably citric acid and aconitic acid.

The tetracarboxylic acids are notably chosen from 1,2,3,4-butanetetracarboxylic acid, pyromellitic acid, oxydisuccinic acid, thiodisuccinic acid, N-[1,2-dicarboxyethyl]-L-aspartic acid, ethylenediaminetetraacetic acid, ethylenediaminetetrapropionic acid and N,N′-ethylenebis(L-aspartic acid).

According to a preferred embodiment, the fatty alcohols HO-AK′2 are chosen from fatty alcohols comprising from 15 to 36 carbon atoms, preferably from 15 to 24 carbon atoms.

The fatty alcohols HO-AK′2 may be mono- or polyunsaturated (presence of one or more ethylenic double bonds).

The fatty alcohols HO-AK′2 may contain several alcohol functions.

The monounsaturated fatty alcohols are notably chosen from oleyl alcohol, docosenol, or unsaturated fatty alcohol ethers, such as polyoxyethylene monooleyl ether, preferably oleyl alcohol. The unsaturated monounsaturated fatty alcohols containing several alcohol functions are notably chosen from 9-octadecene-1,12-diol.

The polyunsaturated fatty alcohols are notably chosen from linoleic alcohol, 2,4-dodecadien-1-ol and dolichols (with n being greater than or equal to 3 and less than or equal to 5).

According to a preferred embodiment, the fatty alcohol HO-AK′2 is chosen from oleyl alcohol, linoleyl alcohol or mixtures thereof.

The following steps are identical to those described previously:
In a second step, all or some of the unsaturation(s) of the radical(s) AK′2 are converted a) via an epoxidation reaction into epoxide to yield derivatives (III′), such as compound a4 illustrated in the scheme below, or b) via a thiol-ene reaction with a (poly)hydroxylated thiol or a thiol bearing acid function(s) to yield derivatives (IV′), such as compound b5 illustrated in the scheme below.
In a third step, all or some of the epoxide groups of the compounds of formula (III′) are opened with nucleophilic compounds, such as i) preferably C₁-C₄ alcohols such as methanol or ethanol or even a polyol to yield compounds (III′i), such as compound b4 illustrated in the scheme below, ii) carboxylic acids preferably of C₁-C₆, such as lactic acid, pyruvic acid or benzoic acid, more preferentially carboxylic acids substituted with at least one hydroxyl radical, such as lactic acid, to yield compounds (III′ii), such as compound b6 illustrated in the scheme below.
In a fourth step, all or some of the alcohol functions —OH of the compounds (III′i) or (III′ii) and all or some of the alcohol functions which may still be present and which are derived from the polyols (H)ₚ—Z(OH)ₘ, used in the first step are converted into radicals —O—C(O)—C(Rₐ)(R_{b})—C(O)—R₄, for example via a transesterification reaction with an ester Gp-C(O)—C(Rₐ)(R_{b})—C(O)—R₄ in which Gp preferably denotes a hindered alkoxy radical such as tBu-O—, to give the corresponding compounds of formula (I).

The compounds of formulae (Ia) to (Ie) such that p is equal to 1, Y denotes —C(O)—O— and m is equal to 1 may also be prepared by combining steps 2, 3 and 4 described previously, from an unsaturated fatty acid such as oleic acid as described in “The synthesis of Bio-based Michael donors from tall oil fatty acids for polymer development”, Polymers, 2022, 14, 4107.

By way of example, the reaction scheme below may be used, in which all the epoxides are opened with methanol and each hydroxyl is then esterified with t-Bu-OCOCH₂CH₃:

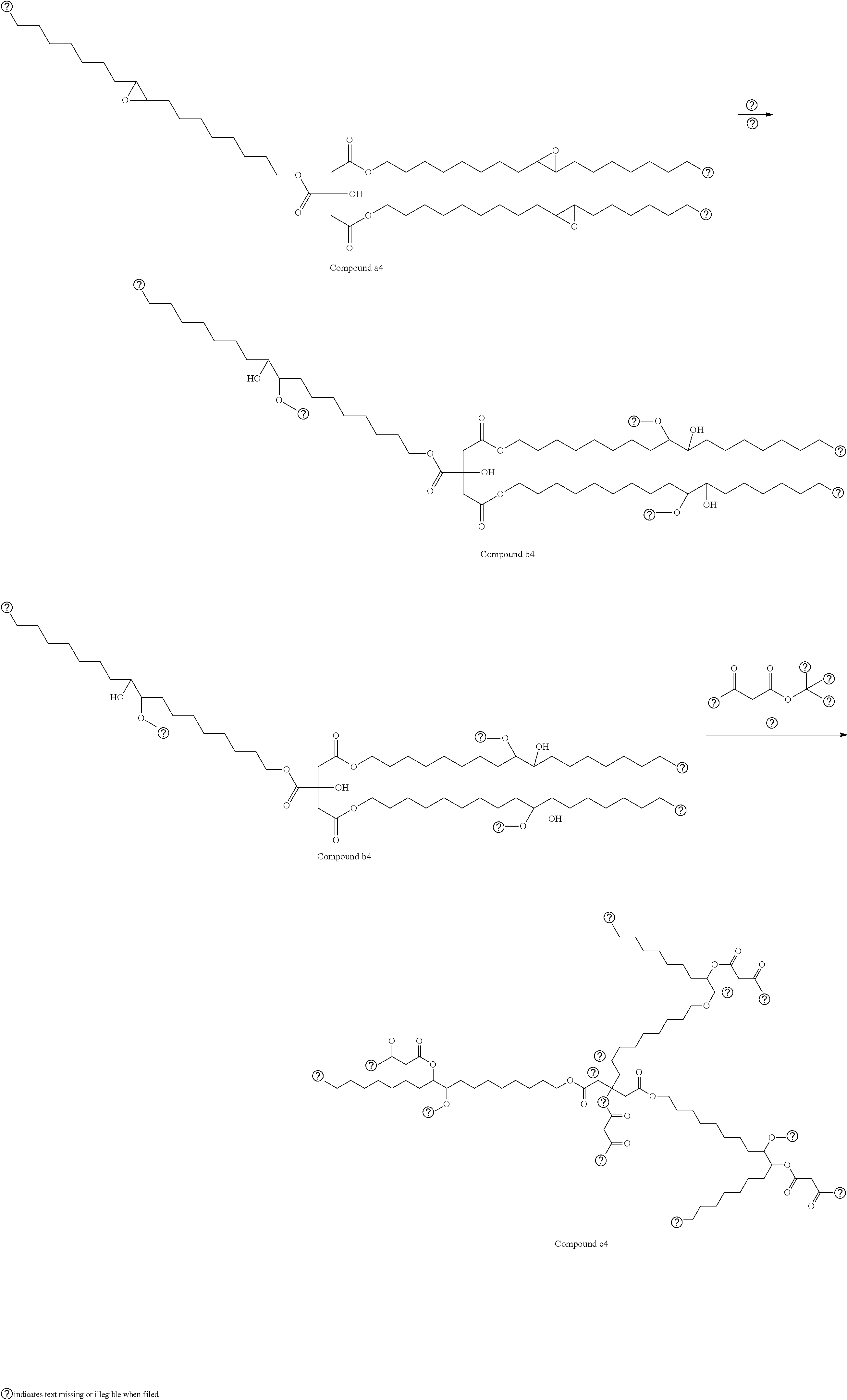

By way of example, the reaction scheme below may be used, in which an unsaturated oil reacts with a (poly)hydroxyl thiol such as SH—(CH₂)ₓOH, with x being an integer preferably from 1 to 4, followed by a transesterification reaction (as described in Desroches, M., Caillol, S., Lapinte, V., Auvergne, R. and Boutevin, B. (2011). Synthesis of biobased polyols by thiol-ene coupling from vegetable oils. Macromolecules, 44(8), 2489-2500).

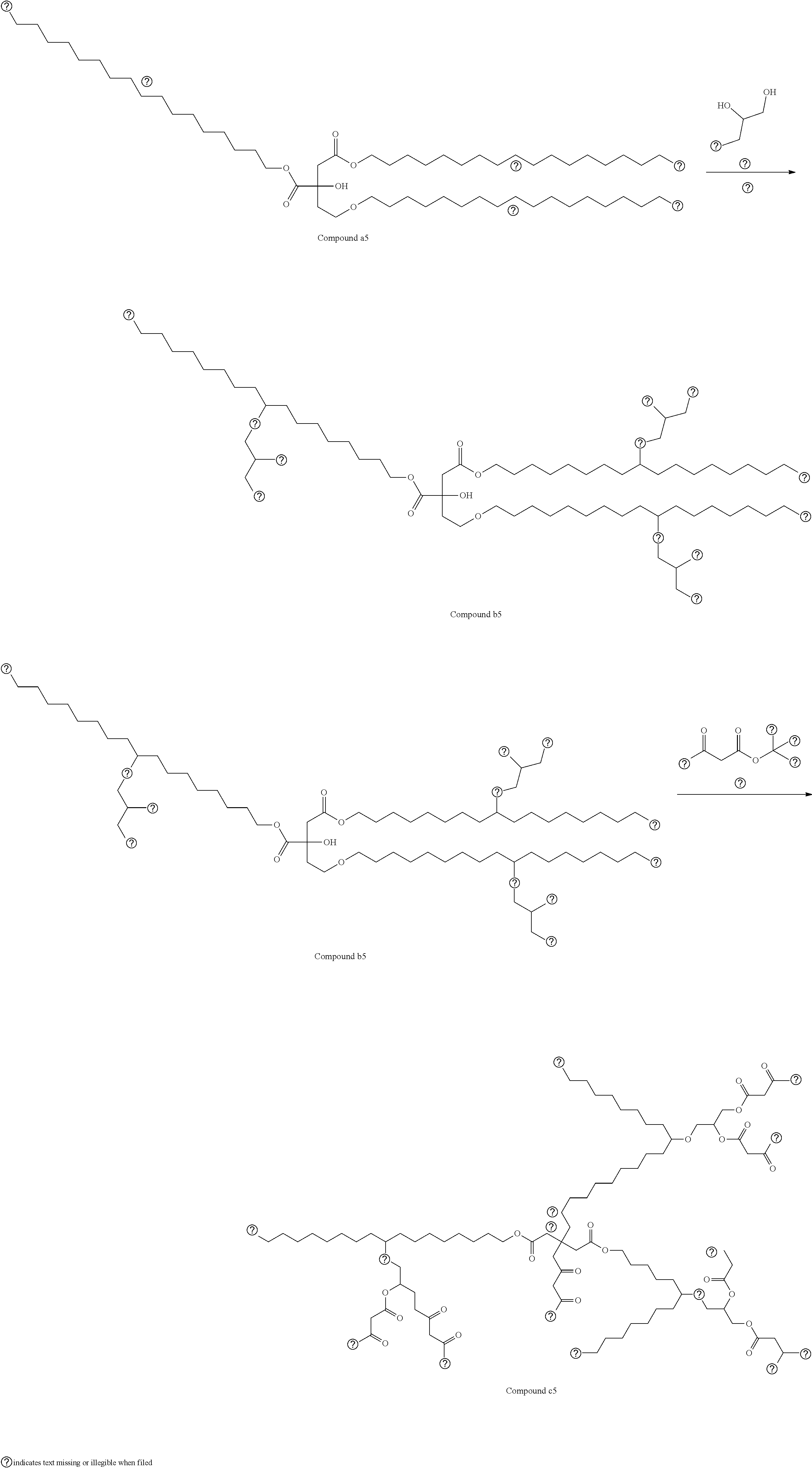

By way of example, the reaction scheme below may be used, in which all the epoxides are opened with lactic acid and each hydroxyl is then esterified with t-Bu-OCOCH₂CH₃:

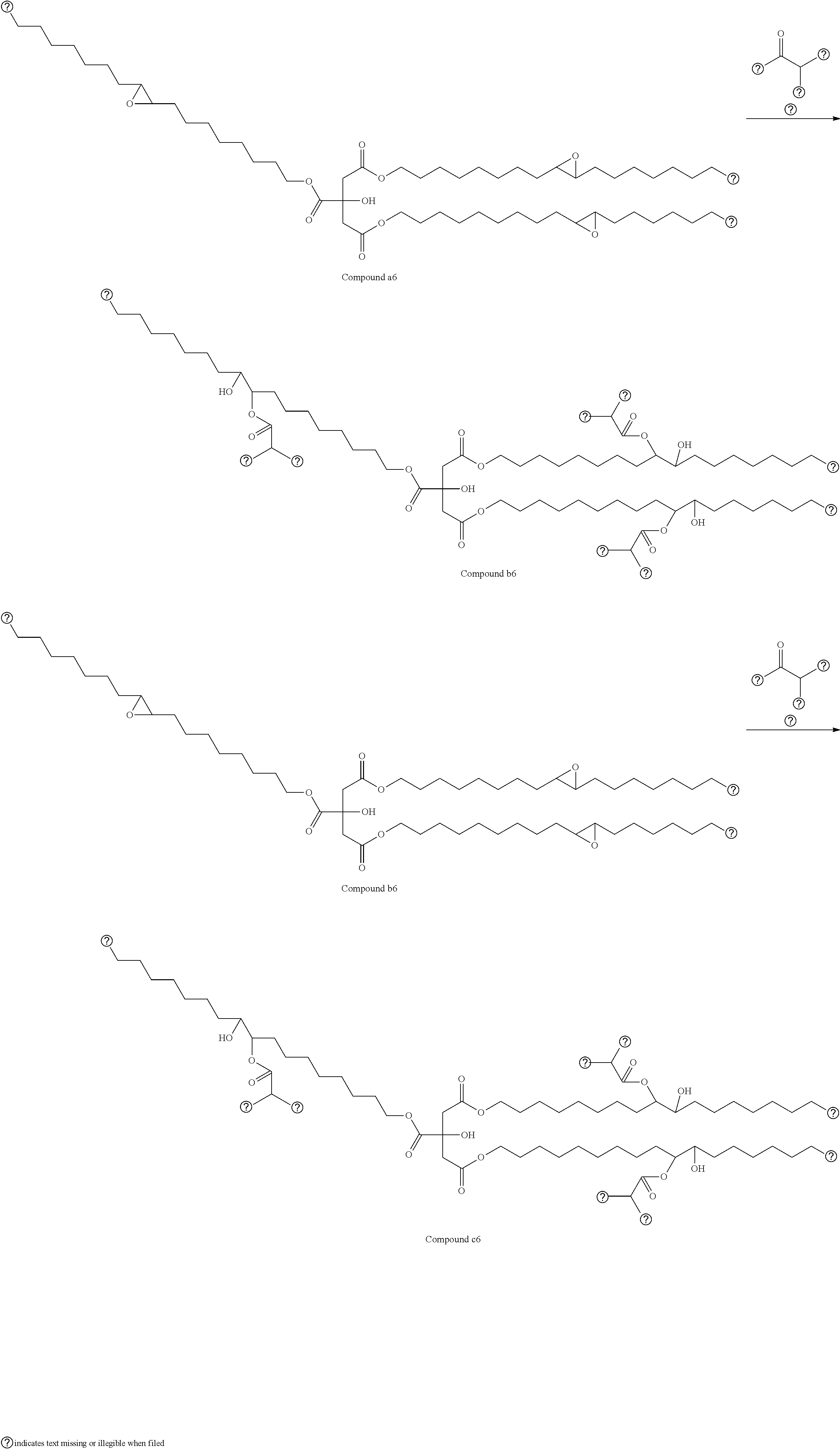

### Crosslinking Agent ii)

According to a particular embodiment, the treatment process according to the invention as described previously comprises the application to keratin fibres, preferably the hair, of (i) at least one compound of formula (I) as defined previously, and (ii) at least one crosslinking agent.

For the purposes of the invention, the term “crosslinking agent”, also termed “R”, denotes a compound that is capable of establishing with at least one acetoacetate function of the compound(s) of formula (I) used in the treatment process according to the invention:
at least one covalent bond,
at least one donor-acceptor (dative) bond, and/or
at least one coordination bond,
   and thus of crosslinking this or these compounds.

Preferably, the term “crosslinking agent”, also termed “R”, refers to a compound that is capable of establishing at least one covalent bond with an acetoacetate function of the compound(s) of formula (I) used in the treatment process according to the invention and thus of crosslinking this or these compounds.

For the purposes of the present invention, it is understood that the terms “crosslinking agent” and “crosslinker” are equivalent.

Compositions “C2”, “C3” and “C4” as defined previously contain at least ii) a crosslinking agent. The compositions of the invention may comprise a fatty phase, an aqueous phase or may be in the form of a direct or inverse emulsion. Composition “C4” may be an aqueous composition.

According to one aspect, the treatment process of the invention uses a composition termed “C3”, in particular a cosmetic composition for treating keratin fibres, in particular for caring for, styling and/or colouring keratin fibres, preferably the hair, i) at least one compound of formula (I), as defined previously and hereinbelow, ii) at least one crosslinking agent, in particular as defined previously and hereinbelow, and optionally iii) at least one cosmetic active agent, in particular as defined previously and hereinbelow.

The crosslinking agent(s) ii) are preferably present in a mass content ranging from 0.2% to 60% by weight, in particular ranging from 0.5% to 40% by weight, more particularly from 1% to 35% by weight, even more particularly from 1% to 30% relative to the total weight of the composition containing same.

In particular, in composition “C2” or “C3”, the crosslinking agent(s) ii) and the compound(s) i) as defined previously are preferably present in a mass content ranging from 1% to 35% by weight, relative to the total weight of the composition comprising them.

When the treatment process involves several steps including a step of using one or more compounds of formula (I) or (Ia), (Ib), (Ic), (Id), (Ie), (Iic), (Iid) and/or (Iie) without crosslinking agent, the compound(s) of formula (I), (Ia), (Ib), (Ic), (Id), (Ie), (Iic), (Iid) or (Iie) may be used on their own (pure), or in a composition “C1” as described previously.

More precisely, the crosslinking agent(s) ii) that are suitable for use in the invention may be chosen from compounds bearing amine, thiol, acrylate and/or carbonyl functions, such as a ketone or aldehyde function. A crosslinking agent R may also denote a metal alkoxide or a metal salt or a rare-earth metal derivative.

Thus, according to a particular embodiment, the crosslinking agent ii) is chosen from (poly)amine, (poly)thiol, (poly)carbonyl, (poly)acrylate, metal alkoxide and metal (poly)(hydroxy)(C₁-C₆)alkylcarboxylate compounds and/or rare-earth metal derivatives and mixtures thereof, and preferably chosen from (poly)amine, (poly)carbonyl, (poly)acrylate and metal alkoxide compounds, and mixtures thereof.

The term “(poly)amine, (poly)thiol, (poly)carbonyl and (poly)acrylate compounds” is intended to denote compounds including at least one primary or secondary amine, thiol, carbonyl (such as a ketone or aldehyde function) or acrylate function, respectively.

The metal alkoxide compounds, metal (poly)(hydroxy)(C₁-C₆)alkylcarboxylates and rare-earth metal derivatives are defined below.

### A) (Poly)amine Compounds

According to a preferred embodiment, the crosslinking agent R is chosen from (poly)amine compounds.

The (poly)amine compound may be chosen in particular from polyamine compounds bearing several primary and/or secondary amine groups or from amino alkoxysilanes, and more particularly from amino alkoxysilane compounds, diamine compounds, triamine compounds, and mixtures thereof.

The (poly)amine compound may be a compound comprising from 2 to 20 carbon atoms, notably a nonpolymeric compound; they may be acyclic or cyclic, linear or branched, saturated or unsaturated, conjugated or non-conjugated, aromatic or non-aromatic, optionally interrupted with one or more heteroatoms or groups chosen from O, S, Si(R′)₂, N(R″) preferably O, Si(R′)₂, or combinations thereof such as —Si(R′)₂—O— or —O—Si(R′)₂—, with R′, which may be identical or different, representing a (C₁-C₄)alkyl group such as methyl, and R″ representing a hydrogen atom or a (C₁-C₄)alkyl group, preferably a hydrogen atom.

The term “non-polymeric compound” means a compound which is not directly obtained via a monomer polymerization reaction.

(Poly)amine compounds that may be mentioned in particular include N-methyl-1,3-diaminopropane, N-propyl-1,3-diaminopropane, N-isopropyl-1,3-diaminopropane, N-cyclohexyl-1,3-diaminopropane, 2-(3-aminopropylamino)ethanol, 3-(2-aminoethyl)aminopropylamine, bis(3-aminopropyl)amine, methylbis(3-aminopropyl)amine, N-(3-aminopropyl)-1,4-diaminobutane, N,N-dimethyldipropylenetriamine, 1,2-bis(3-aminopropylamino)ethane, N,N′-bis(3-aminopropyl)-1,3-propanediamine, ethylenediamine, 1,3-propylenediamine, 1,4-butylenediamine, lysine, cystamine, xylenediamine, tris(2-aminoethyl)amine, 1,3-bis(aminomethyl)cyclohexane, 1,4-bis(aminomethyl)cyclohexane, diaminopropanol, 4,7,10-trioxa-1,13-tridecanediamine, spermidine and C36-alkylenediamines (Priamine™ 1071, 1073, 1074, 1075, respectively), preferably spermidine and/or 4,7,10-trioxa-1,13-tridecanediamine.

According to a particular embodiment of the invention, the (poly)amine compounds are monoamine compounds, i.e. they contain only one primary and/or secondary amine group, preferably a primary amine group (NH₂).

The (poly)amine compound(s) may be chosen from amino alkoxysilanes, notably of formula R′₁Si(OR′₂)_{z}(R′₃), in which:
R′₁ is a linear or branched, saturated or unsaturated, cyclic or acyclic C₁-C₆ hydrocarbon-based chain substituted with a group chosen from primary amine groups NH₂ or secondary amine groups —N(H)R with R representing a C₁-C₄ alkyl, an aryl or a benzyl substituted with an amino group or with a C₁-C₄ aminoalkyl group; R′₁ may be interrupted in its chain with a heteroatom (O, S, NH) or a carbonyl group (CO), R′₁ being linked to the silicon atom directly via a carbon atom,
R′₂ and R′₃, which may be identical or different, represent a linear or branched (C₁-C₆)alkyl group,
z denotes an integer ranging from 1 to 3, and
x denotes an integer ranging from 0 to 2,
   with z+x=3.

In particular, R′₁ is an acyclic chain. Preferably, R′₁ is a linear or branched, saturated or unsaturated C₁-C₆ hydrocarbon-based chain substituted with an amine —NH₂ or —N(H)R group, with R representing a C₁-C₆ alkyl, a C₃-C₆ cycloalkyl or a C₆ aromatic group. More preferentially, R′₁ is a saturated linear C₁-C₆ hydrocarbon-based chain substituted with an amine group NH₂. Even more preferentially, R′₁ is a saturated linear C₂-C₄ hydrocarbon-based chain substituted with an amine group NH₂.

In particular, R′₂ represents an alkyl group comprising from 1 to 4 carbon atoms; preferably, R′₂ represents a linear alkyl group comprising from 1 to 4 carbon atoms and more preferentially R′₂ represents an ethyl group.

In particular, R′₃ represents an alkyl group comprising from 1 to 4 carbon atoms; preferably, R′₃ represents a linear alkyl group comprising from 1 to 4 carbon atoms and more preferentially R′₃ represents methyl or ethyl groups. Preferably, z is equal to 3.

In particular, the (poly)amine compound(s) are chosen from amino alkoxysilanes including only one primary and/or secondary, preferably primary (NH₂), amine group, such as 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane, and N-(2-aminoethylaminomethyl)phenethyltrimethoxysilane.

Preferably, the (poly)amine compound(s) are chosen from 3-aminopropyltriethoxysilane (APTES), 3-aminoethyltriethoxysilane (AETES), 3-aminopropylmethyldiethoxysilane, and N-(2-aminoethyl)-3-aminopropyltriethoxysilane, and more preferentially 3-aminopropyltriethoxysilane (APTES), in particular the product sold by Sigma Aldrich.

The (poly)amine compound may also be chosen from amino polymers, notably having a weight-average molecular weight ranging from 500 g·mol⁻¹ to 1 000 000 g·mol⁻¹, preferably ranging from 500 g·mol⁻¹ to 500 000 g·mol⁻¹, and preferentially ranging from 500 g·mol⁻¹ to 100 000 g·mol⁻¹.

According to a particular embodiment of the invention, the (poly)amine compounds are monoamine compounds and are chosen from polydialkylsiloxanes notably of formula (IV):

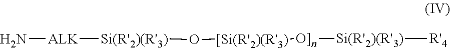

in which formula (IV):
Alk represents a (C₁-C₄) alkylene group which is linear or branched, preferably linear, such as propylene,
R′₂ and R′₃, which may be identical or different, preferably identical, represent a (C₁-C₄)alkyl group, such as methyl, and
R′₄ represents a linear or branched (C₁-C₆)alkyl group, preferably C₄, such as n-butyl, n represents an integer greater than or equal to 2, preferably the value of n is such that the weight-average molecular weight of the polydimethylsiloxane ranges from 500 to 3000 g·mol⁻¹. As examples of polydimethylsiloxanes (V), mention may be made of the products sold under the names MCR-A11 and MCR-A12 by the company Gelest.

According to a particular embodiment of the invention, the (poly)amine compounds are diamine compounds, i.e. they contain two primary and/or secondary amine groups, preferably primary amine groups (NH₂).

More particularly, they are chosen from the compounds of formula (V) or (VI):

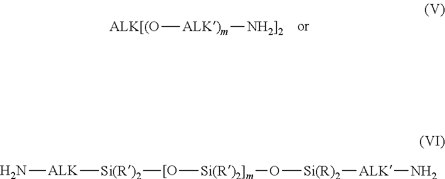

in which formulae (V) and (VI):
ALK and ALK′, which may be identical or different, represent a linear or branched (C₁-C₆)alkylene group, preferably a linear group such as propylene,
R′, which may be identical or different, represents a (C₁-C₄)alkyl group such as methyl,
m represents an integer greater than or equal to 0; preferably, the value of m is such that the weight-average molecular weight of compound (V) or (VI) ranges from 500 g·mol⁻¹ to 55 000 g·mol⁻¹.

As examples of compounds of formula (VI), mention may be made of those sold under the names DMS-A11, DMS-A12, DMS-A15, DMS-A21, DMS-A31, DMS-A32 and DMS-A35 by the company Gelest.

The (poly)amines compounds that are diamines are particularly polyether diamines notably of formula H₂N-ALK-O-[ALK′-O]m-ALK″-NH₂ with ALK, ALK′ and ALK″, which may be identical or different, representing a linear or branched (C₁-C₆))alkylene group, and m representing an integer greater than or equal to 0, such as 4,7,10-trioxa-1,13-tridecanediamine or the compounds known under the reference Jeffamine from the company Hunstman, and more particularly α,ω-diamino polyethylene glycol and/or polypropylene glycol (with an amine function at the end of the chain) such as the products sold under the names Jeffamine D-230, D-400, D-2000, D-4000, ED-600, ED-9000 and ED-2003.

According to a particular embodiment of the invention, the (poly)amine compound(s) are triamine compounds, i.e. they contain three primary and/or secondary amine groups, preferably primary amine groups (NH₂). More particularly, they are chosen from polyether triamines notably of formula ALK′″[(O-ALK′)ₘ—NH₂]₃ with ALK′ as defined previously and ALK′″ representing a linear or branched trivalent (C₁-C₆)alkylene group, and m representing an integer greater than or equal to 0.

As (poly)amine compounds that are triamine compounds, mention may be made in particular of polyether triamines, and notably α,ω-diamino polyethylene glycol and/or polypropylene glycol (with an amine function at the end of the chain) such as the products sold under the names Jeffamine T-403.

According to another particular embodiment of the invention, the (poly)amine compound(s) include more than three primary and/or secondary amine groups, preferably primary amine groups (NH₂).

In this variant, the (poly)amine compound(s) are chosen from poly(meth)acrylates or poly(meth)acrylamides bearing lateral primary or secondary amine functions, such as poly(3-aminopropyl)methacrylamide and poly(2-aminoethyl) methacrylate.

Preferably, the (poly)amine compounds are chosen from chitosans (notably poly(D-glucosamine)) and polydimethylsiloxanes comprising primary amine groups at the end of the chain and/or on side chains.

According to this variant, the (poly)amine compound(s) are chosen in particular from poly((C₂-C₅)alkyleneimines), and preferably polyethylenimines and polypropyleneimines, notably poly(ethyleneimine), in particular the product sold under reference 408700 by the company Aldrich Chemical or under the trade name Lupasol by BASF, notably with a molecular weight of between 1200 and 25 000; poly(allylamine), in particular the product sold under reference 479136 by the company Aldrich Chemical; polyvinylamines and copolymers thereof, notably with vinylamides, in particular vinylamine/vinylformamide copolymers such as those sold under the name Lupamin® 9030 by the company BASF; polyamine acids containing NH₂ groups, such as polylysine, in particular the product sold by the company INC Corporation (formerly Chisso); amino dextran, in particular the product sold by the company CarboMer Inc; amino polyvinyl alcohol, in particular the product sold by the company CarboMer Inc; acrylamido(C₁-C₆)alkylamine-based copolymers, notably acrylamidopropylamine-based copolymers; and poly(D-glucosamine), for example sold under the reference Kionutrime CSG® by the company Kytozyme.

According to a particular embodiment, the polydimethylsiloxanes comprising primary amine groups at the end of the chain and/or on side chains are chosen from the compounds of formula (VII) below:

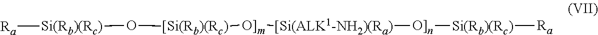

in which formula (VII):
Rₐ, which may be identical or different, represents a hydroxyl or (C₁-C₄)alkyl group,
R_{b} and R, which may be identical or different, preferably identical, represent a (C₁-C₄)alkyl group, such as methyl,
ALK¹ represents a linear or branched (C₁-C₆) alkylene group, optionally interrupted with an N(H) group,
m and n are integers greater than or equal to 1; preferably, m and n are such that the weight-average molecular mass of the compound of formula (VII) ranges from 1000 g·mol⁻¹ to 500 000 g·mol⁻¹.

According to a preferred variant, formula (VII) is such that Rₐ, R_{b} and Rₑ represent a methyl group, ALK¹ represents a propylene group, n and m are such that the weight-average molecular weight of the polydimethylsiloxane ranges from 1000 g·mol⁻¹ to 55 000 g·mol⁻¹.

As examples of polydimethylsiloxanes of formula (VI), mention may be made of those sold under the names AMS-132, AMS-152, AMS-162, AMS-163, AMS-191 and AMS-1203 by the company Gelest.

According to another variant, formula (VII) is such that Rₐ represents a hydroxyl or (C₁-C₄)alkyl group, such as methyl, ALK¹ represents a (C₅-C₆)alkylene group substituted with an NH group; preferably, ALK¹ represents —(CH₂)₃—N(H)—(CH₂)₂—, and m and n are such that the weight-average molecular mass of the compound of formula (VI) ranges from 5000 g·mol⁻¹ to 500 000 g·mol⁻¹.

As amine polymer, mention may also be made of α,ω-diamino polytetrahydrofurans (or polytetramethylene glycol) and α,ω-diamino polybutadienes.

According to a particular embodiment of the invention, the (poly)amine compounds are chosen from hyperbranched polymers comprising at least one amino group and dendrimers bearing at least one amino group, such as PAMAM polyamidoamine dendrimers with an ethylenediamine core and a terminal amine function.

According to a preferred embodiment, the composition comprises a crosslinking agent R chosen from (poly)amine compounds, in particular chosen from chitosans, aminoalkoxysilanes, polydimethylsiloxanes comprising primary amine groups at the end of the chain or on side chains, amodimethicones, polyglucosamines, spermidine and mixtures thereof.

More preferentially, the composition comprises a crosslinking agent chosen from chitosans, aminoalkoxysilanes and polydialkylsiloxanes comprising primary amine groups at the end of the chain or on side chains such as amodimethicones, and even more preferentially chosen from poly(D-glucosamine), 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, spermidine and polydimethylsiloxanes comprising terminal amino groups at the end of the chain, such as bis-cetearyl amodimethicone.

### B) (Poly)thiol Compounds

According to a preferred embodiment, the crosslinking agent R is chosen from (poly)thiol compounds, also known as “(poly)mercapto” compounds.

The (poly)thiol compound may in particular be organic or inorganic, preferably organic.

In a preferred embodiment, the (poly)thiol compound is silicon-based, i.e. it includes one or more thiol groups and it also includes at least one siloxane chain.

In a particular embodiment, the (poly)thiol compound is inorganic. Mention may be made, for example, of polythiol silicones.

The (poly)thiol compound may in particular be chosen from non-polymeric (poly)thiol compounds.

For the purposes of the present invention, the term “non-polymeric compounds” means compounds which are not directly obtained via a monomer polymerization reaction.

According to one embodiment of the invention, the (poly)thiol compound(s) are organic, non-polymeric and of formula (VIII) below and also the solvates thereof such as hydrates:

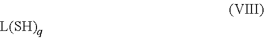

in which formula (VIII):
q denotes an integer greater than or equal to 2; preferably, q is between 2 and 10 inclusive and preferably between 2 and 5;
L denotes a linear or branched, saturated or unsaturated, or (hetero)cyclic, saturated or unsaturated, multivalent (at least divalent) group, in particular comprising between 1 and 500 carbon and/or silicon atoms, more particularly between 2 and 40 carbon and/or silicon atoms, even more particularly between 3 and 30 carbon and/or silicon atoms, preferably between 6 and 20 carbon atoms; L being optionally interrupted and/or terminated with one or more heteroatoms or groups chosen from O, S, N, Si, C(X), and combinations thereof such as —O—, —O—C(X)—, —N(R)—C(X)—, —Si(R_{c})(R_{d})—O— with R representing a hydrogen atom or a (C₁-C₆)alkyl group such as methyl; and/or L being optionally substituted with one or more groups chosen from: —N(Rₐ)R_{b} and —(X′)ₐ—C(X)—(X″)_{b}—Rₐ; with X, X′ and X″, which may be identical or different, representing an oxygen or sulfur atom, or a group N(R_{b}); a and b being 0 or 1, preferably the sum of a+b being 1; Rₐ and R_{b}, which may be identical or different, represent a hydrogen atom or a (C₁-C₆)alkyl or aryl(C₁-C₄)alkyl group, such as benzyl, preferably Rₐ and R_{b} represent a hydrogen atom; and Rₑ and R_{d}, which may be identical or different, represent a (C₁-C₆)alkyl, aryl(C₁-C₄)alkyl or (C₁-C₆)alkoxy group.

According to a particular embodiment of the invention, the (poly)thiol compound(s) are chosen from polythiol compounds, notably polythiol compounds comprising from 2 to 20 carbon atoms.

According to a preferred embodiment, the (poly)thiol compound(s) are non-polymeric and notably of formula (VIII) defined above, in which q is an integer greater than or equal to 2, preferably q is an integer between 2 and 10 inclusive and preferably between 2 and 5.

The (poly)thiol compounds that are suitable for use in the invention are preferably dithiol compounds.

Preferably, L denotes a C₈-C₁₈ multivalent radical, which is notably linear. Preferentially, the liposoluble polythiol is a notably linear C₈-C₁₈ dithiol. Preferably, the C₈-C₁₈ chain is a hydrocarbon-based chain, i.e. formed from carbon and hydrogen. In particular, the liposoluble polythiol is a linear C₈-C₁₆ and notably C₁₀-C₁₄ dithiol. As (poly)thiol compounds of formula (VII), mention may be made more particularly of 1,8-octanedithiol, 1,10-decanedithiol, 1,12-dodecanedithiol, 1,14-tetradecanedithiol, 1,16-hexadecanedithiol and 1,18-octadecanedithiol. Use is preferably made of 1,10-decanedithiol, 1,12-dodecanedithiol and/or 1,14-tetradecanedithiol, preferentially 1,12-dodecanedithiol.

According to another particular embodiment of the invention, the (poly)thiol compound(s) are chosen from thiolated alkoxysiloxanes, such as those of formula (VIII′) below:

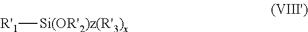

in which formula (VIII′):
R′₁ is a linear or branched, saturated or unsaturated, cyclic or acyclic C₁-C₁₂ hydrocarbon-based chain substituted with one or more groups chosen from thiol groups; and aryl, aryloxy, arylthio, arylamino, the aryl group being substituted with one or more thiol groups, or thiol(C₁-C₆)alkyl, preferably thiol(C₁-C₆)alkyl; and R′₁ is optionally interrupted in its hydrocarbon-based chain with one or more heteroatoms such as O, S, N, a carbonyl group C(O), or a combination thereof such as ester —C(O)—O—, or amide —C(O)—N(H)—, R′₁ being bonded to the silicon atom directly via a carbon atom,
R′₂ and R′₃, which may be identical or different, represent a linear or branched alkyl group comprising from 1 to 6 carbon atoms, preferably from 1 to 4 carbon atoms, such as methyl,
z denotes an integer ranging from 1 to 3, and
x denotes an integer ranging from 0 to 2,
   with z+x=3.

Preferably, R′₂ represents a linear or branched, preferably linear, alkyl group comprising from 1 to 4 carbon atoms, such as ethyl.

Preferably, R′₃ represents a linear or branched, preferably linear, alkyl group comprising from 1 to 4 carbon atoms, such as methyl or ethyl.

Preferably, R′₁ is an acyclic chain, in particular R′₁ is a linear or branched, saturated or unsaturated, preferably saturated, C₁-C₆ hydrocarbon-based chain substituted with one or more thiol groups, preferably substituted with one thiol group.

Preferably, R′₁ is a saturated linear C₁-C₆ hydrocarbon-based chain substituted with a thiol group, and R′₂ represents an alkyl group comprising from 1 to 4 carbon atoms.

Preferably, R′₃ represents an alkyl group comprising from 1 to 4 carbon atoms.

Preferably, z is equal to 3.

According to a more particular embodiment of the invention, the thiolated alkoxysiloxanes are chosen from those of formula (IX) below:

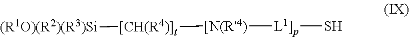

in which formula (IX):
p is 0 or 1;
t is an integer between 1 and 4, preferably 2;
R¹ represents a (C₁-C₆)alkyl radical;
R² and R³, which may be identical or different, preferably identical, are chosen from a (C₁-C₆)alkyl group, in particular a C₁-C₄ alkyl group, such as methyl, and a (C₁-C₆)alkoxy group, in particular a (C₁-C₄)alkoxy group, such as methoxy;
R⁴ and R′⁴, which may be identical or different, represent a hydrogen atom or a (C₁-C₆)alkyl group, such as methyl;
L¹ represents a divalent, saturated, linear or branched C₁-C₂₀ hydrocarbon-based radical.

According to a particular embodiment of the invention, the thiolated alkoxysiloxanes are chosen from those of formula (IX′) below:

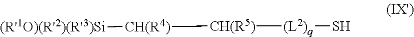

in which formula (IX′):
q is equal to 0 or 1;
X represents an oxygen or sulfur atom, preferably a sulfur atom;
R′¹ denotes a (C₁-C₆)alkyl radical;
R′² and R′³, which may be identical or different, preferably identical, are chosen from a (C₁-C₆)alkoxy group, in particular a C₁-C₄ alkoxy group, and a (C₁-C₆)alkyl radical;
R⁵ represents a hydrogen atom or a C₁-C₄ alkyl group optionally substituted with an amino, thiol or hydroxyl group;
R⁴ represents a hydrogen atom or a C₁-C₄ alkyl group, in particular methyl;
L² represents a linear or branched, saturated C₁-C₂₀ divalent hydrocarbon-based group, optionally interrupted with a heteroatom such as —N(H)—, and/or optionally substituted with one or more hydroxyl, thiol or amino groups.

Preferably, the thiolated alkoxysilane(s) are chosen from 4-(trimethoxysilyl)-1-butanol, 3-(trimethoxysilyl)-1-propanol, 3-(triethoxysilyl)-1-propanol, 11-(trimethoxysilyl)-1-undecanethiol, 4-(trimethoxysilyl)-2-butanethiol, 2-(triethoxysilyl)ethanethiol, 3-(triethoxysilyl)-1-propanethiol, 2-(trimethoxysilyl)ethanethiol, 3-(trimethoxysilyl)-1-propanethiol and 3-(dimethoxymethylsilyl)-1-propanethiol.

More preferentially, the thiolated alkoxysilane(s) are chosen from 2-(triethoxysilyl)ethanethiol (18236-15-2) and 3-(triethoxysilyl)-1-propanethiol (14814-09-6). According to a preferred embodiment of the invention, the (poly)thiol compound(s) are chosen from polymeric (poly)thiol compounds.

The polymeric (poly)thiol compounds may be star, comb, brush and dendritic homopolymers or copolymers bearing thiol units. The polymers may be of natural origin such as polysaccharides or polypeptides, or of synthetic origin such as acrylic polymers, polyesters or polyglycols. The thiol units may be present as terminal and/or side groups.

Examples that may be mentioned include the polymers described in the following articles: Polymers containing groups of biological activity, C. G. Overberger et al., Polytechnic Institute of Brooklyn, http://pac.iupac.org/publications/pac/pdf/1962/pdf/0402x0521.pdf, EP 1 247 515 A2; U.S. Pat. No. 3,676,440; and EP 1 572 778.

The polymeric (poly)thiol compounds of the invention are preferably organic and/or silicone compounds, more preferentially of formula (X):

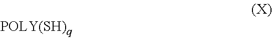

in which formula (X):
q is greater than or equal to 2, preferably greater than or equal to 3;
POLY denotes a polymer-based radical, preferably carbon-based or silicone-based; POLY being optionally interrupted with one or more heteroatoms or groups chosen from O, S, N, Si, C(X), and combinations thereof such as —O—, —O—C(X)—, —N(R)—C(X)—, —Si(R_{c})(R_{d})—O— with R representing a hydrogen atom or a (C₁-C₆)alkyl group such as methyl; and/or POLY being optionally substituted with one or more halogen atoms, or a group chosen from Rₐ(R_{b})N— and —(X′)ₐ—C(X)—(X″)_{b}—Rₐ; X, X′ and X″, which may be identical or different, represent an oxygen or sulfur atom or a group N(R_{b}); a and b being 0 or 1, preferably the sum of a+b being 1; Rₐ and R_{b}, which may be identical or different, represent a hydrogen atom or a (C₁-C₁₀)alkyl or aryl(C₁-C₄)alkyl group, such as benzyl, preferably Rₐ and R_{b} represent a hydrogen atom; and Rₑ and R_{d}, which may be identical or different, represent a (C₁-C₁₀)alkyl, aryl(C₁-C₄)alkyl or (C₁-C₁₀)alkoxy group.

The methods for preparing the polymeric (poly)thiol compounds used according to the invention are known to those skilled in the art; several methods are reported hereinbelow in a non-limiting manner. The polymeric (poly)thiol compounds used according to the invention may be obtained by polymerization or polycondensation of monomer units bearing thiol or protected thiol functions, optionally as a copolymerization or co-polycondensation of monomer units free of thiol or protected thiol functions.

According to one embodiment of the invention, the polymeric (poly)thiol compounds used according to the invention are polymers which are soluble in cosmetic media, particularly in aqueous or aqueous-alcoholic media. They are more preferentially obtained from amino polymers and the ammonium salts thereof or from polyhydroxylated polymers.

According to another embodiment of the invention, the thiolated polymers used according to the invention are polymers that are soluble in lipophilic media.

According to one embodiment of the invention, the polythiol compound is a polymeric compound of formula (X) in which q denotes an integer greater than or equal to 2, and POLY denotes a carbon-based and/or silicon-based, preferably silicon-based, polymeric radical, POLY also possibly containing one or more heteroatoms chosen from O, N or S, and/or one or more functions chosen from (thio)ester, (thio)ketone, (thio)amide, (thio)urea and (thio)carbamate functions, and/or possibly being substituted with one or more linear or branched (C₁-C₁₀)alkyl or linear or branched (C₁-C₁₀)alkoxy groups, it being understood that when POLY is substituted, the thiol functions may be borne by the substituent(s).

The weight-average molecular weight of the polythiol polymer compounds, such as those of formula (X), is generally between 500 and 400 000 g·mol⁻¹, preferably between 500 and 150 000 g·mol⁻¹.

According to a particular embodiment of the invention, the polythiol compounds are chosen from polyorganosiloxanes including thiol groups on end chains, such as those of formula (XI) below:

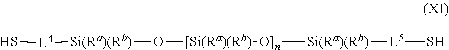

in which formula (XI):
R^{a} and R^{b}, which may be identical or different, preferably identical, represent a group from among: (C₁-C₄)alkyl such as methyl, (C₁-C₄)alkoxy such as methoxy, aryl such as phenyl, aryloxy such as phenoxy, aryl(C₁-C₄)alkyl such as benzyl, or aryl(C₁-C₄)alkoxy such as benzoxy, preferably (C₁-C₄)alkyl such as methyl;
n represents an integer greater than or equal to 1 and more particularly the value of n is such that the weight-average molecular weight of the silicone ranges from 500 to 55 000 g·mol⁻¹; in particular, n is an integer ranging from 1 to 100, preferably ranging from 5 to 50 and preferentially ranging from 10 to 30, and
L⁴ and L⁵, which may be identical or different, preferably identical, represent a linear or branched, saturated or unsaturated, optionally cyclic hydrocarbon-based chain comprising from 1 to 100 carbon atoms, optionally interrupted with one or more heteroatoms such as oxygen, sulfur or nitrogen, in particular oxygen, and in particular represent a covalent bond or a (C₁-C₆)alkylene, (C₁-C₆)alkylenoxy, oxy(C₁-C₆)alkylene, (C₁-C₆)alkylenoxy(C₁-C₆)alkylene, (C₁-C₆)alkylenoxy(C₁-C₆)alkylenoxy or oxy(C₁-C₆)alkylenoxy(C₁-C₆)alkylene group, preferably a (C₁-C₆)alkylene, (C₁-C₆)alkylenoxy, oxy(C₁-C₆)alkylene or (C₁-C₆)alkylenoxy(C₁-C₆)alkylene group.

Preferentially, the (poly)thiol compounds are polythiol polyorganosiloxanes, more preferentially polythiol polydimethylsiloxanes, notably chosen from those of formula (XII):

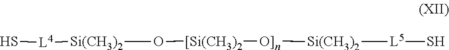

in which formula (XII):
L⁴ and L⁵ are as defined previously in formula (IX), in particular L⁴ and L⁵ represent a (C₁-C₆)alkylene, (C₁-C₆)alkylenoxy, oxy(C₁-C₆)alkylene or (C₁-C₆)alkylenoxy(C₁-C₆)alkylene group, more preferentially a divalent group chosen from —R₂—, —O—R₂—, —R₂—O— and —R₂—O—R₂—, preferably —R₂—O—R₂—, with R₂ representing a linear or branched, preferably linear, (C₂-C₆)alkylene group, such as ethylene or n-propylene, preferably n-propylene; and
n is as defined in formula (XI).

As polythiol compounds of formula (XII), mention may be made of mercaptosiloxanes or thiolated siloxanes in which the thiol functions are at the chain ends, sold by the company Shin-Etsu under the reference X-22-167B, and mercaptosiloxane in which the mercapto functions are pendent, sold by the company Shin-Etsu under the reference KF-2001, or polydimethylsiloxanes in which the thiol functions are at the chain ends, via thio-n-propyl, 80-120 groups, sold by the company Gelest under the name DMS-SM 21.

Preferentially, the polythiol compounds are chosen polyorganosiloxanes including thiol groups on side chains, such as those of formula (XIII):

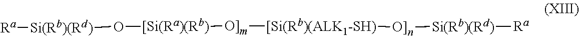

in which formula (XIII):
R^{a} and R^{b} are as defined in formula (XI) and R^{d} is as defined for R^{a} and R^{b}, preferably Rₐ, R^{b} and R^{d}, which are identical, represent a (C₁-C₆)alkyl group, such as methyl;
R^{d} may also represent a (C₁-C₆)alkyl group substituted with a (C₁-C₄)alkylamino or amino or thiol group, preferably (C₁-C₄)alkyl such as methyl;
ALK₁ represents a linear or branched, optionally cyclic, saturated or unsaturated divalent hydrocarbon-based chain comprising from 1 to 100 carbon atoms, optionally interrupted with one or more heteroatoms such as oxygen, sulfur or nitrogen, in particular oxygen, a (thio)carbonyl group C(X) with X representing O or S, or combinations thereof such as —O—, —O—C(O)— or —C(O)—O—; preferably, ALK₁ represents a (C₁-C₆)alkylene and more preferentially (C₁-C₄)alkylene group such as propylene;
n and m, which may be identical or different, represent an integer greater than 2 and more particularly the values of m and n are such that the weight-average molecular weight of said polyorganosiloxane is between 1000 and 55 000 g·mol⁻¹.

As examples of polythiol compounds of formula (XIII), mention may be made of those sold by the company Genesee Polymers under the names GP-367, GP-71-SS, GP-800 and GP-710s, preferably GP-367, sold by the company Genesee Polymers.

The polythiol compounds are notably polydimethylsiloxanes including at least two thiol groups, for instance the products SMS-022, SMS-042 and SMS-992 sold by the company Gelest in https://www.gpcsilicones.com/products/silicone-fluids/mercapto-functional, https://www.shinetsusilicone-global.com/products/type/oil/detail/search/deg07.shtml, and 1053_Reactive_Silicones_Silanes/Silicones—Gelest.

According to a particular embodiment of the invention, the (poly)thiol compounds are chosen from hyperbranched polymers comprising at least one thiol group and dendrimers bearing at least one thiol group, such as thiolated PAMAM dendrimers.

Preferably, the (poly)thiol compounds used according to the invention are chosen from polydiallylsiloxanes, notably polydimethylsiloxanes, including at least two thiol groups such as those of formula (XIII).

### C) (Poly)acrylate Compounds

According to a particular embodiment, the crosslinking agent is a (poly)acrylate compound. The term “(poly)acrylate” means a compound which comprises at least one acrylate ester group H₂C═C(R^{e})—C(O)—Y— with R^{e} representing a hydrogen atom or a (C₁-C₄)alkyl group, such as methyl, preferably R^{e} representing a hydrogen atom, and Y representing an oxygen atom or an amino group —N(H)—, preferably an oxygen atom.

More particularly, the (poly)acrylate(s) of the invention are of formula (XIV):

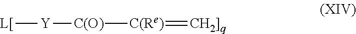

in which formula (XIV) q and L are as defined in formula (VIII), Y and R^{e} being as defined previously, preferably Y=O and R^{e}=H.

According to a preferred embodiment, the compounds of formula (XIV) are such that L represents a di- or trivalent, preferably trivalent, hydrocarbon-based chain comprising from 1 to 8 carbon atoms, q is 2 or 3, preferably 3, Y represents O, and R^{e} represents a hydrogen atom.

According to a particular embodiment, the (poly)acrylate compounds are chosen from polyorganosiloxanes including at least one acrylate group on the side chain, such as those of formula (XV):

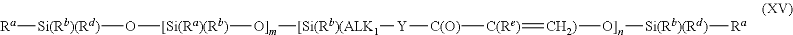

in which formula (XV):
R^{a}, R^{b} and R^{d} are as defined for formula (XIII), preferably R^{a}, R^{b} and R^{d} represent a (C₁-C₆)alkyl group, such as methyl,
ALK₁ is as defined for formula (XIII), preferably ALK₁ represents a (C₁-C₆)alkylene group, more preferentially a (C₁-C₄)alkylene group, such as propylene,
n and m, which may be identical or different, represent an integer greater than 2 and more particularly the values of m and n are such that the weight-average molecular weight of said polyorganosiloxane is between 1000 and 55 000 g·mol⁻¹.
Y is as defined previously, and is preferably an oxygen atom.

More particularly, the (poly)acrylate compound may be chosen from 1,3-butanediol diacrylate, 1,4-butanediol diacrylate, bis(trimethylolpropane) tetraacrylate, glyceryl 1,3-diglycerolate diacrylate, glyceryl propoxylate (1PO/OH) triacrylate, 1,6-hexanediol diacrylate, 1,6-hexanediol ethoxylate diacrylate, hydroxypivalyl hydroxypivalate, neopentyl glycol diacrylate, neopentyl glycol propoxylate (1PO/OH) diacrylate, pentaerythrityl tetraacrylate, pentaerythrityl triacrylate, poly(propylene glycol) diacrylate, tricyclo[5.2.1.02,6]decanedimethanol diacrylate, trimethylolpropane ethoxylate (1EO/OH) methyl ether diacrylate, trimethylolpropane propoxylate triacrylate, trimethylolpropane triacrylate, trimethylolpropane trimethacrylate, tri(propylene glycol) diacrylate, and tris[2-(acryloyloxy)ethyl]isocyanurate.

The (poly)acrylate compound may also be chosen from N,N′-methylenebisacrylamide.

According to this embodiment, the (poly)acrylate compound is combined in its implementation with an amine catalyst as described, for example, in Progress in coating 129, 21-25 (2019) and Progress in coating 135, 510-516 (2019). Preferably, the amine catalyst(s) are chosen from piperidine, DMAP (dimethylaminopyridine), DBU (1,8-diazabicyclo[5.4.0]undec-7-ene), DABCO (1,4-diazabicyclo[2.2.2]octane) and DBN (1,5-diazabicyclo[4.3.0]non-5-ene), more preferentially chosen from DBU (1,8-diazabicyclo[5.4.0]undec-7-ene), DABCO (1,4-diazabicyclo[2.2.2]octane) and DBN (1,5-diazabicyclo[4.3.0]non-5-ene), and in particular the catalyst is DBU (1,8-diazabicyclo[5.4.0]undec-7-ene).

More particularly, the (poly)acrylate compounds are chosen from those of formula (XIV), notably trimethylolpropane triacrylate, and those of formula (XV), more preferentially from those of formula (XIV), notably trimethylolpropane triacrylate.

### D) Metal Alkoxides

According to a particular embodiment, the crosslinking agent R is a compound chosen from the metal alkoxides of formulae (XIVₐ), (XIV_{b}), (XIV_{c}) and (XIV_{d}) below and mixtures thereof:

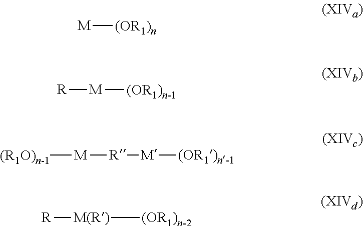

in which formulae (XIVₐ), (XIV_{b}), (XIV_{c}) and (XIV_{d}):
M and M′, which may be identical or different, represent an atom chosen from alkaline-earth metals, transition metals, metals of the lanthanide family, post-transition metals such as aluminium or tin and metalloids such as boron; preferably transition metals such as Ti and post-transition metals such as aluminium;
n and n′ respectively represent the valencies of the atoms represented by M and M′;
R₁ and R₁′, which may be identical or different, represent a linear or branched, saturated or unsaturated hydrocarbon-based group containing from 1 to 30 carbon atoms, preferably from 1 to 6 carbon atoms, said hydrocarbon-based group being optionally interrupted with 1 to 20 heteroatoms chosen from O, N, S and P, notably O or N; and/or said hydrocarbon-based group being optionally substituted with one or more hydroxyl or carbonyl groups;
R and R′, which may be identical or different, represent a hydrogen atom or a linear, branched, acyclic or cyclic, saturated or unsaturated hydrocarbon-based group containing from 1 to 30 carbon atoms, preferably from 2 to 20 carbon atoms, optionally interrupted with 1 to 20 heteroatoms chosen from O, N, S and/or P, notably O or N, and/or said hydrocarbon-based group being optionally substituted with one or more hydroxyl or carbonyl groups;
R″ represents —O—, —N(R2)-, —S— or a linear, cyclic or branched, saturated or unsaturated divalent hydrocarbon-based group containing from 1 to 30 carbon atoms, preferably from 2 to 20 carbon atoms, optionally interrupted with 1 to 20 heteroatoms chosen from O, N, S and P, notably O or N, with R2 representing a linear, cyclic or branched, saturated or unsaturated hydrocarbon-based group containing from 1 to 30 carbon atoms, preferably from 2 to 20 carbon atoms.

Preferably, M and M′, which may be identical or different, represent an atom chosen from transition metals such as titanium or zirconium or alkaline-earth metals such as magnesium, more preferentially chosen from transition metals such as titanium or zirconium, even more preferentially titanium.

Preferably, the organometallic compound(s) are chosen from the alkoxides of formula (XIVₐ) as defined previously. According to this preferred embodiment, the organometallic compound(s) are more particularly chosen from the alkoxides of formula (XIVₐ), in which M represents an atom chosen from transition metals, metals of the lanthanide family, post-transition metals, such as aluminium, tin, metalloids, such as boron, or alkaline-earth metals, such as magnesium or calcium; n represents the valency of the atom represented by M; R₁ represents a saturated, linear or branched, hydrocarbon-based group containing from 1 to 30 carbon atoms, preferably from 1 to 6 carbon atoms.

According to another more preferred embodiment, the organometallic compound(s) are chosen from the alkoxides of formula (XIVₐ), in which M represents an atom chosen from transition metals, such as zirconium or titanium, metals of the lanthanide family, post-transition metals, such as aluminium or tin, metalloids, such as boron, and alkaline-earth metals, such as magnesium, preferably M represents a titanium atom; n represents the valency of the atom represented by M, notably 1, 2, 3 or 4, in particular 4; R₁ represents a methyl, ethyl, 2-ethylhexyl, propyl, isopropyl, n-butyl, isobutyl or t-butyl group.

According to an even more preferred embodiment, the organometallic compound(s) are chosen from zirconium ethoxide (Zr(OC₂H₅)₄), zirconium propoxide (Zr(OCH₂CH₂CH₃)₄), zirconium isopropoxide (Zr(OCH(CH₃)₂)₄), zirconium butoxide Zr(OCH₂CH₂CH₂CH₃)₄, zirconium tert-butoxide (Zr(OC(CH₃)₃)₄), titanium ethoxide (Ti(OC₂H₅)₄), titanium propoxide (Ti(OCH₂CH₂CH₃)₄), titanium isopropoxide (Ti(OCH(CH₃)₂)₄), titanium butoxide (Ti(OCH₂CH₂CH₂CH₃)₄), titanium tert-butoxide (Ti(OC(CH₃)₃)₄), titanium 2-ethylhexyloxide (Ti(OCH₂CH(C₂H₅)(CH₂)₃CH₃)₄), and mixtures thereof, more preferentially chosen from zirconium propoxide, titanium propoxide, titanium butoxide and mixtures thereof.

More preferentially, the crosslinking agent R is a compound of formula (XIVₐ) preferably in which M represents an atom chosen from transition metals, notably titanium such as titanium butoxide.

### E) (Poly)carbonyl Compounds

According to a particular embodiment, the crosslinking agent R is a (poly)carbonyl compound.

In particular, the (poly)carbonyl compound is chosen from terephthalaldehyde, 5,5-dimethyl-1,3-cyclohexanedione, phenylglyoxal, isophthalaldehyde, 4-acetylbenzaldehyde, 4,4-diformyltriphenylamine, 2-acetylbenzaldehyde, 3-(2-furoyl)quinoline-2-carboxaldehyde, 3-(2-furoyl)quinoline-2-carboxaldehyde, 3-acetylbenzaldehyde, 9-(2-ethylhexyl)carbazole-3,6-dicarboxaldehyde, phthaldialdehyde, 1,3-cyclohexanedione, 4,4′-biphenyldicarboxaldehyde, benzene-1,3,5-tricarboxaldehyde, and nonionic or anionic oxidized polysaccharides such as oxidized inulins, notably those of formula (II) as defined hereinbelow. In particular, the (poly)carbonyl compounds include a saturated or unsaturated, aromatic C₅-C₇ carbocycle, preferably aromatic, such as phenyl, or non-aromatic and saturated such as cyclohexyl, more preferentially unsaturated and aromatic, such as terephthaldehyde.

According to a particular embodiment, the (poly)carbonyl compound(s) are chosen from nonionic or anionic oxidized polysaccharides comprising one or more aldehyde groups, and optionally one or more anionic groups.

These anionic groups are preferably carboxyl or carboxylate groups.

The nonionic or anionic oxidized polysaccharides according to the invention may be represented by formula (II) below:

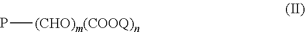

in which formula (II):
P represents a polysaccharide chain preferably consisting of monosaccharides comprising 5 carbon atoms or more than 5 carbon atoms, preferably 6 or more than 6 carbon atoms and more particularly 6 carbon atoms;
Q is chosen from a hydrogen atom, the ions derived from an alkali metal or an alkaline-earth metal such as sodium or potassium, ammonia, organic amines such as monoethanolamine, diethanolamine, triethanolamine and 3-amino-1,2-propanediol and basic amino acids such as lysine, arginine, sarcosine, ornithine and citrulline;
m+n is greater than or equal to 1;
m is such that the degree of substitution of the polysaccharide with one or more aldehyde groups (DS(CHO)) is within the range from 0.001 to 2 and preferably from 0.005 to 1.5;
n is such that the degree of substitution of the polysaccharide with one or more carboxylic groups (DS(COOX)) is within the range from 0 to 2 and preferably from 0.001 to 1.5.

The expression “degree of substitution DS(CHO) or DS(COOX) of the polysaccharides according to the invention” means the ratio between the number of carbons oxidized to give an aldehyde or carboxylic group for all the repeating units and the number of elementary monosaccharides (even opened by preoxidation) constituting the polysaccharide. The groups CHO and COOX may be obtained during the oxidation of certain carbon atoms, for example on the carbon atoms 2, 3 or 6, of a saccharide unit containing 6 carbon atoms.

Preferably, the oxidation may take place on carbons 2 and 3, more particularly of 0.01% to 75% by number, and preferably of 0.1% to 50% by number of the rings that may have been opened.

The polysaccharide chain, represented by P, is preferably chosen from celluloses, starches, maltodextrins, guar gums, xanthan gums, pullulan gums, agar-agar gums, carrageenan gums, gellan gums, acacia gums, polyxylans and tragacanth gums, and derivatives thereof.

The term “derivative” means the compounds obtained by chemical modification of the mentioned compounds. They may be esters, amides or ethers of said compounds.

The oxidation may take place according to a process known in the art, for example according to the process described in FR 2 842 200, in document FR 2 854 161 or in the article “Hydrophobic films from maize bran hemicelluloses” by E. Fredon et al., Carbohydrate Polymers 49, pages 1 to 12 (2002).

Another oxidation process is described in the article “Water soluble oxidized starches by peroxide reaction extrusion” Industrial Crops and Products 7, R. E. Wing, J. L. Willet, 45-52 (1997).

According to this embodiment, the (poly)carbonyl compound is combined in its implementation with an amine catalyst as described in the articles Progress in coating 129, 21-25 (2019) and Progress in coating 135, 510-516 (2019). Preferably, the amine catalyst(s) are chosen from piperidine, DMAP (dimethylaminopyridine), DBU (1,8-diazabicyclo[5.4.0]undec-7-ene), DABCO (1,4-diazabicyclo[2.2.2]octane) and DBN (1,5-diazabicyclo[4.3.0]non-5-ene), more preferentially chosen from DBU (1,8-diazabicyclo[5.4.0]undec-7-ene), DABCO (1,4-diazabicyclo[2.2.2]octane) and DBN (1,5-diazabicyclo[4.3.0]non-5-ene), and in particular the catalyst is DBU (1,8-diazabicyclo[5.4.0]undec-7-ene).

### F) Metal Salts

According to another particular embodiment, the crosslinking agent R is a metal salt chosen from alkali metal salts, alkaline-earth metal salts such as magnesium salts, transition metal salts, post-transition metal salts such as aluminium or tin salts, metalloid salts such as boron salts, hydrates thereof and mixtures thereof.

Preferably, the metal salt(s) are chosen from post-transition metal salts such as aluminium salts, hydrates thereof and mixtures thereof.

The term “metal salt” means a salt resulting notably from the action of an acid on a metal, in particular a transition metal, post-transition metal, metalloid, alkali metal or alkaline-earth metal.

The metal salt(s) may be in the form of hydrates.

The metal salt(s) may be organic or inorganic.

The term “organic metal salt” means a salt resulting notably from the action of an organic acid on a metal, in particular transition metals, post-transition metals, metalloids, alkali metals or alkaline-earth metals, preferably resulting from the action of a carboxylic acid on a metal.

Preferably the metal salt(s) are chosen from organic metal salts, hydrates thereof and mixtures thereof.

The term “inorganic metal salt” means a salt resulting notably from the action of an inorganic acid on a metal, in particular a transition metal, a post-transition metal, a metalloid, an alkali metal or an alkaline-earth metal.

The term “inorganic acid” means an acid which does not include any carbon atoms, apart from carbonic acid.

According to a particular embodiment of the invention, the inorganic metal salt(s) may be chosen from halides such as chlorides, fluorides, iodides and bromides, carbonates, sulfates, phosphates, nitrates, perchlorates, hydrates thereof, and mixtures thereof.

According to a more particular embodiment, the crosslinking agent R is an organic metal salt derived from a carboxylic acid.

More particularly, the crosslinking agent R is an organic metal salt chosen from metal (poly)(hydroxy)(C₁-C₆)alkylcarboxylates of alkali metals, alkaline-earth metals, transition metals, and post-transition metals such as aluminium.

It is understood that the metal (poly)(hydroxy)(C₁-C₆)alkylcarboxylate means that the (C₁-C₆)alkyl group is optionally substituted with one or more hydroxyl groups and one or more carboxyl or carboxylate groups. Preferably, the metal (poly)(hydroxy)(C₁-C₆)alkylcarboxylate represents R^{a}—C(O)-OM with M representing a transition metal such as titanium (Ti), or else a post-transition metal such as aluminium (Al), and Rₐ represents a linear or branched (C₁-C₆)alkyl group optionally substituted with at least one hydroxyl group.

According to a preferred embodiment of the invention, the metal salt(s) are organic, preferably chosen from citrates, lactates, glycolates, gluconates, acetates, propionates, fumarates, oxalates, glycinates and tartrates, hydrates thereof, and mixtures thereof, more preferentially acetates, lactates or mixtures thereof such as aluminium acetate or aluminium lactate.

According to a preferred embodiment, the metal salt(s) are chosen from basic aluminium acetate, aluminium oxalate, hydrated or non-hydrated aluminium citrate, aluminium lactate and aluminium glycinate, and mixtures thereof.

According to an even more preferred embodiment, the metal salt is basic aluminium acetate.

### G) Rare-Earth Metal Derivatives

According to another particular embodiment, the crosslinking agent R is a compound of a metal belonging to the group of the rare-earth metals M″, and notably a salt of a metal belonging to the group of the rare-earth metals.

The term “salt of a metal belonging to the group of the rare-earth metals” means a salt notably derived from the action of an acid on a metal belonging to the group of the rare-earth metals.

The compound(s) of a metal belonging to the group of the rare-earth metals may be in the form of hydrates.

The compound(s) of a metal belonging to the group of the rare-earth metals may be organic or inorganic. They may or may not be in salt form.

The term “organic salt of a metal belonging to the rare-earth metal group” means a salt notably derived from the action of an organic acid (notably a carboxylic acid) on a metal belonging to the group of the rare-earth metals.

The term “inorganic salt of a metal belonging to the group of the rare-earth metals” means a salt notably derived from the action of a inorganic acid on a metal belonging to the group of the rare-earth metals.

The term “inorganic acid” means an acid which does not include any carbon atoms, apart from carbonic acid.

As examples of metals belonging to the group of the rare-earth metals M″, mention may be made of scandium, yttrium, lanthanum, cerium, praseodymium, neodymium, promethium, samarium, europium, gadolinium, terbium, dysprosium, holmium, erbium, thulium, ytterbium and lutetium. Preferably, the metal(s) belonging to the group of the rare-earth metals are chosen from cerium, yttrium, ytterbium, lanthanum and europium; more preferentially, the metal(s) belonging to the group of the rare-earth metals M″ are chosen from cerium and yttrium.

Preferably, the metal belonging to the group of the rare-earth metals M″ is chosen from cerium, yttrium, ytterbium, lanthanum and europium, and mixtures thereof. More preferentially, the metal belonging to the group of the rare-earth metals is chosen from cerium and yttrium, and mixtures thereof.

Preferably, the metal belonging to the group of the rare-earth metals M″ is in the oxidation state +III.

According to the invention, the compound of a metal belonging to the group of the rare-earth metals is chosen from rare-earth metal salts and rare-earth metal complexes.

The term “rare-earth metal complex” refers to the combination of the metal M″ with one or more ligands.

In the text hereinbelow, the term “ligand” refers to an ion or a molecule bearing a group which combines, via an ionic bond and/or a coordination bond, with the metal M″. The same ligand may bear several groups which combine via an ionic bond and/or a coordination bond.

A definition of rare-earth metal salts or complexes may be found in: Progress in the Science and Technology of the Rare Earths, Volume 1, edited by Leroy Eyring in 1964, published by Macmillan Company and written by F. Gaume-Mahn, page 259 et seq.

The rare-earth metal salts and complexes according to the invention are characterized in that they contain at least one metal atom M″ belonging to the group of the rare-earth metals and that said atom is in the +III oxidation state.

The metal belonging to the group of the rare-earth metals M″ may then be associated, via its electron shell, with n1 anionic groups forming an ionic bond with M″ and/or with n2 groups forming a coordination bond with M″. The groups forming a coordination bond are, for example, groups with a donor doublet, such as carbonyl or amine.

If n2=0, the compound of the metal belonging to the group of the rare-earth metals forms a salt and, in this case, the metal M″ belonging to the group of the rare-earth metals is associated with three anionic groups.

If n2>0, the compound of the metal belonging to the group of the rare-earth metals forms a complex and, in this case, the number of anionic groups n1 may range from 0 to 3.

The metal M″ belonging to the group of the rare-earth metals is associated with one or more anionic groups and/or one or more groups forming a coordination bond.

The ligands associated with the metals belonging to the group of the rare-earth metals M″ to form a corresponding rare-earth metal complex are as described below.

a) Typically, the ligand may be a monoanionic ion, which may or may not be monoatomic, such as a nitrate, or a hydroxyl (OH—) or a halide (typically chloride or bromide). By way of example, the resulting rare-earth metal compound may then be M″Cl₃, M″(OH)₃ or M″(NO₃)₃, and in particular CeNO₃, YNO₃, LaNO₃, CeCl₃, YCl₃, LaCl₃, more preferentially rare-earth metal halides, notably Ce and Y halides such as CeCl₃ and YCl₃.

b) The ligand may be a dianionic or trianionic ion, such as phosphate or sulfate. By way of example, mention may be made of rare-earth metal compounds such as MPO₄, or M₂(SO₄)₃ and in particular CePO₄, YPO₄, LaPO₄, Ce₂(SO₄)₃, Y₂(SO₄)₃ and La₂(SO₄)₃.

c) The ligand may contain one or more groups forming a coordination bond and a function forming an ionic bond.

Thus, the ligand may be a monocarboxylate or polycarboxylate molecule, such as acetate or succinate. In this case, it is considered that the carboxylate function acts as an anionic group, by means of the hydroxyl of the carboxylic group, and acts as a group forming a coordination bond by means of the lone pair on the oxygen of the carbonyl function. Thus, the resulting rare-earth metal compound may be M″(R—(COO)ₙ)_{3/n}. In addition to bearing one or more carboxylates, the ligand may include other functions, such as hydroxyls or amines. Thus, the ligand may consist totally or partially of hydroxycarboxylic acids or aminocarboxylic acids. As monocarboxylic or polycarboxylic compound bearing additional functions, mention may be made of tartrate, citrate, glycolate or ethylenediaminetetraacetate (EDTA) ions.

The ligand may bear a non-localized anionic charge, for instance acetylacetonate. The rare-earth metal compound will then be M″(acetylacetonate)₃ or M″(acetylacetonate)₃·7H₂O in which each acetonate bonds to the metal M″ via its two carbonyl functions, one acting as an anionic group, the other as a group bonding by coordination.

The ligand may also be of the aromatic type, such as a phenol, a cyclopentadiene (Progress in the Science and Technology of the Rare Earths, published by Leroy Eyring and written by F. Gaume-Mahn, page 296), or a pyridine.

d) The rare-earth metal compound may include one or more ligands forming a coordination bond and one or more ligands forming an ionic bond. Thus, the rare-earth metal compound may be yttrium dihydroxyacetate Y((OH)₂acetate) (Synthesis and Properties of Yttrium Hydroxyacetate Sols by S. S. Balabanov, E. M. Gavrishchuk, and D. A. Permin, Inorganic Materials, 2012, Vol. 48, No. 5, pages 500-503).

e) The rare-earth metal compound may be a mixed salt in which one of the cations M′″ represents a cation other than a rare-earth metal cation, for instance an alkali metal or alkaline-earth metal or a cationic organic cation, notably a quaternary amine (or ammonium), for example mono/di/tri/tetra(C₁-C₄)alkylammonium, or mono/di(C₁-C₄)alkyl imidazolium, (C₁-C₄)alkylpyridinium; more particularly, the mixed salt rare-earth metal compound is Li,Ce(SO₄)₂.

The compounds belonging to the group of the rare-earth metals, which are often highly hygroscopic, may be in the form of hydrates, for instance CeCl₃·7H₂O, YCl₃·6H₂O, LaCl₃·7H₂O or Ce(acetonate)₃·xH₂O.

According to a particular embodiment of the invention, the compound(s) belonging to the group of the rare-earth metals are chosen from the salts of organic acids such as citrates, lactates, glycolates, gluconates, acetates, propionates, fumarates, oxalates, tartrates, mesylates and methosulfates, notably gluconates, hydrates thereof, and mixtures thereof.

According to a preferred embodiment, the salt(s) of a metal belonging to the group of the rare-earth metals are inorganic salts.

Preferably, the inorganic salt(s) of a metal belonging to the group of the rare-earth metals are chosen from halides such as chlorides, fluorides, iodides and bromides, carbonates, sulfates, phosphates, nitrates and perchlorates, hydrates thereof, and mixtures thereof.

More preferentially, the inorganic salt(s) of a metal belonging to the group of the rare-earth metals are chosen from halides such as chlorides, fluorides, iodides and bromides, and nitrates, hydrates thereof, and mixtures thereof.

Even more preferentially, the inorganic salt(s) of a metal belonging to the group of the rare-earth metals are chosen from chlorides and nitrates, hydrates thereof, and mixtures thereof.

According to a particularly preferred embodiment, the compound(s) belonging to the group of the rare-earth metals are chosen from Ce(NO₃)₃, Y(NO₃)₃, La(NO₃)₃, CeCl₃, YCl₃ and LaCl₃, and mixtures thereof.

According to an even more preferred embodiment, the compound(s) belonging to the group of the rare-earth metals are chosen from CeCl₃ and YCl₃, and mixtures thereof.

According to a preferred embodiment, the crosslinking agent is chosen from (poly)amine compounds A), (poly)thiol compounds B), (poly)acrylate compounds C), metal alkoxides D) and (poly)carbonyl compounds E), and preferably from (poly)amine compounds A), (poly)thiol compounds B) and (poly)acrylate compounds C).

In particular, said (poly)amine compounds A) are chosen from a) chitosans, such as poly(D-glucosamine), b) polyether diamines, particularly polyethylene glycol α,ω-diamine (bearing an amine function at the end of the chain), c) polyether triamines, such as polyetheramine (or Jeffamine), d) aminoalkoxysilanes, such as APTES, and e) polydialkylsiloxanes comprising primary amine groups at the end of the chain or on side chains, in particular polydimethylsiloxanes comprising primary amine groups, such as bis(3-aminopropyl)-terminated poly(dimethoxysiloxane) (PDMS-diNH₂) and amodimethicones comprising amine groups on side chains, such as bis-cetearyl amodimethicone, notably the product sold by Momentive Performance Materials, and f) polyamine compounds NH₂-alk-NH₂, in which alk denotes a divalent C₂-C₂₀ hydrocarbon-based chain, optionally interrupted with one or more heteroatoms chosen from —O—, —N(R)— with R denoting a hydrogen atom or a C₁-C₄ alkyl radical, such as spermidine.

According to a preferred embodiment, said (poly)amine compounds A) are chosen from a) chitosans, such as poly(D-glucosamine), c) polyether triamines, such as polyetheramine (or Jeffamine), and e) polydialkylsiloxanes comprising primary amine groups at the end of the chain or on side chains, in particular polydimethylsiloxanes comprising primary amine groups, such as bis(3-aminopropyl)-terminated poly(dimethoxysiloxane) (PDMS-diNH₂) and amodimethicones comprising amine groups on side chains, such as bis-cetearyl amodimethicone, notably the product sold by Momentive Performance Materials, and f) polyamine compounds NH₂-alk-NH₂, in which alk denotes a divalent C₂-C₂₀ hydrocarbon-based chain, optionally interrupted with one or more heteroatoms chosen from —O—, —N(R)— with R denoting a hydrogen atom or a C₁-C₄ alkyl radical, such as spermidine or 4,7,10-trioxa-1,13-tridecanediamine.

In particular, said (poly)thiol compounds B) are chosen from a) polydialkylsiloxanes bearing thiol functions, and b) alkoxysilanes bearing thiol functions, and in particular are chosen from a) polydialkylsiloxanes bearing thiol functions, preferentially polydimethylsiloxanes comprising thiol groups on the side chain (such as mercaptopropyl), notably those of formula (XIII).

In particular, said (poly)acrylate C) compounds are chosen from those of formula (XIV), notably trimethylolpropane triacrylate, and those of formula (XV), notably copolymers of dimethylsiloxane and acryloxypropylmethylsiloxane, preferably trimethylolpropane triacrylate.

According to a preferred embodiment, the crosslinking agent(s) ii) are chosen from:
A) (poly)amine compounds chosen from:
ia) chitosans such as poly(D-glucosamine),
ib) polyether diamines, in particular polyethylene glycol α,ω-diamines, bearing a chain-end amine function,
ic) polyether triamines, in particular polyetheramines (or Jeffamine),
id) aminoalkoxysilanes, in particular APTES,
ie) polyamine compounds NH₂-alk-NH₂, in which alk denotes a divalent C₂-C₂₀ hydrocarbon-based chain, optionally interrupted with one or more heteroatoms chosen from —O—, —N(R)— with R denoting a hydrogen atom or a C₁-C₄ alkyl radical, notably spermidine and 4,7,10-trioxa-1,13-tridecanediamine, and
if) polydialkylsiloxanes comprising primary amine groups at the end of the chain or on side chains, in particular polydimethylsiloxanes comprising primary amine groups, more particularly bis(3-aminopropyl)-terminated poly(dimethoxysiloxane) (PDMS-diNH₂) and amodimethicones comprising amine groups on side chains, more particularly bis-cetearyl amodimethicone;
B) (poly)thiol compounds chosen from:
iia) polydialkylsiloxanes bearing thiol functions, and
iib) alkoxysilanes bearing thiol functions,
and in particular chosen from iia) polydialkylsiloxanes bearing thiol functions, preferably from polydimethylsiloxanes comprising thiol groups on the side chain, in particular mercaptopropyl, and more particularly chosen from the compounds of formula (XIII):

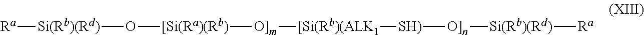

in which formula (XIII):
   R^{a} and R^{b}, which may be identical or different, preferably identical, represent a (C₁-C₄)alkyl group, in particular methyl, a (C₁-C₄)alkoxy group, in particular methoxy, an aryl group, in particular phenyl, an aryloxy group, in particular phenoxy, an aryl(C₁-C₄)alkyl group, in particular benzyl, or an aryl(C₁-C₄)alkoxy group, in particular benzoxy, preferably a (C₁-C₄)alkyl group, more preferentially methyl,
   R^{d} represents a (C₁-C₄)alkyl group, in particular methyl, a (C₁-C₄)alkoxy group, in particular methoxy, an aryl group, in particular phenyl, an aryloxy group, in particular phenoxy, an aryl(C₁-C₄)alkyl group, in particular benzyl, an aryl(C₁-C₄)alkoxy group, in particular benzoxy, or a (C₁-C₆)alkyl group substituted with a (C₁-C₄)alkylamino, amino or thiol group, and preferably a (C₁-C₄)alkyl group, more preferentially methyl,
and preferably R^{a}, R^{b} and R^{d} are identical and represent a (C₁-C₆)alkyl group, more preferentially methyl,
   ALK₁ represents a linear or branched, optionally cyclic, saturated or unsaturated divalent hydrocarbon-based chain comprising from 1 to 100 carbon atoms, optionally interrupted with one or more heteroatoms such as oxygen, sulfur or nitrogen, in particular oxygen, a (thio)carbonyl group C(X) with X representing O or S, or combinations thereof, in particular —O—, —O—C(O)— or —C(O)—O—; preferably, ALK₁ represents a (C₁-C₆)alkylene and more preferentially (C₁-C₄)alkylene group, even more preferentially propylene,
   n and m, which may be identical or different, represent an integer greater than 2, and in particular the values of m and n are such that the weight-average molecular weight of said polyorganosiloxane is between 1000 and 55 000 g·mol⁻¹; and
C) the (poly)acrylate compounds of formula (XIV):

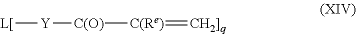

in which formula (XIV):
   q represents an integer greater than or equal to 2, in particular n is between 2 and 10 and preferably between 2 and 5 inclusive,
   L denotes a linear or branched, saturated or unsaturated, or (hetero)cyclic, saturated or unsaturated, multivalent (at least divalent) group, in particular comprising between 1 and 500 carbon and/or silicon atoms, more particularly between 2 and 40 carbon and/or silicon atoms, even more particularly between 3 and 30 carbon and/or silicon atoms, preferably between 6 and 20 carbon atoms; L being optionally interrupted and/or terminated with one or more heteroatoms or groups chosen from O, S, N, Si, C(X), and combinations thereof, in particular —O—, —O—C(X)—, —N(R)—C(X)—, —Si(R_{c})(R_{d})—O— with R representing a hydrogen atom or a (C₁-C₆)alkyl group, in particular methyl; and/or L being optionally substituted with one or more groups chosen from: —N(Rₐ)R_{b} and —(X′)ₐ—C(X)—(X″)_{b}—Rₐ; with X, X′ and X″, which may be identical or different, representing an oxygen or sulfur atom, or a group N(R_{b}); a and b being 0 or 1, preferably the sum of a+b being 1; Rₐ and R_{b}, which may be identical or different, represent a hydrogen atom, a (C₁-C₆)alkyl group or an aryl(C₁-C₄)alkyl group, in particular benzyl, preferably Rₐ and R_{b} represent a hydrogen atom, and R_{c} and R_{d}, which may be identical or different, represent a (C₁-C₆)alkyl, aryl(C₁-C₄)alkyl or (C₁-C₆)alkoxy group,
   R^{e} represents a hydrogen atom or a (C₁-C₄)alkyl group, in particular methyl; preferably, R^{e} represents a hydrogen atom, and
   Y represents an oxygen atom or an amino group —N(H)—, preferably an oxygen atom, preferably Y is an oxygen atom and R^{e} is a hydrogen atom, preferably L represents a di- or trivalent, preferably trivalent, hydrocarbon-based chain comprising from 1 to 8 carbon atoms, q is 2 or 3, preferably 3, and
more preferentially, the compounds of formula (XIV) are trimethylolpropane triacrylate;
D) metal alkoxides chosen from: zirconium ethoxide (Zr(OC₂H₅)₄), zirconium propoxide (Zr(OCH₂CH₂CH₃)₄), zirconium isopropoxide (Zr(OCH(CH₃)₂)₄), zirconium butoxide Zr(OCH₂CH₂CH₂CH₃)₄, zirconium tert-butoxide (Zr(OC(CH₃)₃)₄), titanium ethoxide (Ti(OC₂H₅)₄), titanium propoxide (Ti(OCH₂CH₂CH₃)₄), titanium isopropoxide (Ti(OCH(CH₃)₂)₄), titanium butoxide (Ti(OCH₂CH₂CH₂CH₃)₄), titanium tert-butoxide (Ti(OC(CH₃)₃)₄), titanium 2-ethylhexyloxide (Ti(OCH₂CH(C₂H₅)(CH₂)₃CH₃)₄), and mixtures thereof, more preferentially chosen from zirconium propoxide, titanium propoxide, titanium butoxide and mixtures thereof,
E) (poly)carbonyl compounds chosen from (poly)carbonyl compounds including a saturated or unsaturated, aromatic C₅-C₇ carbocycle, preferably aromatic, such as phenyl, or non-aromatic and saturated such as cyclohexyl, more preferentially unsaturated and aromatic, such as terephthaldehyde.
   Cosmetic Active Agents iii)

According to a particular embodiment, the process of the invention also comprises the application of iii) at least one cosmetic active agent to keratin fibres, preferably the hair.

More particularly, in the treatment process according to the invention, at least one of the compositions “C1”, “C2”, “C3”, “C4” or “C5” used comprises one or more cosmetic active agents.

In particular, the cosmetic active agent(s) iii) are chosen from:
a) dyestuffs (or colouring agents), in particular chosen from pigments, direct dyes and mixtures thereof,
b) active agents for caring for keratin fibres, preferably the hair,
c) UV-screening agents, and
d) mixtures thereof.

According to a particular embodiment, the at least one cosmetic active agent iii) is chosen from dyestuffs, preferably chosen from pigments, direct dyes and mixtures thereof, more preferentially pigments.

According to another embodiment, at least one of the compositions “C1”, “C2”, “C3”, “C4” or “C5” used comprises one or more cosmetic active agent(s) chosen from a) dyestuffs (or colouring agents), in particular chosen from pigments, direct dyes and mixtures thereof, preferably chosen from pigments.

According to a particular embodiment, composition “C1” contains at least one cosmetic active agent iii) as defined previously and hereinbelow, preferably chosen from a) dyestuffs, and more preferentially chosen from pigments.

According to a particular embodiment, composition “C2” contains at least one cosmetic active agent iii) as defined previously and hereinbelow, preferably chosen from a) dyestuffs, and more preferentially chosen from pigments.

According to a particular embodiment, composition “C3” contains at least one cosmetic active agent iii) as defined previously and hereinbelow, preferably chosen from a) dyestuffs, and more preferentially chosen from pigments.

Needless to say, a person skilled in the art will take care to select this or these optional cosmetic active agent(s), and/or the amount thereof, such that the advantageous properties of the corresponding composition according to the invention are not, or are not substantially, adversely affected by the envisaged addition.

### Dyestuffs

According to a particular embodiment, the process of the invention uses one or more dyestuffs.

More particularly, in the process of the invention, at least one of the compositions “C1”, “C2”, “C3”, “C4” or “C5” used comprises at least one particulate or non-particulate, water-soluble or water-insoluble dyestuff, preferably in a proportion of at least 0.01% by weight relative to the total weight of the composition considered.

For obvious reasons, this amount is liable to vary significantly with regard to the intensity of the desired colour effect and of the colour intensity afforded by the dyestuffs under consideration, and its adjustment clearly falls within the competence of a person skilled in the art.

Preferably, at least one of the compositions “C1”, “C2”, “C3”, “C4” or “C5” comprises at least one dyestuff chosen from pigments, direct dyes and mixtures thereof, more preferentially pigments.

More preferentially, the pigment(s) of the invention are chosen from carbon black, iron oxides, in particular yellow, red and black iron oxides, and micas coated with iron oxide, triarylmethane pigments, in particular blue and violet triarylmethane pigments, in particular Blue 1 Lake, azo pigments, in particular red azo pigments, more particularly D&C Red 7, an alkali metal salt of lithol red, in particular the calcium salt of lithol red B, and even more preferentially chosen from red iron oxides, yellow iron oxides and azo pigments, in particular red azo pigments, more particularly D&C Red 7.

### Pigments

For the purposes of the invention, the term “pigment” means any compound that is capable of imparting colour to keratin materials. These compounds have a solubility in water at 25° C. and at atmospheric pressure (760 mmHg) of less than 0.05% by weight, and preferably less than 0.01% by weight.

As pigments that are suitable for use in the invention, mention may notably be made of the organic and/or mineral pigments known in the art, notably those described in Kirk-Othmer's Encyclopedia of Chemical Technology and in Ullmann's Encyclopedia of Industrial Chemistry.

These pigments may be synthetic or natural.

These pigments may be in pigment powder or paste form.

They may be coated or uncoated.

These pigments may be chosen, for example, from mineral pigments, organic pigments, lakes, pigments with special effects such as nacres or glitter flakes, and mixtures thereof.

A pigment that is suitable for use in the invention may be chosen from mineral pigments.

The term “mineral pigment” means any pigment that satisfies the definition in Ullmann's encyclopedia in the chapter on inorganic pigments. Among the mineral pigments that are useful in the present invention, mention may be made of manganese violet, ultramarine blue, chromium hydrate, ferric blue and titanium, zirconium or cerium oxides or dioxides, and also of zinc, iron or chromium oxides.

It may also be a pigment having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30. They may also be pigments having a structure that may be, for example, of silica microsphere type containing iron oxide. An example of a pigment having this structure is the product sold by the company Miyoshi under the reference PC Ball PC-LL-100 P, this pigment consisting of silica microspheres containing yellow iron oxide.

Advantageously, the pigments may be iron oxides and/or titanium dioxides.

A pigment that is suitable for use in the invention may be chosen from organic pigments.

The term “organic pigment” means any pigment that satisfies the definition in Ullmann's encyclopedia in the chapter on organic pigments. Among the organic pigments that are useful in the present invention, mention may be made of nitroso, nitro, azo, xanthene, pyrene, quinoline, anthraquinone, triphenylmethane, fluorane, phthalocyanine, metal-complex, isoindolinone, isoindoline, quinacridone, perinone, perylene, diketopyrrolopyrrole, indigo, thioindigo, dioxazine, triphenylmethane and quinophthalone compounds. In particular, the white or coloured organic pigments may be chosen from carmine, carbon black, aniline black, azo yellow, quinacridone, phthalocyanine blue, the blue pigments codified in the Color Index under the references CI 42090, 69800, 69825, 74100, 74160, the yellow pigments codified in the Color Index under the references CI 11680, 11710, 19140, 20040, 21100, 21108, 47000, 47005, the green pigments codified in the Color Index under the references CI 61565, 61570, 74260, the orange pigments codified in the Color Index under the references CI 11725, 45370, 71105, the red pigments codified in the Color Index under the references CI 12085, 12120, 12370, 12420, 12490, 14700, 15525, 15580, 15620, 15630, 15800, 15850, 15865, 15880, 26100, 45380, 45410, 58000, 73360, 73915, 75470, the pigments obtained by oxidative polymerization of indole or phenol derivatives as described in patent FR 2 679 771.

Examples that may also be mentioned include pigment pastes of organic pigments, such as the products sold by the company Hoechst under the names: Cosmenyl Yellow IOG: Yellow 3 pigment (CI 11710); Cosmenyl G yellow: Yellow 1 pigment (CI 11680); Cosmenyl GR orange: Orange 43 pigment (CI 71105); Cosmenyl R red: Red 4 pigment (CI 12085); Cosmenyl FB carmine: Red 5 pigment (CI 12490); Cosmenyl RL violet: Violet 23 pigment (CI 51319); Cosmenyl A2R blue: Blue 15.1 pigment (CI 74160); Cosmenyl GG green: Green 7 pigment (CI 74260); Cosmenyl R black: Black 7 pigment (CI 77266).

The pigments in accordance with the invention may also be in the form of composite pigments, as described in patent EP 1 184 426. These composite pigments may particularly be composed of particles including an inorganic core, at least one binder for attaching the organic pigments to the core, and at least one organic pigment which at least partially covers the core.

The organic pigment may also be a lake.

The term “lake” refers to dyes adsorbed onto insoluble particles, the assembly thus obtained remaining insoluble during use.

The inorganic substrates onto which the dyes are adsorbed are, for example, alumina, silica, calcium sodium borosilicate or calcium aluminium borosilicate and aluminium. Among the dyes adsorbed on organic substrates, mention may be made of carminic acid. Mention may also be made of the dyes known under the following names: D & C Red 21 (CI 45 380), D & C Orange 5 (CI 45 370), D & C Red 27 (CI 45 410), D & C Orange 10 (CI 45 425), D & C Red 3 (CI 45 430), D & C Red 4 (CI 15 510), D & C Red 33 (CI 17 200), D & C Yellow 5 (CI 19 140), D & C Yellow 6 (CI 15 985) D & C Green 5 (CI 61 570), D & C Yellow 10 (CI 77 002), D & C Green 3 (CI 42 053), D & C Blue 1 (CI 42 090), FDC Red 4, D & C Red 6, D & C Red 22, D & C Red 28, D & C Red 30, D & C Orange 4, D & C Yellow 8, D & C Green 5, D & C Red 17, D & C Green 6, D & C Yellow 11, D & C Violet 2, Sudan red, carotenes (0-carotene, lycopene), xanthophylls (capsanthin, capsorubin, lutein), palm oil, Sudan brown, quinoline yellow, annatto, curcumin, betanin (beet), carmine, copper chlorophyllin, methylene blue, anthocyanins (enocianine, black carrot, hibiscus, elderberry), caramel, riboflavin, beet juice and caramel.

An example of a lake that may be mentioned is the product known under the following name: D&C Red 7 (CI 15 850:1).

The pigment may also be a pigment with special effects.

The term “pigments with special effects” refers to pigments that generally create a coloured appearance (characterized by a certain shade, a certain vivacity and a certain level of luminance) that is non-uniform and that changes as a function of the conditions of observation (light, temperature, angles of observation, etc.). They thereby differ from coloured pigments, which afford a standard uniform opaque, semi-transparent or transparent shade.

Several types of special effect pigments exist: those with a low refractive index, such as fluorescent or photochromic pigments, and those with a higher refractive index, such as nacres, interference pigments or glitter flakes.

The size of the pigment used in the composition according to the present invention is generally between 10 nm and 200 μm, preferably between 20 nm and 80 μm and more preferentially between 30 nm and 50 μm.

The pigments may be dispersed in the composition by means of a dispersant.

This dispersant may be a surfactant, an oligomer, a polymer or a mixture of several thereof, bearing one or more functionalities with strong affinity for the surface of the particles to be dispersed. In particular, they may become physically or chemically attached to the surface of the pigments. These dispersants also contain at least one functional group that is compatible with or soluble in the continuous medium. In particular, esters of 12-hydroxystearic acid in particular and of C₈ to C₂₀ fatty acid and of polyols such as glycerol or diglycerol are used, such as poly(12-hydroxystearic acid) stearate with a molecular weight of approximately 750 g/mol, such as the product sold under the name Solsperse 21 000 by the company Avecia, polyglyceryl-2 dipolyhydroxystearate (CTFA name) sold under the reference Dehymyls PGPH by the company Henkel, or polyhydroxystearic acid such as the product sold under the reference Arlacel P100 by the company Uniqema, and mixtures thereof. As other dispersants that may be used in the compositions of the invention, mention may be made of quaternary ammonium derivatives of polycondensed fatty acids, for instance Solsperse 17 000 sold by the company Avecia, and polydimethylsiloxane/oxypropylene mixtures such as those sold by the company Dow Corning under the references DC2-5185 and DC2-5225 C. The pigments used in the composition may be surface-treated with an organic agent. According to a particular embodiment, the dispersant(s) are of amino silicone type other than the alkoxysilanes described previously and are cationic. Preferably, the pigment(s) is (are) chosen from mineral, mixed mineral-organic, or organic pigments.

According to a particular embodiment, the pigment(s) according to the invention are organic pigments, preferentially organic pigments surface-treated with an organic agent chosen from silicone compounds.

According to another embodiment of the invention, the pigment(s) according to the invention are mineral pigments.

### Direct Dyes

According to a particular embodiment of the invention, the cosmetic active agent is a dyestuff chosen from one or more direct dyes.

The term “direct dye” means natural and/or synthetic dyes, other than oxidation dyes. These are dyes which will spread superficially over the fibre. They may be ionic or nonionic, preferably cationic or nonionic.

Among the direct dyes that are suitable for use in the invention, mention may be made of azo direct dyes; (poly)methine dyes such as cyanines, hemicyanines and styryls; carbonyl dyes; azine dyes; nitro(hetero)aryl dyes; tri(hetero)arylmethane dyes; porphyrin dyes; phthalocyanine dyes and natural direct dyes, alone or in the form of mixtures.

The direct dyes are preferably cationic direct dyes. Mention may be made of the hydrazono cationic dyes of formulae (A) and (B) below and the azo cationic dyes of formulae (C) and (D) below:

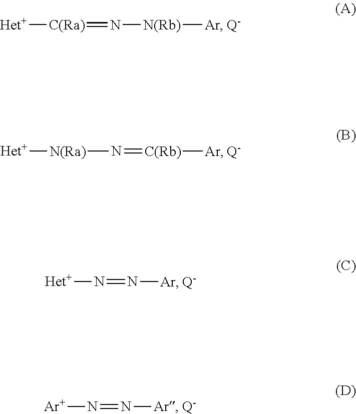

in which formulae (A) to (D):
Het⁺ represents a cationic heteroaryl radical, preferentially bearing an endocyclic cationic charge, such as imidazolium, indolium or pyridinium, which is optionally substituted, preferentially with at least one (C₁-C₈)alkyl group such as methyl;
Ar⁺ represents an aryl radical, such as phenyl or naphthyl, bearing an exocyclic cationic charge, preferentially ammonium, particularly tri(C₁-C₈)alkylammonium, such as trimethylammonium;
Ar represents an aryl group, notably phenyl, which is optionally substituted, preferentially with one or more electron-donating groups such as optionally substituted (C₁-C₈)alkyl, optionally substituted (C₁-C₈)alkoxy, (di)(C₁-C₈)(alkyl)amino optionally substituted on the alkyl group(s) with a hydroxyl group, aryl(C₁-C₈)alkylamino, and optionally substituted N—(C₁-C₈)alkyl-N-aryl(C₁-C₈)alkylamino or alternatively Ar represents a julolidine group;
Ar″ represents an optionally substituted (hetero)aryl group, such as phenyl or pyrazolyl, which are optionally substituted, preferentially with one or more (C₁-C₈)alkyl, hydroxyl, (di)(C₁-C₈)(alkyl)amino, (C₁-C₈)alkoxy or phenyl groups;
Ra and Rb, which may be identical or different, represent a hydrogen atom or a (C₁-C₈)alkyl group, which is optionally substituted, preferentially with a hydroxyl group;
   or else the substituent Rₐ with a substituent of Het⁺ and/or R_{b} with a substituent of Ar form, together with the atoms that bear them, a (hetero)cycloalkyl; in particular, Ra and Rb represent a hydrogen atom or a (C₁-C₄)alkyl group optionally substituted with a hydroxyl group;
Q⁻ represents an organic or mineral anionic counterion, such as a halide or an alkyl sulfate.

In particular, mention may be made of the azo and hydrazono direct dyes bearing an endocyclic cationic charge of formulae (A) to (D) as defined previously,

more particularly the cationic direct dyes bearing an endocyclic cationic charge described in patent applications WO 95/15144, WO 95/01772 and EP 714 954,

Preferably, the direct dyes are chosen from the compounds of formulae (E) and (F) below:

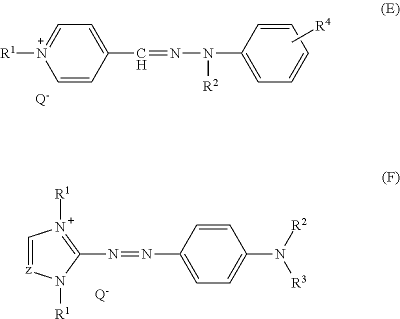

in which formulae (E) and (F).
R¹ represents a (C₁-C₄)alkyl group such as methyl;
R² and R³, which may be identical or different, represent a hydrogen atom or a (C₁-C₄)alkyl group, such as methyl;
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; in particular, R⁴ is a hydrogen atom;
Z represents a CH group or a nitrogen atom, preferentially CH;
Q⁻ is an anionic counterion as defined previously, in particular a halide, such as chloride, or an alkyl sulfate, such as methyl sulfate or mesyl.

In particular, the dyes of formulae (E) and (F) are chosen from Basic Red 51, Basic Yellow 87 and Basic Orange 31 or derivatives thereof with Q′ being an anionic counterion as defined previously, particularly a halide such as chloride, or an alkyl sulfate such as methyl sulfate or mesyl. The direct dyes may be chosen from anionic direct dyes. The anionic direct dyes of the invention are dyes commonly referred to as “acid” direct dyes owing to their affinity for alkaline substances.

The term “anionic direct dye” means any direct dye including in its structure at least one CO₂R′ or SO₃R′ substituent with R′ denoting a hydrogen atom or a cation originating from a metal or an amine, or an ammonium ion.

The anionic direct dyes may be chosen from direct nitro acid dyes, azo acid dyes, azine acid dyes, triarylmethane acid dyes, indoamine acid dyes, anthraquinone acid dyes, indigoid dyes and natural acid dyes.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin and orceins. Use may also be made of extracts or decoctions containing these natural dyes and particularly henna-based poultices or extracts.

Preferably, the direct dyes are chosen from anionic direct dyes.

The dyestuffs, preferably the pigments, may be present in concentrations ranging from 0.01% to 30% by weight, preferably from 0.02% to 20% by weight and more particularly from 0.05% to 15% by weight relative to the total weight of the composition containing them.

The direct dye(s) may be present in concentrations ranging from 0.001% to 10% by weight and preferably from 0.005% to 5% by weight relative to the total weight of the composition containing them.

Preferably, the cosmetic active agent(s), in particular the dyestuff(s) and more particularly the pigment(s), are introduced into at least one of the compositions “C1”, “C2”, “C3”, “C4” or “C5”.

### Active Agents for Caring for Keratin Fibres

According to a particular embodiment, the process of the invention uses b) one or more active agents for caring for keratin fibres, notably the hair.

More particularly, in the process of the invention, according to one embodiment, at least one of the compositions “C1”, “C2”, “C3”, “C4” or “C5” used comprises one or more active agents for caring for keratin fibres, preferably in a proportion of at least 0.01% by weight relative to the total weight of the composition considered. In particular, the care active agent may be at least one hydrophilic active agent and/or one lipophilic active agent, and preferably a hydrophilic care active agent.

The term “hydrophilic active agent” means a water-soluble or water-dispersible active agent that is capable of forming hydrogen bonds.

The cosmetic active agent(s) for haircare may notably be chosen from active agents having an action on the barrier function, antioxidant active agents, moisturizing active agents, sebum-regulating active agents, active agents intended for combating the effects of pollution, antimicrobial or bactericidal active agents, fragrances, vitamins and derivatives thereof, notably esters thereof, in particular tocopherol (vitamin E) and esters thereof (such as tocopheryl acetate), and mixtures thereof.

Among the active agents for caring for keratin fibres, mention may be made in particular of antioxidant active agents, moisturizing active agents and sebum-regulating active agents.

The active agent(s) for caring for keratin fibres may in particular be present, in the composition containing same, in a content ranging from 0.01% to 30% by weight, relative to the weight of the composition, and preferably from 0.02% to 25% by weight.

### UV-Screening Agents

According to one embodiment of the process of the invention, at least one of the compositions “C1”, “C2”, “C3”, “C4” or “C5” used comprises, as cosmetic active agent, at least one UV-screening agent.

The UV-screening agent is a UV-screening agent normally used in cosmetics.

It may be chosen from the positive list contained in Annex VI of (EC) Regulation No. 1223/2009, which specifies the list of UV-screening agents permitted in cosmetics.

The UV-screening agents that are suitable for use in the invention may be of varied nature.

They may be lipophilic, hydrophilic or insoluble organic agents.

The term “lipophilic UV-screening agent” means any cosmetic or dermatological screening agent that can be fully dissolved in molecular form in a liquid fatty phase or that can be dissolved in colloidal form (for example in micellar form) in a liquid fatty phase.

The term “hydrophilic UV-screening agent” means any cosmetic or dermatological screening agent that can be fully dissolved in molecular form in a liquid aqueous phase or that can be dissolved in colloidal form (for example in micellar form) in a liquid aqueous phase.

The term “insoluble UV-screening agent” means any cosmetic or dermatological screening agent which is not defined either as a lipophilic UV-screening agent or as a hydrophilic UV-screening agent, and which is in the form of particles in aqueous phase or liquid fatty phase.

The UV-screening agents of the composition according to the invention may afford UVA and/or UVB photoprotection.

According to a preferred embodiment, the compositions, which are preferably cosmetic compositions, may comprise at least one organic and/or mineral UV-screening agent (for screening out the UV radiation of sunlight).

In particular, the UV-screening agent(s) are chosen from bis-resorcinyl triazine derivatives, dibenzoylmethane derivatives, benzylidene camphor derivatives, and mixtures thereof. The organic UV-screening agents may also be chosen from anthranilic derivatives; cinnamic derivatives; salicylic derivatives; benzophenone derivatives; phenylbenzotriazole derivatives; benzalmalonate derivatives, notably those mentioned in patent U.S. Pat. No. 5,624,663; phenylbenzimidazole derivatives; imidazolines; 4,4-diarylbutadiene derivatives; bis-benzazolyl derivatives, as described in patents EP 6 693 23 and U.S. Pat. No. 2,463,264; p-aminobenzoic acid (PABA) derivatives; methylenebis(hydroxyphenylbenzotriazole) derivatives, as described in patent applications U.S. Pat. Nos. 5,237,071, 5,166,355, GB 2 303 549, DE 197 26, 184 and EP 893 119; benzoxazole derivatives, such as those described in patent applications EP 0 832 642, EP 1 027 883, EP 1 300 137 and DE 101 62 844; screening polymers and screening silicones such as those notably described in patent application WO 93/04665; α-alkylstyrene-based dimers such as those described in patent application DE 198 55 649; 4,4-diarylbutadienes such as those described in patent applications EP 0 967 200, DE 197 46 654, DE 197 55 649, EP 1 008 586, EP 1 133 980 and EP 133 981; other merocyanine derivatives such as those described in patent applications WO 04/006878, WO 05/058269 and WO 06/032741, and mixtures thereof.

According to a particular embodiment, the concentration of the organic UV-screening agents in the compositions ranges from 1% to 50%, preferably from 1% to 40% by weight, for example from 5% to 35% by weight, relative to the total weight of the composition.

The UV-screening agent(s) may be mineral UV-screening agents, which are generally pigments. The pigments may or may not be coated.

Thus, the mineral UV-screening agents may be chosen from coated or uncoated pigments, and in particular from coated titanium oxide pigments, silicone-treated titanium oxides, uncoated titanium oxide pigments, uncoated zinc oxide pigments, coated zinc oxide pigments, uncoated cerium oxide pigments, uncoated iron oxide pigments, coated iron oxide pigments, and mixtures thereof.

According to a particular embodiment, compositions “C1” to “C5” are free of mineral UV-screening agents.

According to a particular embodiment, the amount of the mineral UV-screening agent(s) present in compositions “C1”, “C2”, “C3”, “C4” or “C5” according to the invention may range from 0.01% to 20% by weight relative to the total weight of the composition containing same. It ranges, for example, from 1% to 15% by weight, relative to the total weight of the composition.

According to a particular embodiment, at least one of the compositions “C1” to “C5” also comprises one or more organic UV-screening agents and one or more mineral UV-screening agents.

According to a particular embodiment, the compositions comprise a combination of UV-screening agents as described in patent FR 2 977 490, patent application WO 2013/004777 or patent application US 2014/0134120.

Preferably, the process for treating keratin fibres, preferably the hair, and compositions “C1”, “C2”, “C3”, “C4” or “C5” use or comprise one or more dyestuffs chosen from pigments, direct dyes and mixtures thereof, preferably pigments; more preferentially, the pigment(s) of the invention are chosen from carbon black, iron oxides, notably yellow, red and black iron oxides, and micas coated with iron oxide, triarylmethane pigments, notably blue and violet triarylmethane pigments, such as Blue 1 Lake, azo pigments, notably red azo pigments, such as D&C Red 7, an alkali metal salt of lithol red, such as the calcium salt of lithol red B, even more preferentially red iron oxides, yellow iron oxides and azo pigments, notably red azo pigments, such as D&C Red 7.

### Fatty Phase—Fatty Substances

According to a particular embodiment, the process of the invention also uses iv) one or more fatty substances, in particular one or more oils, preferably volatile oils.

In particular, at least one of the compositions “C1”, “C2”, “C3”, “C4” or “C5” used in the process of the invention contains a fatty phase.

In particular, at least one of the compositions “C1”, “C2”, “C3”, “C4” or “C5” used in the process of the invention comprises one or more fatty substances, different from the compounds i), in particular one or more oils, preferably volatile oils.

The term “oil” refers to a fatty substance that is liquid at room temperature (20° C.) and atmospheric pressure (760 mmHg).

The term “hydrocarbon-based oil” means an oil formed essentially from, or even constituted of, carbon and hydrogen atoms, and possibly oxygen and nitrogen atoms, and not containing any silicon or fluorine atoms. It may contain alcohol, ester, ether, carboxylic acid, amine and/or amide groups.

According to one embodiment of the invention, the oil(s) iv) are chosen from volatile oils, in particular:
hydrocarbon-based oils containing from 8 to 16 carbon atoms, in particular branched C₈-C₁₆ alkanes, in particular isoalkanes, more particularly isoalkanes (also known as isoparaffins), preferably C₁₃-C₁₆ isoparaffins, isododecane, isodecane, isohexadecane, for example the oils sold under the trade names Isopar or Permethyl, alone or as mixtures, preferably isododecane (also known as 2,2,4,4,6-pentamethylheptane), linear alkanes, in particular C₁₁-C₁₆ alkanes, alone or as mixtures, in particular hexane, decane, undecane, tridecane, isoparaffins, in particular n-dodecane (C₁₂) and n-tetradecane (C₁₄), the undecane-tridecane mixture, mixtures of n-undecane (C₁₁) and n-tridecane (C₁₃), and mixtures thereof and also mixtures of n-undecane (C₁₁) and n-tridecane (C₁₃), and volatile C₅-C₁₂ cyclic, non-aromatic alkanes;
short-chain esters containing from 3 to 8 carbon atoms in total, in particular ethyl acetate, methyl acetate, propyl acetate or n-butyl acetate;
carbonate hydrocarbon-based oils of structure R′₁—O—C(O)—O—R′₂ in which R′₁ and R′₂ independently denote a linear, branched or cyclic C₄-C₈ alkyl group, preferably a C₄-C₈ alkyl group, advantageously chosen more preferentially from dibutyl carbonate or dipentyl carbonate;
ether oils of formula R₁—O—R₂ in which R₁ and R₂, independently of each other, denote a linear, branched or cyclic C₄-C₈ alkyl group, preferably a C₄-C₈ alkyl group;
silicone oils, in particular comprising from 2 to 7 silicon atoms, and optionally including alkyl or alkoxy groups containing from 1 to 10 carbon atoms, in particular dimethicones of viscosity 5 and 6 cSt, cyclopentadimethylsiloxane, dodecamethylpentasiloxane, cyclohexadimethylsiloxane, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof, more preferentially, the volatile oil(s) iv) are chosen from C₈-C₁₆ alkanes, in particular branched alkanes, preferably isododecane.

In particular, at least one of the compositions “C1”, “C2”, “C3”, “C4” or “C5” used in the process according to the invention comprises one or more non-volatile oils, preferably chosen from:
non-volatile fluoro oils, in particular chosen from fluorinated polyethers, fluorosilicone oils and fluorosilicones;
non-volatile silicone oils, in particular chosen from non-volatile silicones having the following INCI names: dimethicone, dimethiconol, trimethyl pentaphenyl trisiloxane, tetramethyl tetraphenyl trisiloxane, diphenyl dimethicone, trimethylsiloxyphenyl dimethicone, phenyl trimethicone, diphenylsiloxy phenyl trimethicone; and also mixtures thereof;
non-volatile apolar hydrocarbon-based oils, in particular chosen from linear or branched compounds of mineral or synthetic origin: i) liquid paraffin, ii) squalane, isoeicosane, iii) mixtures of linear, saturated hydrocarbons, more particularly C₁₅-C₂₈ hydrocarbons, in particular mixtures whose INCI names are (C₁₅-C₁₉)Alkane, (C₁₈-C₂₁)Alkane, (C₂₁-C₂₈)Alkane, iv) hydrogenated or non-hydrogenated polybutenes; v) hydrogenated or non-hydrogenated polyisobutenes, preferably hydrogenated, vi) hydrogenated or non-hydrogenated polydecenes, vii) decene/butene copolymers, butene/isobutene copolymers and viii) mixtures thereof;
non-volatile polar hydrocarbon-based oils, which may be chosen from:
i) saturated, unsaturated, linear or branched C₁₀-C₂₆ fatty alcohols, preferably monoalcohols; in particular, the C₁₀-C₂₆ alcohols are fatty alcohols, which are preferably branched when they comprise at least 16 carbon atoms; preferably, the fatty alcohol comprises from 10 to 24 carbon atoms, and more preferentially from 12 to 22 carbon atoms, in particular such as lauryl alcohol, isostearyl alcohol, oleyl alcohol, 2-butyloctanol, 2-undecylpentadecanol, 2-hexyldecyl alcohol, isocetyl alcohol, octyldodecanol and mixtures thereof,
ii) triglycerides consisting of fatty acid esters of glycerol, in particular the fatty acids of which may have chain lengths ranging from C₄ to C₃₆, and notably from C₁₈ to C₃₆, these oils possibly being linear or branched, and saturated or unsaturated; by way of example, mention may notably be made of heptanoic or octanoic triglycerides, caprylic/capric acid triglycerides, plant oils such as wheatgerm oil, sunflower oil, grapeseed oil, sesame seed oil, corn oil, apricot kernel oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil, musk rose oil, groundnut oil, coconut oil, argan oil, passionflower oil, kaya oil; the liquid fraction of shea butter, and the liquid fraction of cocoa butter; and also mixtures thereof;
iii) linear aliphatic hydrocarbon-based esters of formula R—C(O)—OR′ in which R—C(O)—O— represents the carboxylic acid residue containing from 2 to 40 carbon atoms, and R′ represents a hydrocarbon-based chain containing from 1 to 40 carbon atoms, aliphatic hydrocarbon-based esters of alkylene glycol, in particular ethylene glycol or propylene glycol; the total number of carbon atoms in particular being at least 10; notably chosen from isoamyl laurate, cetostearyl octanoate, isopropyl myristate, isopropyl palmitate, isopropyl stearate or isostearate, ethyl palmitate, 2-ethylhexyl palmitate, isostearyl isostearate, octyl stearate, isostearyl heptanoate, octanoates, decanoates or ricinoleates of alcohols or of polyalcohols, such as propylene glycol dioctanoate, cetyl octanoate or tridecyl octanoate, 2-ethylhexyl palmitate, alkyl benzoate, polyethylene glycol diheptanoate, propylene glycol bis(2-ethylhexanoate) and mixtures thereof, hexyl laurate, neopentanoic acid esters, such as isodecyl neopentanoate, isotridecyl neopentanoate, isostearyl neopentanoate or 2-octyldodecyl neopentanoate, isononanoic acid esters, in particular isononyl isononanoate, isotridecyl isononanoate or octyl isononanoate, oleyl erucate, isopropyl lauroyl sarcosinate, diisopropyl sebacate, isocetyl stearate, isodecyl neopentanoate, isostearyl behenate or myristyl myristate;
iv) hydroxylated esters, in particular polyglyceryl-2 triisostearate;
v) aromatic esters, in particular tridecyl trimellitate, C₁₂-C₁₅ alcohol benzoate, the 2-phenylethyl ester of benzoic acid, and butyloctyl salicylate;
vi) linear fatty acid esters with a total carbon number ranging from 35 to 70, in particular pentaerythrityl tetrapelargonate;
vii) esters of C₂₄-C₂₈ branched fatty acids or fatty alcohols, in particular triisoarachidyl citrate, pentaerythrityl tetraisononanoate, glyceryl triisostearate, glyceryl tris(2-decyltetradecanoate), pentaerythrityl tetraisostearate, polyglyceryl-2 tetraisostearate or pentaerythrityl tetrakis(2-decyltetradecanoate);
viii) the polyesters obtained by condensation of dimer and/or trimer of unsaturated fatty acid and of diol, in particular those with the INCI name Dilinoleic Acid/Butanediol Copolymer or Dilinoleic Acid/Propanediol Copolymer; the polyesters obtained by condensation of fatty acid dimer and of diol dimer, in particular dimer dilinoleyl dimer dilinoleate;
ix) synthetic ethers containing from 10 to 40 carbon atoms, in particular dicaprylyl ether;
x) dialkyl carbonates, the two alkyl chains possibly being identical or different, in particular dicaprylyl carbonate;
xi) vinylpyrrolidone copolymers, in particular vinylpyrrolidone/1-hexadecene copolymer; and
xii) mixtures thereof,
the non-volatile carbonate oils may be chosen from the carbonates of formula R₈—O—C(O)—O—R₉, with R₈ and R₉, which may be identical or different, representing a linear or branched C₄ to C₁₂ and preferentially C₆ to C₁₀ alkyl chain; the carbonate oils may be dicaprylyl carbonate (or dioctyl carbonate), bis(2-ethylhexyl) carbonate, dipropylheptyl carbonate, dibutyl carbonate, dineopentyl carbonate, dipentyl carbonate, dineoheptyl carbonate, diheptyl carbonate, diisononyl carbonate or dinonyl carbonate and preferably dioctyl carbonate;
oils known as non-volatile ether oils of formula R₁—O—R₂ in which R₁ and R₂ independently denote a linear, branched or cyclic C₆-C₂₄ alkyl group, preferably a C₆-C₁₈ alkyl group, and preferably C₈-C₁₂ alkyl group. It may be preferable for R₁ and R₂ to be identical. Linear alkyl groups that may be mentioned include a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, an eicosyl group, a behenyl group, a docosyl group, a tricosyl group and a tetracosyl group. Branched alkyl groups that may be mentioned include a 1,1-dimethylpropyl group, a 3-methylhexyl group, a 5-methylhexyl group, an ethylhexyl group, a 2-ethylhexyl group, a 5-methyloctyl group, a 1-ethylhexyl group, a 1-butylpentyl group, a 2-butyloctyl group, an isotridecyl group, a 2-pentylnonyl group, a 2-hexyldecyl group, an isostearyl group, a 2-heptylundecyl group, a 2-octyldodecyl group, a 1,3-dimethylbutyl group, a 1-(1-methylethyl)-2-methylpropyl group, a 1,1,3,3-tetramethylbutyl group, a 3,5,5-trimethylhexyl group, a 1-(2-methylpropyl)-3-methylbutyl group, a 3,7-dimethyloctyl group and a 2-(1,3,3-trimethylbutyl)-5,7,7-trimethyloctyl group. As cyclic alkyl groups, mention may be made of a cyclohexyl group, a 3-methylcyclohexyl group and a 3,3,5-trimethylcyclohexyl group, dilauryl ether, diisostearyl ether, dioctyl ether, nonylphenyl ether, dodecyl dimethylbutyl ether, cetyl dimethylbutyl ether and mixtures thereof.

Preferably, the non-volatile oil(s) are chosen from hydrogenated or non-hydrogenated polyisobutenes, preferably hydrogenated, in particular the non-volatile compounds of the Parleam® range; mixtures of C₁₅-C₁₉ alkanes, and from linear aliphatic hydrocarbon-based esters of formula R—C(O)—OR′ in which R—C(O)—O represents a carboxylic acid residue containing from 2 to 40 carbon atoms, and R′ represents a hydrocarbon-based chain containing from 1 to 40 carbon atoms, as defined previously, in particular isononyl isononanoate.

More preferentially, the process of the invention uses one or more hydrocarbon-based oils containing from 8 to 16 carbon atoms, and notably branched C₈-C₁₆ alkanes, in particular isoalkanes, preferably C₁₃-C₁₆ isoparaffins, isododecane, isodecane, isohexadecane, alone or as mixtures, and more preferentially isododecane.

According to a preferred embodiment, the process of the invention uses one or more fatty substances iv), different from the compounds of formula (I), in particular at least one oil, preferably at least one volatile oil, more preferentially at least one hydrocarbon-based volatile oil.

In particular, the amount of oil(s), different from the compounds i), in at least one of the compositions “C1”, “C2”, “C3”, “C4” or “C5” used in the process according to the invention ranges from 1% to 99% by weight, relative to the total weight of the composition, more particularly from 2% to 98% by weight, preferentially from 3% to 97%, better still from 5% to 96% by weight, relative to the total weight of the composition.

According to another particular embodiment, the process of the invention does not use any fatty substances iv) different from the compounds of formula (I).

According to yet another embodiment, at least one of the compositions “C1”, “C2”, “C3”, “C4” or “C5” used in the process according to the invention contains a mixture of fatty substances v) different from the compounds i), and chosen from mixtures of at least one volatile oil, preferably a hydrocarbon-based oil, and at least one non-volatile oil, such as octyldodecanol.

### Compositions

Another object of the invention is compositions, in particular cosmetic compositions, containing at least one compound chosen from the compounds of formulae (Ia), (Ib), (Ic), (Id) and (Ie), as described previously, and also the salts, isomers and/or solvates thereof, such as hydrates, more particularly compositions, notably cosmetic compositions, containing at least one compound of formulae (Iic), (Iid) and (Iie), as described previously.

Another subject of the invention is compositions, notably cosmetic compositions, containing at least one compound chosen from compounds (8), (9), (10), (11), (12), (13) and (14), as described previously, and also the salts, isomers and solvates thereof.

Another subject of the invention is compositions, in particular cosmetic compositions, containing at least one compound chosen from the compounds of formulae (I), (Ia), (Ib), (Ic), (Id) and/or (Ie), as described previously, and also the salts, isomers and/or solvates thereof, such as hydrates, other than the compounds (1) to (7) as defined previously.

According to a particular embodiment, the process for treating keratin fibres, preferably the hair, according to the invention uses water. Thus, according to this variant, at least one of the compositions “C1” to “C5” used in the process of the invention comprises water. According to this variant, the compositions “C1”, “C2”, “C3”, “C4” and/or “C5” are aqueous or aqueous-alcoholic compositions.

According to a preferred embodiment, the process for treating keratin fibres, preferably the hair, according to the invention does not use any water. Thus, according to this variant, the compositions “C1”, “C2”, “C3”, “C4” and/or “C5” are anhydrous compositions.

According to a particular embodiment, at least one of the compositions “C1”, “C2”, “C3”, “C4” and/or “C5” comprises an isododecane/ethanol mixture, in particular in a volume ratio of between 1/99 and 99/1, more particularly between 5/95 and 95/5.

According to a particular embodiment, at least one of the compositions “C1”, “C2”, “C3”, “C4” and/or “C5” comprises an isododecane/octyldodecanol mixture, in particular in a volume ratio of between 1/99 and 99/1, more particularly between 5/95 and 95/5.

According to a particular embodiment, at least one of the compositions “C1”, “C2”, “C3”, “C4” or “C5” may be in the form of a water-in-oil emulsion or an oil-in-water emulsion.

According to a particular embodiment, the compositions “C1”, “C4” or “C5” may be in anhydrous, water-in-oil emulsion or oil-in-water emulsion form.

According to a particular embodiment, composition “C4” is anhydrous. In particular, composition “C4” is anhydrous, and it comprises at least one oil, in particular a volatile oil, preferably isododecane.

According to another particular embodiment, composition “C4” comprises water. In particular, composition “C4” comprises water, and optionally at least one volatile or non-volatile oil, and/or at least one C₁-C₄ alcohol such as ethanol or isopropanol, and/or at least one non-volatile oil.

The compositions “C1” to “C5” used in the process of the invention may also comprise one or more solvents, other than water.

According to a particular embodiment of the invention, the compound(s) of formula (I) as defined previously are used in a medium containing at least one solvent, preferably at least one polar and/or protic solvent, other than water.

According to this embodiment, composition “C1”, “C2”, “C3”, “C4” and/or “C5” used in the process of the invention comprises one or more solvents, preferably one or more polar and/or protic solvents other than water.

Preferably, composition “C1”, “C2”, “C3”, “C4” and/or “C5” used in the process of the invention comprises at least one polar and/or protic solvent chosen from monoalcohols containing from 2 to 6 carbon atoms, in particular chosen from ethanol, propanol, n-butanol, isopropanol, isobutanol, tert-butanol, pentanol and hexanol, preferably n-butanol or ethanol, and even more preferentially ethanol.

According to one embodiment, composition “C1”, “C2”, “C3”, “C4” and/or “C5” used in the process of the invention comprises one or more solvents other than water, in a content of less than 95% by weight, more preferentially less than 92% by weight, relative to the total weight of the composition.

According to one embodiment, composition “C2”, “C3” or “C4” is anhydrous.

According to another embodiment, composition “C4” is aqueous.

Organic solvents that may also be mentioned include polyols that are miscible with water at room temperature (25° C.), notably chosen from polyols notably containing from 2 to 10 carbon atoms, preferably containing from 2 to 6 carbon atoms, such as glycerol, propylene glycol, 1,3-propanediol, butylene glycol, pentylene glycol, hexylene glycol, dipropylene glycol, diethylene glycol or diglycerol; polyol ethers, such as 2-butoxyethanol, propylene glycol monomethyl ether, diethylene glycol monoethyl ether or diethylene glycol monomethyl ether; and also aromatic alcohols, such as benzyl alcohol, and mixtures thereof.

According to a particular embodiment, the composition also comprises one or more polyols notably chosen from polyols notably containing from 2 to 10 carbon atoms, preferably containing from 2 to 6 carbon atoms, preferably glycerol.

The compositions “C1”, “C2”, “C3”, “C4” or “C5” used in the process of the invention may also comprise one or more adjuvants commonly used in cosmetics, in particular chosen from thickeners, film-forming agents, gelling agents, trace elements, softeners, sequestrants, fragrances, basifying or acidifying agents, dispersants, preserving agents, fillers, surfactants, hair-loss counteractants, anti-dandruff agents, free-radical scavengers, propellants, polar additives and polymers, or mixtures thereof.

Another subject of the invention is a composition, termed “C3” composition, notably a cosmetic composition, comprising i) at least one compound of formula (I), or (Ia), (Ib), (Ic), (Id) and/or (Ie), and/or even (Iic), (Iid) and/or (Iie), as defined previously, ii) at least one crosslinking agent, in particular as defined previously, and iii) at least one cosmetic active agent, in particular as defined previously, preferably chosen from a) dyestuffs, preferably chosen from pigments, direct dyes and mixtures thereof, and more preferentially from pigments.

Another subject of the invention is the cosmetic use of composition “C3” as defined previously, for treating keratin fibres, in particular for caring for, styling and/or colouring keratin fibres, preferably the hair.

A composition “C1”, “C2”, “C3”, “C4” or “C5” is generally suitable for application to keratin fibres, in particular application to the hair, and thus generally comprises a physiologically acceptable medium, i.e. a medium that is compatible with keratin fibres, notably human keratin fibres such as the hair.

It is preferably a cosmetically acceptable medium, i.e. a medium which has a pleasant colour, odour and feel and which does not cause any unacceptable discomfort, i.e. stinging or tautness, liable to discourage the user from applying this composition.

According to a particular embodiment, a composition “C1”, “C2”, “C3”, “C4” or “C5” is in the form of a haircare product, in particular a hair dyeing product or a styling product notably free of dyestuff, such as a lacquer, or a “styling” product of the mousse or gel type.

### Kit

According to yet another of its aspects, the present invention is also directed towards a multi-compartment kit or device, notably a cosmetic kit or device, comprising:
at least one compartment containing i) at least one compound of formula (I), or (Ia), (Ib), (Ic), (Id) and/or (Ie), as defined previously, and optionally iii) at least one cosmetic active agent, in particular as defined previously, in particular comprising composition “C1” as defined previously;
at least one compartment different from the one which contains i) and containing ii) at least one crosslinking agent, in particular as defined previously, and optionally iii) at least one cosmetic active agent, in particular as defined previously, in particular comprising composition “C4” as defined previously; and
optionally, at least one compartment different from those containing i) and ii), and containing iii) at least one cosmetic active agent, in particular as defined previously, which may be identical to or different from that/those optionally contained in the compartments comprising i) and ii).

Preferably, the compartment comprising at least i), in particular a composition “C1”, does not comprise any crosslinking agent ii).

Preferably, the compartment comprising at least one crosslinking agent ii), in particular a composition “C4”, does not contain i) any compound of formula (I).

The invention is illustrated in greater detail by the examples presented below. Unless otherwise indicated, the amounts indicated are expressed as mass percentages.

### EXAMPLES

### Methods and Measurements

### Hair Application

The hair dyeing evaluation protocol is detailed below:

The evaluations are conducted according to three different protocols: in 1, 2 or 3 step(s), each on locks of natural hair containing 90% white hair strands.

### a) One-Step Protocol

The compounds of formula (I) bearing acetoacetate functions and the crosslinking agents are mixed together before application. The system remains fluid long enough to allow application to the substrate.

The composition is applied to locks of dry natural hair containing 90% white hairs, at a rate of 1 g of composition per gram of lock.

The locks of hair are left for 5 minutes at room temperature.

The locks of hair are then combed and dried with a hairdryer for 3 minutes.

The locks of hair are left at room temperature for 18 hours at 80% relative humidity. The hair is dyed uniformly and intensely.

### b) Two-Step Protocol

A first composition, termed the “base coat”, is applied to locks of dry natural hair containing 90% white hairs, in a proportion of 0.5 g of composition per gram of lock. The locks are then combed. This deposit is applied to the keratin materials, and then left for 30 minutes at room temperature (25° C.).

After this first gesture, a second composition, termed the “top coat”, is applied to the lock of hair, at a rate of 0.5 g per gram of lock.

After application, the locks of hair are combed and dried with a hairdryer.

The locks of hair are left at room temperature for 24 hours under a fume cupboard. The hair is dyed uniformly and intensely.

The locks of hair thus coloured are then subjected to a test of several repeated shampoo washes so as to evaluate the fastness (persistence) of the colouring obtained with respect to shampoo washing, according to the shampoo washing protocol described below.

### c) Three-Step Protocol

A first composition containing pigments conveyed in a cosmetic solvent is applied to locks of dry natural hair containing 90% white hair strands, at a rate of 0.5 g of composition per gram of lock, and then left for 1 minute at room temperature (25° C.).

A second composition, termed the “base coat”, is applied to this coloured lock, at a rate of 0.5 g of composition per gram of lock. The locks are then combed and left for 30 minutes at room temperature (25° C.).

After this second gesture, a third composition, termed the “top coat”, is applied to the lock of hair, at a rate of 0.5 g per gram of lock.

After application, the locks of hair are combed and dried with a hairdryer.

The locks of hair are left at room temperature for 24 hours under a fume cupboard. The hair is dyed uniformly and intensely.

The locks of hair thus coloured are then subjected to a test of several repeated shampoo washes so as to evaluate the fastness (persistence) of the colouring obtained with respect to shampoo washes, according to the shampoo washing protocol described below.

### Shampoo Washing Protocol

The locks of dyed hair are combed, moistened with water at 35° C. and then passed between the fingers five times for 5 seconds. The locks of hair are then squeezed dry between two fingers.

A standard shampoo (Garnier Ultra Doux) is applied uniformly to the dyed locks, in a proportion of 0.4 g of standard shampoo per gram of locks, the locks of hair being massaged gently along the length (6 passes) for 15 seconds, from the root to the end.

The locks of hair are then placed on a watch glass and left to stand for 1 minute.

Next, the locks of hair are rinsed with water while passing the locks between the fingers (15 passes). The locks of hair are then squeezed dry between two fingers before the next shampoo wash.

Once the tests of several shampoo washes have been performed, the locks of hair are combed and dried with a hairdryer.

### Persistence Protocol

The persistence of the colour of the locks was evaluated in the CIE L*a*b* system, using a Minolta Spectrophotometer CM3600A colorimeter (illuminant D65, angle 10°, specular component included).

In this L*a*b* system, L* represents the intensity of the colour, a* indicates the green/red colour axis and b* the blue/yellow colour axis.

The persistence of the colouring is evaluated 24 hours after the last application of the composition of formula (I) for the one-gesture protocol 1, or after the application of the “Top Coat” for the 2-gesture and 3-gesture protocols, by the difference in colour ΔE between the dyed locks before shampoo washing, and then after undergoing three or five shampoo washes according to the protocol described above.

The lower the ΔE value, the more persistent the colour with respect to shampoo washing.

The ΔE value is calculated according to the following equation:

$$Δ E = \sqrt{{( {L}^{⋆} - {L}_{o}^{⋆} )}^{2} + {( {a}^{⋆} - {a}_{o}^{⋆} )}^{2} + {( {b}^{⋆} - {b}_{o}^{⋆} )}^{2}} \qquad [ Math . 1 ]$$

In this equation, L*a*b* represent the values measured after dyeing the hair and after performing the shampoo washes, and L₀*a₀*b₀* represent the values measured after dyeing the hair but before shampoo washing.

### Styling Application: Styling Evaluation Protocol

The styling evaluation protocol is detailed below:

2 g of a solution containing the formulae described below are sprayed onto a lock (90% Natural White (NW) hair, length 20.5 cm). The lock is weighed before and after application.

About 0.5 g of the composition is effectively deposited on the lock.

The lock is wound around a brush (approximately 2 cm in diameter, over a length of 3 cm).

The lock is left at room temperature, at 25° C.

After 24 hours, the lock is released from the brush and suspended. The length of the lock is then measured. After 24 hours of suspension, the length of the lock is again measured.

A comparison of the curl maintenance is made between the compositions according to the invention and the comparative compositions.

### Example 1: Synthesis of Castor Oil Bearing an Acetoacetate Function (Compound 1)

The synthesis is represented by the following preparation scheme:

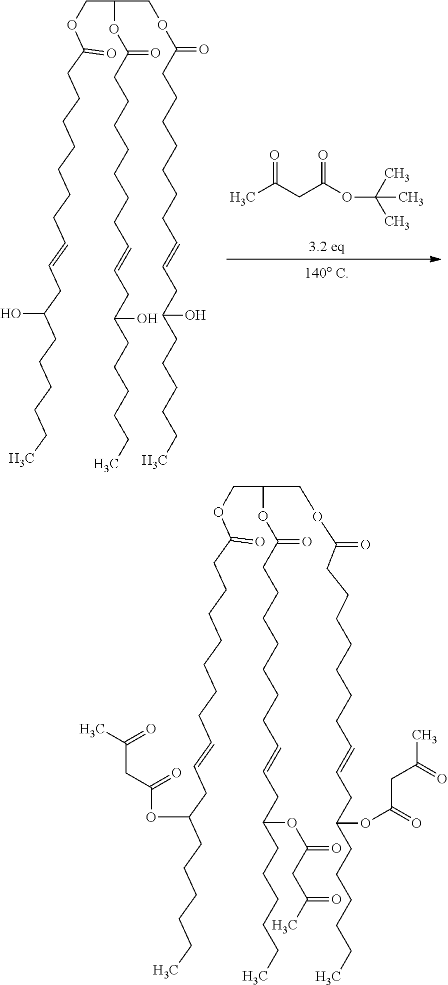 [SMILES: CCCCCCC(O)C/C=C/CCCCCCCC(=O)OCC(COC(=O)CCCCCCC/C=C/CC(O)CCCCCC)OC(=O)CCCCCCC/C=C/CC(O)CCCCCC ; CC(=O)CC(=O)OC(C)(C)C ; CCCCCCC(C/C=C/CCCCCCCC(=O)OCC(COC(=O)CCCCCCC/C=C/CC(CCCCCC)OC(=O)CC(C)=O)OC(=O)CCCCCCC/C=C/CC(CCCCCC)OC(=O)CC(C)=O)OC(=O)CC(C)=O]

50 g of castor oil (sold by the company Interchimie) and 25.40 g of tert-butyl acetoacetate are placed in a 250 mL SVL® reactor equipped with a mechanical stirrer, a thermometer and a distillation column. The reaction medium is heated at 130° C. on an oil bath. After 7 hours, the reaction medium is cooled, placed in a 250 mL pear-shaped flask and heated at 120° C. under continuous vacuum on a rotavapor for 1 hour.

### Example 2: Synthesis of Hydroxylated Castor Oil Bearing an Acetoacetate Function (Compound 2)

The synthesis is represented by the following preparation scheme.

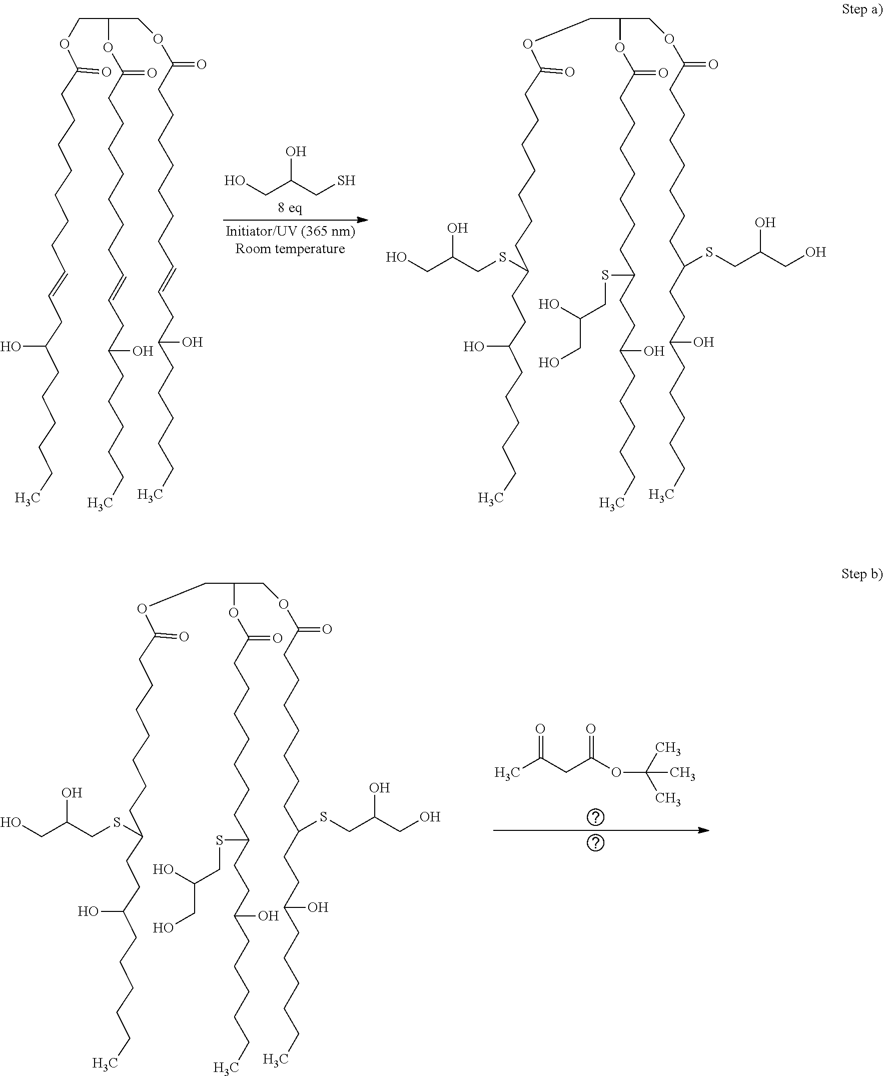 [SMILES: CCCCCCC(O)C/C=C/CCCCCCCC(=O)OCC(COC(=O)CCCCCCC/C=C/CC(O)CCCCCC)OC(=O)CCCCCCC/C=C/CC(O)CCCCCC ; OCC(O)CS ; CCCCCCC(O)CCC(CCCCCCCC(=O)OCC(COC(=O)CCCCCCCC(CCC(O)CCCCCC)SCC(O)CO)OC(=O)CCCCCCCC(CCC(O)CCCCCC)SCC(O)CO)SCC(O)CO ; CCCCCCC(O)CCC(CCCCCCCC(=O)OCC(COC(=O)CCCCCCCC(CCC(O)CCCCCC)SCC(O)CO)OC(=O)CCCCCCCC(CCC(O)CCCCCC)SCC(O)CO)SCC(O)CO ; CC(=O)CC(=O)OC(C)(C)C]

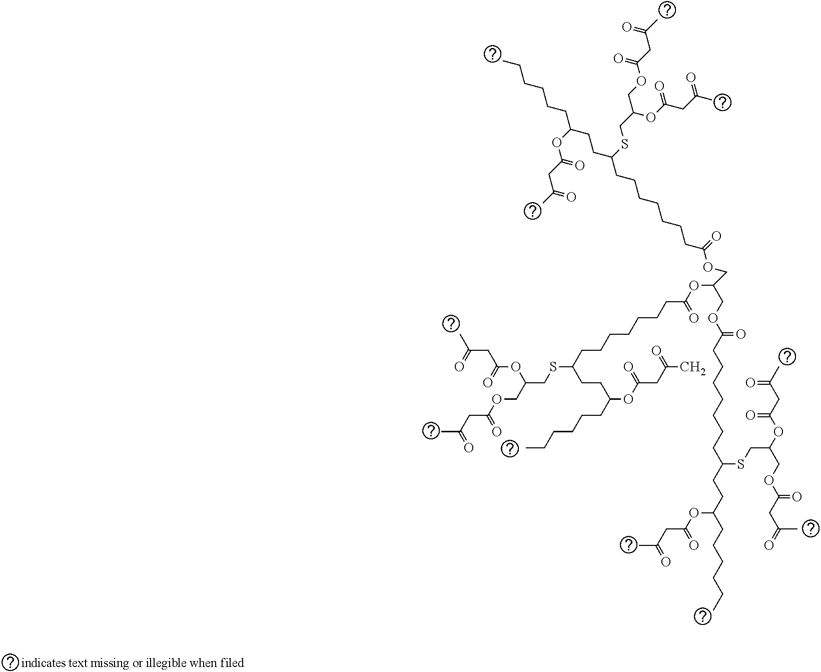 [SMILES: CCCCCCC(CCC(CCCCCCCC(=O)OCC(COC(=O)CCCCCCCC(CCC(CCCCCC)OC(=O)CC(C)=O)SCC(COC(=O)CC(C)=O)OC(=O)CC(C)=O)OC(=O)CCCCCCCC(CCC(CCCCCC)OC(=O)CC(C)=O)SCC(COC(=O)CC(C)=O)OC(=O)CC(C)=O)SCC(COC(=O)CC(C)=O)OC(=O)CC(C)=O)OC(=O)CC(C)=O]

In step a), the hydroxylated castor oil is prepared: 10 g of castor oil (sold by the company Interchimie), 9 g of 3-mercapto-1,2-propanediol (sold by the company Sigma-Aldrich) and 0.7 g of 2-hydroxy-2-methylpropiophenone (sold by the company Sigma-Aldrich) are added to 20 mL of ethyl acetate in a 250 mL round-bottomed flask equipped with a magnetic stirrer, and stirred for 5 minutes under argon. The reaction medium is then irradiated under a UV lamp (100 W, 365 nm) for 4 hours, and then washed three times with NaCl-saturated water. The organic phase is separated out, dried over MgSO₄ and then concentrated under vacuum. Finally, the solvent and the traces of thiol and of photoinitiator (2-hydroxy-2-methylpropiophenone) are removed by distillation under reduced pressure. The product obtained is used as is in the next step.

In step b), the hydroxylated castor oil is functionalized with acetoacetate groups: 10 g of hydroxylated castor oil obtained in step a) and 12 g of tert-butyl acetoacetate are placed in a 250 mL round-bottomed flask equipped with a magnetic stirrer. The reaction medium is heated at 130° C. on an oil bath. After 4 hours, the reaction medium is placed under continuous vacuum for 1 hour. A yellow viscous oil is obtained.

### Example 3: Synthesis of Epoxidized Oil Bearing an Acetoacetate Function (Compound 3)

The synthesis is represented by the following preparation scheme:

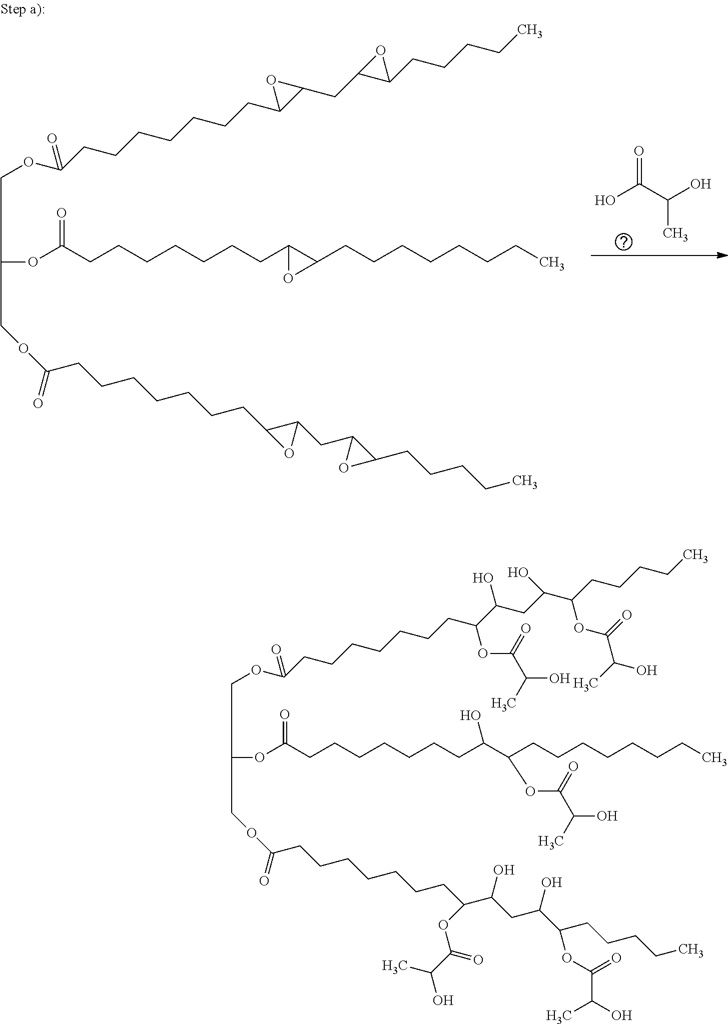

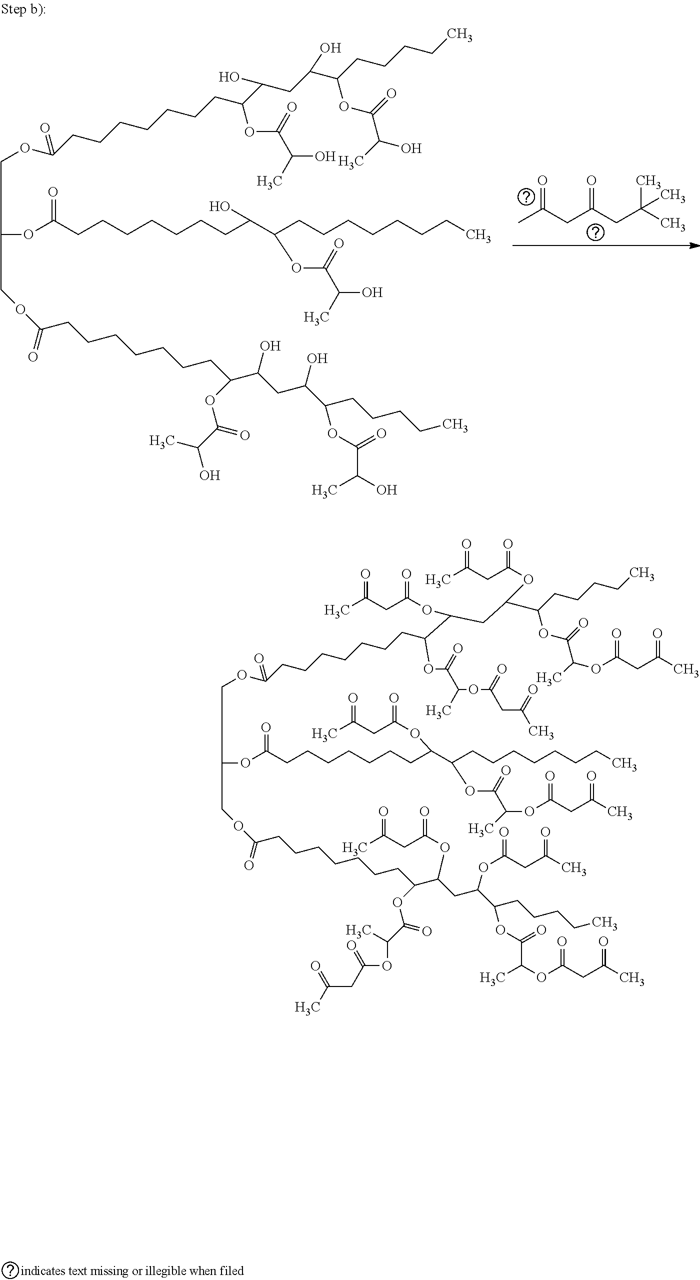 [SMILES: CCCCCCCCC(OC(=O)C(C)O)C(O)CCCCCCCC(=O)OC(COC(=O)CCCCCCCC(OC(=O)C(C)O)C(O)CC(O)C(CCCCC)OC(=O)C(C)O)COC(=O)CCCCCCCC(OC(=O)C(C)O)C(O)CC(O)C(CCCCC)OC(=O)C(C)O ; CC(=O)CC(=O)CC(C)(C)C ; CCCCCCCCC(OC(=O)C(C)OC(=O)CC(C)=O)C(CCCCCCCC(=O)OC(COC(=O)CCCCCCCC(OC(=O)C(C)OC(=O)CC(C)=O)C(CC(OC(=O)CC(C)=O)C(CCCCC)OC(=O)C(C)OC(=O)CC(C)=O)OC(=O)CC(C)=O)COC(=O)CCCCCCCC(OC(=O)C(C)OC(=O)CC(C)=O)C(CC(OC(=O)CC(C)=O)C(CCCCC)OC(=O)C(C)OC(=O)CC(C)=O)OC(=O)CC(C)=O)OC(=O)CC(C)=O]

In step a), 200 g of epoxidized oil (Vikoflex 7170 sold by Arkema), 50.4 g of lactic acid and 6.52 g of caffeine are placed in a 500 mL SVL® reactor equipped with a mechanical stirrer, an argon inlet and a distillation column. The reaction mixture is heated at 120° C. for 5 hours. The polyhydroxylated compound is obtained. A slightly yellow viscous oil is obtained.

In step b), 165 g of the polyhydroxylated compound obtained in step a) and 116.74 g of tert-butyl acetoacetate are added to a 500 mL SVL® reactor equipped with a mechanical stirrer, an argon inlet and a distillation column. The reaction medium is heated at between 130° C. and 140° C. for 5 hours. After 5 hours of heating, the medium is placed under vacuum at 50 mbar at 140° C. An orange viscous oil is obtained.

The oil obtained is diluted with ethyl acetate and washed three times with saturated sodium bicarbonate solution, and then twice with water. The organic phase is filtered over MgSO₄, and the ethyl acetate is then distilled off on a rotavapor at 100° C. under reduced pressure. A slightly orange viscous oil is obtained.

### Example 4: Synthesis of Soybean Oil Bearing an Acetoacetate Function (Compound 4)

The synthesis is represented by the following preparation scheme:

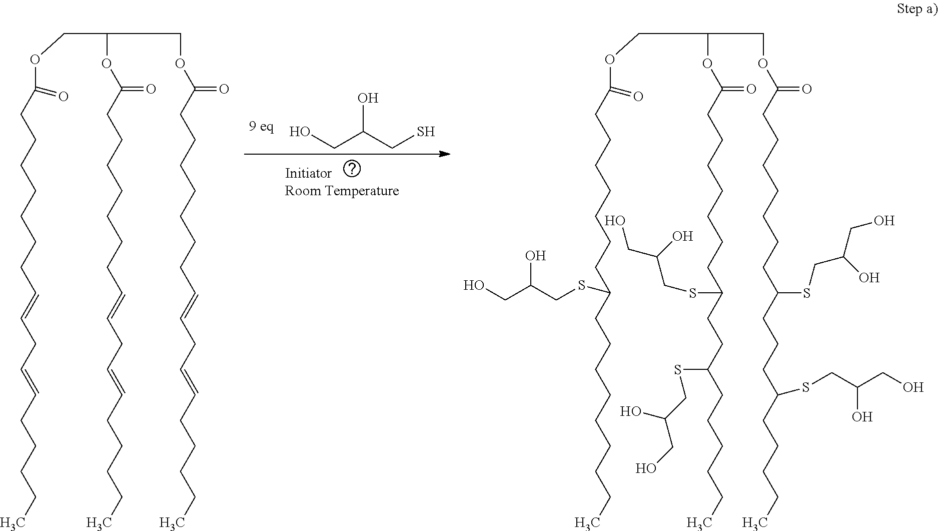 [SMILES: CCCCC/C=C/C/C=C/CCCCCCCC(=O)OCC(COC(=O)CCCCCCC/C=C/C/C=C/CCCCC)OC(=O)CCCCCCC/C=C/C/C=C/CCCCC ; OCC(O)CS ; CCCCCCCCCC(CCCCCCCC(=O)OCC(COC(=O)CCCCCCCC(CCCC(CCCCC)SCC(O)CO)SCC(O)CO)OC(=O)CCCCCCCC(CCC(CCCCCC)SCC(O)CO)SCC(O)CO)SCC(O)CO]

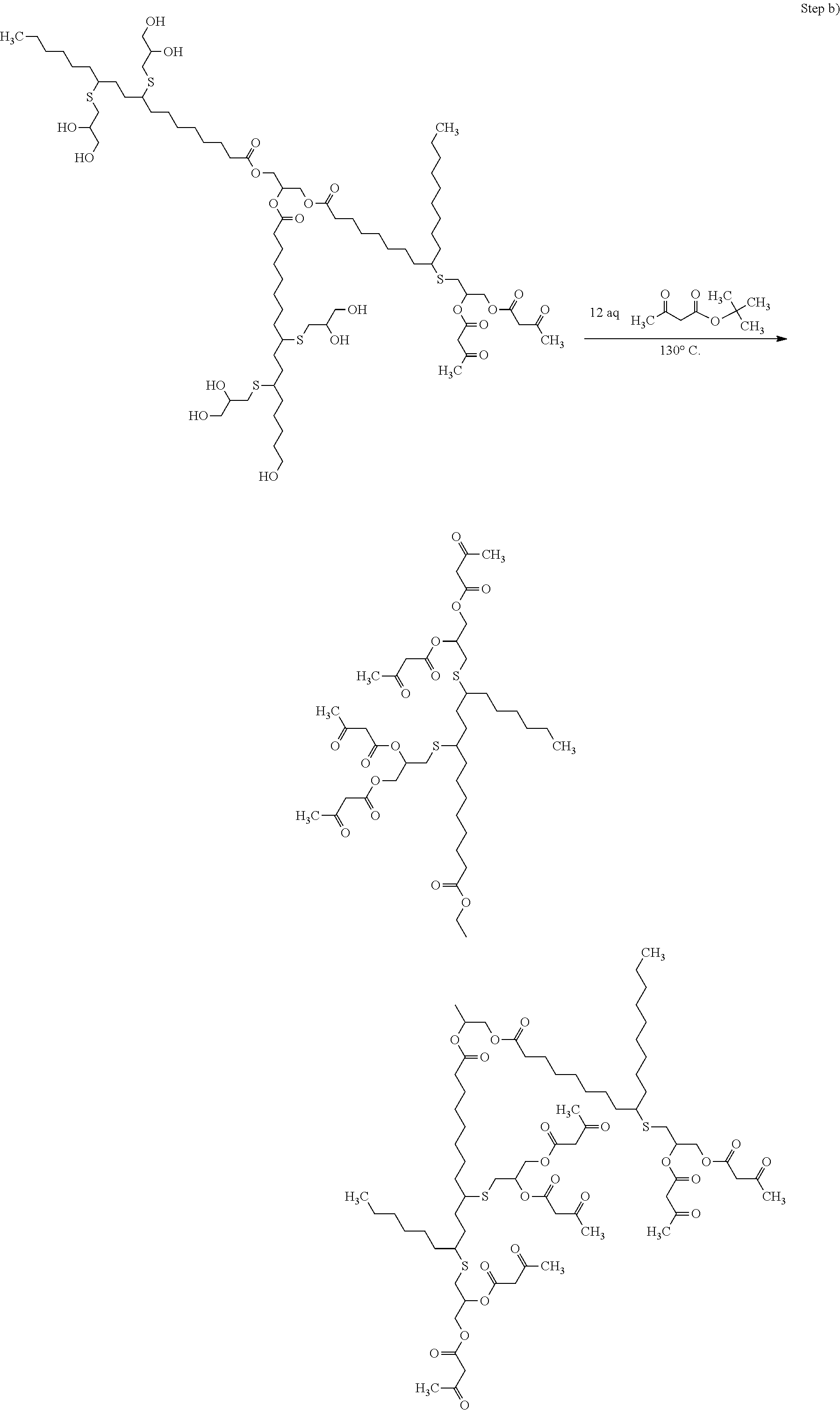 [SMILES: CCCCCCCCCC(CCCCCCCC(=O)OCC(COC(=O)CCCCCCCC(CCC(CCCCCC)SCC(O)CO)SCC(O)CO)OC(=O)CCCCCCCC(CCC(CCCCCO)SCC(O)CO)SCC(O)CO)SCC(COC(=O)CC(C)=O)OC(=O)CC(C)=O ; CC(=O)CC(=O)OC(C)(C)C ; CCCCCCC(CCC(CCCCCCCC(=O)OCC)SCC(COC(=O)CC(C)=O)OC(=O)CC(C)=O)SCC(COC(=O)CC(C)=O)OC(=O)CC(C)=O ; CCCCCCCCCC(CCCCCCCC(=O)OCC(C)OC(=O)CCCCCCCC(CCC(CCCCCC)SCC(COC(=O)CC(C)=O)OC(=O)CC(C)=O)SCC(COC(=O)CC(C)=O)OC(=O)CC(C)=O)SCC(COC(=O)CC(C)=O)OC(=O)CC(C)=O]

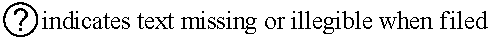

In step a), 10 g of soybean oil (Henry Lamotte Oils), 10 g of 3-mercapto-1,2-propanediol (sold by the company Sigma-Aldrich) and 0.8 g of 2-hydroxy-2-methylpropiophenone (sold by the company Sigma-Aldrich) are added to 25 mL of ethyl acetate in a 250 mL round-bottomed flask equipped with a magnetic stirrer, and stirred for 5 minutes under argon. The reaction medium is then irradiated under a UV lamp (100 W, 365 nm) for 4 hours, and then washed three times with NaCl-saturated water. The organic phase is separated out, dried over MgSO₄ and then concentrated under vacuum. The solvent and the traces of thiol and of photoinitiator are removed by distillation under reduced pressure.

In step b), 10 g of hydroxylated soybean oil obtained in step a) and 14 g of tert-butyl acetoacetate are placed in a 250 mL round-bottomed flask equipped with a magnetic stirrer. The reaction medium is then heated at 130° C. on an oil bath. After 4 hours, the reaction medium is cooled and placed under continuous vacuum for 1 hour so as to remove the excess tert-butyl acetoacetate. A yellow viscous oil is obtained.

### Example 5: Synthesis of an “Acidic” Oil Bearing an Acetoacetate Function (Compound 5)

The synthesis is represented by the following preparation scheme:

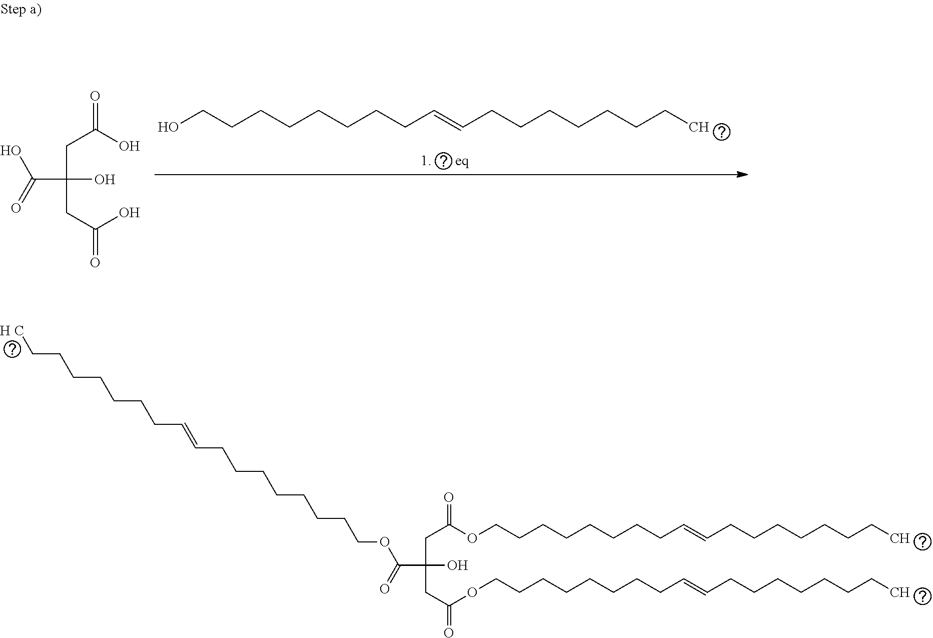

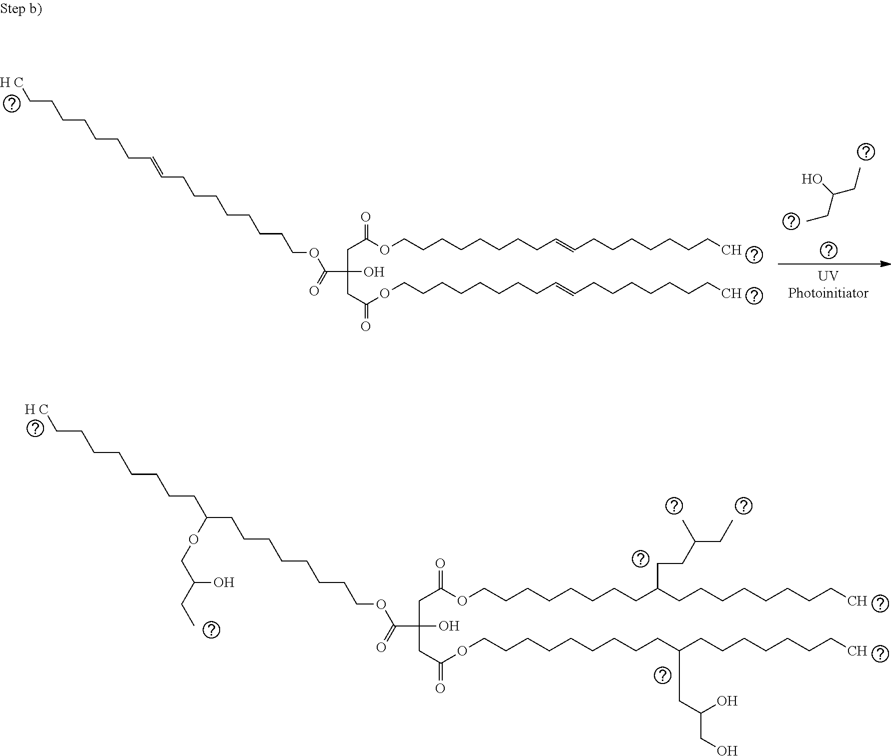

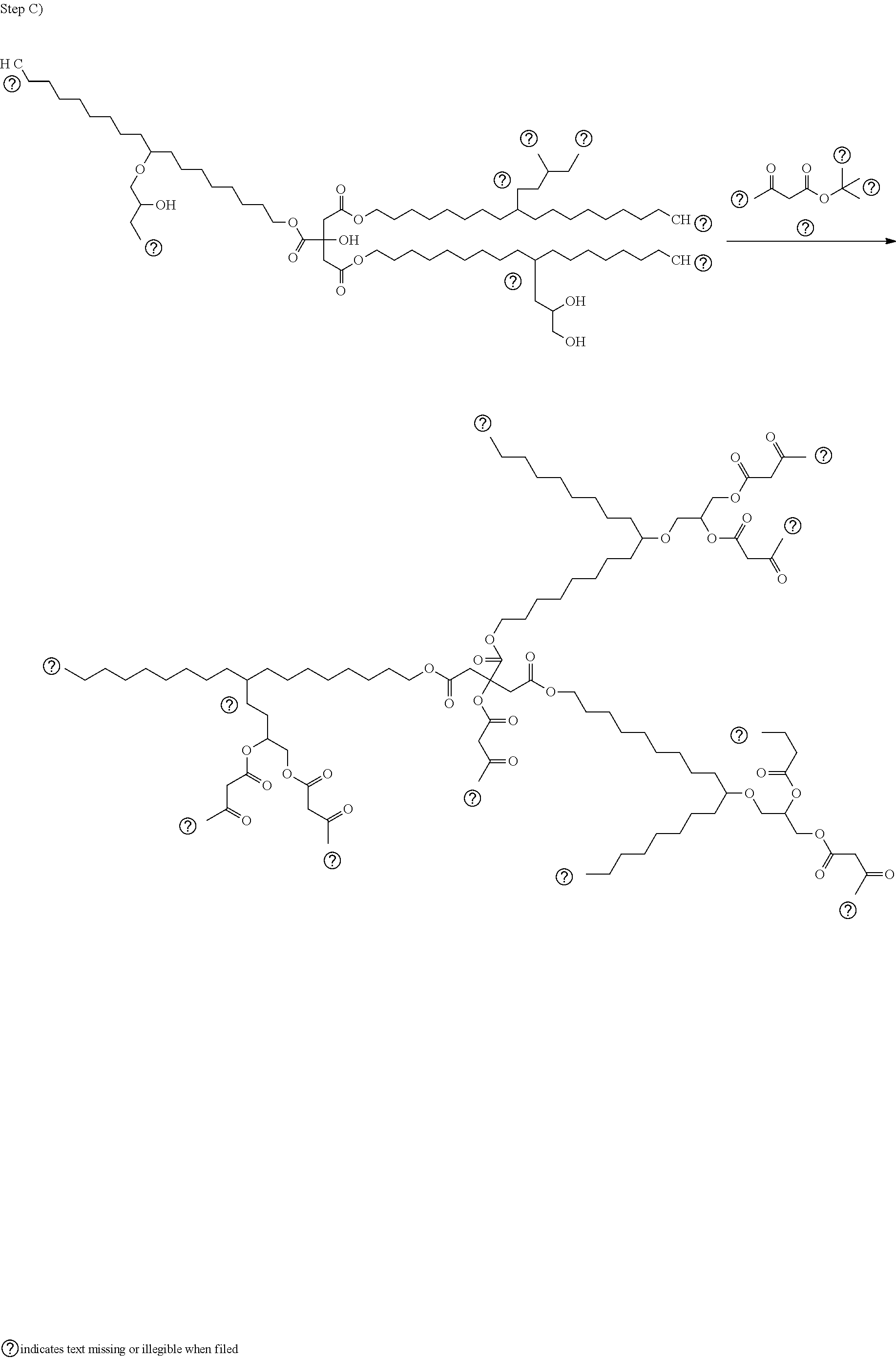

In step a), 100 g of citric acid and 419 g of oleyl alcohol are placed in a 500 mL SVL© reactor equipped with a mechanical stirrer and a distillation column. The reaction medium is heated using a heating mantle, at 190° C. for 7 hours and then at 220° C. for 5 hours. The reaction is monitored by the acid number. When the acid number is less than 5, the reaction is stopped.

In step b), 100 g of acidic oil obtained in a), 70 g of 3-mercapto-1,2-propanediol (sold by the company Sigma-Aldrich) and 5 g of 2-hydroxy-2-methylpropiophenone (sold by the company Sigma-Aldrich) are added to 15 mL of ethyl acetate in a 250 mL round-bottomed flask equipped with a magnetic stirrer, and stirred for 5 minutes under argon. The reaction medium is then irradiated under a UV lamp (100 W, 365 nm) for 4 hours and then washed three times with NaCl-saturated water. The organic phase is separated out, dried over MgSO₄ and then concentrated under vacuum. The solvent and the traces of thiol and of photoinitiator (2-hydroxy-2-methylpropiophenone) are removed by distillation under reduced pressure.

In step c), 100 g of hydroxylated acidic oil obtained in step b) and 100 g of tert-butyl acetoacetate are placed in a 250 mL round-bottomed flask equipped with a magnetic stirrer. The reaction medium is heated at 130° C. on an oil bath. After 4 hours of reaction, the reaction medium is placed under continuous vacuum for 1 hour. A brown viscous oil is obtained.

### Example 6: Synthesis of Castor Oil Bearing an Acetoacetate Function (Compound 6)

The synthesis is represented by the following preparation scheme:

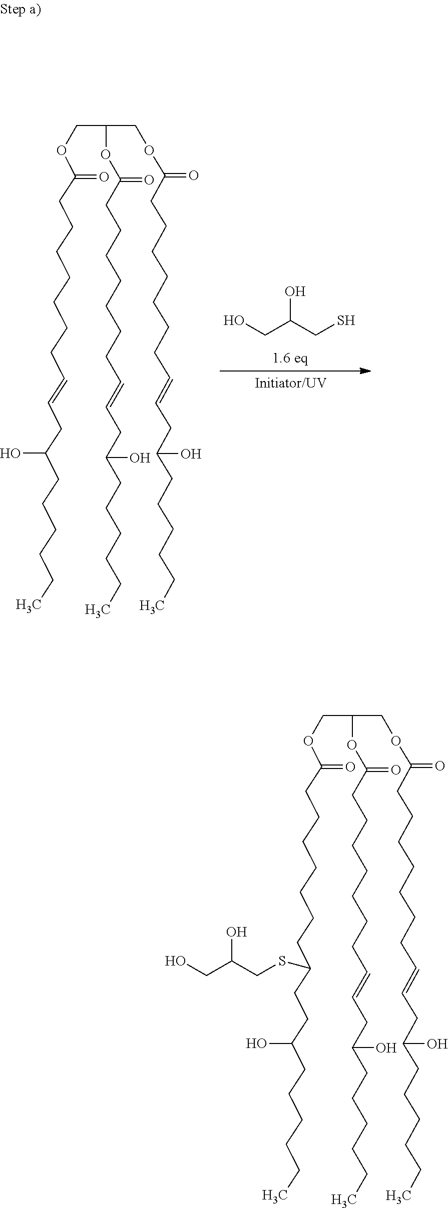 [SMILES: CCCCCCC(O)C/C=C/CCCCCCCC(=O)OCC(COC(=O)CCCCCCC/C=C/CC(O)CCCCCC)OC(=O)CCCCCCC/C=C/CC(O)CCCCCC ; OCC(O)CS ; CCCCCCC(O)C/C=C/CCCCCCCC(=O)OCC(COC(=O)CCCCCCCC(CCC(O)CCCCCC)SCC(O)CO)OC(=O)CCCCCCC/C=C/CC(O)CCCCCC]

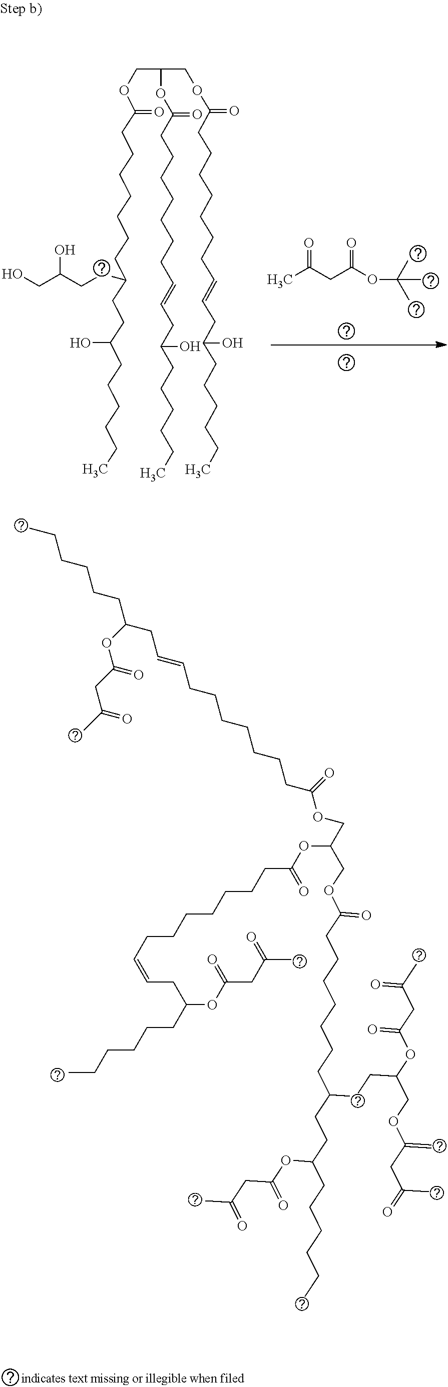 [SMILES: CCCCCCC(O)C/C=C/CCCCCCCC(=O)OCC(COC(=O)CCCCCCCC(CCC(O)CO)CCC(O)CCCCCC)OC(=O)CCCCCCC/C=C/CC(O)CCCCCC ; CC(=O)CC(=O)OC(C)(C)C ; C=C(CC(C)=O)OCC(CCC(CCCCCCCC(=O)OCC(COC(=O)CCCCCCC/C=C/CC(CCCCCC)OC(=O)CC(C)=O)OC(=O)CCCCCCC/C=C\CC(CCCCCC)OC(=O)CC(C)=O)CCC(CCCCCC)OC(=O)CC(C)=O)OC(=O)CC(C)=O]

In step a), 100 g of castor oil (sold by the company Interchimie), 17 g of 3-mercapto-1,2-propanediol (sold by the company Sigma-Aldrich) and 1 g of 2-hydroxy-2-methylpropiophenone (sold by the company Sigma-Aldrich) are added to 20 mL of ethyl acetate in a 250 mL round-bottomed flask equipped with a magnetic stirrer, and stirred for minutes under argon. The reaction medium is then irradiated under a UV lamp (100 W, 365 nm) for 4 hours and then washed three times with NaCl-saturated water. The organic phase is separated out, dried over MgSO₄ and then concentrated under vacuum. The solvent and the traces of thiol and of photoinitiator (2-hydroxy-2-methylpropiophenone) are removed by distillation under reduced pressure.

In step b), 100 g of hydroxylated castor oil obtained in step a) and 99 g of tert-butyl acetoacetate are placed in a 250 mL round-bottomed flask equipped with a magnetic stirrer. The reaction medium is then heated at 130° C. on an oil bath (oil bath nominal temperature of 140° C.). After 4 hours, the reaction medium is placed under continuous vacuum for 1 hour. A yellow viscous oil is obtained.

### Example 7: Synthesis of Mannitol Dihexanoate Bearing an Acetoacetate Function (Compound 7)

The synthesis is represented by the following preparation scheme:

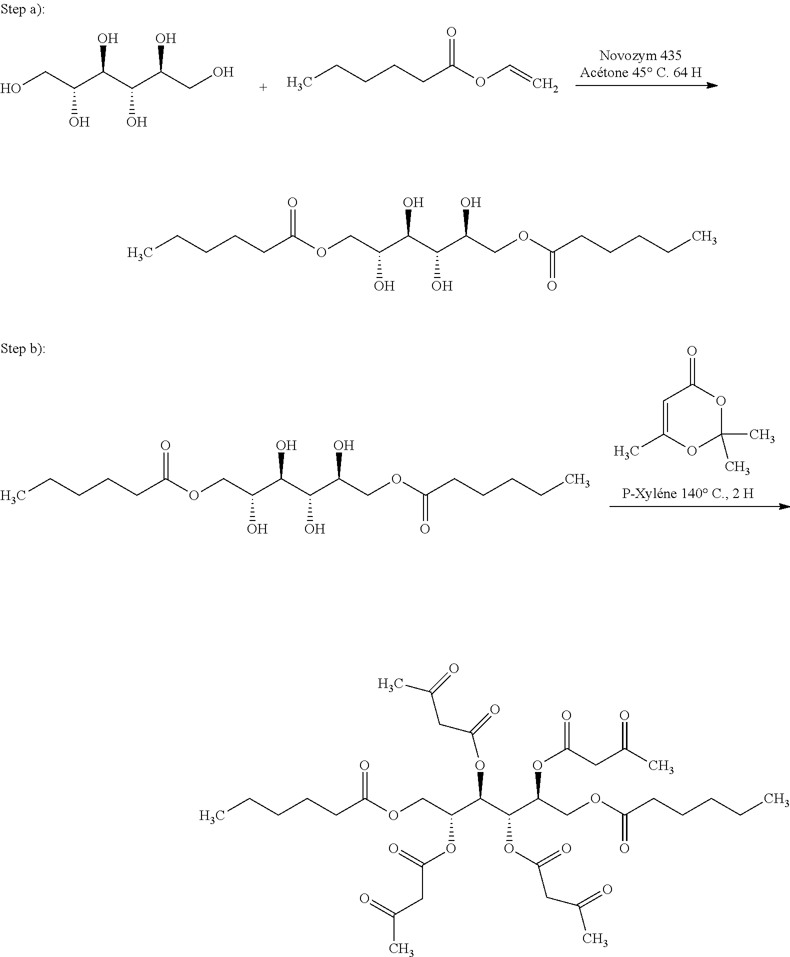

In step a), mannitol dihexanoate is obtained from mannitol under the conditions described in the publication Angew. Chem. Int. Ed. 2010, 49, 7695-7698.

After 64 hours at 45° C., the reaction medium was filtered and then concentrated under reduced pressure. The crude compound obtained after filtration and evaporation is then triturated in dichloromethane.

In step b), 4 equivalents of 2,2,6-trimethyl-1,3-dioxine-4-one in p-xylene are heated at 140° C. for 2 hours. Purification by chromatography on silica gel leads to the compound mannitol dihexanoate acetoacetate.

### Example 8: Synthesis of Sucrose Palmitate Bearing an Acetoacetate Function (Compound 8)

100 g of sucrose palmitate ester (Surfhope® SE Cosme C-1616 marketed by the company Mitsubishi-Kagaku Foods Corporation) and 110 g of tertbutylacetoacetate are introduced into a 500 mL three-necked flask equipped with mechanical stirring and a distillation column. The mixture is heated using an oil bath to a temperature between 140° C.-150° C. (setpoint on the oil bath) for 2 hours. Then the reaction medium is concentrated in a rotavapor at 140° C. under continuous vacuum until constant weight.

### Example 9: Synthesis of Castor Oil Bearing an Acetoacetate Function (Compound 9)

The synthesis is represented by the following preparation scheme:

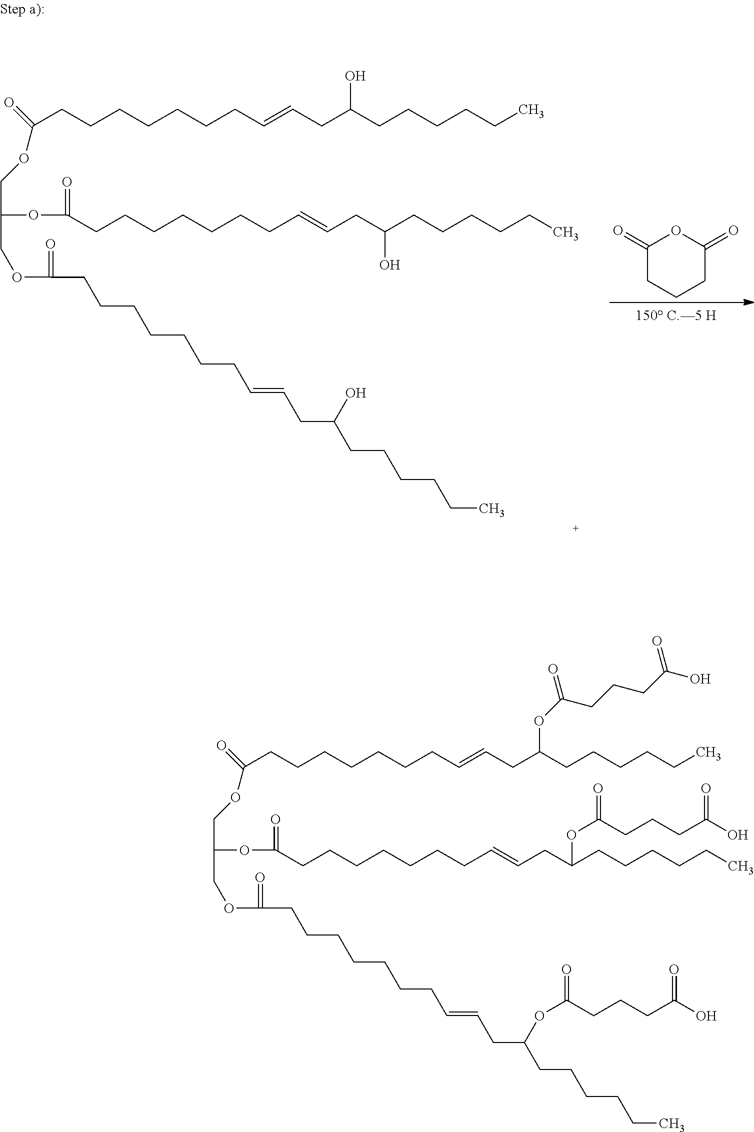 [SMILES: CCCCCCC(O)C/C=C/CCCCCCCC(=O)OCC(COC(=O)CCCCCCC/C=C/CC(O)CCCCCC)OC(=O)CCCCCCC/C=C/CC(O)CCCCCC ; O=C1CCCC(=O)O1 ; CCCCCCC(C/C=C/CCCCCCCC(=O)OCC(COC(=O)CCCCCCC/C=C/CC(CCCCCC)OC(=O)CCCC(=O)O)OC(=O)CCCCCCC/C=C/CC(CCCCCC)OC(=O)CCCC(=O)O)OC(=O)CCCC(=O)O]

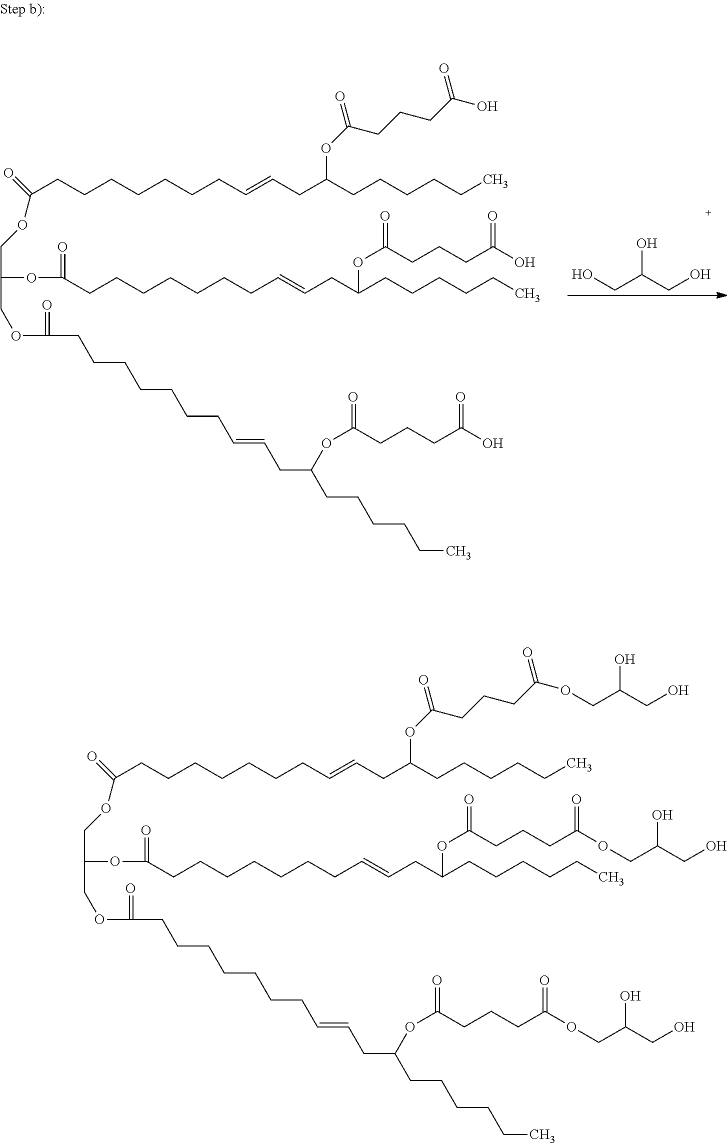 [SMILES: CCCCCCC(C/C=C/CCCCCCCC(=O)OCC(COC(=O)CCCCCCC/C=C/CC(CCCCCC)OC(=O)CCCC(=O)O)OC(=O)CCCCCCC/C=C/CC(CCCCCC)OC(=O)CCCC(=O)O)OC(=O)CCCC(=O)O ; OCC(O)CO ; CCCCCCC(C/C=C/CCCCCCCC(=O)OCC(COC(=O)CCCCCCC/C=C/CC(CCCCCC)OC(=O)CCCC(=O)OCC(O)CO)OC(=O)CCCCCCC/C=C/CC(CCCCCC)OC(=O)CCCC(=O)OCC(O)CO)OC(=O)CCCC(=O)OCC(O)CO]

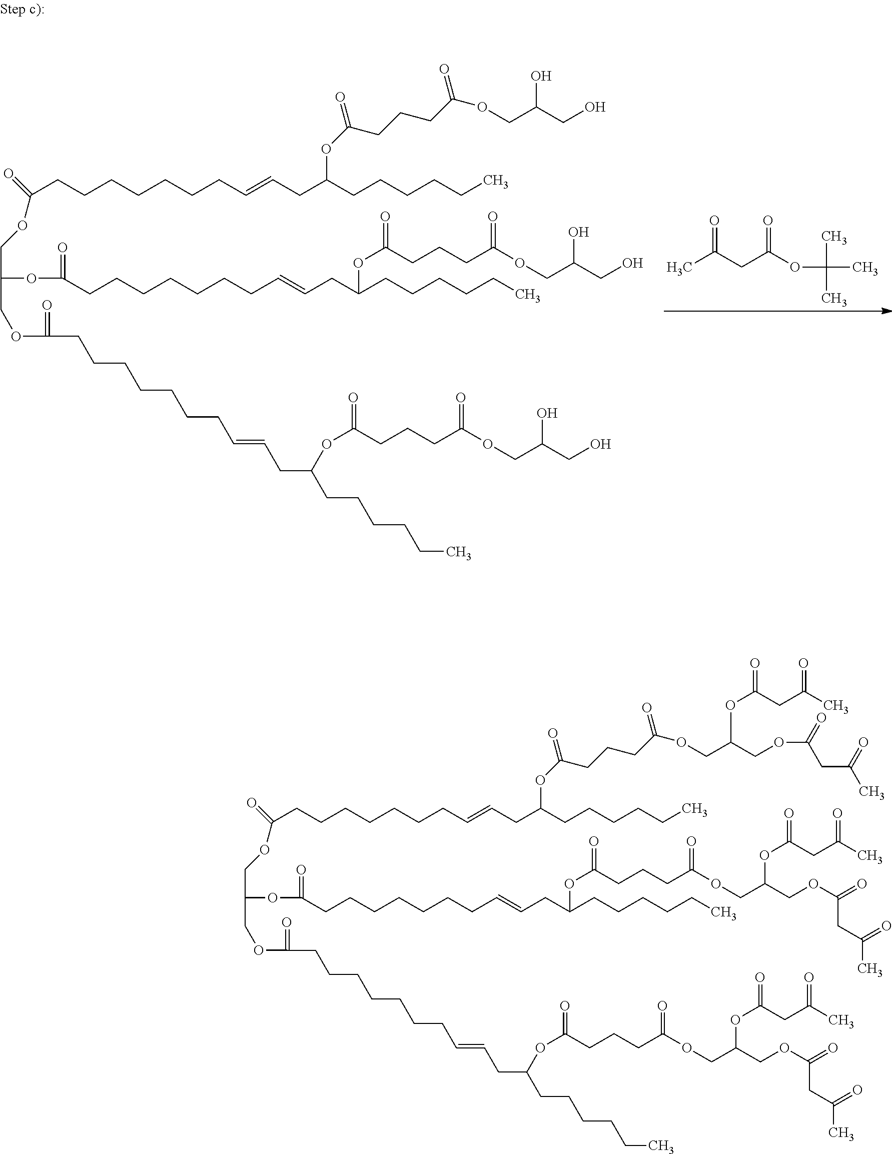 [SMILES: CCCCCCC(C/C=C/CCCCCCCC(=O)OCC(COC(=O)CCCCCCC/C=C/CC(CCCCCC)OC(=O)CCCC(=O)OCC(O)CO)OC(=O)CCCCCCC/C=C/CC(CCCCCC)OC(=O)CCCC(=O)OCC(O)CO)OC(=O)CCCC(=O)OCC(O)CO ; CC(=O)CC(=O)OC(C)(C)C ; CCCCCCC(C/C=C/CCCCCCCC(=O)OCC(COC(=O)CCCCCCC/C=C/CC(CCCCCC)OC(=O)CCCC(=O)OCC(COC(=O)CC(C)=O)OC(=O)CC(C)=O)OC(=O)CCCCCCC/C=C/CC(CCCCCC)OC(=O)CCCC(=O)OCC(COC(=O)CC(C)=O)OC(=O)CC(C)=O)OC(=O)CCCC(=O)OCC(COC(=O)CC(C)=O)OC(=O)CC(C)=O]

In step a), 451 g of castor oil (sold by the company Interchimie) and 171 g of glutaric anhydride are placed in a 750 mL SVL® reactor equipped with a condenser, a mechanical stirrer and an argon inlet. The reaction medium is heated in an oil bath to 150° C. (setpoint on the oil bath) for 5 hours. The reaction is monitored by NMR.

In step b), 600 g of castor oil modified with glutaric acid and 134 g of glycerol are introduced in a 500 mL SVL® reactor equipped with a distillation assembly with mechanical stirring and an argon inlet. The reaction medium is heated using a heating mantle for 4 hours to 190° C. then 11 hours to 230° C. The reaction is monitored via acid value.

In step c), 600 g of glycerol-modified castor oil and 418 g of tertbutyl acetoacetate are introduced in a 1000 mL SVL® reactor equipped with a distillation assembly, a mechanical stirrer and an argon inlet. The reaction medium is heated in an oil bath to 150° C. (setpoint on the oil bath) for 5 hours. Then the reaction medium is placed in a rotavapor at 150° C. under continuous vacuum in order to eliminate the tertbutyl acetoacetate which will not have reacted.

### Example 10: Preparation of Formula A

Formula A is prepared using the contents indicated in the table below. The contents are expressed as weight percentages relative to the total weight of the composition.

TABLE 1

| | Formula A |
|---|---|
| Compounds | (invention) |
| Oil bearing acetoacetate functions according to Example 1 | 20 |
| Red iron oxide | 6 |
| (CI: 77491, Sunpuro Red Iron Oxide C33-8001, sold by | |
| Sun) | |
| Bis-cetearyl amodimethicone | 5 |
| (sold by Momentive Performance Materials) | |
| Isododecane/ethanol (90/10) | qs 100 |

Formula A is prepared by simply mixing the ingredients detailed in Table 1 using a Speed Mixer (mixing equipment which uses centrifugal force) for 2 minutes, at 3500 rpm and at room temperature.

### Example 11: One-Step Hair Dyeing Application of Formula A

Formula A is applied according to the one-step hair application protocol described previously. The evaluation results are summarized in the table below:

TABLE 2
Colorimetric measurements

| | Number of | | | | |
|---|---|---|---|---|---|
| Composition | shampoo washes | L* | a* | b* | ΔE |
| Formula A | 0 | 32.85 | 22.19 | 15.52 | — |
| (Invention) | 3 | 32.52 | 18.66 | 12.9 | 1.7 |

The locks of hair dyed according to the invention have a ΔE value of less than 2. It is thus seen that the application of Formula A according to the process of the invention allows very good colour persistence to be obtained.

### Example 12: Preparation of the Formulas B

The formulas B are prepared using the contents indicated in the table below. The contents are expressed as weight percentages relative to the total weight of the composition under consideration.

TABLE 3

| | | Formula B |
|---|---|---|
| | | (Base coat + |
| | | Top coat) |
| | Compounds | (invention) |
| Base | Oil bearing acetoacetate functions according to | 20 |
| coat | Example 1 | |
| | Red iron oxide | 6 |
| | (CI: 77491, Sunpuro Red Iron Oxide C33-8001, sold | |
| | by Sun) | |
| | Isododecane | qs 100 |
| Top | 4,7,10-Trioxa-1,13-tridecanediamine (CAS No.: | 5 |
| coat | 4246-51-9, sold by Sigma-Aldrich) | |
| | Isododecane | qs 100 |

The “base coat” and “top coat” compositions are prepared by simply mixing the ingredients detailed in Table 3 using a Speed Mixer (mixing equipment which uses centrifugal force) for 2 minutes, at 3500 rpm and at room temperature.

### Example 13: Two-Step Hair Dyeing Application of the Formulas B

The formulas B are applied according to the two-step hair application protocol described previously.

The evaluation results are summarized in the table below:

TABLE 4
Colorimetric measurements

| | Number of | | | | |
|---|---|---|---|---|---|
| Composition | shampoo washes | L* | a* | b* | ΔE |
| Formulas B | 0 | 32.15 | 26.77 | 19.53 | — |
| (Base coat + Top | 3 | 32.62 | 26.77 | 20.59 | 0.2 |
| coat) (invention) | 5 | 34.16 | 25.02 | 19.02 | 2.7 |

The locks of hair dyed according to the invention have a ΔE value of 0.2 after three shampoo washes, and of 2.7 after five shampoo washes. It is thus seen that the application of the formulas B according to the process of the invention allows very good colour persistence to be obtained.

In particular, visually, the red colouring appears unchanged between the colouring obtained just after having performed the process of the invention and after three and five successive shampoo washes. In addition, the hair strands appear separated after treatment, and have a very pleasant feel.

### Example 14: Preparation of the Formulas C

The formulas C are prepared using the contents indicated in the table below. The contents are expressed as weight percentages relative to the total weight of the composition under consideration.

TABLE 5

| | | Formulas C |
|---|---|---|
| | | (Base coat + |
| | | Top coat) |
| | Compounds | (invention) |
| Base | Oil bearing acetoacetate functions | 20 |
| coat | according to Example 1 | |
| | Red iron oxide | 6 |
| | (CI: 77491, Sunpuro Red Iron | |
| | Oxide C33-8001, sold by Sun) | |
| | Poly(dimethylsiloxane), | 5 |
| | bis(3-aminopropyl) terminated | |
| | (PDMS-diNH2) (CAS No.: 106214-84-0, | |
| | sold by the company | |
| | Gelest, Mn = 50 000 g/mol) | |
| | Isododecane | qs 100 |
| Top | [4-6% (mercaptopropyl)methylsiloxane] - | 5 |
| coat | dimethylsiloxane copolymer | |
| | (CAS No. 102783-03-9), sold by the company | |
| | Gelest, molecular weight 6000-8000) | |
| | Isododecane | qs 100 |

The “base coat” and “top coat” compositions are prepared by simply mixing the ingredients detailed in Table 5 using a Speed Mixer (mixing equipment which uses centrifugal force) for 2 minutes, at 3500 rpm and at room temperature.

### Example 15: Two-Step Hair Dyeing Application of the Formulas C

The formulas C are applied according to the two-step hair application protocol described previously.

The evaluation results are summarized in the table below:

TABLE 6
Colorimetric measurements

| | Number of | | | | |
|---|---|---|---|---|---|
| Composition | shampoo washes | L* | a* | b* | ΔE |
| Formulas C (Base coat + | 0 | 32.74 | 26.47 | 19.64 | — |
| Top coat) (invention) | 3 | 33.35 | 24.85 | 17.7 | 1.75 |

The locks of hair dyed according to the invention have a ΔE value of less than 2. It is thus seen that the application of the formulas C according to the process of the invention allows very good colour persistence to be obtained.

In particular, visually, the red colouring appears unchanged between the colouring obtained just after having performed the process of the invention and after three successive shampoo washes.

### Example 16: Preparation of the Formulas D

The formulas D are prepared using the contents indicated in the table below. The contents are expressed as weight percentages relative to the total weight of the composition under consideration.

TABLE 7

| | | Formulas D |
|---|---|---|
| | | (Base coat + |
| | | Top coat) |
| | Compounds | (invention) |
| Base | Oil bearing acetoacetate functions | 20 |
| coat | according to Example 6 | |
| | Red iron oxide | 6 |
| | (CI: 77491, Sunpuro Red Iron | |
| | Oxide C33-8001, sold by Sun) | |
| | Isododecane/ethanol (50/50) | qs 100 |
| Top | Bis-cetearyl amodimethicone | 5 |
| coat | (sold by Momentive | |
| | Performance Materials) | |
| | Spermidine (CAS No. 124-20-9, | 3.5 |
| | sold by Sigma-Aldrich) | |
| | Isododecane/ethanol (90/10) | qs 100 |

The “base coat” and “top coat” compositions are prepared by simply mixing the ingredients detailed in Table 7 using a Speed Mixer (mixing equipment which uses centrifugal force) for 2 minutes, at 3500 rpm and at room temperature.

### Example 17: Two-Step Hair Dyeing Application of the Formulas D

The formulas D are applied according to the two-step hair application protocol described previously.

The evaluation results are summarized in the table below:

TABLE 8
Colorimetric measurements

| | Number of | | | | |
|---|---|---|---|---|---|
| Composition | shampoo washes | L* | a* | b* | ΔE |
| Formulas D | 0 | 29.95 | 25.08 | 18.19 | — |
| (Base coat + Top | 3 | 30.14 | 24.98 | 18.05 | 0.23 |
| coat) (invention) | 5 | 29.72 | 24.47 | 17.72 | 0.28 |

The locks of hair dyed according to the invention have a ΔE value of less than 2. It is thus seen that the application of the formulas D according to the process of the invention allows very good colour persistence to be obtained.

### Example 18: Preparation of the Formulas E

The formulas E are prepared using the contents indicated in the table below. The contents are expressed as weight percentages relative to the total weight of the composition under consideration.

TABLE 9

| | | Formulas E |
|---|---|---|
| | | (Base coat + |
| | | Top coat) |
| | Compounds | (invention) |
| Base | Oil bearing acetoacetate functions | 20 |
| coat | according to Example 5 | |
| | Yellow iron oxide | 6 |
| | (Sunpuro Yellow Iron Oxide C33-9001) | |
| | Spermidine (CAS No. 124-20-9, | 2 |
| | sold by Sigma-Aldrich) | |
| | Isododecane/ethanol (50/50) | qs 100 |
| Top | Bis-cetearyl amodimethicone | 5 |
| coat | (sold by Momentive | |
| | Performance Materials) | |
| | Isododecane/ethanol (90/10) | qs 100 |

The “base coat” and “top coat” compositions are prepared by simply mixing the ingredients detailed in Table 9 using a Speed Mixer (mixing equipment which uses centrifugal force) for 2 minutes, at 3500 rpm and at room temperature.

### Example 19: Two-Step Hair Dyeing Application of the Formulas E

The formulas E are applied according to the two-step hair application protocol described previously.

The evaluation results are summarized in the table below:

TABLE 10
Colorimetric measurements

| | Number of | | | | |
|---|---|---|---|---|---|
| Composition | shampoo washes | L* | a* | b* | ΔE |
| Formulas E (Base coat + | 0 | 47.45 | 12.03 | 30.1 | — |
| Top coat) | 3 | 51.41 | 11.67 | 30.1 | 1.93 |
| (invention) | | | | | |

The locks of hair dyed according to the invention have a ΔE value of less than 2. It is thus seen that the application of the formulas E according to the process of the invention allows very good colour persistence to be obtained.

### Example 20: Preparation of the Formulas F

The formulas F are prepared using the contents indicated in the table below. The contents are expressed as weight percentages relative to the total weight of the composition under consideration.

TABLE 11

| | | Formulas F |
|---|---|---|
| | | (Base coat + |
| | | Top coat) |
| | Compounds | (invention) |
| First | Black iron oxide (C33-7001 Sun Puro) | 6 |
| gesture | Spermidine (CAS No. 124-20-9, | 2 |
| | sold by Sigma-Aldrich) | |
| | Water | qs 100 |
| Second | Oil bearing acetoacetate functions | 20 |
| gesture | according to Example 2 | |
| | Isododecane/ethanol (50/50) | qs 100 |
| Third | Spermidine (CAS No. 124-20-9, | 5.5 |
| gesture | sold by Sigma-Aldrich) | |
| | Water | qs 100 |

The compositions are prepared by simply mixing the ingredients detailed in Table 11 using a Speed Mixer (mixing equipment which uses centrifugal force) for 2 minutes, at 3500 rpm and at room temperature.

### Example 21: Three-Step Hair Dyeing Application of the Formulas F

The formulas F are applied according to the three-step hair application protocol described previously.

The evaluation results are summarized in the table below:

TABLE 12
Colorimetric measurements

| | Number of | | | | |
|---|---|---|---|---|---|
| Composition | shampoo washes | L* | a* | b* | ΔE |
| Formulas F (three | 0 | 24.37 | −0.41 | 0.95 | — |
| compositions) (invention) | 3 | 24.69 | 0.03 | 1.46 | 0.45 |
| | 5 | 25.11 | −0.14 | 0.7 | 0.64 |

The locks of hair dyed according to the invention have a ΔE value of less than 2. It is thus seen that the application of the formulas F according to the process of the invention allows very good colour persistence to be obtained.

### Example 22: Preparation of Compositions G to Q

Compositions G to Q are prepared using the contents indicated in the table below. The contents are expressed as weight percentages relative to the total weight of the composition under consideration.

TABLE 13
Table 13

| | Formula | Formula | Formula | Formula | Formula | Formula | Formula | Formula | Formula | Formula | Formula |
|---|---|---|---|---|---|---|---|---|---|---|---|
| | G | H | I | J | K | L | M | N | O | P | Q |
| | (compara- | (inven- | (compara- | (inven- | (compara- | (inven- | (compara- | (inven- | (inven- | (compara- | (inven- |
| Compounds | tive) | tion) | tive) | tion) | tive) | tion) | tive) | tion) | tion) | tive) | tion) |
| Oil bearing | — | 10 | — | 5.5 | — | — | — | — | — | — | — |
| acetoacetate | | | | | | | | | | | |
| functions according | | | | | | | | | | | |
| to Example 1 | | | | | | | | | | | |
| Oil bearing | — | — | — | — | — | 10 | — | 7 | — | — | — |
| acetoacetate | | | | | | | | | | | |
| functions according | | | | | | | | | | | |
| to Example 5 | | | | | | | | | | | |
| Oil bearing | — | — | — | — | — | — | — | — | 10 | — | 8 |
| acetoacetate | | | | | | | | | | | |
| functions according | | | | | | | | | | | |
| to Example 6 | | | | | | | | | | | |
| Trimethylolpropane | 1 | — | 10 | 4.5 | — | — | — | — | 1 | — | — |
| triacrylate (CAS No.: | | | | | | | | | | | |
| 15625-89-5, sold by | | | | | | | | | | | |
| Sigma-Aldrich) | | | | | | | | | | | |
| Poly | — | — | — | — | — | — | 10 | 3 | — | — | — |
| (dimethylsiloxane) | | | | | | | | | | | |
| bis(3- | | | | | | | | | | | |
| aminopropyl) | | | | | | | | | | | |
| terminated (PDMS- | | | | | | | | | | | |
| diNH₂) (CAS No.: | | | | | | | | | | | |
| 106214-84-0, sold | | | | | | | | | | | |
| by the company | | | | | | | | | | | |
| Gelest, Mn = | | | | | | | | | | | |
| 50 000 g/mol) | | | | | | | | | | | |
| Spermidine (CAS | — | — | — | — | — | — | — | — | — | 10 | 2 |
| No. 124-20-9, sold | | | | | | | | | | | |
| by Sigma-Aldrich) | | | | | | | | | | | |
| 1,8- | — | — | — | 0.5 | — | — | — | — | — | — | — |
| Diazabicyclo[5.4.0]— | | | | | | | | | | | |
| undec-7-ene | | | | | | | | | | | |
| Isododecane/ethanol | 100 | qs | qs | qs | — | — | — | — | — | — | — |
| (90/10) | | 100 | 100 | 100 | | | | | | | |
| Isododecane/ethanol | — | — | — | — | 100 | qs | qs | qs | qs | qs | qs |
| (50/50) | | | | | | 100 | 100 | 100 | 100 | 100 | 100 |

### Example 23: Hairstyling Application of Compositions G to Q—Curl Evaluation

Compositions G to Q are applied according to the hairstyling protocol described previously. The results of the curl evaluations are summarized in the table below:

TABLE 14

| | Value after | Value after 24 hours |
|---|---|---|
| | application | of suspension |
| Formulas | (in cm) | (in cm) |
| Formula G (comparative) | 12 | 16 |
| Formula H (invention) | 10 | 15 |
| Formula I (comparative) | 11 | 15 |
| Formula J (invention) | 5 | 6 |
| Formula K (comparative) | 13 | 17 |
| Formula L (invention) | 7 | 13 |
| Formula M (comparative) | 8 | 12 |
| Formula N (invention) | 4.5 | 9 |
| Formula O (invention) | 7 | Not evaluated |
| Formula P (comparative) | 8.5 | 13 |
| Formula Q (invention) | 4 | 4.5 |

It is seen that the locks treated with the formulas H, J, L, N, O and Q according to the process of the invention significantly improve the shape of the curls, which appear sharper and closer together, unlike the locks treated with the comparative formulas G, I, K, M and P.

In addition, even after 24 hours, the locks treated with the formulas according to the process of the invention underwent significantly less loss of curl shape than the comparative locks.

### Example 24: Preparation of Formulas R to T

Formulas R, R1, R2, S, S1, T, T1 and T2 comprising a compound of formula (I) according to the invention, and a crosslinking agent, are prepared using the contents indicated in the table below by simply mixing the ingredients detailed in table 15 using a Speed Mixer (mixing equipment which uses centrifugal force) for 2 minutes, at 3500 rpm and at room temperature.

The contents are expressed as weight percentages relative to the total weight of the composition.

TABLE 15
Table 15

| | Formula | Formula | Formula | Formula | Formula | Formula | Formula | Formula |
|---|---|---|---|---|---|---|---|---|
| | R | R1 | R2 | S | S1 | T | T1 | T2 |
| Compounds | (invention) | (comparative) | (comparative) | (invention) | (comparative) | (invention) | (comparative) | (comparative) |
| Oil bearing acetoacetate | — | — | — | — | — | 20 | 20 | — |
| functions according to Example | | | | | | | | |
| 1 | | | | | | | | |
| Oil bearing acetoacetate | 20 | 20 | — | 20 | — | — | — | — |
| functions according to Example | | | | | | | | |
| 6 | | | | | | | | |
| Iron oxide (CI: 77491, Sunpuro | 6 | 6 | 6 | 6 | 6 | 6 | 6 | 6 |
| Red Iron Oxide C33-8001, sold | | | | | | | | |
| by Sun) | | | | | | | | |
| Spermidine (CAS No. 124-20- | 4.6 | — | 4.6 | — | — | 5 | — | 5 |
| 9, sold by Sigma Aldrich) | | | | | | | | |
| (3-Aminopropyl) | — | — | — | 13 | 13 | 0 | 0 | 0 |
| triethoxysilane (APTES) (CAS | | | | | | | | |
| No. 919-30-2, sold by Sigma | | | | | | | | |
| Aldrich) | | | | | | | | |
| Isododecane/Ethanol (90/10) | — | — | — | — | — | qs | qs | qs |
| | | | | | | 100 | 100 | 100 |
| Isododecane/Butyl | qs | qs | qs | qs | qs | — | — | — |
| Acetate/Ethanol (70/20/10) | 100 | 100 | 100 | 100 | 100 | | | |

### Example 25: One-Step Hair Dyeing Application of Compositions R to T

Compositions R, R1, R2, S, S1, T, T1 and T2 are applied to locks of dry natural hair containing 90% white hairs, at a rate of 1 g of composition per gram of lock.

The locks of hair are then combed and dried with a hairdryer for 1 minute and 30 seconds, then a steam straightener (Rowenta brand Steampod) is applied to said locks of hair with 5 passes at a temperature of 210° C.

The locks of hair thus coloured are then subjected to a test of several repeated shampoo washes so as to evaluate the fastness (persistence) of the colouring obtained with respect to shampoo washing, according to the shampoo washing protocol described above.

The results of the evaluations are summarized in the table below:

TABLE 16
Colorimetric measurements

| | Number of | | | | |
|---|---|---|---|---|---|
| | shampoo | | | | |
| | washes | L* | a* | b* | ΔE |
| Formula R | 0 | 32.38 | 26.3 | 18.55 | — |
| (invention) | 3 | 37.09 | 23.23 | 16.69 | 5.92 |
| Formula R1 | 0 | 32.29 | 27.55 | 20.33 | — |
| (comparative) | 1 | 50.41 | 10.66 | 14.84 | 25.37 |
| Formula R2 | 0 | 31.97 | 27.6 | 20.34 | — |
| (comparative) | 1 | 52.93 | 13.87 | 16.86 | 25.29 |
| Formula S | 0 | 30.05 | 24.86 | 17.59 | — |
| (invention) | 3 | 34.16 | 19.41 | 13.22 | 8.10 |
| Formula S1 | 0 | 33.59 | 24.19 | 18.03 | — |
| (comparative) | 3 | 52.15 | 11.45 | 16.98 | 22.54 |
| Formula T | 0 | 31.83 | 20.8 | 14.55 | — |
| (invention) | 3 | 33.61 | 21.84 | 16.18 | 2.62 |
| Formula T1 | 0 | 32.7 | 25.88 | 19.11 | — |
| (comparative) | 1 | 50.99 | 15.69 | 18.27 | 20.95 |
| Formula T2 | 0 | 32.84 | 27.8 | 20.25 | — |
| (comparative) | 1 | 53.53 | 13.2 | 16.68 | 25.57 |

The locks of hair dyed with compositions R, S, T according to the invention and the process according to the invention have lower ΔE values than the locks of hair dyed with comparative compositions R1-R2, S1-S2, T1-T2.

Thus, the compositions according to the invention and the process according to the invention make it possible to obtain a coloured coating on the hair which affords an improved persistence to shampoo washes.

### Example 26: Preparation of Composition U

Composition U is prepared using the contents indicated in the table below. The contents are expressed as weight percentages relative to the total weight of the composition under consideration.

TABLE 17

| Ingredients | Composition U |
|---|---|
| Compound sucrose palmitate | 20 |
| bearing acetoacetate | |
| functions according to Example 8 | |
| Yellow iron oxide (Sunpuro Yellow | 6 |
| Iron Oxide C33-9001) | |
| Polyetheramine (Mw = 440 | 6.36 |
| g/mol, AHEW (81 g/eq) | |
| (Jeffamine T-403) | |
| Isododecane/Ethanol (90/10) | qs |

### Example 27: One-Step Hair Dyeing Application of the Composition U

The composition U is applied to locks of dry natural hair containing 90% white hairs, at a rate of 1 g of composition per gram of lock.

The evaluation results are summarized in the table below:

TABLE 18
Colorimetric measurements

| | Number of | | | | |
|---|---|---|---|---|---|
| Composition | shampoo washes | L* | a* | b* | ΔE |
| Composition U | 0 | 30.98 | 24.81 | 17.69 | — |
| (invention) | 3 | 32.21 | 25.41 | 18.21 | 1.46 |

The locks of hair dyed according to the invention have a ΔE value of less than 2. It is thus seen that the application of the composition U according to the process of the invention allows very good colour persistence to be obtained.

### Example 28: Preparation of Compositions V to Y

Compositions V to Y comprising a compound of formula (I) according to the invention, and a crosslinking agent, are prepared using the contents indicated in the table below by simply mixing the ingredients detailed in table 19 using a Speed Mixer (mixing equipment which uses centrifugal force) for 2 minutes, at 3500 rpm and at room temperature.

The contents are expressed as weight percentages relative to the total weight of the composition.

TABLE 19

| Composition | V | W | X | Y |
|---|---|---|---|---|
| Oil bearing acetoacetate functions | 10 | — | 6.4 | — |
| according to Example 6 | | | | |
| Oil bearing acetoacetate functions | — | 10.5 | — | — |
| according to Example 9 | | | | |
| Mannitol dihexanoate acetoacetate | — | — | — | 9.8 |
| according to Example 7 | | | | |
| Polyetheramine (Mw = 440 g/mol, | 5.5 | 5.1 | — | — |
| AHEW (81 g/eq) (Jeffamine T-403) | | | | |
| Priamine 1075-LQ-(GD) sold by | — | — | 6.8 | — |
| company CRODA | | | | |
| Polylysine (PL-25) sold by company JNC | — | — | — | 10 |
| Iron oxide (CI: 77491, Tarox Iron Oxide | 2 | 2 | 3 | — |
| R-516 HP) of Titan Kogyo | | | | |
| Iron oxide (CI: 77491, Sunpuro Red Iron | — | — | — | 6 |
| Oxide C33-8001, sold by Sun) | | | | |
| Water | — | — | — | 37 |
| Water/Ethanol (50/50) | — | — | — | qs 100 |
| Ethanol | qs 100 | — | qs 100 | qs 100 |
| Ethyl lactate/Ethanol (70/30) | — | qs 100 | — | — |

### Example 29: One-Step Hair Dyeing Application of the Composition V to X

Compositions V, W, or X are applied to sensitised keratin fibres (bleached natural hair of alkaline solubility equal to 20% named SA20), at a rate of 1 g of composition per gram of lock, then combed and dried using a hair dryer, then left at rest for 24 hours under a fume cupboard at room temperature.

The hair is dyed uniformly and intensely.

The locks of hair thus coloured are then subjected to a test of several repeated shampoo washes so as to evaluate the fastness (persistence) of the colouring obtained with respect to shampoo washing, according to the shampoo washing protocol described above.

The evaluation results are summarized in the table below:

TABLE 20
Colorimetric measurements

| | Number of | | | | |
|---|---|---|---|---|---|
| | shampoo | | | | |
| Compositions | washes | L* | a* | b* | ΔE |
| V | 0 | 44.14 | 15.72 | 25.14 | — |
| | 5 | 51.29 | 11.52 | 20.84 | 9.34 |
| W | 0 | 41.71 | 17.25 | 34.72 | — |
| | 5 | 41.76 | 16.98 | 37.33 | 2.62 |
| X | 0 | 36.53 | 19.82 | 33.87 | — |
| | 5 | 38.32 | 19.15 | 37.29 | 3.92 |

The locks of hair dyed with compositions V, W and X according to the invention have low ΔE values after five shampoo washes. It is thus seen that the application of the formulas V, W, and X according to the process of the invention allows very good colour persistence to be obtained.

1-31. (canceled)

32. A method for treating keratin fibers, comprising applying to the keratin fibers a composition comprising:
i) one or more compounds of formula (I), optical or geometrical isomers thereof, and/or solvates thereof, or mixtures thereof: 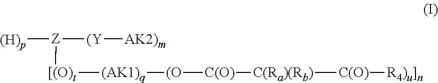
wherein in formula (I):
Z represents a linear or branched, saturated or unsaturated, conjugated or non-conjugated, acyclic or cyclic, aromatic or non-aromatic multivalent C₂ to C₄₀ hydrocarbon-based radical, Z being optionally interrupted with one or more groups or heteroatoms chosen from —O—, —N(Rₐ)—, —S—, —C(O)—, (hetero)cycles, or combinations thereof;
AK1 represents a divalent saturated or unsaturated, linear or branched C₁-C₁₂ hydrocarbon-based chain;
AK2 represents a linear or branched, saturated or unsaturated, C₁-C₄₀ monovalent hydrocarbon-based chain, optionally substituted with one or more identical or different radicals chosen from —OR or —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), or combinations thereof;
AK3 represents a divalent linear or branched, saturated or unsaturated C₁-C₁₂ hydrocarbon-based chain;
R represents a hydrogen atom, a linear or branched, saturated or unsaturated C₁-C₆ alkyl radical or a radical —C(O)—C(Rₐ)(R_{b})—C(O)—R₄;
X represents —O—, —S—, —O—C(O)— or —C(O)—O—;
Y represents —O—C(O)— or —C(O)—O—;
R₄ represents a linear or branched, saturated or unsaturated C₁-C₆ monovalent hydrocarbon-based radical;
Rₐ and R_{b} are independently chosen from a hydrogen atom or a (C₁-C₄)alkyl group;
p denotes an integer equal to 0 or 1;
m denotes an integer equal to 0, 1, 2, 3 or 4; with the proviso that when m is greater than 1, then the radicals —Y-AK2 are identical or different;
n denotes an integer equal to 0, 1, 2, 3 or 4; with the proviso that when n is greater than 1, then the radicals —(O)ₜ-(AK1)_{q}-(O—C(O)—C(Rₐ)(R_{b})—C(O)—R₄)ᵤ are identical or different;
t denotes an integer equal to 0 or 1;
q denotes an integer equal to 0 or 1;
u denotes an integer equal to 1, 2 or 3;
v denotes an integer equal to 1, 2 or 3;
with the proviso that if q is equal to 0 then t is equal to 0, (m+n) is greater than or equal to 1, and said compound comprises at least two groups —C(O)—C(Rₐ)(R_{b})—C(O)—R₄; and
optionally ii) one or more crosslinking agents.

33. The method of claim 32, wherein in formula (I):
Z represents the trivalent radical —CH₂—CH—CH₂—;
p is equal to 0;
n is equal to 0;
m is equal to 3;
AK2 represents a linear or branched, saturated or unsaturated, C₁₂-C₂₄ monovalent hydrocarbon-based chain, optionally substituted with one or more identical or different radicals chosen from —OR or —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, epoxides, or combinations thereof;
with the proviso that said compound comprises at least two radicals —C(O)—C(Rₐ)(R_{b})—C(O)—R₄.

34. The method of claim 32, wherein in formula (I):
p is equal to 0;
Z represents a linear or branched, saturated or unsaturated, acyclic, multivalent C₄ to C₂₄, hydrocarbon-based radical, Z being optionally substituted with one or more radicals 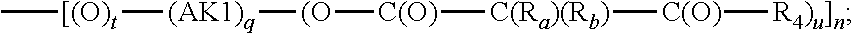
n denotes an integer equal to 0, 1, 2, 3 or 4; with the proviso that when n is greater than 1, then the radicals —(O)ₜ-(AK1)_{q}—(O—C(O)—C(Rₐ)(R_{b})—C(O)—R₄)ᵤ are identical or different;
m is equal to 1;
AK2 represents a linear or branched, saturated or unsaturated, C₃-C₈ monovalent hydrocarbon-based chain, substituted with one or more identical or different radicals chosen from —OR, —X-AK₃-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), or combinations thereof;
with the proviso that said compound comprises at least two radicals —C(O)—C(Rₐ)(R_{b})—C(O)—R₄.

35. The method of claim 32, wherein in formula (I):
p is equal to 0;
n+m is equal to 4, m being non-zero;
Z represents an unsubstituted and uninterrupted, linear or branched, saturated or unsaturated, and acyclic, C₂ to C₂₄ tetravalent hydrocarbon-based radical;
AK2 independently represent a linear or branched, saturated or unsaturated, C₁₂-C₂₄ monovalent hydrocarbon-based chain, optionally substituted with one or more identical or different radicals chosen from —OR and —X-AK3-(O—C(O)—C(Rₐ)(R_{b})—C(O)—R₄)ᵥ; and/or
said chain being optionally interrupted with one or more heteroatoms or groups chosen from —O—, —S—, —C(O)—, (hetero)cycle(s), and combinations thereof;
AK3 represents a linear or branched, saturated or unsaturated C₁-C₁₂ divalent hydrocarbon-based chain;
with the proviso that said compound comprises at least two radicals —C(O)—C(Rₐ)(R_{b})—C(O)—R₄.

36. The method of claim 32, wherein in formula (I):
p is equal to 1;
Z represents a linear or branched, saturated or unsaturated, and acyclic, multivalent C₁₄ to C₂₄ hydrocarbon-based radical, Z being optionally substituted with one or more radicals —[(O)ₜ-(AK1)_{q}-(O—C(O)—C(Rₐ)(R_{b})—C(O)—R₄)ᵤ]ₙ;
AK2 represents a linear or branched, saturated or unsaturated C₁₄-C₃₀ monovalent hydrocarbon-based chain, substituted with one or more identical or different radicals chosen from —OR, —X-AK3-(O—C(O)—C(Rₐ)(R_{b})—C(O)—R₄)ᵥ; and/or said chain being optionally interrupted with one or more heteroatoms or groups chosen from —O—, —S—, with the proviso that said compound comprises at least two radicals —C(O)—C(Rₐ)(R_{b})—C(O)—R₄.

37. The method of claim 32, wherein in formula (I):
p is equal to 0;
Z represents a linear or branched, saturated or unsaturated, and acyclic C₂ to C₂₄ tetravalent hydrocarbon-based radical, Z being substituted with one or more radicals —[(O)ₜ-(AK1)_{q}—(O—C(O)—C(Rₐ)(R_{b})—C(O)—R₄)ᵤ]ₙ;
n+m is equal to 4;
AK2 independently represents a linear or branched, saturated or unsaturated, C₁₄ to C₃₀ hydrocarbon-based chain, said chain being optionally substituted with one or more identical or different radicals chosen from —OR, —X-AK₃-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), or combinations thereof;
with the proviso that said compound comprises at least two radicals —C(O)—C(Rₐ)(R_{b})—C(O)—R₄.

38. The method of claim 32, wherein in formula (I):
p is equal to 0;
m is non-zero;
Z represents a linear or branched, saturated or unsaturated, conjugated or non-conjugated acyclic or cyclic, aromatic or non-aromatic divalent C₂ to C₂₄ hydrocarbon-based radicals, said radical Z being interrupted with one or more heteroatoms or groups chosen from —O—, —N(Rₐ)—, —S—, —C(O)—, 5- or 6-membered heterocycloalkyl(s), or combinations thereof;
AK2 represents a linear or branched, saturated or unsaturated, C₁₄-C₃₀ monovalent hydrocarbon-based chain, said chain being optionally substituted with one or more identical or different radicals chosen from —OR and —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycles, or combinations thereof;
with the proviso that said compound comprises at least two radicals —C(O)—C(Rₐ)(R_{b})—C(O)—R₄.

39. The method of claim 32, wherein in formula (I):
p is equal to 0;
Z represents a linear or branched, saturated or unsaturated, and acyclic, C₂ to C₂₄ tetravalent hydrocarbon-based radical, Z being substituted with one or more radicals —[(O)ₜ-(AK1)_{q}—(O—C(O)—C(Rₐ)(R_{b})—C(O)—R₄)ᵤ]ₙ;
n+m is equal to 6;
AK2 represents a linear or branched, saturated or unsaturated, C₃-C₈ monovalent hydrocarbon-based chain;
with the proviso that said compound comprises at least two radicals —C(O)—C(Rₐ)(R_{b})—C(O)—R₄.

40. The method of claim 32, wherein the i) one or more compounds of formula (I), optical or geometrical isomers thereof, and/or solvates thereof, or mixtures thereof is chosen from compounds of formula (Ia), optical or geometrical isomers thereof, solvates thereof, or mixtures thereof: 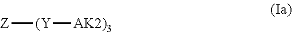
wherein in formula (Ia):
Z represents —CH₂—CH—CH₂—;
AK2 independently denotes a linear or branched, saturated or unsaturated, C₁₂-C₂₄ monovalent hydrocarbon-based chain, optionally substituted with one or more identical or different radicals chosen from —OR and —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, epoxides, or combinations thereof;
with the proviso that that said compound comprises at least two radicals —C(O)—C(Rₐ)(R_{b})—C(O)—R₄.

41. The method of claim 32, wherein the i) the one or more compounds of formula (I), optical or geometrical isomers thereof, and/or solvates thereof, or mixtures thereof is chosen from compounds of formula (Ib), optical or geometrical isomers thereof, solvates thereof, or mixtures thereof: 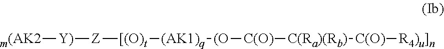
wherein in formula (Ib):
n+m is equal to 4, m being non-zero;
Z represents an unsubstituted and uninterrupted, linear or branched, saturated or unsaturated, and acyclic C₂ to C₂₄ tetravalent hydrocarbon-based radical;
AK2 independently represent a linear or branched, saturated or unsaturated, C₁₂-C₂₄ monovalent hydrocarbon-based chain, optionally substituted with one or more identical or different radicals chosen from —OR and —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), or combinations thereof;
AK3 represents a linear or branched, saturated or unsaturated C₁-C₁₂ divalent hydrocarbon-based chain;
with the proviso that said compound comprises at least two radicals —C(O)—C(Rₐ)(R_{b})—C(O)—R₄.

42. The method of claim 32, in which the compound(s) of formula (I) are chosen from the following compounds of formula (Ic), and also the optical or geometrical isomers thereof and/or the solvates thereof, such as hydrates: 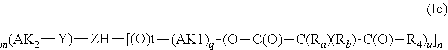
wherein in formula (Ic):
Z represents a linear or branched, saturated or unsaturated and acyclic multivalent C₁₄ to C₂₄ hydrocarbon-based radical, Z also being optionally substituted with one or more radicals —[(O)ₜ-(AK1)_{q}—(O—C(O)—C(Rₐ)(R_{b})—C(O)—R₄)ᵤ]ₙ;
AK2 represents a linear or branched, saturated or unsaturated C₁₄-C₃₀ monovalent hydrocarbon-based chain, substituted with one or more identical or different radicals chosen from —OR and —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), or combinations thereof;
with the proviso that that said compound contains at least two radicals —C(O)—C(Rₐ)(R_{b})—C(O)—R₄.

43. The method of claim 32, wherein the i) one or more compounds of formula (I), optical or geometrical isomers thereof, and/or solvates thereof, or mixtures thereof is chosen from compounds of formula (Id), optical or geometrical isomers thereof, solvates thereof, or mixtures thereof: 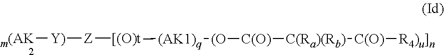
wherein in formula (Id):
n+m is equal to 4;
Z represents a linear or branched, saturated or unsaturated or acyclic C₂ to C₂₄ tetravalent hydrocarbon-based radical, Z being substituted with one or more radicals —[(O)ₜ-(AK1)_{q}—(O—C(O)—C(Rₐ)(R_{b})—C(O)—R₄)ᵤ]ₙ;
AK2 independently represents a linear or branched, saturated or unsaturated, C₁₄-C₃₀ monovalent hydrocarbon-based chain, said chain being optionally substituted with one or more identical or different radicals chosen from —OR and —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), or combinations thereof;
with the proviso that said compound comprises at least two radicals —C(O)—C(Rₐ)(R_{b})—C(O)—R₄.

44. The method of claim 32, wherein the i) one or more compounds of formula (I), optical or geometrical isomers thereof, and/or solvates thereof, or mixtures thereof is are chosen from compounds of formula (Ie), optical or geometrical isomers thereof, solvates thereof or mixtures thereof: 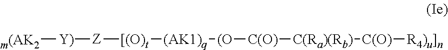
wherein in formula (Ie):
n+m is equal to 2, m being non-zero;
Z represents a linear or branched, saturated or unsaturated, conjugated or non-conjugated, acyclic or cyclic, aromatic or non-conjugated, C₂ to C₂₄ divalent hydrocarbon-based radical, said radical Z being interrupted with one or more heteroatoms or groups chosen from —O—, —N(Rₐ)—, —S—, —C(O)—, (hetero)cycle(s), or combinations thereof;
AK2 represents a linear or branched, saturated or unsaturated, C₁₄-C₃₀ monovalent hydrocarbon-based chain, said chain being optionally substituted with one or more identical or different radicals chosen from —OR and —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycles, or combinations thereof;
with the proviso that said compound comprises at least two radicals —C(O)—C(Rₐ)(R_{b})—C(O)—R₄.

45. The method of claim 32, wherein the i) one or more compounds of formula (I), optical or geometrical isomers thereof, and/or solvates thereof, or mixtures thereof: 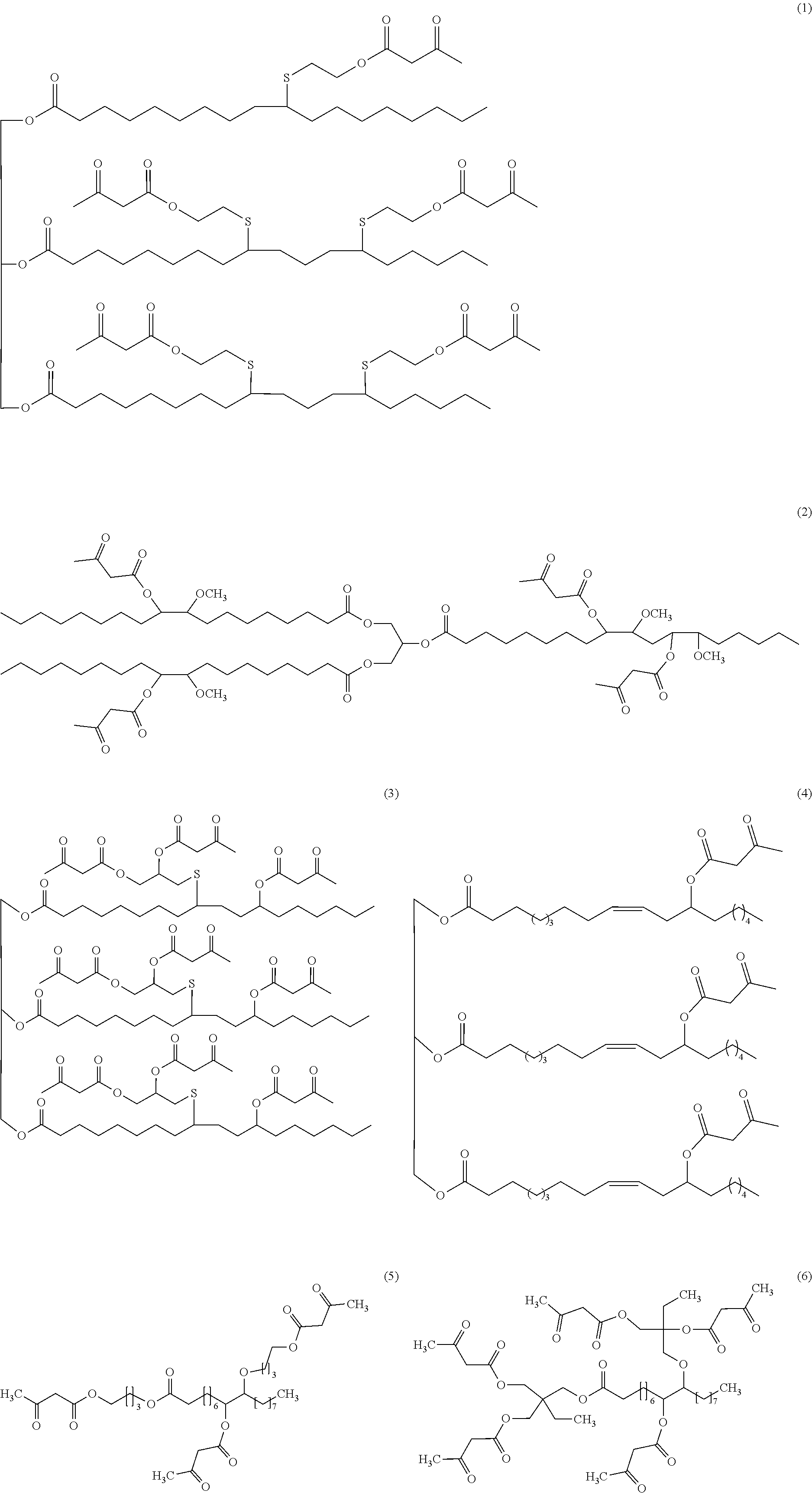 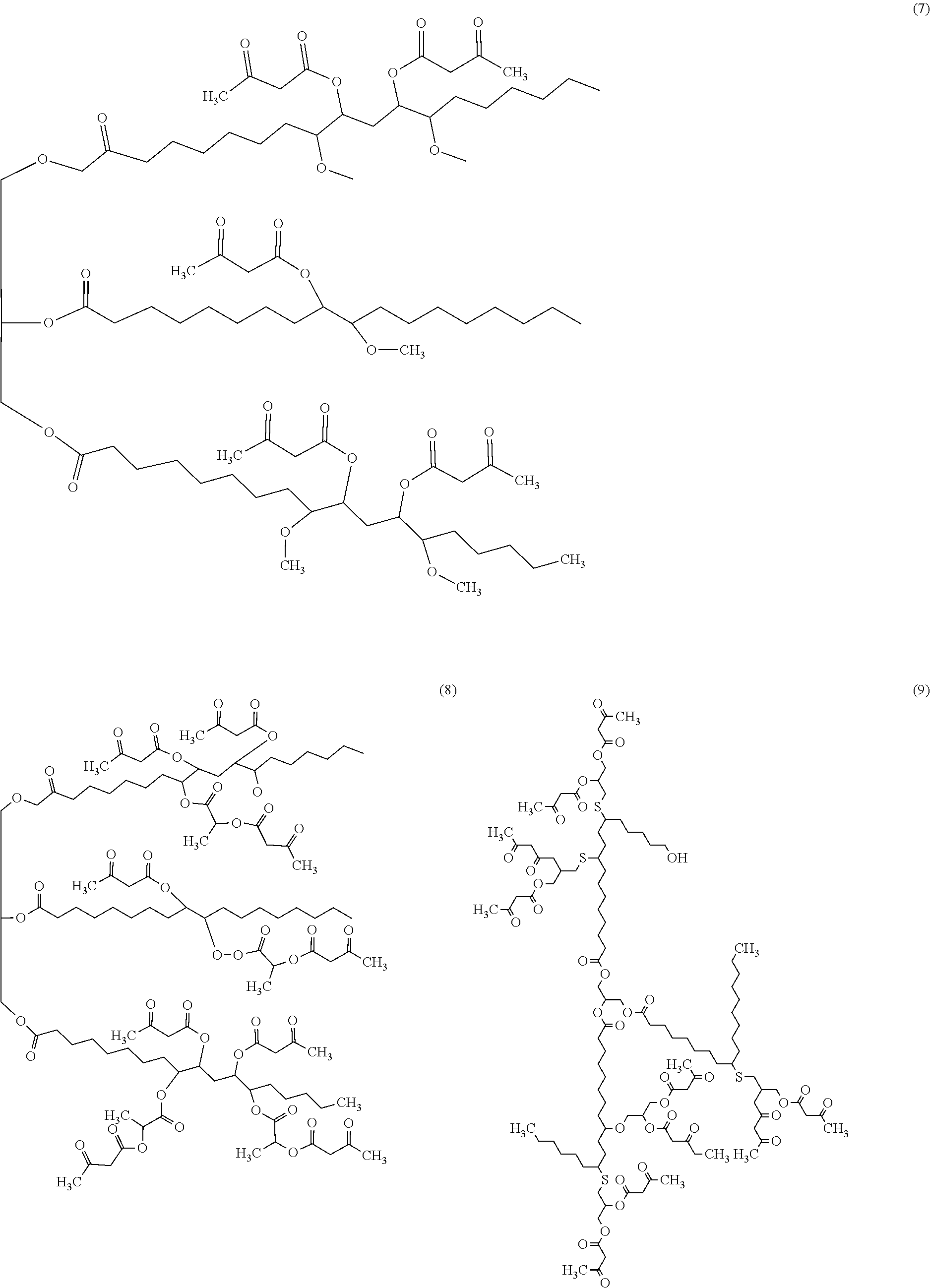 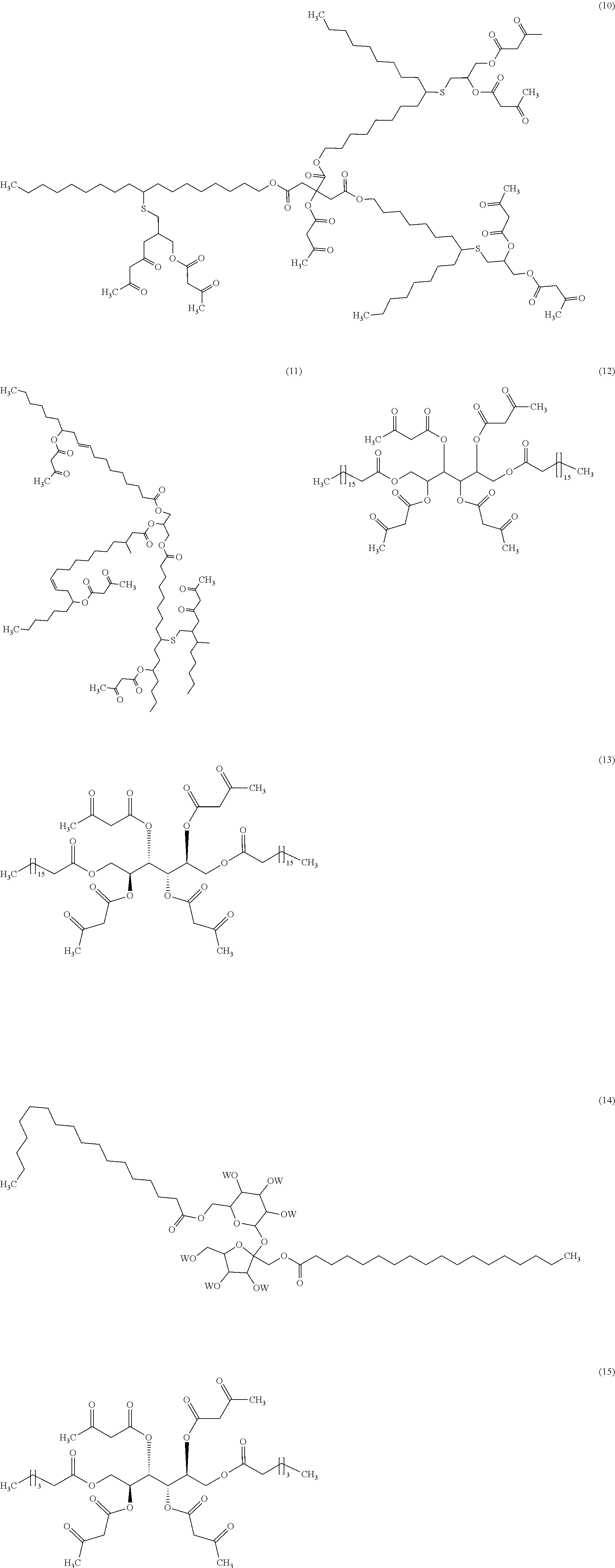 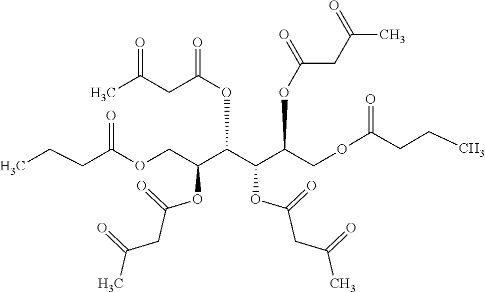 [SMILES: CCCC(=O)OC[C@H](OC(=O)CC(C)=O)[C@H](OC(=O)CC(C)=O)[C@@H](OC(=O)CC(C)=O)[C@H](COC(=O)CCC)OC(=O)CC(C)=O] 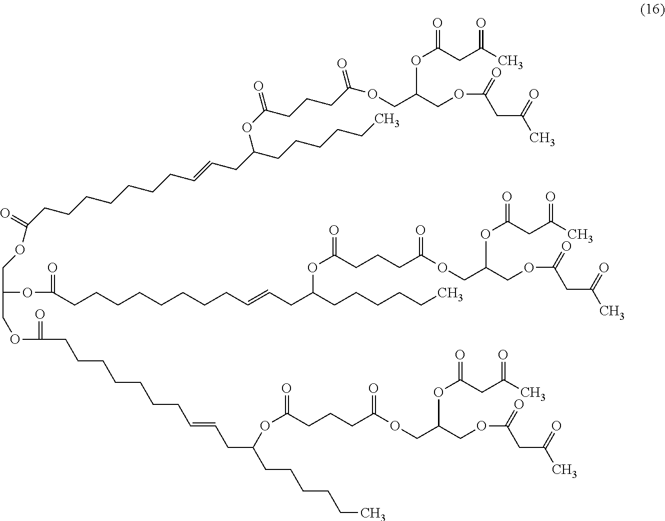 [SMILES: CCCCCCC(C/C=C/CCCCCCCCC(=O)OC(COC(=O)CCCCCCC/C=C/CC(CCCCCC)OC(=O)CCCC(=O)OCC(COC(=O)CC(C)=O)OC(=O)CC(C)=O)COC(=O)CCCCCCC/C=C/CC(CCCCCC)OC(=O)CCCC(=O)OCC(COC(=O)CC(C)=O)OC(=O)CC(C)=O)OC(=O)CCCC(=O)OCC(COC(=O)CC(C)=O)OC(=O)CC(C)=O]
with W being a hydrogen atom or —C(O)—C(Rₐ)(R_{b})—C(O)—R₄, and said compound comprising at least two groups —C(O)—C(Rₐ)(R_{b})—C(O)—R₄.

46. The method of claim 32, wherein the total amount of compounds of formula (I), optical or geometrical isomers thereof, and/or solvates thereof, or mixtures thereof ranges from 0.1% to 99% by weight, relative to the total weight of the composition.

47. The method of claim 32, wherein the ii) one or more crosslinking agents is chosen from (poly)amine, (poly)thiol, (poly)carbonyl, (poly)acrylate, metal alkoxide, metal (poly)(hydroxy)(C₁-C₆)alkylcarboxylate compounds, rare-earth metal derivatives thereof, or mixtures thereof.

48. The method of claim 32, wherein the ii) one or more crosslinking agent is chosen from:
A) (poly)amine compounds chosen from:
ia) chitosans such as poly(D-glucosamine),
ib) polyether diamines,
ic) polyether triamines,
id) aminoalkoxysilanes,
ie) polyamine compounds NH₂-alk-NH₂, wherein alk denotes a divalent C₂-C₂₀ hydrocarbon-based chain, optionally interrupted with one or more heteroatoms chosen from —O—, —N(R)— with R denoting a hydrogen atom or a C₁-C₄ alkyl radical,
if) polydialkylsiloxanes comprising primary amine groups at the end of the chain or on side chains, or
mixtures thereof;
B) (poly)thiol compounds chosen from:
iia) polydialkylsiloxanes bearing thiol functions,
iib) alkoxysilanes bearing thiol functions, or
mixtures thereof;
C) (poly)acrylate compounds of formula (XIV): 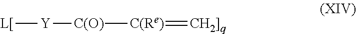
wherein in formula (XIV):
q represents an integer greater than or equal to 2,
L denotes a linear or branched, saturated or unsaturated, or (hetero)cyclic, saturated or unsaturated, multivalent group; L being optionally interrupted and/or terminated with one or more heteroatoms or groups chosen from O, S, N, Si, C(X), or combinations thereof,
R^{e} represents a hydrogen atom or a (C₁-C₄)alkyl group; and
Y represents an oxygen atom or an amino group —N(H)—;
D) metal alkoxides chosen from: zirconium ethoxide (Zr(OC₂H₅)₄), zirconium propoxide (Zr(OCH₂CH₂CH₃)₄), zirconium isopropoxide (Zr(OCH(CH₃)₂)₄), zirconium butoxide Zr(OCH₂CH₂CH₂CH₃)₄, zirconium tert-butoxide (Zr(OC(CH₃)₃)₄), titanium ethoxide (Ti(OC₂H₅)₄), titanium propoxide (Ti(OCH₂CH₂CH₃)₄), titanium isopropoxide (Ti(OCH(CH₃)₂)₄), titanium butoxide (Ti(OCH₂CH₂CH₂CH₃)₄), titanium tert-butoxide (Ti(OC(CH₃)₃)₄), titanium 2-ethylhexyloxide (Ti(OCH₂CH(C₂H₅)(CH₂)₃CH₃)₄), or mixtures thereof;
E) (poly)carbonyl compounds chosen from (poly)carbonyl compounds comprising a saturated or unsaturated, aromatic C₅-C₇ carbocycle; or
mixtures thereof.

49. The method of claim 32, wherein the composition comprises iii) one or more cosmetic active agents chosen from:
a) dyestuffs,
b) active agents for caring for keratin fibres,
c) UV-screening agents, or
mixtures thereof.

50. The method of claim 32, wherein the composition further comprises iv) one or more fatty substances.

51. A composition for treating keratin fibers comprising:
i) one or more compounds of formula (I), optical or geometrical isomers thereof, and/or solvates thereof, or mixtures thereof: 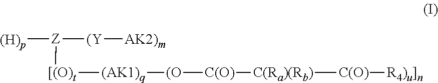
wherein in formula (I):
Z represents a linear or branched, saturated or unsaturated, conjugated or non-conjugated, acyclic or cyclic, aromatic or non-aromatic multivalent C₂ to C₄₀ hydrocarbon-based radical, Z being optionally interrupted with one or more groups or heteroatoms chosen from —O—, —N(Rₐ)—, —S—, —C(O)—, (hetero)cycles, or combinations thereof;
AK1 represents a divalent saturated or unsaturated, linear or branched C₁-C₁₂ hydrocarbon-based chain;
AK2 represents a linear or branched, saturated or unsaturated, C₁-C₄₀ monovalent hydrocarbon-based chain, optionally substituted with one or more identical or different radicals chosen from —OR or —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), or combinations thereof;
AK3 represents a divalent linear or branched, saturated or unsaturated C₁-C₁₂ hydrocarbon-based chain;
R represents a hydrogen atom, a linear or branched, saturated or unsaturated C₁-C₆ alkyl radical or a radical —C(O)—C(Rₐ)(R_{b})—C(O)—R₄;
X represents —O—, —S—, —O—C(O)— or —C(O)—O—;
Y represents —O—C(O)— or —C(O)—O—;
R₄ represents a linear or branched, saturated or unsaturated C₁-C₆ monovalent hydrocarbon-based radical;
Rₐ and R_{b} are independently chosen from a hydrogen atom or a (C₁-C₄)alkyl group;
p denotes an integer equal to 0 or 1;
m denotes an integer equal to 0, 1, 2, 3 or 4; with the proviso that when m is greater than 1, then the radicals —Y-AK₂ are identical or different;
n denotes an integer equal to 0, 1, 2, 3 or 4; with the proviso that when n is greater than 1, then the radicals —(O)ₜ-(AK1)_{q}—(O—C(O)—C(Rₐ)(R_{b})—C(O)—R₄)ᵤ are identical or different;
t denotes an integer equal to 0 or 1;
q denotes an integer equal to 0 or 1;
u denotes an integer equal to 1, 2 or 3;
v denotes an integer equal to 1, 2 or 3;
with the proviso that if q is equal to 0 then t is equal to 0, (m+n) is greater than or equal to 1, and said compound comprises at least two groups —C(O)—C(Rₐ)(R_{b})—C(O)—R₄; and optionally ii) one or more crosslinking agents.
